SEQUENCE LISTING (139 sequences)

SEQ ID NO: 1 - RNA, 1527 nt, Streptomyces sp.
  source 1..1527: mol_type = genomic RNA
acggagagtttgatcctggctcaggacgaacgctggcggcgtgcttaacacatgcaagtcgaacgatgaacttccttcgggaggggattagtggcgaacgggtgagtaacacgtgggcaatctgccctgcactctgggacaagccctggaaacggggtctaataccggatacgacacgggatcgcatgatcttccgtgtggaaagctccggcggtgcaggatgagcccgcggcctatcagcttgttggtggggtaatggcctaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgcaagtgacggtacctgcagaagaagcgccggctaactacgtgccagcagccgcggtaatacgtagggcgcaagcgttgtccggaattattgggcgtaaagagctcgtaggcggcttgtcacgtcggatgtgaaagcccggggcttaaccccgggtctgcattcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacgttgggaactaggtgtgggcgacattccacgtcgtccgtgccgcagctaacgcattaagttccccgcctggggagtacggccgcaaggctaaaactcaaaggaattgacgggggcccgcacaagcggcggagcatgtggcttaattcgacgcaacgcgaagaaccttaccaaggcttgacatacaccggaaaaccctggagacagggtcccccttgtggtcggtgtacaggtggtgcatggctgtcgtcagctcgtgtcgtgagatgttgggttaagtcccgcaacgagcgcaacccttgttctgtgttgccagcatgcccttcggggtgatggggactcacaggagactgccggggtcaactcggaggaaggtggggacgacgtcaagtcatcatgccccttatgtcttgggctgcacacgtgctacaatggccggtacaatgagctgcgataccgtgaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcccaaccccttgtgggagggaatcgtcgaaggtgggactggcgattgggacgaagtcgtaacaaggtagccgtaccggaaggtgcggctggatcacctccttt

SEQ ID NO: 2 - DNA, 681 bp, Streptomyces sp.
  source 1..681: mol_type = genomic DNA
atgagggagcagaagatcatgtcgcggttgtctgcatccaattcagttatgtatgaactcgcgtctcaaaatccgaacatgtcggacctggatcggctcacgaacctctactgggcgctgtgttccgtacttcgcgccgaagtgcccggcgaagtggtggagctggggtgtaacgcgggactcaccagcgtgatgctggaaatggtgttggaggccgagtccccggaccgtacgcttcatctgtacgattccttcgacggcctgcccgctccttcgcatgctgatcaatatttgcgggagggggactgcaaagcgaccgaggcagacgtctgcgccgcatttgagcgctggggcctgcgccggccggagatccacgcggggtggttcgatacgacgctgccgcaacagctccccgaccgcgtggcattcgcctacctcgacggggacttctacgaatccatcctcacgagcctgacacacgtctggccgcggctgtccgtcggcggctccgtgctggtggacgactactgcgacaggctggcaagtccacgcgcttgggacgggctgcccggcgtgaagaaggcatgcgacgcgttcttcgcggacgttcccgtgcgttcacgcgtcctcgtcggcgcaggggacctggctttcgccgagttccgcaagaccgaagcctga

SEQ ID NO: 3 - DNA, 669 bp, Streptomyces sp.
  source 1..669: mol_type = genomic DNA
gtgctgcacgattcgttgaagatcggaagcgtcgaaggcctgctgtctccggatgaggtcacccggctgacggcctgtgtcaccgacgggctgggcgccgaccgccgtttcacggtgtccgaacgccggcggtcggtgcactcgctggaaggtgagagcctggcgcgcgtcaaagaagtctatgaaccgcacggccgcgtggaatacgacgagttgccggacgagatcaccggtgtcctggagggcgcggtggcccgtcgtctcggtgaactgcgaagaatctttccgaccgtccgccgttccatggactggctctacgtggaatacggagccggtcagtacgtcaccccgcatgtcgactacccgttcaatgagatcgcaccggaccggcccaaggtcgccgccataggaatcgtcctgcacaaggccgaagcaggtggcgaattcttcgtggagaccggcggcggcgacgccctctggaacgatgaaggtgaggtcaaggaaggcgccaacgaccacagcccgtggttcaacgccatgcgccgtacccggtggcacgtcaccagccgcgccggggacgcggtgtgctggggtactcaactcgtgcacggtacggaaccggtggccgcgggtcggtccggcaagttcatcggttttctcggctcctga

SEQ ID NO: 4 - DNA, 891 bp, Streptomyces sp.
  source 1..891: mol_type = genomic DNA
gtgaagacggacttcccttaccacccctcacaggaccgccgcggcgaccggctgacgccgcccgtcgccgaacgcaacgcccgccgttcacgaggtgtggccggccgcatcgtatgggcgcactcgctgtggatcgacccggagctggagcggctgtacggcggcgaccggcatgctcccgacgtcaaccgcgaacgggcggtgcggctgtcggacggccgcctggacccgctcgccatgcgccccctggattctctcgtctcggtcaccggccccttcctgagccggcacaccagcaccgtcggcgtcgatccggccgacgaagccttcgacctggtccgccgcacgcacagcggttacctgtgcgaactggccatcggggtcatccggccgggctccccacagcgaggggtgacgccgtcggggccggaccaccgcgaacgctcgcccttcaccgccgtcgtcccccgcgaccacgatgcggtgagccgggccgccgggctcctcgaatccagcgcgaacgagctgctcagccagtggtacgaagccttcacccacgcgcgcggcaccacgcacggcatcctgctgccgccactgcgctgggcacagggcgtgttcgcgctctccatacgctacgaatactgccgccgggtacgggacgagccgggtttcacggccggggagcggcagcacgccgggctgctgctcgacgaggtcaccgaagccctcccgcaacccggcgccttcagggacagcctgcccggtggcgtccagggatcggtggtcaacgaactgctgtcctgctcctaccccttgtgggaccgggtccggttctgcctggactcccccttcgacggactccggtccctcgcggaacattcgacggcctga

SEQ ID NO: 5 - DNA, 720 bp, Streptomyces sp.
  source 1..720: mol_type = genomic DNA
atgagtcacgccgcatttccggcggtggaatccgccacgcacaacgcggaaggcgagcacggccatcaccgcgccaggtttccctatgtcgcgctggaggggctcaacggggcgggaaaatccactctcaggaacagcctggaggaatccttctgggccctgggcatgaatgtgtacaccataggccagcattcctggcagtcaccgggggcggcccgcgtcatcctggacatcaaggaacgccgccacacgcacccgccggagcgcatccaactcgcctacctcgacgaccggcgagcccagggtgacaacatcctctcccgagtactgcacgaagtgccggtcatcggggaccgctccctggtgagcgacatcgtgtatctcgaagccctggacggcatccccgccgaggacaccttcgcgctctaccgccggcatgcggtgatcctgcctgatctgatcgtccacgtcgacgtgtccgcagccaccgccctggcgaacctggggaaacgagaacgccggcgcaagccgcacgagaatcctgaacagatgcggtcgctggccgctacctatgaatccgtgctgtgggcgcagcggttgccgggtctgccacccgtgctgcgatatcagcccgaagtggccgcggacgggacgtcgtacacgaacccggccgcctccatcggcgaccgcatcctcgcttctctgggcctggcctcctga

SEQ ID NO: 6 - DNA, 585 bp, Streptomyces sp.
  source 1..585: mol_type = genomic DNA
atgagcgcaccagggcgggcgatcgtactcgccggaacatcgggagccggcaagagctcaacggccgaagaactccagaagacacttcccgatgccttcctcgtcctgggcctggacaccttcttcggcatggtgccgtaccggtggaccagcgcgggaacggccagtgccgagggtttctcctacatccgggacaccgatccgcacgacggtgccgcgcgtctgcggatcggctacggcccggtcggcgcgcatctcttggccggcatgcgccgggcgacggaggccctgctgcgggagggaaacaacgtcatcgtcgacgagatgccgatcgactccggagtcgttccggcctggaagcgggacctgctggggcacagcgtgttgtgggtccacctcacggcgccgttgccggtgctggaagcaagagaagccgaacgcttccccgagaagttccgtggactcagcagagggcacttcgacgtgtgtgtcccggcggacttcgacatcgtcgtcaactccgccgagctcgatccgagggaacgcgccgaccgtatcgcggcagcgcttccgccccgtcgttga

SEQ ID NO: 7 - DNA, 1227 bp, Streptomyces sp.
  source 1..1227: mol_type = genomic DNA
atgtccgacgcccggtccgcgacaccggccgtgccgttgcgacgcaaccgtgactacaacctgtggtggtcgggcgcggcagcttcccagaccggtagtttcatggtgaccgtggccgtccccatgctcattctgaccaacagcgggtcggccggtcaggccgggctggtcaccggtgcgtcggccgtcgccaaattcgtcgttgcctttccggcgggtgcggtggccgatcgctgttcccgccgccgcctgctcctcctgtcctctctcatgcaattggccgccgtggcctcgatcctgatcgcggtggcctcgcagcacgtccggctcgtccatctggcagcggccgcgcttgtcgagggggcgggttatgcccttcattcggcaacggagctaccgctgttgcgaaaagtcgtgcccgagccgcagcgccgggccgcgctcggtcgcgagcaagggcgaaaggcagcggcacagctgaccggtcctccgctgggaggcacgctcttctcatgggtcccgtgggcgccgttcctcgcggcctcggtctccttcctcggggtgaccacggccgcgctgggaatacgggccccgctgggcccgcagcgccggcgtaaggaagccgcgccccccttctttcccgaaatgctcgaaggagtgcggtatatccgccgcagtgccttcatgagctacatgctcctgtggttcgcgctcgtcaacggcgcattcgcgggcctgagctttttgctcgtcgtgctctgccacgaccgcggcgcgtcagcctccgccgtgggcacggcccagggcgtcggctcggccggggcggttctcggagccctcgcctcggcgtggctgaccgcacgcctgacgcaccggcagcttctgaagctcgcttcgtgggtgctggtcatcggctccgccgccatggccgtgcccaccctttccccgctccccaccggcctggcatacggcgtctccctgttcctgacccccgcggtgaacgtctcgttcacgcatcacatggtcgggaccgttcccgacgctttgaccggacgcgtgtcgatgaccatgatgacggccgcacgcagcctgaactggctgttcgtcatagccgtggggttggcggccgagaggtgggacccgctcgtccccctcttcaccctgtccagcctgttcctggccctggcctgcgccaaccacctccggcgtgtgcgcacacaacggtga

SEQ ID NO: 8 - DNA, 129 bp, Streptomyces sp.
  source 1..129: mol_type = genomic DNA
atggccgtccgccccgtcatcaaggcccgtaacacccatctgatctgcgaaaacgcggccagaggcggggtgcgtgacgcgcgcggtgtcaccgtcgtcccggccttgccgctgcgggcgcaacggtga

SEQ ID NO: 9 - DNA, 3378 bp, Streptomyces sp.
  source 1..3378: mol_type = genomic DNA
ttgaccacatcgttatggagcagacgccgcttactcagcacactcgccggagcggccgcggtccccgccctgccgtccatcctcacgctgaacgccggtgtcgcccatgctgccgggccggccgacggcaacgggccgggggacgtcctgcccacgaccgagcgcggtcaggcggtgtgggcgtacaagaccggaggccgggccgtccgtgaggcggccgccgccgcccttgtcggcagtccggccgccgtgaccgccttcctcacccgcgaactgcccgcggcgagagcggaagacaaccgcttcgcgatcatcatgggtctctcccagggcggcaaggccgtcaacgaaacggccggcgcggcactgaccgcgggagacgaagcggtcgccgcctacctgcgggacggatacaagaagcccgtcgaagaggacctgcgggtggcggtgttcaccgtcatgaacaacagcggtgtcgccgtgcggcgggaggccgacaaggcactgcgggagaacacccggtcggccttccagaagttcctgggcgagacccagtacaccgcgcagcaggaagacgagcgggtggaggtcttctccatcctcaacaacgcctcgcccgaggtgaagaagtacgcgcagcgcgccctgagcgagggaacgcccgaggcgatacgccagttcctcagctcgggccagcacatcgcccgtgcgcgcgacgaagagcgcgcgacgatcgaccagttggtcgagatcgtgcagcgtgagagcaggcaggcgaagctgcagacggagaacgcggtcaccgcctccgaacgcgcgaagagcgccgccgagaaggcgaaggcggccgcgctgctggcggcggagcacgccaaggcggccgagcaggacgtacgcaagtcggccgatgcggcgaccagcgcggcgagagccgccagtggagcggccgatgccgccggcgccgccatcgcggcctcgcgcaccgcgcagtccgcctcccaccgcgcggctcaggccgcccaggccgccgctgccgccgccgcgatggcgggacacgccgcttcccgcgcctacaacgccgccatcgccgcttccaaggatgcctccaaggcgtccgccgcccgggtggccgcccagggcgcacggaacgccgccgcacaggcgcgtaccgcggccaaggcggccgaccaggcgaccatcgcctcccagcaggccgctgtcgccggcaccgccgccgcgggcgcggcccggagcgccgcctcggccgcgaacgccgccgccgagtccgccgccgccgcgggcgccgcccagtccgaggcggccaaggcgaggcaggcggccgccgaggccgatgccgccgccgcacgggccaccagggccgcggctacggcccggtccttggcgaaccaggccgcggaggccgccggcgtggcacgcgacgccgccaacagcgccgccgaccatgcggagaaggccgccgccgcggccgaggaggccgcgaagaacgcgggcaaggcagtggagtacgccaagcgctccgccgcctacgccgaagcggcgatggaagcggccaccaaggccgacaacgcggtcaagaaggctctgcaggtggagaaggcggcgcgcgaggcggaagccgcccggatcgccgcggacaccgaactcggggtgctggaggcccgcctccgggcgaaggcggagaccgaggacgccaagcgcgccgagaggcagcgggcgcaggcggaccgggccacgcagcgggtcaaggacctgatcgccgctgccgaggcggcgctgcgggccggtgacaccgccaaggccgtggcgtccggccgccaggcagcggtcgggctgctggagtcttccggcacctggaccagggaagccgcggagttcgcgctcgccggcaccgaccaggacaccgtcaactggatcgacaccgaccgccttctcgcccagcagcaggacgaccgggagaccgtcctgacgatcgccacgtcgtccacggccgacgtggccgaggccgcccacaaggtgctggccggcgattccccggaggcggtacgcgacttcctcgaccgcggcgtcctcgaagccgcggccacggacaaccgcgtcgccttgttcagggtcctcgacgagaacccgggcagagccgtcagggagaaggccgaagcggcactgaaggacgggagtgcccaggccctgcaccgtttcctcaccgaggaactgacggaagcggtgaaggaggacgacagcgtcgaggtgttccggctcctcggctccggcggcccgtacatgcagtcggcggcgaagatcgtcctggagggctccgcccgtatgcggcgtggcttcgtcagccacgaccagtacaacatcgcgcggctcgatcacgaccgggcgacccatgtcgcagccatccagtcgtccatcgccctcgccgccaaggtcgccgcggaagcgctggagagcgccgcccgggcctccaaagcggccgcggaggcccgcgacgcggccgcggacgcgaccgactgggcgaacaaggcggacgcctacgcccagagggccaccgattcggccacggaggcgaggcgcaacgcggacgccgccgacaagtcggccgccgccgcggcccagtcggcgaaggaggcgagcaacgccgcggtcacggcacgtggtgcggcgcgggcagccaacttctcgatgcggcaggcgtcggcgtccgcccagcaggcggtggcgtccgcgtggcaggcacaggcttccgcctcccgggcccaggcctccgcgacccaggccggcaaggccgccaaggccgctgccgccgccgccagcgaggcacgccggatcgtcttcgtgaagcggaaggccgaggccgaggccgcggcgaagagggcggccgagaaggcgaaagagcaccagtcgaacgggaccaacccctcccagacctccgacaacgacaagccctggtacgtggagaacggggtctggcccgaggacgtgaagagcgccaaggactgggctcaggtcaccgggcactggagcacgatcgccggcggggccgcggtggcgctcggcgtcggtggcttcttcttcgctccccttgggggagccgccctggcagtcggtctggtttcctggggactgcaaggagcgagcgccttgctgaactacatcgggaatgggccggacagctccgaattccattcggcactcggtatgttcgcggtcggcggtatccttctgggaacggggcagttgctcagcaagttcggcaaaatgccggcattcgggcccgcggtcgaacaggtcggcgagaagatagccaacgtcgcccatgacatcacgaccacggtggccggctggctcacctggtag

SEQ ID NO: 10 - DNA, 378 bp, Streptomyces sp.
  source 1..378: mol_type = genomic DNA
atgctgacatccacactgtcagagacccaatggtgggtggccgccgtcctgtccggattcgctctcgcggtggcggcacagtccatggccaccaccgcgcgggacaaactcatcgcggcatcgctcccgctggccggagcgatcgcgatcggtgtgagttcggcggcgcgcggttacacggacacactgccgctgctgctctacaccaccaccatgatcgtcttggtgattctgcgactgctcttcgccaagtacatcaaccgacagctcgccctgaagcgctccgggaaaccgatggagaacatgtcgggcaagcaaaccgccatctttctcctcgccttcatggccacgctcgtcctcgtgtccttcgtcatctga

SEQ ID NO: 11 - DNA, 504 bp, Streptomyces sp.
  source 1..504: mol_type = genomic DNA
atgagaatcacacggcgcctcatcgtcacgggcacagtcctcgcagcgagtgccgcaaccgccgtcaccgtggcccaggcgacgggtaatccggcaccggccacggtcaccgtcgccggtgctgagcccggctccgccgtcgaggacttcagttacccgaatgccgacaagatcaaaaaggaactcggcatcgacctcaagcgcggcgacgggcacatcatgctcgtcgactgcaacaatgacggccgcctgatgaccatctggtcgcgcaccatcgggcaggtctgcttccgcgtcaccggcgacaccggatatctgagcctgtcgattcccaaggtgtacggagtgaaggcagacgccgatcacaaggccaacctgacgctgaccgctgacggcagcaagcagaacatcgccttgggcaagaatgagtcggggtcggccggagagaccgccgacccggagcgtcgtccgcacgacctcgtcgaaatatccacctccaagtag

SEQ ID NO: 12 - DNA, 150 bp, Streptomyces sp.
  source 1..150: mol_type = genomic DNA
atggacgtccagatgagcaccgaggccgacagcgacctcggtgaactagaggtacttgagatggatgaactgcaagtcagcggttcgtccacggcctacaactacatcgccaagaacgcggaccgctcttcgggtataccggcgggctga

SEQ ID NO: 13 - DNA, 840 bp, Streptomyces sp.
  source 1..840: mol_type = genomic DNA
atgatctgctgcccgccgaggcagatggatgtgcccgggcagagccgccggatccaccgggcgatcagcagagccggcgccaactggctgaagaaggcaaccgatatgccgaccgcccgcggcttggacgcgtcaagcgcctccgccagcgctgcgcgcaggttccgggtcgcgaagtcgagagagccggcctgcggctcggacaaccggagcacggcctggtcaagtgcgtcgaagtcgtcccatacctgcgacagcgccgattcgcggagtatcagctgggtggcctgccgggccagacgcaccgcacgcacgggaccgaggcggggcgccgggttcttcgtcaggaaggtgaactcctacctcaatgacaccgttgtcctctccatggtgtcctgtgtgggctgttactggggacgctgcatcttctgttcctacggaaaccgctctctcgaacgcggggcctatcagcaggcttccccggggcaactcgccgacacggtgtgccggatcgtgaaaagtaccgggatcgacttcgtcaccgttgtcgatgagaatacgaacctgctactgctcgtccgggccatgcggttggtgcgtgagcgtggccttcgggtccgtttcaatacccgcaaccgactggaaccgatcctgctcgactctgccttctgccaggaactggccgagctgggctgcgagggaatggctgtcggatacgagggggtcacccagcgactgctgttcaccgtgctcgatctgcggatcgccgatttcgggcggcaggtgggtgttacggactggccggcccggtacgccgtgctccggagacaggaggactga

SEQ ID NO: 14 - DNA, 774 bp, Streptomyces sp.
  source 1..774: mol_type = genomic DNA
atggacgacacgaccttgatatcccgattcctgcgcgacggcttcgtgaagctggagggtgccgtcgcgccgcgcgtggccgcggactgcgcgcggctgctgtggcgggagacgggctgcgacccggacgacccgacgacgtggacgcagcccgtgcactgggtgcccggcctggcgcaggggccgttcgccgccgcccccaactccccggccctgcaccacgcgtacgacctgctcgtcggcgcgggacgctgggagccgcgctactcgctgggcaccttcccgctgcgcttcccgcacgagcaggagccggacgacgcgggctggcacatcgaggggagctatctgccggagggcgagagctggtacttcaccaatctgcgctcccggggccgggcgctgctgatgctgttcctgttcagcgaggtcggtgaccaggacgccccgacccgcatccgggtcggctcacacctcgacgtgccgaaggtactggaggggtacggggaggacggtgccagcgggctgaccctggcgcccgatctggtggcggcgtccgaccaccggccgctcaccctggccaccgggtccccgggcgatgtcttcctgtgccacccgttcctggtgcacgcggcgcagccgcaccacgggaggcggccgcgtttcatggcccagccaccgctgatgccggccgcgccgtacgaactggagcgggccgacggcgcgtactcgcccgtggagatcgcgatccgccggggcttggggcgggacaggtga

SEQ ID NO: 15 - DNA, 180 bp, Streptomyces sp.
  source 1..180: mol_type = genomic DNA
atggcacgtcggcaaccccacgcacccgagcgactgctcgtcgccgcccatcctgaactgcgcgaacgcgaggcactgaaggaactcggcctcatcgcctcgatgaccgacgcgctcaagcgccgcggcgttcccgacctgaccgcacgcgtggccgccgagctgggcgcgctcgcctga

SEQ ID NO: 16 - DNA, 945 bp, Streptomyces sp.
  source 1..945: mol_type = genomic DNA
atgagccgtgctgtggtctacgagtcgttcggtggtcccgaggtgctggagctgcgggaggttccggagccccatgccggcccgggcgaggtccgcgtccgtgtcacggccgccggcctgaatcccatggactggggcatcgcctcacagcccgaggccgccgcccggttcggcatcgccgtgccggccggcttcgggttcgacttcgccggggccgtcgacgaggtcggcgccggtgccgaggggttcgccgtgggggaccgtgtccacggcggcgcgctgggaagggccgttgccgatttcgtggtggtcaagacgcccaccgggacgaccgacccgctctggcacacgccggagggcatcagcgacgaggtggcggccacactgccggtggccgggatgaccgccgctgccgcgttggccgcgatcgacctgcgttccggtgacaccgtcctggtcggcggcgccgcgggcggcgtgggtgtcttcgtcgtacagctcgctcagctcgccggggccagggtgatcgggaccgccggccctggcaccttcccgttcctccgccggttcggtgtcgagccggtgacctacggccccggactggcggaccgggtacgcaccctggctccggagggcgtgaccgcggcgaccgacctgttcggcatcgaaacggccgagaccgcgctcgcgctcggcgtgccggccgagcggatctccacgatcgccgcgggccccaaccctcccggcggcgcccgcgcgaccggcggtttcgacgcgagcccggacgccatggagcggatcggcgacgcgatcctcgccggtgcgatcacggtaccgatcgccgccgccttcccggtcgagcagatccgcgaagccgtgacgttgcaggccggacggcacgtccacggcaaagtcgtgatcacgctgcggccggggccgcactga

SEQ ID NO: 17 - DNA, 201 bp, Streptomyces sp.
  source 1..201: mol_type = genomic DNA
gtgcagacgctcgctcccctgctggcagagggcagcggagtggtgctcacggcctcggccgtcaacgtactgggcctgccgatgctcagcgcgtacgcggccggcaaggcggcactgcgctcgatgacccgcagtctggcccgggaactgctcccgcgaaaccgtggacggcggtggcactcagctctgacacccgtctga

SEQ ID NO: 18 - DNA, 867 bp, Streptomyces sp.
  source 1..867: mol_type = genomic DNA
atgcatactgcagcggtgagcgacgtgccggtggggaacagaacggagctgggtgacttcctgcgcagccgacggggccgcgtcgcaccgcaggaggccggtctgccgccgtcggcgggccgccggcgtacggccgggctgcggcgtgaggaggtcgcgatcctggccggtgtcagcatcgagtactaccgacgcctggaacaaggcaaacagcgacggccgagtcccgcggtactggaggcgatcgggcaggtgctcaagctgaccgacgacgagcgccgccacctcgcggagctgagccggggccgcccgccgtcaccggtcaccggcgcgcggacgggcagggcgtcgcgggcggtacgggaggaggtccgccggatgctggagatcgcgcacccgtaccccgcctgtgtgctgagccggtccagtgacctgctgaccgccaaccagggtgcgctgcgcctctttcccggcatcgaggactggcccgagccggagcgcaacaccgcgcgctacgccttcgtccacccccgcgcccgggaactgtacgccgactgggacgacgtggcccgcggcgtcgtggcccatctgcgcgccgccgtggtggtgcaccggggcgcaccggacatccgcgaagtcgtggacgaactctccgcccgcagcaccgcattcgcttctctctggcagcgtcacgacgtccgggcgcgcaccaacgggcgcaaggtgtaccaccacccggacgcggggcggctgacgctgacgtacgaggcgtacgacgtggcccgcagcgaaggccagcggctgatcgtctaccaggcgccgccgggaacaccggaccacgaggcgctgctccgcctgtcggcctga

SEQ ID NO: 19 - DNA, 1485 bp, Streptomyces sp.
  source 1..1485: mol_type = genomic DNA
atgggcggcgcggaggcccacgaagaagggacggtgggtccggccgtgtccactcaagccgaaccgacccgccggggacgccaactcgtcctggcggtctgctgcacgagcgtcttcctcagcagcctcaacaacacggtgctcaacgtggcactgccggcgatgcagcgtgatctgcgcagcccgctctccggactccagtggaccaccgacgcctacctgatcgtgctggccgcgctgctgatgctcgccgggtccgccggggaccggctcggccgccgccgggtcttccggaccggcctggccctgttcaccctcggctcgctcctgtgcgcattcgcccccgggctgggctggctcatcgccttccgtacggtccaggcggtcggggcctcgatgctcaacccggtggccatgtccatcatcaccaacaccttcaccgcaccacgtgaacgcgcgcgggcgatcggtgtctggggcgctgtgatgggtgtggcgatggcggtgggcccgttggtgggcggggtgctcgtccagggtctgggctggcggtcgatcttctggctcaacctgccggtcggggcggccgcgttcgtctgcgccgcgcgcttcgtaccggaatcccgcgcaccccggccgcgccgggtggacccggtcggacaggccctggtcgtcgtgctgctgggctgcctgacgtacgcgatcatcgagggccccggcaccggctggaccgccccgcgcaccctcgcctgcgccctggccgcaccggccgcgctcctcgcactcgtcctccacgagcggcggcgcaccgaaccactggtggacctgcgggtctttcgcggcgccgggttcagcggagccagtgccatggcggtcctctcgttcttcgtactcggcggcttcctcttcctcaacacgctgtacctgcagaacgtacgtggcctgtcggccctggcggcgggcctgtgcctgctgcccatggcggtcatgaacatcgtcacctcgccgctctccggacggctgaccgccacccgcggcccccggctgccgctgctcctggccggtgccgccaccgccgcgggcggtgtcctgctcgccaccggcggcgcgctcacgacggacgcgctgctcttcaccgcgtacgtcctgatcggcgtcggggccggactggccaacacgccggtcaccgacgcggccatgtccggcatgccgcgggaacaggcaggcgtggccgcggcggtcgtcgccaccagccgccagttcgggcaggcgctgggggtcgcggtgctcggcgcggtactcaccgccggtctgcaccgctccggtacgccgttcacccgggcggtgctcccggggtactggatcgtcaccgcctgcggcctggccatcgtcgtcctcgccctggccaccacccgccgtgtcgtgcctgccgcctcaccggccgggcgacgtcgccggcaggcggccggcgcgcgcctgggctag

SEQ ID NO: 20 - DNA, 615 bp, Streptomyces sp.
  source 1..615: mol_type = genomic DNA
atgcccaagacggtggaccgggacgaacagcgccggcagatcggcgccgccctgctgtccctggcgtccgaacgcggcctggacgaggtgagcgtccgtacggtggccacgtactgcggccgctcccccggagcggtgcagaagtacttccgttccaaggacgagatgctcgccttcgccgccgagatggcgggcgagcgcatcgaggcacggatggccgccgtcgaccccgggctgccgccgcgcgcggccctgcgggccctggtactgaccaccctgcccctggacgccgaacgccgggccgaggcagcggcccaactggccttcgccgtccgcgccgcacaccacgaacccctcgccgaagtacgccgccgggtcgaccaggacatccgggaggcactcgcgcactggctcacctccgccgggtacggccacggccccgacccggccacggactgcctggcgatcgccgacgcggtgatcgcactctccgacggactggcactgcgcctgctctacacacctcaggcccacgacgacctggtacgcgccctggaccgcgggctggaggcgctggtcccggcacaggcccccggcgagccggacgcacgctag

SEQ ID NO: 21 - DNA, 279 bp, Streptomyces sp.
  source 1..279: mol_type = genomic DNA
atggacgaccggctcgatcctgtcacgcgcgcatcggaccaggaccgggagaaggcgctcgcccgggaacgcgaccgcaaggacctgcgggagtggctggccgagtggcgctgctggctcggcggcggggtgatcttgtcggcggtctggggcgtgcggtgcctgcggaagggcgaactgggcttctactggcccgtgacgccgttggggatctgggccgcggtgctggtcgccatcgcggtgtggccgcggagcgggggtgggggcggggaggggtga

SEQ ID NO: 22 - DNA, 23097 bp, Streptomyces sp.
  source 1..23097: mol_type = genomic DNA
atgggaagggtagtgcccggtactccgcaccacagccggcccctgtcgtcggcgcagcgtggcatctggttcgcgcagcagctcgacccggacagccccagctatcacaccgccgaatacgtggagatcgaaggccccctcgatctgccggcgttcgagaccgcggtgcgccgggcggtgacggagaccgactcgctcaacgtccgcttcaccgacgacggtcaggggccctgccagacgatccagcgcgggggcacctggtcgtaccgcgtgttcgacgtcggcgcggagccggacccgagggcggcggccgaggcctggatgcgggcggagacggaccggccgggaggcctggaggcgggccggacggtcactctggcgctgctgcgggccggggccgaccgtgccttcttctacctgcgtgcccatcacctcgtcaccgacggctacatgtgcgcgctgttcacacaccgggtggcggagctctacacggccctggtcaccggggcgccgcagcgcgaggacagcagcggttcgctggacctgctgctggccgacgacgccgcgtaccgggagtcggagcgcttcgcgcaggaccgggcccactggctggaccggctggcgggccgccccgagccggtgaacctcgcgggccggacggcccgggtctcgcggagcttcctgcggtacggcacccgcgtaccggacgccaccgccgacggcctgcgcgcgctggcgcgccgggcgggcgtgggcctgccggtccccgtgatcgccgccacggcgctctacctcggccggatgaccggcgccgacgaggtggccctcggcatcccggtgaccgcgcgcagcggcggcggacggcacatccgcgggccgatgtccaacgagctgccgctgtgggtggaggttcggcccggcatgagcggccgggacttattacgccaagtgtcggccggagcgcggcagttgctcaggcatcagcggtaccggtacgaggacatcgtccgcgatgtgcggacggtcggcggcgagcgccgcctcttcggcccgaccgtcaacatcatgtccttcgccaccgaactgaccttcggcgaggcccgggcgaccgcccacaacctgtccgtcgggcccgtcaccgacctctccttccgcctcgacgaccgcctgggcggcggcgcgctgcacctcgacgtcgacgccaaccccgccctgtacgacgcggcggagaccgccgcgcatcagcggcgcttcctgcgcgtactggagagcttcgccgcggccgacccggaacgtcccgtcggccgtatcgacttcctgctgcccggtgagcgcgccgaggtgctgcccgtacggccggcggcgacggacgctccggagcccgacgagcctggcccgacgctgccgacgctgttcgaagcgcaggccgcgcgcacccccgacgcggtcgccgtcgtcactgaagacggcgagactgaagacggcgagctgacctacgccggactgaaccaccgggccaaccgcctggcgcgcctgctgctgaagcggggcgcgggcccggagcggttggtggccgtggcgctgccgcgctccgccgacctggtggtggcgctgctcgcggtggtgaagacgggcgccgcgtacctgccggtcgaccccgcccacccggcggagcggatcgcactcatgctccgggacgccgacccggtgtgcctggtcaccacgggcgaaccggccgccggggccccgtacggagcggacgtcgtacggctggacgacgacgccaccgtacgcgccctcgccgacgggcccggagacgacctgaccgacgccgaccgcgccggacagctcgaccccgcgcacccggcgtacgtcatctacacctccggatcgaccggcacccccaagggcgtcgcggtcccgcaccgcaacgtcgtgcggctgttccggcagaccgaggcatggttcggcttcggccccgacgacgtgtggacgctgttccactcctacgccttcgacttctccgtgtgggaactgtggggggcgctcctgcacggcggccggctggtcgtcgtcccgcaggacgtgagccgttcccccgagcgcttcctcgacctgctcgtacgccaccgggtgacggtgctcaaccagacgccgtccgccttctacccgctgatacgggccgaccacgaccacccggagctgtcggcgcggctggcgctgcgtaccgtcgtcttcggcggtgaggcgctggatcccggcaggctgcgcgcgtggtacgagcgccatccggccgacgcgccgcgcctggtgaacatgtacggcatcaccgagaccaccgtgcacgtcacccacgccccgctggacgagcgggacgccgccgccgacccgccccgcagcgtcatcggcgagccgatacccgacctgcgggcctacgtgctcgacaacgccctgcggccggtgccgccgggcgtcaccggcgagctgtacgtggcgggccccggcctcgcccgcggctacctcgggcggccgggcctgagtgcgcagcgtttcgtggccgacccgttcggccgccccggggagcggatgtaccgcaccggggaccgggcgcgccggcacgcggacggcaggctcgaccacgccggacgggcggacgaccaggtcaagctgcgcgggttccgtatcgagccgggagaggtcgaggccgtcctggcccggcacggggcggtggcgcaggccgtggtgctgctgcgcgaggaccggcccggcgaccggcggctggtggcctacgcggttcccgccggcgggccccgtccggagtccgccgcactgcgcgagttcgccgggcggcacctgccggggcacatggtcccggcggccgtcgtcgtcctggaccggctgcccctgacggccaacggcaagctggaccgggccgccctgccgtccccggagtacaccccgggtgcggcgcaccgcccggcgcgcgacgcggccgagcgggtgctgtgcgacctgttcgccgaaatcctcggcgtgcggcgggtgggcgtcgacgacgggttcttcgacctcggcggggactccctgctggcgacccggctgctgagccggatacggaccgcgctcggcgccgaactgccgatccgcgtcctgttcgacacgccgaccgtggccggactggccacggccgtacgggagtcgggcggggaagcccggccggtggtgcggcgcacggagcgccccgaggcgctgccgctgtcgttcgcccagcggcggctgtggttcctcaaccggctggagggccgcgaggccggagccgactacaacatgccgttcgtcctgcgactggaaggtgcactcgaccgggcggcgctggaagcggcgctcaccgatgtcgtcacgcggcacgagagtctgcgtacggtcttccccgaggtcgacggcgtgccacgacaggtggtgcgtgaccccggagccgccaccgtcaccctgccggcacgcgacgtcgacgcggccgagctggcggaggtactggcgcggagcgccgccaccggcttcgacctggccgccgaactgccgctgcgcgccgcgctgttcgcgctcgccccggatcggcacgtgctgtcgctggtggtgcaccacatcgcgggcgacgcctggtccatggtgcccctgggccgcgacctgtccaccgcctacgcggcccgcctgcgcggcgaggagccgtcctgggccccgctgccggtgacctacgcggactacacgctgtggcagcgggacctgctgggggaggagcgcgacgaggacagcctcatcgcgcggcaggtggcgtactgggcccgtacgctggccgggctccccgacgaactcgccctgcccgccgaccgcccccgccccggcgtggcgagccaccgtggcgggaccgtgccgctgcgcctggacgccgccctgcacgtgcggctcgcggcgctggccgaggcccatggggtgacgctgttcatggtcctccaggccgggctggccgcgctgctgacccggcacggcgcgggcaccgacatcccgctcggctcgcccgtcgcgggacgcaccgacgacgccctggacgacctgatcggctgcttcgtcaacaccctggtcctgcgcaccgacacctccggcgacccgggcttcggcgaactgctcgcccgcgtacgggagacggacctggcggcctacgcccaccaggacgtgcccttcgaacacctcgtggaggtgctcaaccccaccaggtcgctgagccggcacccgctgttccaggtgctgctgacgctcaccggccggacggcggcgccggacgcgctgcccggactgcgggccgaggccgagcgggtggagtcgggcaccgccaagttcgacctgacgttcaacctgaccgaggccgtggatgccgggagcggccccggtggtgtcgaggggtacgtcggctacagcgccgacctgttcgagcgaggcaccgtcgaggcgcttgcggcccgtttcgtacggctgctggaggcagcggcggccgacccggacctgccgctgggcgccctggacatcctcggcgaggccgagcggcaccgcctgctgaccgagtggagcggcaccggcggccgtaccggtaccggagccgccgctgggggcgcccggtccaccgcgtcgctgccggagctgttcgagcggcaggccgcagccaccccggaggcccgtgcgatcgccgcccccgacgccgagctgtcctacgcgcaggccgacgcccgggccaaccgcctggcccgcctgctgatctcccggggcatcggcccggagcaggtggtcgccgtcgcgctgcccggctccgccgaactggtcgtcgcgctgctcgcggtgctcaagaccggcgccgcctacctgccgctcgacctggcgcagccgggggcccggatcgccgggctgctcgacgacgccacccccgcgctgctgctgacgaccagtcacgccgcgaccgggcttcccgatgacaccggcgcaggcaccggtactgacaccggccccgcccgccttctgctcgacgccccggacaccgtaagggcgctggagcggcagtccgcggcccgccccaccgacgccgaccggagcgccccgcagctgccggaccatcccgcgtacgtgatctacacctccggctccacgggccgccccaagggcgtggtggtcacccaccgcggcctcgcgcactacgtggcccgcgcccggcgcgtgtacacgggcttggacgggacggtcctcgtcccgacctccctggcctacgacctggccgtcaccggcctgtacggggcgctgacctccggcggctgcgcggtcgtggccgggctggacgacgacccttggctgacggcgctgctggcccgcaccccggcgacgttcatgaaggcgaccccgagccacctggtcctgctggccgcgctgcccgccgaattcgccccggtgcggcagctcatgctcggcggcgaggcgctgcccgcggcccaccttgaggagtggcgcgcacgccaccccgaggtggcggtcgtcaaccactacgggccgacagaggcgaccgtcggctgcttggacctgcatctggctcctggtgaagcgccgggcggcgccaccgttcccgtcggccggccgatggccggtatgcgggcgtatgtcctcgacgccgggctgcgtccggtgccgccgggcgtggcgggggagctgtacgtcgccgggccgcagctcgcgcgcggctacctcggccgtcccggcctgacggcggagcgcttcgtggcctgccccttcgcgccgggcatgggggtccccccggccgaaggctgggggaggatgtaccggaccggcgacctcgcccggtggaccgcggacggtgtgctggtcttcgcgggccgcgccgacgagcaggtcaagatccggggccaccgggtcgagcccggggaggtcgaggccgcgctggccggtcacccctcggtggcgcgggcggcggtggcggtacgggaggaccggcccggcgaccagcggctcgcggcgtacgtcgtaccggccggtgacgcggacggcgtggacccggcggagctgcgcgcgtacctggcgcggcggctgcccgagcccatggtcccggcgctgttcaccaccctgggcgcgctgccgctgacccgtaacggcaaggtggaccggcgggccctgcccgcccccgcgctggcgccggcgagcaccggccgcccgccgcgcagcccggaggaggagatcctctgcggcctgttcgccgaggtgctcggcgtgccccgggccggtgccgacgacgggttcttcgacctcggcggccactccctgctcgccacccgcctcgtcagccgggtgcggaccgtgctcggcgccgaactgcccgtacgggccgtgttcgagacgccgacggtggccgggctcgcggcccggctgaaggacgggtccggccgcaggcgccccccgctccgtccctttccccggcgcccggagacggtgccgctgtcgttcgcgcagcgccggctgtggttcctgcaccagctcgaaggggctcccaactacaacctgcccttcgtcgtacggctggagggcccgctcgaccgcgccgcgctcaccgccgcgctcggcgacgtggtcgcccggcacgagagcctgcgcacggtcttcccggacagcggtggtacgccgcggcaggccgtcctggacgccggtcctgccgccgcctccgtcgagctgccggtacggcacgtcgcggacgaggacgcactcgccgcgaccgtcgtcgcggcggcggcccaccgcttcgagctgacccgtgagctgccgctgcgggcagagctgtgcgcgctcggcgccgacgtgcacgtactgatcgtggtcgtccaccacatcgcgggcgacggctggtcgaacggcccgctggcccgggacctgtccgaggcgtacgcggcccgccggaccgggcaggcaccgcgctgggagccgctgccggtccagtacgcggactacaccctgtggcagcaggagttgctgggcagcgaggacgacccggacagcctggctgcccggcagatcgcctactggtcccgtacgctcgacgggctgcccgaggagctggacctgcccgccgaccggctccgcccggcggttcccggctacaccggcggagtggtgaccgtctccgtcgacgccggtctgcacgcccgcctggaggagctggcccgtacctcgggcgcgagcctgttcatggtgctccaggccggcctcgccgccctgttcacccgcctcggcgcgggcaccgacattccgctcggcagtgccatcgccgggcgcaccgacgacgcgctcgacgacctcgtcgggttcttcctcaacaccctcgtgctgcgcctggacacctccggcgacccggccttcgaggagctgctgggccgggtacgggagaccgcgctggccgcctacgcccaccaggacgtgcccttcgagcacctggtggaggtgctgaacccggcccgctcggccgcccggcacccgctgttccagaccatgctgaccctgcacaacaacgcccggccccggctcgacctgcccggactgcgagtgagcccggacatggccgaggtcggcaccgccaagttcgacctgtccttcgggctggtcgaggagcgcggcccggccggcgagcccgcgggcctcgccggcatcctggagtacagcaccgacctgttcgaccacgccaccgccgaggaactggtggcccgcttcgtccggctgctcggcgccgcggccgacgggcccggacggcgcatcggggagctggacatcctctccccgtcggagcgggtacggattctgggtgactgggccggtacctcgcaggacgcgcccgccctgaccgttcccgagctgttcgaacggcaggcgacgctgactcctgacgcgaccgccgtcgtcgcggccggtacgacgttgacgttcgccgaactgcgggcgcgcgcggatgggttggcccgtcacctggcagagcgcggcgtcggccccgacacgctggtcgggctgtgcgtgtcccggtcggcgtggatggtcccggcgatgctggggatctggaaggccggcggcgcgtgcgtgcctctggacccggactatccggccgagcggctggcgttcatggccgccgactccggggtgcgggtgctcgtcgctgagcccgacctggccgacgtgctggagggccaggccgcggaaaccgtccttctgggttcgccggacgcggacggcgagccgttcccgatgcccgccgtacgggccgaccacgccgccttcatcgtctacacctccggctcgaccggcgttcccaagggcgtgaccgtacggcacggcggcatcgcgaacctgttcgccaaccaccgcgagcgcgtcttcgccccggcggcgcgggctgcgggcggccggctgcgggcggcgcacaccgcgtcgctgtcgttcgacctgtgcgtcgggcagctcctcgccctcctcgcgggccacgaactgcacgtcgtcgacgaggacacccgccgcgacgccgtcgcgctgggcgcctacgtccgggaccacggcatcggctacctgtccatcaccccgaccctcacccaggaactgctcggcctcggcctgctcggcgcggaggggcaccagcccgccgcgatcctgcacggcgcggaggccaccggcccggggctgtggcagcggctgcggggcctgtccgccaccgaggtgcacaactactacgcgcccagcgagtgcaccgtcgacgcgatcggcgccaccgtctccggcagcgaacggccccgcatcgggcagccggtcgcgggcatggcggcgtacgtgctcgacgctgggctgagtccagtgccgccgggggtacggggcgagctgtacctggcgggcgcgcagctcgcccggggctacctcaaccggccggtcctgaccgcggagcggttcgtggccaaccccttcagcagcgtgccgggcgcccggatgtaccggaccggggacctagcgcggtggaccgccgacggcgccctggagtacgccggacgggccgacgaccaggtcaagctgcgcggtttccggatcgaactcggcgaggtcgaggcggtactggcgcggcacccggacgtggcccacgccgtggccgtcgtccgcgaggaccggcccggcgaccgcagactggtcgcctacacggtcgcggcacgcggcgccgtgccggacccggccgggctgcgcgcccacgccgcccggacgctgccggagttcatggtcccctccgccgtggtgccgctggacgcgctgccgctcaccccgagcggcaaggtggcccgccgcgccctgcccgcccccggctccgccgtaccccagggcggccgggagccgcgcacggcccgcgaagagatcctgtgcgggctgttcgccgaggtgctgggctgcgcacggcccggcgtcgacgaggggttcttcgacctcggcgggcactccctgctggcgacccggctgctgtcccgcgtccgcgccgtactggacgtggagctgacggtacgcgacgtgttcgaggcgcccacggtggccgccctcgccgcgcggctcgacgccggaggcgcggcgcggcccgccctgcgtcccgtcgaacgtcccggcacgctcccgctgtcgttcgcgcaacgacgcctgtggttcctcaaccggctggacggcgccggcgccgcgtacgccatgccgctcgcgctgcgcctgcggggggatctcgaccgggacgcgctcgccgccgccctcgccgatgtcgtcacccggcacgaagtcctgcgcacggtctaccccgagactgccggcacccccgagcaggtcgtactcgccccggagcgggccgcgcccggactgtcgtgccgcgccgtggccgagaccggcctcgcggcggcggtggccgccgcggcagcgcacccgttcgacctggccgccgagcccccgctgcacgcggaactgctcgccctcgaccccgacgaccacgtcctggtgctcgtactgcaccacatcgcgggcgacggctggtccaacctgccgctcacccgggacgtcaccgccgcctacgccgcccgccacgcgggcgaggccccgtcctggacgccgctgcccgtccagtacgccgactacaccctctggcagcaccggctcttcggcgaccccgccgacccggacagtttgatcgcccggcagaccgcgtactggaccgagacgctggcaggtctgcccgaggaactgccgctccccaccgaccggccgcgccccgccgtggcccgccaccggggcggccgggtgccgttcaccctggacgccgccgcgcacgccgggctcgccgacctcgcccgcgcctgcgacgcgagcctgttcatggtcggccaggccgcactcgccgccctgctcagcaggctcggagcgggcgacgacatccccatcggcacgccggtggcgggacgcaccgacgacgcgctggacgacgcggtcggctgcttcgtcaacaccctcgtgctgcgcaccgcgaccggcggcgaccccgccttcaccgacctggtggcacgggcccgcgaaacagcgctggccgcctacgaccaccaggacgtgccgttcgagcacctggtggagcatctggaaccggtcaggtcgctggcccgtcacccgctcttccaggtgatgctggcgctgcggaacaccggtgaggcgccgctgggcctgccggggctggcggtgagcgcggtgccggtggagaccacggcggtgaaattcgacctggcgttcacggtgagcgagcggaccggcccgcagggcgaggccgccgggctcaccggcgaactcgactacgcgagcgacctgttcgacgcggaaacggcgcgccggatcgccgcccgcttcgtgcgggtgctggaggcggtggcggccgacccggcacggaagctcgaagacctgccggtcctggacgcggccgagcgggagcgggtgctgaccgggtggaacgccaccgagcggcccctgcccccggtgacgctgccggagctgttcgcggcgcaggcggcccgtactccggacgcggtggccgtggccggcgagggcggcaccctgtcgtacgcggagctggacgcgtgggcggaccggctggcccgggccctcgtccagcggggcgcggggccggagcgggtggtggccgtggccctgccgcgttccgtggagctggtggtggcgctgctggcggtgctcaagagcggcgcggcctatctgcccgtcgatcccgggcaccctgccgagcgcacccgggcgatgctggacgaggcgcgtccggtgtgtgtcctggacgatccggagggcgtacggaacccccggccggagacggcgatcactgcgcctcccgtaccgctctccccgtcgcacccggcctacgtgctctacacctcgggctccaccggcacgcccaagggcgtcgtcgtcccgcacgcgggcgtcgtcaaccggctgctgtgggcgcaggccgagtacgggctggacgcctccgacgccgtgctgcagaagaccccgtacggcttcgacgtgtccgtctgggagttcttctggccgctgatcaccggcgcccggctggtcgtcgcccggccggacggacaccgggacccggcgtacctggccgcgctgatccgcgaacacgccgtgacgaccgtgcacttcgtgccgtcgatgctgcgggccttcctggacgagcccgccgccgcgcggtgcacgagcctgcggcgggtgctgtgcagcggtgaggcgctgcccgcacaactggccgcacacggccgggacatcttgggcggctcgaagctgcacaacctctacgggccgaccgaggcgtccgtcgacgtcacctcctgggcctgcgcggaccggccgggcccggtgccgatcggccgcccggtgcacaacacccgggcgtacgtgctggacgccgccctgcggccggtcccgccgggcgtgaccggggagttgtacctggccggtgtgcagctggcccgtggttacctggggcggccggggctgacggcggaacggttcgtcgcctgcccgttcgcgccgggcacgggggtccccccggccgaaggctgggggaggatgtaccgtaccggtgacctcgcgcggtggaccgcggacggagtgctggagtacgccgggcgcgccgacgaccaggtcaaactgcgcggcttccggatcgaacccggcgagatcgagaccgccctggcccggcacggcgatgtcgctcaggtcgccgtcgtcctccgcgaggaccgtcccggtgaccagcggctggtggcgtacgtgacctccgcgccgggagccgtaccggaaccgagggagctgcgcgagtccgtcgcccgcacgctgcccgcctccatggtgccggccgagatcgtcgtcctcgacgcgctgccgctgacgccgaacggaaagctggaccgccgcgccctgcccgccccggacgccaccgcccaggcccccggacgcgctccgcgcaccctccacgaagagatcctgtgcggcatcttcgccgacgtgctcggccgtcccctgcccggcatcgacgacgggttcttcgacctgggcggacactcgctgctcgccacccggctcgtctcccgcgcccgggccgccctcggcgccgaactgtccgtacgggacgtgttcgaggcgcctacggtggccggcctggccgcccgcatcggttccggaggcagccgacggcccgccctccggcccgccgcgcaccgcccggacgtcgtaccgctgtccttcgcccagcgccggctgtggttcctgcacaggttggagggccccagcccgacctacaacatgcccttcgcgctgcggctgtccggcccgctggacaccgccgccctgcgcgccgccctggacgacgtggtcgcccggcacgagaccctgcgcaccgtcttccccgagatcaagggcgctcccggccagatcgtactcggcccggacgagaccgccgtggagctgcgcacccaccgcaccgacgaggccgggctcaccggggcgctggccgaggcggccgggtaccgcttcgagctgaccgcagaaccgcccctgcgggccgacctgttcgccctcgccgacgacgatcacgtcctgctcctggtcctccaccacatcgccggggacggctggtcgaacgtcccgttcgcccgggacctgtcgaccgcctacacggcccggtgcgcgggccacgccccggcgtgggcgccgctgccggtccagtacgccgactacgcgctgtggcagcacgacctgctcggcagcgagcacgaccccgacagcctgatctccgcccaggtcgcccactggaccgaggcgctggcgggcctgcccgaggagctggcgctccccgccgaccgcccccgcccggcggtctccgcccaccggggcggacgggtggtcttcgcgctggacgcggagctgcacacccggctcgccgggctggcccgcgagcacggcgcgaccctgttcatggtcgtgcaggccggtctcgccgccctgttgacccggctcggcgcgggcaccgacatccccctcggctccgccgtcgccggccggacggacgcggccctggacgacctcgtcggcttcttcgtcaacaccctcgtgctgcgcctggacacctccggcgacccggctttcagcgccctgctggaccgggcccgcgagaccgacctggccgcctacgcccaccaggacgtgcccttcgaccacctcgtggaactgctcaacccggcccgctcggcggcccggcacccgctcttccaggtgatgctgacgctccagaacaacgccacgcccgagttcgccatggccggcctgcgggtgacgcccgacgtcgccgagctggccgcggccaagttcgacctgctgttcagcctcaccgaccactacaccgaggacggcgcccccgaccgggtcgacggagtgctggagtacagcgccgacctcttcgaccacggcaccgccgaagccctcacagcacgcttcgtccggctgctgtgcgccgtggcggaggccccggaccggccgatcggcaccgtcgacctcaccgacgccgccgagcgcgagcggctgctggagacatggaacggcgccgccctcaccacgcctccggtgacgctgcccgaggcgttcgaggcgcagacgcggcgcacgccggacgcggtggccgtcaccgacggctccacaaacctcacctacgccgcactgaacgcgcgggccaaccaactggcgcggctgctcctcgggcgcggcatcggcccggaacagcttgtcgcggtcgccctgccgcgctcggccgagctggtggtggcgctgctggccgtcctcaagaccggtgccgcctacctgcccctcgacccggcgcacccggccgaacggctgcgcttcctgctcggcgacgcccgcgccgcgtacgggatcaccggctccgccctggtcggggacctgccggagacggacacccgctggctcacgcccgacagcgacgaggcgcggcgggaagccggggaccagccggaacacgacgtgacggacgccgagcgcaccgcgccgctgcgcccggagcaccccgcgtacgtcatccacacctccggctccaccggcacacccaagggcgtactcgtcgagcaccgcgcgctcgccgcctacctggcgttcgcccgcgaggcgtaccccggcaccgcgggcaccaccctgctgcactcgcccgtctccttcgacctcaccgtcaccgcgctgtacgcgccgctcaccgcgggcggccgggtacggatcgcggaactctccgccgacgcgccggccccgggcccgtacaccttcctcaaggtgacgcccagccacctcgcgctgctcggtgccaccccgcccggcccggagacggcggtcgcggacctcgtcgcgggcggcgaacagctcaccggtgaacagctcgcgccctggcggcgggcgcagccgcaggccgccgtggtcaacgagtacgggccgacggagacgaccgtgggctgcgtcgtcctgcggatgccgccgggcgccacggccggggacggcccgctgccgatcgggcggccggtgcccggcacccgggcgtacgtgctggacgagcggctgcggccggtaccgcccggcgtcacgggcgagctgtacctggcgggcgcgcagctcgcacgcggctacctgaaccggcccgcgctgaccgccgagcggttcacggccgacccgttcggtgggccgggcgggaggatgtatcgcaccggcgacctggcccggtggtccaccgagggcgaactgcacttcgccggccgcgccgacgagcaggtcaaggtgcggggccaccgcatcgagccgggggagatcgaaaccgcgctgctgcgccacccggccgtcgcccgggccgccgtcgtcgcacgggaggaccggacgtccggcacccagctggtggcctacgtcgtcccggcggacggcgaacggagcggcgacgcggccgcgcgggcccagctcgacgagtggcaggaggtctacgactccctgtacgggggcgacggccaccaggacgcggagcccgcgccgttcggcgccgacttcaggggctggaccagcagttacgacgggcggctcctgccggtcgagcagatgcgccgatggcgggaggccaccgtcgaccgcatccgcgaactgcgcccgcgccgggtgctggagatcggcgtgggcagcggactgctgctctcccggctcgccccggactgcgagacctactggggcaccgacatcgccgccgacgccgtcgaccggctgcggacccaggtgcccgagcggccggacctggccggacgggtggaattgcgcgtacagcccgcggacgacctcaccggactgccggaaggtttcttcgacaccgtcgtgatcaactcggtcgcccagtacttcccggacgccggctacctcaccgacgtgctcggcacggcgctgggcctgctcgcacccggaggcgctgtcttcctcggtgacgtgcgcgacctgcggctgctgcggtgcatgcacacggcggtgcagctgcgccgcgccacgccgtccgccgaccccgccgccgtacgccgcgccgtggaacaggccgtcctgctggagaaggaactcctgctcgaccccgcgttcttcgccgcgctggcggcacggaacccggacgtcgccggcgtcgatctgcgggtcaagcgcggcgcgtacgtcaacgagctgaaccgctaccggtacgacgtcgtcctccacaagcggtccgccgacggaccgacggacctggcggccgcaccggaactgcgctggggccacgacgtgaccggacccgacgacctcgcccggcgcctcaccacggagcgcgtgcccgcgctgcgggtgaccggggtgcccgacgaacggctggcggctgaagccggtgccgcgcgcggactggacgagggacggccgctggccgacgtcctcgccctcctcggccccgccgagcagccgaccgcgccggatctcggtccggacaccttccacgagctgggcgaacgcgccgggtaccaggtcgcggtcacccgctccggcgatgacgccgaactcctcgacgtgctcttcaccgatccggcggtgaccgccctcagcggcgtccaccgcccggcggccgagccgtcgcacccgctcgccaacgacccggcggcctcccggcagcacggcgaactggtcgcctcgctcaccgcgtcgctgcgcgcggaactgcccgctcacatggtccccgccgccgtcctggtcctcaccgaactcccgctgaccggcaacggaaagctcgaccgggcggcgctgcccgccccctacctggggtccgtcgagggttcgcgcgggccctcgaccccgcgcgagcgagtgctgtgcgacctgttcgcgcaggtactgggcctggcggcggccggcgtcgacgacaacttcttccacctcggcggacactcgctgctggcgacccggctgatcagccgaatccgtaccgccctcggtgtcgaactgcccgtccgcgccctgttcgagacacccacggtcgccggactggccgcgcggctggacgacgaggagagccgggacgcgacacggcccgctctgcggccgatgtcccggcccgacaccctgccgctgtcgttcgcccagcagcggctgtggttcctgcaccggctggaaggcccgaacgccacctacaacatgccgatcgccctgcgcatgcgcggcgccgtggaccgtacggcactgcgcgcagcgctggccgacgtggcggcccggcacgaaagcctgcgcactgtcttcccggtgacggacggagtgccccggcagctcgtcctcgacgcggacggcgcgcgccctgaactgctcgtacggcacaccgacgagaccgggctgcgcgaggccgtccaggcggcggcgcgctaccccttcgacctcgcggccgacctgccgctacgggccgaactgttcgacctgggcgagcgggagcacctgctggtgctggtggtgcaccacatcgcaggcgacggctggtcgatggcccccctggcgagggacctggccaccgcctacgccgcgcggacccggggacgggcgccgtcgtggacgccactgcccgtccagtacgccgactacacgctgtggcagcgcgcgctgctcggcgacgagaccgacccggacagcccgttcgcccggcagatcgcctactggagcgagaccctggcggacctgccccaggaactcgcgctcccgacggaccgtccccggcccgccgtgtccacctaccgcggcgagatgctcggcttcgacctgagtcccgagctgcaccgtcgcctggcggacctcgcccgggacaccggaaccagcctgcacatggtgctgcacgccggcctcgccgccctgctgacccggctcggcgcgggcaccgacctgccgatcggcagcggcgtcgccgggcgccacgacgaggcgctcgacgacgcggtcggactcttcgtcaacacgctcgtgatgcgcaccgacacctccggcgacccgtcgttcggcgaactgctgcggcgcgtacgggagacggccctgaacgcctactcacaccaggacgtgcccttcgagcacctggtggaggcgctcaacccggtccgctccccgaaccggcacccgctgttccaggtggcgatggtgctccagaacaccccggaggccggactggcgctgcccggcgtcgaggtgacgcccgaaccggccgcctccgggacctcccggttcgacctgttcttcagcgtcaccgaacgcggctcttcagggggcgtgcgcgggacgctggagtacagcaccgacctgttcgaccggcggacggccgagacgttggtggagcggtgggcacggctgctggaggcggtcaccgccgacccggcgcgtcccatcggtgaggccgacgtgctcaccccggccgagcacgcccgtacagcccgtgcatcgggcgccgacggtccgcaggtgccgcccgccaccgtgcccgagctgttcgcacgccaggtggcccggcaccccggagcccttgcggtgctgtccgattccggcacgctgacctaccaggagctgaacgtccgggccaaccgcctggcgcacctgctgctgtcgcgcggcatcggtcccggacacgtcgtcgccctggcattcccgcgcggcgccggcctgatcgtggcgctcctcgcgacgctcaaggcgggtgcggcctacctccccctcgacaccggctacccggccgaacgcctggccttcatgctcgacgacgcccgcccgtcgctgctgctgacgggtgtcgcgggccaggagatacccggctccggccgcctgtcccggctcgacctggacgacggcgacgggcacctggccgggcagccggacaccgaccccggcgccgagcaggccggacggcccgaccccgcacacccggcctacgtgatctacacctccggctcgaccggcgtacccaagggcgtggccgtcacccacgcgggcgcggccgccctggcggcgacgcaggcggactccttcggcgccgggccgggcagccgggtcctccagttcgcctcgccgagcttcgacgcggcgttctgggagctgtgcatggcgatgctgaccggcgccgccctggtggtggccccggccgaacggctcctgccgggcccggacctcgccgccgtactggccgagcacgaggtgacgcacctgaccgtgccgcccgtggcgctgggcgtcatgacgcccgaccagctcgcaccggtgacctgcctggtggtcgcgggagagagctgcccgccgcagacggcggcggccttcgccgccggccgccggatggtcaacgcctacgggccgaccgagtccacggtctgcggcacgatgagccggccgctgaccggtgacggcgccgtcgcgcccatcggcacccccatcaccggcacccgcgcgtacgtcttggacgcggcgctgcgtccggtgcccaccggcgtggcgggcgagctgtacctggcgggcgcgggcctggcccagggctacctgggacggccgggagtgaccgccgagcggttcaccgccgacccgttcggcgcgccgggcgcccggatgtaccggaccggggacctcgcgcggtggaacgccgaaggcgagttggagttcgccgggcggaccgacgaccaggtcaaggtccgtggccaccgggtcgaactcggcgaggtggaggccgcgatggccgcccaccccgaggtggcgcaggccgtcgcgacggtccgcgaggacggcccgggcggtacccgcctgctcgggtacgtggtcccggcggcggacggcccgcgattcacggaggccaccggcgccgccgaggccgcgcaggccgccgacggcgcgccggaacgggccctcggcgagtggcggcagctctacgacaccctgtacggcacgacgcagaccgccccgttcggcgaggacttcaccggatggaacagcacctacgacggggaacccctgcccgccgacgagatgcggcagtggcgggacgccaccgtcgcgcgcatccgggacctgcggccccggcgggtgctggagatcggtgtcggccgcggcctgctgctgtcgaagctcgccggggagtgcgcggcgtactggggtacggacctgtcggcggccgtgatcgagtcgctgacggcacaggtcgccgaacggcccgcgctcgccggccgggtggagctccgcacgcagccggcggacgacctcaccggactgccggaaggtttcttcgacaccgtcgtcctcaactcggtcgcccagtacttcccggacgccgcctacctgaccgacgtgatcggcgccgcgctgcggctgctgacgccgggcggcgccctgttcctcggcgacctccgcaacctgcggctgctgccgagcctgcgggcggccaccggcaccaccgcacccgagcgggaactgctggtcgacccggacttcttcccggccctgcccgccaccgtccccgagatcggcgccgtcgacctgcgcctcaagcgcggggcgtaccacaacgaactgtcccggtaccgctacgacgtcgtcctgcaccgggccccgctgaccgcaccggcccggccgttcgccgacgcacccaccctgccctggcacccgggcctgggcggaacggcggagatcgccgcgctgctgacggaacggcagccggaccggctgcgcctcaccggcatccccaatgcgcggctggcgggcgaggcggccaccgggcacctcgtgaccgtggaccccgagacgctgtgcgcgacgggggagcggctggggttctggacggccgtcacctggtcgggcacggccggtgacgactgcttcgactccttgttcgccgctcaggacgccgccgcaccggacgacaccgccccgtacggcagtgccccggtcaccggggtgtacgtgccggcggacggccgcaccgcaccgccgtccttcaccaacgacccggcggcggccgtcgcgccgtcggcggccctgggctcttcggtacgcgcctggctgcgccggcgcctgccggagtacctggtgccctcggtggtcattccgctcgacgtcctgccgctgaccccgagtggcaaggccgaccgtacggcgctgcccgcgccggagacgacggccgggaccacggcgggccgtcctccggcgaccgcccgggagcggctgctgtgcgacatcttcgcggacgtgctcggccttccgtccgtcggcgccgacgacgacttcttcgacctcggcggcgacagcatcgtctccatccaggtggtcagccgggcccgggccgccggtatggcgatcggcccgcaggacgtgttccggcacaagaccgtggcggccctggccgtcgccgccggaacctcggacgcggcgcccgccgccacggaggcaccgcaggcgggcatcggcgtggtgccgctgacaccggacctgcacgccctgcgggagcgcgacgccgaccccggcaccgggtacgaggcgatgaccgtacccgccccggccgggctcggcgccgaagcgctggaggccgccctccaggcggtcctggaccaccacgacatgctgcgggcgcgcctgacctctggcgggcccgtggcggcggacgggccggggtggctgctcgaagtccggccgcccggcaccgtccgggccgcggaccatgtgcgccgcaccggcaccgcaccgcacgaggccgccgtggccgaggaagagcgggccgccgcggcccgcctggcgccggagtccggggtgctgctccaagccgtccacttcgacgccccacccgggagccccggcttgctgctgctcgtcctgcaccgtctcgtcaccgacccgcagacctggcgcatcctgctgcgggacgtcacggacgcctgcgaggcagccgctgacgggcggcagccccgactcgccccggtcggcacgtcgttccggcgctgggcccagcacctgcgcacccaggcacgggacccccggcgaaccggagaactgtcccggtggaccggcatgctcgccggacccggcagcccggacgatgccccggacgcgcgggcgaccgggaacccggacggcatcccggcgcacgaagccggggacgccatgctgacgaccctcctgtccgccgaacgcaccgggcaggtgctcaccgacgtaccggcggccttccacgccgacgccgaggacgtcctgctcaccgcgttcgcgctggcggtcgccgattggcagcgccgcagcagcgccgccgacggcaccgtgctcgccgacgtacggcgcccgggccgcgtaccgctcggcgagggcatcgacctgtcccgcaccgtgggccgctgcacgaccacccacccggtccgcctggacctgggcgacccgggcggggaagcgctgcgggaggacggcgggcacgccgccgcgacggctctcaaacgcgtcaaggaggaactgcggtccctgccggacggcggtctgggccacggcctgctgcgccacctccaccccggcaccgcccccgaactggcggccctgcccgtaccccggttcgccttccactacctcggagagctgccgcaggccccgtccggcccggacccggccggcgaaccggcgtgctactgggccgaggtggccatcactgccgtggacggccccggcgggccacggctggccgcgacctggcgccgggcccacgcgcccctgaccggccgcgacctgaacgacctggccaccgcctggacccgcgcgctcgaaacgctggccgcccaggccgcccgccccggcgccggtggccgcaccccgtccgacgtaccgctggtccggctcagccaggacaagctcgaacagctcgaagccgcatggaggcgatcacagtga

SEQ ID NO: 23 - DNA, 7131 bp, Streptomyces sp.
  source 1..7131: mol_type = genomic DNA
gtgacgcagccggaactggaggacatcctccccctggcaccactccaggaggggctgctgttccacgcgctgtacgacacggcagcgctcgacctctacaccttgcagagcgcctacgacctggacggggacctcgacgcgacggcactgcgcgaggccgcgcgcacgctgctgcggcgccacgccgccctgcgcgccggcttctggcacgagggcctggacgaaccggtgcagatcgtcccgcgcgaggtcgccctggcgtggcagcaggccgacctcaccgggaccgcccccgaggagcgggaggcggaggcggagcgcatcgcggccgaggaccggctccgccgcttcgacccggcccgcccgcccctgctgcgcttcacgctgctgcggctcgggacggaacggcaccgtttcgtcgtcaccgcgcaccacttggtgctcgacggctggtccgtacaactgctgctggaggagctgttccagctgtacgtccggggcggccgggaggacgggatgccgccggtggcccgctaccgcgactacctggcctggctggccggacaggaccgggccgccgccgaggacgtgtggcgccgggcgctcaagggcatcgaggagcccacccgtgtggcgcccgccgacccgtcccgggaaccggccctgcccggccagatcctgcgcgacctgccggacggcctcgccaacgcgctggacgccctcgcccgccgcgaacggctgaccctgagcacggtcttcctgggcgcctgggccgtcgtcctcggccggctgaccggcaaggacgacgtggtcttcggcaccaccgtctccggccgcccgccccaggtgcccggcgtcgagcggatgatcggcctgttcgccaacaccctgccggtacgggtccgcctcgacccggaacagccgctgaacgggatgctggccaggctccaggaagaacagacacacctgctggcctgccagcacgtggggctctccggcatccagcgcacgctcggctcgggcgaactgttcgacacggtgaccgtgttcgagaacctgcccgccgggcagagcggactcgccgccaccgcgcacggcggcggaccgcgggtgacggcgagccggagccagggcgtcacccactacccgctcaacttcatggtgatgcccggccgcccgctgcggctgcggctggaccaccagaccgggctgttcgaccgggacgccgccgaggcgatcatggcgcgggtggtccgggtcctggagaacctggtcgcggaccccggccggcgcctcggcgagctggagatcctggccccggccgaacgggcccgggtgctgggggagtggagcggcacggccggagagccgccggccgagctgatgccgcaggcgttcgcggcccgggtggcgacgaccccggacgcggtggccctggtctgcggcgaggtgtcctggacgttcgccgaactggacgcgcgcgcggaccgcctcgcgcggcgcctggccgcccggggtgccggacccgacgccctggtggggctgtgcgtgcgccggtcggcggacatgatcgccgccgtgctggccatctggaaggccggcggagtgtgcgtaccgctggaccccggctacccggcggagcggctggcgttcatgctggccgacgcggccgtacccctggtgctgaccgaacgggagctggccgggcgcctcggggaggaccgggcggcgtccttggtgctgctggacgacgacaccccgtccgaaaccggtgttgccctccgtacgccgcccctgcacggcgaccacgccgcgtacatcgtctacacctccggctccacaggcgtgcccaagggcgtcgtcgtctcccacctgggcctggcgaacctgtacgccgaccaccgcgaccgggtgttcgccccggccgccgacgccgtggccgcgtcggccggacggcggctgcgcgtggcccacacggcgtcggtgtcgttcgacgcctccatggggcaactggccggcctcgtcgccggccacgaactgcacgtcgtggacgaggacacccgccgcgacgccaccgcgctgctggcctacgtacgcgagcggagcgtcgactacctgggcatcgccccctcgctgatgcgggaactgaccgcgctcgggctgctggacgggcccggacaccgcccggcggtgatcctcctcggcgcggaggccgtcgacgcggccctctgggaggcgctgcgggcccacccggagaccgaggtgcacaactactacgggccgagcgagtgcacggtggacgtcgtcggcgccctggtacgcgacagcgcccggcccgtgatcggccggccggtcgccaacacccgggcctacgtgctcgacgcacggctgcgccccgtaccgcccggtgtggtgggcgaggtctacctggccggcatccagctcgcccgcggctacctgggccggcccggcctgaccgccgcacgcttcgtcgccgacccgttcggtacgccggggacgcggatgtaccgcaccggagacctcgcccgctggacggccgacggccggctggacttcgccgggcggaccgacgaccaggtcaagctgcgcggctaccgggtcgaacccggcgagatcgcgacggtgctggcccgccacgccaccgtcgcgcacgcggccgccgccgtacgcgaggaccggcccggcgaccagcgcctgaccgcctacgtggtgccggcgcccggcgcccgggcgcacgcggaggaactgcgcgagttctgcgccgggacgctgcccgagttcatggtgcccgcccagatcgtcgtgctcgacgccctgccgatgacccccagcggcaaactggaccgccgcgcgctgcccgcccccggcgcgcgcaccaccggcggccgggagccgcgcgacccggtcgaggacatcctgtgcgggctgttcgccgaggtcctcggcgtggaccgggtcggcgtcgacgacggcttcttcaccctcggcggccactccctgctcgcgacccggctggtctcgcgcgtccgcgcggccctcggcgcggaactcgccgtacgggacgtcttcgaggccccgaccgtcgccgggctcgcggcccggctcggcaccccgcgcgaggacacccggcgacggccgccgctgcggcccgccggcgagcgcccggccgaactgccgctgtccttcgcccagcggcgcctgtggttcctgcaccagatggaaggccccacgccgacctaccacatgccgttcgcgctgcgcctgaccggcgggctcgacgtcgcggccctgcgcgccgccctgaacgacgtggtggcccggcacgaggtactgcgcaccgtgtacccggacaccgggggcaccccgcgccagcacgtactagggcccgccgaagcccccgcaccgctgcccgtgcggacggtcgccgaggacgccctggcggcggccctcgcggacgcgtacgcgcgccccttcgacctcacccgcgaaccgcccctgcgcgccgaactgttcgcgctcggccccgacgaccacgtcctgctgctcgtactgcaccacatcgcgggcgacgcctggtccaacctcccgctcgcccgcgacctgtccgccgcctacgcggcccgccgtacaggcgaggccccggcctgggcgccgctgccggtccagtacgccgactacaccctgtggcagcgcggcttcctcggcgaggagagcgacccggacagcgcgctctcccgccaggtggcgtactggcggacggcgctcgacggactgcccgccgaactggaactgcggcacgaccggccccggccggaggtgtccgaccaccgtggcgggacggtgccgctgcacgtcggccccgccacccacgcccggctcgccgggctggcgcgggcgcacggcgcgagcctgttcatggtcgtccaggcggccctcgcggcactcctgacccgcgagggcgcgggcaccgacatcccgatcggcacaccggtcgcggggcgcaccgacgagggcctggacgatctcgtcgggtgcttcgtcaacaccctggtgctgcgcaccgacaccgggggcgacccggtcttcacggacctcctcgaccgggtccgccggaccggcctggccgcgtacagccaccaggacgtgcccttcgagcacctggtggaggtgctcaacccggcccgctcggcggcccggcacccgctgttccaggtgctgctctccctccaccacgccgcggacctgccggtggagctgcccggactgcgcgtccgggccgaggagacccggtcccgctcggccaagttcgacctgacgttccggctggccgagcggaagggcccggacggcgggcccggcggcctcgaaggcggtgtcgagtacagcaccgacctgttcgaccggagcacggtggagggcctggccgcccgcttcgcgcggctgctcgacgccgtcgccgccgaccccggccggcggatccggcggctcgacgtcctgtccccggccgagcgggagcgggtgctgaccgggtggaacgccaccgagcggccgttgcctccggtcacgctgccggagctgttcgcggcgcaggtggcccgtactccggacgcggtggccgtggtcggtgagagggacactctgtcgtacgcggaactcgacgcgtgggcacagcggttggcgcgggctctgaccggtcggggggcggggccggagcgggtggtggccgtggtgctgccgcgttcggtggagctggtggtggcgctgctggcggtgctcaagagtggcgcggcctacctgcccatcgaccccgccctgcccgccgaacgcatccgcctcctgctcgacgaggcgcgtcccgtgtacgtcctggacgatgcggacggcgtgcggaacccccggccggagacggcgggtaccgcgcgtcccgtaccgctctctccgtcgcacccggcctacgtgctctacacctcgggctcgaccggcgcccccaagggcgtcgtcgtcccgcacgcggccgtcgtcaaccggctcctcggcatacaggaggagttcggcctcgaaccctccgacatctgcctgtacaaggcaccggccgccttcgacgcgtccgtgtgggagctgttctggccgctgaccaccggcgcacgcatcgtggtggccgctcccgacgggcaccgtgacccggcgtacctggccgcgctgatccgcgaacacgccgtgacgacggtgcacttcgtcccgtcgatgctgcgcgccttcctggacgaacccggcgccgcccgctgcacgagcctgcggcgcgtccagaccggcggcgaaccgctgcccgccgagttgcgtgaccgtgccacccacctcctcggcgcgcccctgaaccatctgtacggaccgacggaaaccaccatccacgccacgacctggtcctgcgggcggccctcggccgccgcaccggacaccactgccgcccgtccggtgcccatcggccggcccgtccgcaacacccgggcgtacgtgctggatgcggcgctcggtcctgtgccgccgggtgcgacgggggagttgtacctggcgggtgtgcagctggcccgtggttatctggcaaggccgggcctgacggcggaacggttcatcgcttgcccgtacggcgcaccgggggagcagatgtaccgtaccggtgacctcgcgcggtggaccgccaacggggtgctggagttcgccgggcgggccgacgaccaggtgaaactgcgcggcttccggatcgaactcggcgagatcgaggccgccctgtcccggcacgaggacgtcgccgcggcggccgtcgtcctccgtgaggaccgtcccggtgagcagcggctggtggcgtacgtcgtgcccgcgccggaaaccggaccgcaccccgccgacctgcggcgccacgccgcgctgcacctgccggagttcatggtgccggccgccgtggtggtcctggccgcgctgcccctgacctccagcggcaagctcgaccggcgcgccctcccggagccgggcggcccgctcaccgtgaccgccaagcggcccgcccgcaccccggaggaggaggtgctctgcggcctgttcgccgaagtcctcggcctgaccggaaccgggatcgacgacgacttcttcgacctcggcggggactcgctggcggccatccggctggtccgccgcatccgggaggcggcgggcgtggagctgtcggtcagcgacctgttcgcggcgcccacggtcagccgcctgatggaccggctgcgcctcggcgtccggggcgacgccttcgacgtactgctcccgctgcgcgaatccggcgggctgccgccgctgttctgcgtccaccccgcaggcggcctgagctgggcctacaccggattgctgcggcacctgggccccgaccggcccgtgtacggactgcaggcccgcggcctcgcccgtcccgagacgtacccgacctcaatggacgagctggtcaccgactacgtcaggcagctccgctcggtccggcccaccggcccctaccacctgctgggctggtccttcggcggcctcgccgcccacgcgatcgccgtccgcctccaggccgagggcgagaaggtcgccttcctcgccctgctcgactcctacccccgcgacccggaggccgcggtcccgcccgtggaggagcgggacgtcctggcgacgctgctcgacttcgcgggccacggcgaggacgtcaccgacgccgaacagctgcgcgtcgaggacgtcatggagatcctgcgccgcggcggcgggcccctggcggacatcgaggaacgccgcgtgggggccctggccgacgtcttcgtcaacagcgcggaactcgcgtcggcgcatgcgcccggccggttcgacggcgacctcctgcacttcaccgcgaccgccggccgcccggacggagcgcccacggcggccgtctgggagcccttcgtcaccggacgcgtacgccggtacaccgtggactgtacccaccacgccatgtgccgccccgagccgctggccgagatcggccgggtggtcgccgagcgcctggacggcgcggaccggccgaggcggcaggacggccccaaccggtaa

SEQ ID NO: 24 - DNA, 231 bp, Streptomyces sp.
  source 1..231: mol_type = genomic DNA
atgaccaatcctttcgagaacgccgacagcacctacctggtgctggtcaacgacgagggccagcactcgctgtggccgtcgttcgccgaagtcccggccggctggacggtcgcgcacggcgaggacacgcgggacgcctgcctcgcccacgtcgaggagcactggaccgacatgcgcccgaagagcctcgtccgcgtcatggacgaagccggggcgccgtccgcccgttga

SEQ ID NO: 25 - DNA, 1116 bp, Streptomyces sp.
  source 1..1116: mol_type = genomic DNA
ttgaccgccgcggaccgcgaggagagagacaccgctgtgctgacgtaccaggaaccccccgccggcggcggcgcctgggccgagacggtggagctgacgccgcaggaagccgagcagttccgcaaggtcgccgacgagatcaccggcatgctgccgcacgggaagccggccgggcccggacagctcgacgaccccgggctgctcgcggagatcgaggtggcggcgcgctggctcccgccgcgcctggcccgcgcgctcagccgcttccgcaggcagggcaacccgcacggcgtcctgctcgtacgcaacgtaccgctcgacgccgacctgcccccgacccccgccgacggcgccgcacccgtctggtcgcggctcgggaccgcgaccgtcggccagctcgccgtggtctcctggctgggcgacgtcatcgcctacgccgacgagaaggccggccggctggtccaggacatcgtcccggtcagcggcgccgaggagtgccaggagaacagcggcagcgtgctgctcgaactgcacaccgaggacggcttccacccctacaaaccggacttcgtgacgctgctgtgcctgcgcgccgaccacgagcgccgggcccgcaccacctacggtgcgatcacccgggcgctgccccgcctgtccgccgagtgcgtccgcgtcctgcgccgcccgctgttccgtatccacttcagcagttccttcggggggccgggcgccggggcgaaggggagcggtacgggtactggtaccggcgtggtctcgccgccgctgcccgtgctgtccggaccggacggtgacccggagatcgtcgcggacttccacgccatggaacccctggacgagaccgcggcgcgcgccttcggcgaactgcgtgacgccatgctcgccgtcctcgacggcgcggtcctcgaagccggcgacctgatcgtcgtggacaaccgcaccgccgtgcacggccgcaccgccttccggccccgctacgacggacaggaccggtggctgcggcgctgcttcgcggtcgccgacctgcgcccgtcccgcgccgcccggaccccgggctcccaggtgtgcgcgccgctgaagccggagggcgggccgtga

SEQ ID NO: 26 - DNA, 171 bp, Streptomyces sp.
  source 1..171: mol_type = genomic DNA
gtgacggcggaccgccgcgccgtcgccccggcggccgcgggccgggcgcccggcagacaggcgtccggctgtccgacgcccggcagaccgaccagcactccggaagcagcagtgatcggcacggacatgagcgcgcaggtccgggcgctggccgcgaagggtggacggtga

SEQ ID NO: 27 - DNA, 363 bp, Streptomyces sp.
  source 1..363: mol_type = genomic DNA
atggagcccatggaacccatggccgggaacgatccgcacattcacgtccaggcacaggcgggccgcgacgacgccgctgcccgcaacctgatagccgccacgttcggcctggtcagtaacctgccgagcgtcgtcgccacgggatgcgggctgcgggtgccgtacgcgatgacctcgtcgcgtccggaccgtgtcacctgcctcgcctgccgtgagcacgcacggcaggcgcacctgcgcttcgcggtggaggtggaacgcctgagccgcatgctgggatcggtcatcacccccgcccagggcaagttggccgccgaccggcaccgcgacctcgcgcggcggttctccgacgacgagggctga

SEQ ID NO: 28 - DNA, 390 bp, Streptomyces sp.
  source 1..390: mol_type = genomic DNA
atgagaatctacgtgaccagcgtctttgtcgacgaccaggccaaggcgctgagcttctacaccgacaagctgggcttcgtgaagaagcacgacgtcccgctgggcggagaggcccggtggctgaccgtggtctcccccgaagacccggacggcaccgaactgctgctggagccctccggccacccggcggtgcagccgtacaagacggcgctggtccaggacggcatcccggccacctccttcgccgtggacgacgtgcgggcggagttcgagcggctgcgcgggctcggcgtgaccttcacccaggagcccctggagatgggcacggtcaccaccgcggtcctggacgacacctgcggcaacctgatccagatcgtgcacaccgcgtag

SEQ ID NO: 29 - DNA, 294 bp, Streptomyces sp.
  source 1..294: mol_type = genomic DNA
atggcagaggatgttttcaaggcgctggccgaccccacccgccggcgcatcctcgacgagctggcggagcggaacggtcagacgctgttcgagatatgcgcccggctggtgaccaagcacggcctggggctgtcccggcaggcgatcagccagcacctggccgtcctcgaatccgccggcctggtccgcacccggcgccagggccgctacaagttccacgacctggacaccgcaccccttgagcacatcatgacccgatggctcagcaccgacacgccggagagcaccccatga

SEQ ID NO: 30 - DNA, 693 bp, Streptomyces sp.
  source 1..693: mol_type = genomic DNA
atgcacccttccgtgcggaacaatgcccggatggaagatcgaatagccccgacccccctgcttcccgtccgcggagcggcccgctgcgccgtggccgccctcgtcctcgccggtgccgcctgggcggcccgcgccctgtggcagatccggctcgccaccgccgggatgccggcttccggaccaccggaccaaggcgaagggaagcaccgcccgctgaccgcgctggaggacggctaccacatcgtcaccactgtgggcagcgcggccacggtgctctgcgcgatcaccttcctgatctggctctcgcgtgtccgggacaacgcccgcgccctctccggacagtcgccccgctacgccaggccgtggctctacgcgggctggatcgtgcccgtcgcgaacctgtgggtgccccgtggcgtcgtcgccgacatccaccgcgcgagcgccccgggagaacggctgccgcacgccgtgaactggtggtgggggctgtggctcgtcggaatgctgagcgggacgggcctgatgtacaccgtccccacggacgacgtcatcgaacgtgcctaccaggcggtgtatccgctcctggccgccgacgcggccgtcgtcgcggcggcggtggccggtgtcttcgtcgtccgcgccctcaccgcggcacagcggcggcgcggcgtcgattccgtacgggcctga

SEQ ID NO: 31 - DNA, 1014 bp, Streptomyces sp.
  source 1..1014: mol_type = genomic DNA
gtgcatcagctcaccggcgcggccggttcggccgtgtcggaggcgacgtccgcggcgtactacgcgtcgatcgcacaggccgaggagagcggcgacggcacggtgcggcaccggctgcgtccggaggtcggcgacggcaccatcgaggtgacgtcgctgcgcagcgggttcgacatcgtccggtacgacgtcgcgttcgccggcggacaccaggtgggattccggttcccgggggaccacttcgagctggagcactgtgtcgacgggcgcatgcacatcagcgaggccggggccggcgccggtgacctgcgcgcccgctcggtggcgctcagccctcgcggcgccatcgacggcgtcgtggcgcacggcgacggtgagcggtaccgcgcggtgtcggtcaccggcacctggtccgggctggagtcctacctcggcggcctcggcgcgggcgccctgccgcccaccggaccggagagcgtcgcccgcgagcggtacctgggccggtccgccgcgctgcgcccggccgcgggaccgctggaggagatcttcctgcaccggcgctcctcaccggggcgcctgctgttcatggagtcgcggctggtggcggcgctcgccgcgctgatcgacgggctcgcgtccgacgcgtcgcccgccgacgcccgggaccgcgccgcgccggccctggccgaccacgagacggccgcgctgcgccgggtgcccgaactgctgtggcgggagcgccacgcaccaccgacactcgccgagctggcccgggcgtcgtcgatgtcggtacggcgcctgaccaccggattccgcgcgctgtacgggacgtcggtgatggagtaccaccggcggcactgcctggaccgggccgccgcgctgctggtcgagaccgactggccggtctcccggatcgcgcacgagatcggctacgcctcggccggcaacttcggctatgccttccggcgcgaccggggcacgtcgcccgcccggttcaggcgggcgcactcctga

SEQ ID NO: 32 - DNA, 1185 bp, Streptomyces sp.
  source 1..1185: mol_type = genomic DNA
atggtcctcggcgggctgtcggcggtctcaccgttcgccatcgacatgtacgcgcccggcttcccggagatcgtcacgtccttcgggacctcgaagacggccgtccagctctccctgacggcgtgcctcgtcgggctcgccgtcggacaggtcgtcctcggcccggtcagcgacgcgacagggcggcggcgcctgctcctggccggttccggcctgttcagcgtgctgtccctggtgtgcgcggcggccccgtccatcgccgtgttcgacgcggcccggctgctgcagggggtcgcgggcggcgcgggcatcgtcatcgggcgggcggtggtcagcgacctgttcaccggcgcccccgcggcccgccggctcaccacgctgagcgtcatcgggtccaccgcgccggtactcgctcccgtggccggcggcctcgtcctcggtgtcggctcgtggcggctggtgttcgtggcgctggccgtgaccgggctgctgttgctgaccgcgatctgggcgtgggtgcccgaatcgctgcccgccgagcggcgccggaccggcggcctgccggccgtgctccgcgcgatgggcggactgctccggcgccgggagatggccggccacctcctggtcatgggatgcgggctggccgccctgttcgcctacatcaccggctcgcccttcgtcttcgagaacggctacggcctgtcgcccacccagtacagcctcgtcttcgccaccaacgccgtcggcacggtgctggccgggacggtcttcgggcggctggccgggcgggtacgtctcaacctcctgctcaccgtcggcgtggccgtcaccgtggtcgccctggtcacgctggtgaccctgctcgccgtcggcatcagcacgttcctcaccacgtgggcctgcctgttcggcctgacctccgggttcgggctgctgctgcccgcctcgaccaccatcgtcctcgcggtcggcggcgacgcgcccggagccgcttccgggatgctcggcggcgcccagttcgtcctcggggccgcggccgcgcccctgcccggcgtactgggggacaccaccgcgatgtcgacggcggtcgtcgtcctgggctgcgtactgctgtcggcgacagccctgctcacgctcgcccggccgtgggagggccatggcgagccggccggctcacccgagtga

SEQ ID NO: 33 - DNA, 561 bp, Streptomyces sp.
  source 1..561: mol_type = genomic DNA
atgcaccggtccatcgaccttcaccacaccgccgacacgttcgagaaatgtgcgcagttgtgccgctcggcccgggaggcgaccgaccgggccgtccgcatgcgggccgaggcggcggaggccagactccgggccgtacggatgcgccggaccatggagcggatgaaagccgggcaccgttccgacgacgcccccggatgccggcggcttccggccacggaaacccgccgcggcgccaccgcccgagcggcgcagctcgcgctgctgaccgctttcgtgcacgcccgcctggacgaggagggcggcaccgcggacctgttccacgagaccggctgcccgggcgccggggcgtcggagtgccactgtcccgtcccggagcgggtacgccacgacgtcgccgtccggcacggcctcgcccgggactgcgaggaggcgctgcacgaccccgatcacgacgcgtgcgactggccgcacagcgaactggccgccctgctgcggttgaaggtgctcgcgctgccgtacgaactgcaccggcagtggcgggaggagtggagaccgtga

SEQ ID NO: 34 - DNA, 759 bp, Streptomyces sp.
  source 1..759: mol_type = genomic DNA
atgaaccagaaacgggagatcgcggcgctgatcggcgatgtcttcggccccgcggccgtcggcagctacctgtacggctccgccgtgctcggcggcctccggccggccagcgacctggacgtcctcgtcgtctcccggcggcgcgcgaccgtaccgcaacggcgcgccctgctcgacgggctgctcggcatctccggcttcacgggaggggcccgcccggtcgaactcaccgtcgtcgtccaggaccaggtacacccgtggcggtacccgccgatctgcgacttcctctacggcgagtggctgcgcgacacgtacgccgccggtgtggtccccgagccggaaccgctgccggacctggcgctgctcgtcaccatggcgctggccggtgaccatcccctcacgggcccgcccccggcacaggttctcgaccccgtcccgtgggcggacgtggcccgggcgagcgtgcacggcatccccggcctgctggacgaggtggacagcgacacccgcaacgtcgtcctcaccctcgcgcgcgtctggaccaccctcgccaccggcgagatcaggacgaaggacgccgccgccgactgggccctcggccacctcccgccccggcaccgcccggtcctgcgccacgccagggagctgtacctcacccgccggtactccgaggagagctggagcgacgacctcagatcgcgggtgcgcccgcacgtggaccacgtgctcgccgagatccgcgaactgccctacgcctga

SEQ ID NO: 35 - DNA, 1008 bp, Streptomyces sp.
  source 1..1008: mol_type = genomic DNA
atgacggaagcgggccggagcgttccgccccggcggcgcttaccgttccggcagttcgtgctcaaggtgcacagcaggtgcaatctcgcctgtacctactgctacgtgtatgaggggcccgaccagagctggcgcgagcgccccgcccgggtggccgaggagaccgtacgccgcaccgcggaacggatcgcccggcacgtcaccacgcacgggctgcgttcgatacgggtcgatctgcacggcggggagccccttctcctcggccccgccgcgctgatcgaccacgcgcgggccatccggaacgcggtgcccggagactgcgccgtcgccgtctcggtgcagaccaacggcacgctgctcaccgaggacgccgtgcggcggtgcagcgcggccggcatcagcgtcggtctgagcctcgacggcggcagtgcccggctcaaccaccggcgtgtcgaccgcgcgggccgctcctcctggcccgcggtacggcgggcggccggtctgctggccggtgtgcctgacgcgtacgggggcatcctgtgcaccgtcgacgtggaggccgaccccgccgaggtctaccggtcgctgatcgcgctggcgcccaccggcctcgacttcctgctcccgtacgggaactggagcgcgccgccgcccggcaccggcccgttcgccgggcccgtcggccgggcagcgcgtcccgtcccgtacggtgcgtggctcgcccgggtcttcgacctgtggtgggacacgccgccgggccgacgggccgtaccggtccgcctgttcggcgagatcgtggcgctgctgctcggggcggcgagccggtccgaggcggtggggctgtccccgctggccgtggtggtgatcgacaccgaccgttacctgcacggctccggcttcggtcacccttcggtgtactgcgcggatctggagcatctgatccgccacatcggcgagcggcttcaggaagaggtacgaggggtgccgggcgatccggtacgtccgtga

SEQ ID NO: 36 - DNA, 663 bp, Streptomyces sp.
  source 1..663: mol_type = genomic DNA
atgaacgcagaggcggaagcgcaggcgcaggcagcggcagcggcgcggactgagtcaccagtacaggcgcagacagcggcagacctcatcgggagcgagcccgcggcgaccggctggccggacgtaccgggcgacaccgccctcacgatcaaggtcccggaagccgacccgctggtccgcgcgggcttccccgcccacgtgaccgtgctctacccgttcctgcacgagagccgcatcgacgccacggcgcagcacgacctcacccgcctcttcgccgggcacgacgccttcaccctgaccctcgactcgttcggccgctaccccggcgtgctctacctcgacccgtggccccacgcccccgtgaccgcgctgaccaaggacctcacccggcgctggccggaagccgtgccctaccgcgggatattcggcgccggactcgccccgcacctgacggtcgcgaaccacgagggccccgcgacccaggaggccgcgtacgacgcgttgcaggccgagctggctccgtcgctgccgctgacctgttccgtacgggaggtccacctcatcgtctgggacgggcggcgctggcgggaccggacggcataccgtctgggaccccccgcaccgtgtcgcttgaccttgtcgcagcgacaacgtttctactga

SEQ ID NO: 37 - DNA, 798 bp, Streptomyces sp.
  source 1..798: mol_type = genomic DNA
atgcgcatcggagaactcgccggcatcgtcggcgtcaccacccgtgccgtacggcactaccaccgcaccgggctgctgcccgagccgccgcggcagcccaacggctaccgcgagtacgcgctgcgggacgccatcgagctggcccggatccggcggctggtcgagctcggcctgagcctggacgaagtcaaggacgtactggcggacgacgcgggcaaggacctcgtcgagatcctcaccgagctggacgccgacctggcacgccaggaggaggccatccggcagcgccgcgcccgcctggcccggctgttcgaacaggccgagcggaccggccggctgccggaggaggcccccgtctcgcccgagctcgcggccgtcttcgacgacatggcccgcgcctccgcgcaggtacccgggccggagccggccatggcggccaaggaacggcagctcctggctctgctggagaccggctcgccgggcggcgaccgcggctggttcgacggtctggtgcacgccttccgggccgacccggcggcgatgcggcgcgcgtacgacgtctatgcccggatggacgagctggccgaggccgccgaggacgacccccgggtggagctgacggcccgggccatcgtcgacagcatcccggacgaggcgctgcgggcgatgcgtctgccggccgacggcgccgaggaagcgggcgacgggttctccgacgccttcttctccgacttcgcacccgcccaggccgccgccgtgtaccgcgccatcgaactgctcacggagcgtgccgggtga

SEQ ID NO: 38 - DNA, 924 bp, Streptomyces sp.
  source 1..924: mol_type = genomic DNA
gtgaacacgctgaggaaggccgtgcgctggtccttcgccgcactcgtgcccggcgagctgatactcgtcctgtgtgtggccggcggagtgcgcatcgccccggccgtccggctcgccacggagccggccatgctcacgctcgcggccgcggcggcggtgctgatcgctctcgaccaccgccgccaccgccgcgccgggctcggccaccgcgccgccttcgccgccgcgatcgccgacaccgtcccggcgccggtgcgcaagctcaccgcccacgaggtgttcctctccaccagcttcctgcgctgggtgacccgccgcggcccgcacggggtgcgcgacggcgacctgtccgtcccgtacgcgccgggacagacggcgaccatgtacggcttcctgttcgtctgcgtcgtcgagaccgtggggttcgccttcctgatcccgtggccggtggtgcacgccgtgacgctcgtcctcgacatctggggctgctacttcgtcatcgccctgcacgcctcgtgcgtggtgcgcccgcacgtggtcggcgccgacggctcgctgcgcctgcgtcacggagccctgctggacatccgtatacccgccggccgggtggccgccgtccgccgggaacagaagttcccggacggcaagctggcagtcgtggacaagaacggtgtcgccgacatggccgtggccggccggacgacggtcctcgtcgaactcaccgaaccggtgcgcttcgtacgcgccctgggcaagaccgccgaggcccgcgccttccgcttctacgcggaggaccccgcggccgtcgtcacggcgctgaaggccggtgccgcgtctccctgcttggcctcgacccgcccgagcgcgtccgggccggacgccagcgcctcgcacgccgcctcccaggccgggtccccggggtag

SEQ ID NO: 39 - DNA, 1017 bp, Streptomyces sp.
  source 1..1017: mol_type = genomic DNA
atgcatgcatcgaattccccggccggcaccctcggctcacccacccccgtcctctcggtcggcccggtggtgctgcccgtgcccggccgggccgtggacctccagctgcggatctccgcgccggtcaccgggagcgacctgccggtcgtcctcctctcgcacggccagggttactcccaccacctctcctcgctgaacggctacgcgcccctcgccaactactgggcggcgcacggcttcgccgtcatccagcccacccacctcagttcccggaccctcgacctggaccccgccaccccgggcgcgcccctgcactggcgctcgcgggccgaggacatgcggcacatcctcgaccggctcgacctgatcgaagcggcggtcccgcagcttccggggcgcctggaccgcgagcggatcgccgtggccgggcactcgatgggcgggcacaccgcgagcctgctgctcggcgcccggctcaccgacccggacgacggaacggtggtggacctggccgagccccggatcaaggcgggtgtcctgctcgccgcgcccggcaggggcggtgacgccctcaccgcgtccgtggccgaggcgttcccgttcttcgcgaccacggacttctcccggatgacgacgcccacgctcgtggtcgccggggaccaggacgacaccgcccacctgacggtccggggtcccgcctggcacgccgacccgtacgtcctctcccccggccccaagtccctgctcaccctgttcggcgcggagcacgggctcggcggggtctccgggtacgacgtcgccgagaccaccgacgagagccccgagcgggtggccgcggtgcagcggctcacctgggcctacctccggtcgcggctctaccccggggacccggcctgggaggcggcgtgcgaggcgctggcgtccggcccggacgcgctcgggcgggtcgaggccaagcagggagacgcggcaccggccttcagcgccgtgacgacggccgcggggtcctccgcgtag

SEQ ID NO: 40 - DNA, 192 bp, Streptomyces sp.
  source 1..192: mol_type = genomic DNA
atgaacaccggcaacacgacggacaccccggcagccgcccggcaggccaagttcggcaagctgcccgagcgcatccgattcgaggacatggtcacggagcggccctcaccggagagccgcacgccgaacgcgtacacccccgaaggcacgtggaaccacttctcgtgcctggccctggatctcggcctgtag

SEQ ID NO: 41 - DNA, 564 bp, Streptomyces sp.
  source 1..564: mol_type = genomic DNA
gtgaacaccgaccatgtcttcgtctgcgggcaccccgccctcgacttcgccgcgacgctccgcgcccggcgcaccgtccgcttcgagatgttcgcgacgccggagcggctggaggcgtggtacgtggagtccgggctggtggattccgtgacgcccgcccggccgtccgacgtggagcgcgccaagtccgtacgggaggcggcctacgagctgatcaccgcgcgtcgcctcggggagccgttcggcgcccgggccttggacgtactcaacaaggccgcacgaaagccctcggccgcgccgcaactgacgagcgccggccgccgcaccgacgccaccccggaggaagcgctgtccgccgttgcccgccaggcggtggagttgctgagtgggccggacgtgccgctgctcaaggagtgcgggaatcccgagtgcacgcgggtctacatcgaccgttcgcggggcgcgcggcggcagtggtgcggtatggagtcctgcggcaacaagatcaaggccgccgcctaccgtgcgcgcaagaggggcgcacccgccacgaccgcgcgctga

SEQ ID NO: 42 - DNA, 1758 bp, Streptomyces sp.
  source 1..1758: mol_type = genomic DNA
atgccccgacccgccctgctcgcccatgatctcgtccgtacgctggggacccgacgggtcctggacggcgtctcgctcaccgtctcccccggccggcgcgtcggtatcgtcggcgagaacggggtcggcaaatcgaccctgctgcgcctgctcgcgggcgtggacgaacccgactccggccaggtcacccgcccggacgacctgggcttcctgcaccaggagatgccgttcgacggtgccacgacgatcgcgcgggtgctggccgacgccctgcgggaggcccgcgccgccctggccgaactcgaccggctgacccggctgctcgccggcaccccggacgaccaccccggccacgccgaactgctggacgcgtacgggaagcagctcgaacacgcccaggagagcgaagcgtgggacgccgaccggcgggccgcgatcgtcctggacgggctcggcctcggcgggttcggacacggccggacgctcggttcgctgtccggcgggcagcgcggccggctggcgctggccgcgctcctcgtgcgccggcccgccgccctcctgctggacgagccgaccaaccacctcgacgacgaagcggccgccttcgtggaggagcaggtccgcgtacagcccggcgtcgtcgtggcggccagccacgaccgggcgttcctcgacgcggtctgcaccgacctgatcgatctcgacccggcggtgcacggtccggtccgcttcggtggcaactacagcgcctaccgtgccgagaaacaggcggaacgcgagcgctgggaacagcggtacgcggcggagcaggaggagttggaagcgctgcgccgctcggccggggtgaccgcgcgccgcgtcgccccggaccgcgggccccgggacaacgagaagatggggtacggccaccgggccggccgggtgcagcaacaggtctcccggcgggtacgcaacgccgcccgccggctggacgaactggaacgcacccaagtcggcagaccgccgaagccgctgcgcctgcacgccggggcgctgacgggcgcgctggtggacggtgctccggcggacagcggtcaggtgcacggcgctacggcggacagcgctcccgtggacagcccgctcgtcgcgctgtgtgacgcgcgggtgcccggtcgcctggacgtcgcgcgcctggacatccccgccggggaccggcttctggtcacgggagacaacggggccgggaagtcgaccctgctgtccctgctggccgggcggctcgacgtcgagcgcgagggcgcggtacgcagacggcccggcctgaccgtgggcctgctggcgcaggacaccgtgttcgagcacccggaccgctcggcccgcgacacgtacacccatgtgctgggtgccgagcgggcggaggcggtgccgctcggctcgttgggactgctgggcgtcacggatctcgaccagccggtggggcggttgtcggccggccagcgccgcaggctcgcgctggccctcctggtggcgcacccgccgcacctcctgctgctcgacgaaccgacgaaccacctctccccgcgcctgtgcgacgaactggaggacgccctcggcgccgggccgggcgcgatcgtcgtggccagccacgaccggtggctgcgcgggcgctggaccggccgccggctgcggctgcgcgcgggccgcctggcgcctccggacgacgtgtacggcatacgcagtacgggaggcaggctccggcccggcacctccgaccggtga

SEQ ID NO: 43 - DNA, 1635 bp, Streptomyces sp.
  source 1..1635: mol_type = genomic DNA
atgtccgacgctttcgtcgtctgctccagcctgtccttttcctggcccgacgacacccccgtcttccaggacctgtccttcaccgtgggcaccggccgtacgggtctggtggcgcccaacggctccggcaagagcaccctgctcaagctgatcgccggtgaactgcgccccgccgccgggaccgtcaccgtctccggcaccctcggtcacctcccgcagagcctcccgctcgccgccggcctcaccgtggccgaggtgctgggcgtcgccgaggtcatccgcgccctggacgccgtggagtccggcgacgtggacgagaagcacttcgcgaccatcggcgacgactgggacatcgaggagcgcacccgcgcccagctcgaccggctcggcctcaccgaactgaccctggaccggggcctgagcaccctcagcggcggtcagatcgtctccctcggcctggccgcgcagctgctcaagcggcccgacgtgctgctgctcgacgagccgacgaacaacctggacctcgacgcgcggcacaagctctacgacgtactggaggactggaacggctgtctgctcctggtgagtcacgaccgggccctgctcgaccgcatggaccgcatcgccgagctggagccgggtgccctgcggctgtacggcggcaacttcaccgcgtacgaggaagccgtacaggccgaacaggaggtcgccgagaagaacgtccgcaacgccgagcaggaactcaagcgggagaagcgggagttgcagcaggcccgcgagcgcgccgaccgccgggcgagcaacgcctcacgcaacctcaagaacgcggggctgccccgtatcttcgccgggaacatgaagcgcggggcccaggagtcggcgggccgggccggccagatgcacgcctcgcgggtcagcgaggccaaggcccggctggacgaggcgggccgcgccgtacgcgacgagcagcgcatcacgctggacctgcccgacaccgacgtgcccgcggggcgcaacctgttcctcggcgagggcctgcgcgtcagccacggcgagcggccgctgttcgccgacggcggggtcgacctgacgatcagggggcccgagcgcatcgcgctgaccggccccaacggcgccggcaagaccacgctgctacgcctgttgcagggcggtctcgaacccgacgcggggcagatcaagcgggccgacagccggatcgcgtacctctcgcagcgcctcgacgtgctcgatctgtcccggaccgtggccgagaacttcgccgcgttcgcaccccagcgtacggcggcggaacggatgaacctgctcgcgcgcttcctcttccggggctcccgggcgcacctccccgtccaggtgctgtccggcggcgagcggctgcgcgccaccctggcctgtgtgctgtgcgccgaaccggcgccgcagctgctgctcctggacgagccgacgaacaacctcgacctggtcagcgccggccagctggagagcgcgctggcgtcctaccagggcgcgttcgtcgtggtcagccatgacgaacggttcctcgcccggatcgggatcgaccggtggctgcggctggccgacggggaactgcgggagacggcggtccccgaggtgtga

SEQ ID NO: 44 - DNA, 963 bp, Streptomyces sp.
  source 1..963: mol_type = genomic DNA
atgcacgcgatccgtatggaccggcacggtggccccgaggccatgaactggaccgccttaccggacccggcccccgggaagggcgaggtgctcgtgcggctcgccgtcgcgggagtgaacttcatggacgtgggggcccggacgtccggcggccccggctgggccgctccggcgattctcggggacgagggcgcgggccgggtgacagccctcggggagggcgttgagggcttcgccccaggtgaccgcgtcgcctggttctaccaccccggcagctatgccgaactgctcgccgttcccccggaatcgctggtcaagctgcccgacgaggtgtccgacgagaccgcggccgcggtcatgatgcagggcctgacggccagtcacctcaccaccgagacctaccccgtagggccgggggacaccgccgtcgtgcacgccgcggcgggcggcgtcggactactgctgacccagatgatcaaagcgcgcggcggccgggtgatcgggctggtgtcccgggaggagaaggtggcggcggcgcgggaagccggcgcggaccatgtcctggtctcccgtggagggggcttcgaagaccgggtcagggagctgacggacggcatgggtgcgcaggtggtgtacgacgggggcggcagtgcgacgttccgctcctcgcaacgggcgctgcgcccgcacggagttcacgcctactacgggccgttcatgggcgtcccgtccgtcgctccgaccgacctccccgacagcatttttctgacgtaccccgtggtgcaccaccatgtgcccacacgtgcggccctggtccagcgcacgggagaggtcttcgacctgatccgggacgggcggctgaccccgcgcatcggggggcgttacgccctgtcggacgccgcgcgggcgcatgccgacctggaatcccggcggaccatgggcaagctcctgctcgtaccctga

SEQ ID NO: 45 - DNA, 600 bp, Streptomyces sp.
  source 1..600: mol_type = genomic DNA
atgccgctgacccgtaagggcgccgccacccggcagcgcatcgtcgagggcgcggcctccgagatgcgccggctgggcatcttcgccctgacgctggacgacgtcatgcggcgcaccgcgaccagcaagagtcagctgttccactacttccccggcgggaaggacgagctgttgccggccgtggcccgccacgaggcgtccggcgtgatcagcgaccagcagccggaactggcctcgctgaactcctggagcgcctggcggagttggcgggacaagatcgtcgagcgctaccgcgagcagggtcccaactgtccgctcaacgtggtgatggggcagctcggcccgacgacggagggggcccaggccgtggtgcgggaactgtacgcccggtgggagggcgaactggccgccggcatccgcggtatgcagagccagggcgagatcgacgccgccctggacgccgggcgcggcgcacgggccctcctggccggcattcaggggggcgtgctgatccacctcgcgaccgggagcctggagtacctaaaagcggccctggaccaggggatcgagttcctccgccggccggcggcgagggcgtag

SEQ ID NO: 46 - DNA, 369 bp, Streptomyces sp.
  source 1..369: mol_type = genomic DNA
atggatggactgaccgagtcgaacaacggggtgcccgaaccgcacagcggctggacatttgtgaccaatcacgcccgggtgctggccgctatcgccgacgaccgccacgcccgtatccgtgacatcgcggcacactgccggttgaccgagcgcgccgttcagaagatcatctcggatctggagcgcgacggttacctctcgcacacccgcgagggccgcagcaacacctaccggatcgaccccggcaaggtgctgcgccacccggccgaagcgggcctgaccgtggcgtccctgctgtccctgctcgtccaggacgagcaggaccgcagcacgcaaccggacggccgccggcaccccagtacggcctga

SEQ ID NO: 47 - DNA, 738 bp, Streptomyces sp.
  source 1..738: mol_type = genomic DNA
atgcgacattccatgagggccgcgaagggatacgtcatgtccgcagtcattgaggtgcagcatctccgcaagtcctacggtgacctcgtagcagtcgacgacgtgtccttcagcgtcgaggaaggcgaggtcttcggcattctcggcccgagcggctcggggaagagcacgatcgtggaatgcgtcaaggggctgcggggccgggacggcggcgacatcaacgtgctcggcatcgacccccacgaggaccgggccgaggtgacgcggaagctggacgcccaactgcgggccgggtgctcgccggagcagatgcgcctcgccgaggcgctgtccctctacagctccttctaccgcgatccggcggactggggacgcctgctggccgcgctgggcctggccccggaggacgggccggacgaggcggccggcggggcggcggagctctccaccgcgctgcccctgatcggcaacccgcaggtggccgtcttcgacgagctgaccaccgaactggacccgcggtcccggcaggacacctgggagctgatcgagagcgtgcgggccagcgggctcaccatcctgatggtcacccggttcctggaggaggccgtgcggctgtgcgaccggatcgcggtgatcgaccagggccgtatcaaggcctccggcaccccggccgggctggccgaccacctcagggccgagcgttcggttgggccggacctgcgcctttcaccgcgctga

SEQ ID NO: 48 - DNA, 1005 bp, Streptomyces sp.
  source 1..1005: mol_type = genomic DNA
gtgagtctcgcacagacagtcggaacagcggccggagtcgccggccggggaggtggcgagccggagattgtctcggtcccgatcatccaggtcctgcccggagactcaccccgcctgacaggtgaggacaaagggcatgtgctgcggctggccgcgatagagggcgcgctgccccccatattggtcgaccggcgcaccatgtgcgtcatcgacggcttgcaccgcttgatggccgcctccctcaagggtcagaaagacatcgatgtcagattcttcgacggaagccgcgaagaggctttcctcaaaagcgtcgaggcaaacgtcgaacacggcctcccgttgtcgcaggccgaccgcacggcggcagcgctgcgcatactcaagtcgcaccccaccatggccgaccgggccatcgccaagtcgaccgggctgggcgcccggacggtcgcacagctgcggcgttcgtccgactccgtaccgcagtcggacttccgcgtcggacgggacggcaaggtccgcccgctggacggggcgcagggccggctgcgggtggcggagctgatgcgggacaaccccgacgcctcgctgcgccaagtggcccggtcggcaggggtgtcgccggccaccgcgctggacgtacgccgccggctggaacgcggtgaccagcccgtccccagccagcggagccgtagcaggggcgaggcgggacccgccaagcccgagccggtgggcccggtccccgtaccggtcgccgccccgccgaccgccgccacactcgacaaactcatgcgcgacccgtcactgcgtcagagcgagctgggccgacggctgttgcggatgttccgggagaatgtgctgggggccagcgagatgaacgacatctgtgcctcggtgccttcgcactgcatcgagctgatcgtggagctgtcccggcgcaactcgcagatctgggacgggttcgcgcaggagctgaacaggcgcgcccggatcgtgaatccggactga

SEQ ID NO: 49 - DNA, 324 bp, Streptomyces sp.
  source 1..324: mol_type = genomic DNA
atggggcacaaattcaccctggtcctgagtcgcgagatcacggacgaggagggggaacttctgaaggaagcgggtttctccggcgcgactttcgcgaccgacacgcttcccaccgacgccgatgtcaccgtgacgaaggtcgacttcgacgacacggccgcgccctcgctggccgaggcgatcgaaacggccctggaaacggtgaagaaggtccctgacctcaccgtgcccggcctcaccgtgccggccgttccgcagccccccgccgccgaggacggggacgtgctggtcggcgaggtggtcgcggagaccgccggcaagtaa

SEQ ID NO: 50 - DNA, 747 bp, Streptomyces sp.
  source 1..747: mol_type = genomic DNA
atgacccagccttcgcccggagacgaagtgtggtgcaggcgttttcaccctgccgacaagccgaccgcccggctggtgtgccttccgcacgcgggcgggtccgcgcccttcttcctgccggtggcggcggagctgagcccggacgtcgacgtcgtcgcgatccagtaccccggccggcaggaccgccgcgccgaggcgcccgtcgacgacctgtcggtgctcgtcgaccgtatccaccaggtactccggcgccagcccgacctcccgctcaccctcttcggccacagcatgggtgccgtcatcggcttcgaactggcccgccgtctggaggccgacggccggcctccggtgcggctgttcgcctccgggcggcgcgcgcccggcacgcaccgggacgagtcgaccttcaccagcgatgcggcgatcatggcggaggtgcgccggctcagcggcaccgccgccgccctgctggacgacgaggagatgatgcgggccgcgctgcccgcactgcgcgccgactaccgggcgatcgaggcgtaccggtgcgggccggacgccgtcgtggacactccgatcaccgtgctgaccggggacaacgaccccaagaccacgctggacgaggcgaaggcgtgggaccggcacacccggggcggctgcgatctgcgggtgttcgccggcggccacttcttcatcagcgagcgcggtgcggaggtcatgcaggttctgaaggagcacttcggcaagtaa

SEQ ID NO: 51 - DNA, 456 bp, Streptomyces sp.
  source 1..456: mol_type = genomic DNA
atggcggacaccaccgcgaccacgacggccccggagacctacgaggacccccgcgtccaggacaccttcaagacggtgcacaagttcatcgggctgtacggcgtgatcagcgtcatcgtgctcggcacggtggccgccatggccatcaccgggaacgaggccagccccttcatgtggatccggggatccatcctgctgctcgtctggccgctcttccaccggatggccacccgctcggcacagggggacgcgcgccagtacgaccggctggccacggtgaccgtgatcctgccgatcgcgatcatcgtcgtcgacctgatccccgggctctgcccgccctggtacgccgccatgcagggcgtctccgccctggcgctggtcggtgccgcggtcctcacccgcaaggccgaggtgcgcaacgtcttccccaagctggccaagaagaagaaggcctga

SEQ ID NO: 52 - DNA, 1326 bp, Streptomyces sp.
  source 1..1326: mol_type = genomic DNA
atgaggatacctccgcgaggcggcccgctgcgctggtgtgccgaggtcggcctcgggctgtaccggtccgtggtgctggtcgggctcacctgcgtggtgccgctcgtcgcggtgcccgtcggggtcggcggtgcccggctggccggctcctggaccgcgggtctggaggcgccgctggcggtgcgggtactggccaccctcgggcaggggctggcggtggccttctggatcgccgctgccctgttcatcgtctacccgctgaccagccgtgccctggccagggccgcccgccggtgcgccgaggactggctggaggtacggcacgaggtgcagtaccgcacggtgccgccggtgacccgtatggccaccggttcctggtgggacggggaggagtaccacaagaccgagcgggaggcccgccggcgggccgcggtgcatgcccgcttccacgacccgcagctgcactgggacgggctgtgggcggtggtcgcctcggtgaccgtgctgccggtctccgccttgccgctgctcgccctcggcctcggcatctacctggcggtgaccccggccctcctcggttacggcgtcgccttgatcgtggcgggcctggccgccgcccccttcgcctggcggatcctgcggccggtggcgacccggctgctggggccggtcccccgcacccggctgggccggcgggtcgaggaactggaggccatccgggccgacctgacgcaggcccaggccgcggagctggagcggatcgagcgcggcctgcacgacggggcgcaggcccggatggtcgcgatgggcatgtcgatgggcgccgccgagcagctggtggactccaacccggacgccgccaaggcggtgctggccgaggcccgcgccacctccgccaccgccctggccgaactgcgcaccctggtccgcgggatcaatcccccggtgctcgccgaacgcggactggtggacgccgtccaggcgctcgcgctggacgccgccgtcgtcgtggacgtgctcacccgggtgccctcccggccggagcgcccggtggagtcggcggtctacttcgccgtcgcggaactgctcgcgaacgtggccaagcacgccgaggcggagcgcgtcacggtggaactcacttatgccacgcggacgctgaccgcgaccgtgcaggacgacggggtcggcggggccgatcccgcctccggctccggactgagcggcatcgaacgccgcgtgcacgcgttcggcggccgtctggagatcgacagtccggccggaggacctacccgtatcactgtggcggtgccgtgcgaattgtcgtag

SEQ ID NO: 53 - DNA, 618 bp, Streptomyces sp.
  source 1..618: mol_type = genomic DNA
atggtgcagctcgtcgaggcgttcgggcacaccgtggtcgccgccgtcgagtccgggcccgccaccttggaggcgctgctcgaacaccggccggacgtcgcgatcgtggacgtccggctgccgccgaccttctccgacgaggggctgcaggccgccctcgccgcccgccgcgccgtgcccgggctgccggtgctgatcctctcccagcacgtcgagcagctgtacgcgcgggagttgctggccgacgggcacggcggcatcggctaccagctcaaggaccgggtgctggacgcggagcagttcctggacgccctgacccgggtggcgggcggcggcaccgcgctggatcccacggtcgtctccaagctgctggcccggcccacccgggccgatccgctcgccgccctcaccgagcgcgagcgctcggtgctggagctgatggccgaggggctgtccaacgcggccatcgccgggcggctgttcctgagcgagggcgccatcagcaagtacaccaccacgatcttcgccaagctgggtgtggtcaccgacgacgacaccaaccgcagggtccgcgccgtcctggcctacctggaggcgaccggccgcagcgaacagtga

SEQ ID NO: 54 - DNA, 3177 bp, Streptomyces sp.
  source 1..3177: mol_type = genomic DNA
gtgaacgattttgcgcaggaccacacgcccggggcggcggacggcggggcacccgcccccggcacggcggagaccgccgtctccccggccgaagaggcgctgtggcggctggaggaactggcgccgggcaccccggtgaacaacgtctcggtggccttcgccgtcgccgggcgcgtcgacgccgaacggctccaggcggcgctggccgccgtggcggaacgtcaccaggcgctgcgcaccggctaccgcgacgcaggttcccggctggtcaagcgggtgctggcggcagccgagtgcccgctggtgctggaagagacggagctgcccggtgaacccacgccggccgacctcgcgccggtgaccgccgcccccttcccgctggacggcaccccacccgtacgggcggcactgttccgctccccggacaccgacgtcgtctgcgtcaccgcgcaccggctcgtcctggacgagatctccggtccgctgctgcgcgaccacctgaccgccgcctacgaggagaacattggaacggtgggtacggcggagaccggcccggccgccgaggcgccgagcctggccttctggcggacggagctggccggcgcgaacaccgacgcccaggagctgtcctgcaccgcccctgacccggaccggccgaccctggccgccgcacagctcgaccgcaccctgcccgcgtccgccgaggccggcctccgccggctcgccgatgacctcggcgtccccccgtccgcgatcctgctggccgcctactacgtactgctggagacccacggcgccggccccgacctgctggtcggcctccccgtcgacctccgtcccgtacccgccgcccgcgcgctcggccggtacaccggcgaggtgccgctgcggctgcggaccgagcacaaggagagcgtccgcgacctggtgctgcgcagcgccgaacggctggccgccgccaccgctcacgccgcggccgccgaggcggagctggccggcctgctgcccccggccgcccccggcaggcccccgctgcccttccggcacaccttcgaacaccgtacggtcacctgcccggccgccttcaccttcggcggcctggacgcccggcccttccccgtcaccagcggcaccgcccgcttcgacctgcacctgacggcaacggccccgcaggacccgcacggcgccaccgcgctgcgcctgcgctaccgcaccgagctgttcgaccccgcgctggccgaactcctgctggcccgctacgagacggtgctgcgcgcgttcaccgccgacccggaccgggccgtcggcgacgtgtcctggtggagcggacgggaccacgagatcattggcgcggccaacgacaccgcgggtcccgtcgagccggcaaccgtcctggacgccgtccgcctgcaagtccggcgcgcgcccgacgcggtggtcgtggtggacggtgaacgcaccgtcggatacggacagttgtggaacgccgcacacgccatggctactcgcctgaaggacatcggggtgcggccgggggacgtggtggcgatcgccctgccacgtggcgccgaactggtcgcggcagtcctgggcacctggctggcgggcgccgcgtacgtaccggtggacgcggcccacccgcgcgagcggatccgttaccagctctccgactccgccgccacggtgctggtggccgacgagcacaccgccgccctggccggccccgacggcccggcggtgctgcacgcgccgccggcggaccacgccccgcaggtgcctgccaccgacgccccggtcggggaccccgccgcctgcgcctacctgatgtacacctccggatcgaccgggcgcccgaaggggacgctgatcggccacgcagccctggccaacctggccgcgcacttcgccgcccaacttgccgcggcacccggcgacaccatgctctggacgacaaccttcgccttcgacatgtccggggtggaactgttcgtaccactggtctccggcggccggctggtggcggccccggaccgggccaggagcgacggccgggtactgcgcgaactgctggagcggcacgacgtacgcttcgtccaggcgaccccgacggcctggcggctggtcgccgaccgggcggcggaccgcctgcgcggccgcagcgtcctgatcggcggggagccggtgccgctcccgctggcccggcggctgcgggcggccgggtgcgagctgcaccacgcctacggccccaccgagacgaccatctggtccacctcccgcgtgatcgatcaggacccgggcgcccggctggacgtgggccgcccgatccgcaacacccgggtccacgtgagcgacgcccacggccgcgacctgccggtgggcgtccggggggagctgtgcatctccggaaccggcgtcgcgctgggctaccacggccgtccggagctggacgcggagcgcttcggcgtccaccccgaatacggccgtttctaccgcaccggcgatgtggcctgctggcgggcggacggcctgatcgacctgttcggccggatcgaccggcaggtcaaactccgcggcaaccgcatcgagttgggcgagatcgaggcgacgctgacggcccacccgcaggtcgccgccgccgcggtgatcatggtgggtgacccgagcgacgacggtgtgctcatcggctgcctggagccgtccggtgacgtactggacaccgccgacgtccacgagcacgcccgcgcccggctgtcccgctcgatgctccccggcgacctcgtcaccgtcgacccgctgccggtcaacgccaacggcaaggtggacctccgggaactggagcggatcgtcaccgcccgccgcacggcgccgggacccaccggcaccagcccttcggacgccgccggccgaccgggaacggcggccggagcggacgccctcacggaccagctggtgcaggtgtggcgcaccgtcctgcggcgcgcggacgccgacgcggacaccgacttcttcgccgccggcggccactccatgctggccgccgtggcgatgcaggagttccagaacctcagcggccgctcgctgggcctctccgagatcttcgagcagtccaccccgcggcagctggccgcccgcatccgcgcgacggacacggcagccgagggggcgccgcagtcatga

SEQ ID NO: 55 - DNA, 12117 bp, Streptomyces sp.
  source 1..12117: mol_type = genomic DNA
atggcgaacgaagagaagcttcgcgactatctcaagcgggtcaccaccgacctcgctcagacccgggagcgtctgcgggagatcgaggaccgccggccggagccgatagccattgtcggcatggcctgccggttccccggcggggtgtcctcggcggaggggctgtgggagctggtggagtcgcggggtgacgggatcggtcctttcccgtcggaccgtggctggccggaggatgtggtggatcctgacccggacgcggtgggtaagagcgtctccggtgagggcggcttcctgtatgacgcggctgagttcgatgcggagttcttcgggatcaacccgcgtgaggcgctggcgatggatccgcagcagcggttgctgttggagacttcctgggaggcgttggagcacgccggcatcgacgccaccgcactgcgcggcagcccgaccggcgtgttcaccgggctcttctaccacgactatctgccgacccacccgatgccggaagagatcgaaggctacgtgggcgtcggggcgacggcgtcggtggcttccgggcgggtggcgtacgtgctgggcctggagggcccggcggtgacggtggatacggcctgctcgtcctcgctggtggcgttgcacctggctgcccagtcgctgcggtccggcgaaagcaccctggcactggcaggcggggtgaaggtgatggccacccctaacacgttcgtcgacttctcccgtcagcgggccgtggctctggacggccgctgcaagtcgtacgcggagggcgcggacggtactgcctggggcgagggcgtcggcgtgctggtcgtggaacgcctgtccgatgcccggcgcaacggtcacaaggtgctggcggtggtgcggggcagtgcgatcaaccaggacggtgcctccaacggcctgacggcaccgagtggtccggcacagcagcgggtgatccgggcggcgttggccgacgcgggcctggagccggttgatgtggatgtggtggaggggcacggtacggggacgcggctgggtgatccgatcgaggcgcaggcgctgctggccacctacggtcaggaccgcccggaagggcgaccgctgtggctgggctcggtgaagtccaacatcggacatgcccaggcggctgcgggcgtggcgagcatcgtgaagatggtggccgcgatggagcgaggggtggttccggcgacgttgtatgcggagcggccgtcttcgcaggtggactgggatgcgggggcggtgcggctggccactgaggcggtgccgtggccggagacgggtgagcgtccgcgtcgggccggcgtctcctcgttcgggttctccgggaccaatgctcacgtgattatcgagcaggccccggccgctgatgaggaggtccctgccccgggggcggctcccgaagccgcggacggtgttgacggtacggagttgcctctgttgccgtgggtggtttccggtcgttcggaggcggggcttgcgggtcaggcggggcggctggcggaggttgtcgaggcgtcggggcctggtgtgcggccggtggatgtgggctggtcgttggcgtcttcgcgtgcggtgctggagcatcgggcggtggtgctgggctcaagccgggaggagttggtcaccgctctgggcgccctcgcggagggtcgtgatctgccgggtgtggtgagtggggctgcgggcggtgcgggcaaggttggttttgttttcaccgggcagggtgcgcagcgggttgggatgggccgtcagctgtacgaatcgttcccggtgtttgcggcggcgttcgatgaggtgtgtgcggggctggatgcgcatcttgagggttcgttggcggcggtggtgcggggtgagggggacccgtcgtggccgggtgggggccgtattgatgagacggtgtgggcgcagcctgcgctgttcgcggtggaggtggcgctgttccgcttgctcgcgtcgtggggtgtggtgccgcaggtggtggcgggtcattcgatcggcgagctggctgcggcgcatgtggcaggggtgtggtcgctggaggatgcctgcgcggtggtggcggctcggggccgtctgatgcaggagttgccgtctggtggggcgatggtggcggtggaggccgccgaggaacgggtccgcgaagtcatcgcgggtcgtccgggggtcgggatcgccgcggtgaacgggccgcgcgcagtcgtggtgtccggcgtcgaggacgacgtgctggcggtggcggaggagctggcgcaggccgggtcgcgcacgcgtcgtttgcaggtctcgcacgcgttccactcaccgctgatggagccgatgctggaccggttcgctcaggcgatcgccgaggtcacgtaccacaccccgacggtgacgatggtctctgcgttgaccggcggccttgtttcggaagaggtcaccgatccgtcgtactgggtccggcacgtccgtgaggcggtccgcttcgcggacgcggcggagtcgatgcgcgggctcagggtggggaccttcatcgaactcggcccggacggcgtcttgtccggcatgggaccgcagacccgctccgacagtgagggcgaaggcgattcgggttctgaggcgtggctgccggtgctgcgccgtggtcgtgacgaaccgcgctctctgctgaccgcgctggcgaaggtgttcgtccgtggggtcccggttgcctgggacgcgctgtacaccggtaccggcgctcagcgcgtggatctgcccacgtacgccttccagcgtcatcgctactggttgaaggtggccgttgacagtcgggccgaggggctggggctggcgccgtcgggccatccgttgttgggggcggcggtggggttgccggccagtggtggtgtggtgttgacggggcggctttcgctttcggggcagtcctggcttgcggatcatgtggtggcgggccaggtggtggtgccgggcgcggcgttggtcgagatggtggtccgtgccggtgatgaggtggggtgctcgcgggtcgaggagctgttgattgaggcaccgttggtgctgcctgcgcggggtggggtgcgggtgcaggtcatggtggaggagccggacgcgtccgggcgccgcgtggtcagtgtctatgcgcaggacgaggacgtcccgccggagggggagtggacccggcacgcaaccggcacgctctccccagccgaggcggtggcggagggcagtgccgacggcttccggcagtggccgccgtccggtgcgcaaaccctcgacccggatctgttctacccggagctggcggctcgcggactgagttacggaccggccttccagggtgtgcgggcggcctggcgccggggcgaggaactgttcgccgagatcgccctgccgcaggacgtaacggccaacgggttcgggctgcatcccgccctgctggacgcggcactgcatgtggcggccgcgaacaccgaaggtgacggcctggcgctgccgttcgcctggaacaacgtcatggtgcacgcctccgacgccgttgcggcacgggtccgggtgacctctgccgcctcgggcgaggggctgacggtcatgctggccgacgcgtccggccagctgatcgcctccgtcgacaggctggacctgcgtaccctctcgccgcaggacctcgcagcgggcgggcaggcggcggacgacaccctgttcgcggtggagtggatgcccgcctcggccacatccgacgcgggcccggactcggacccggcggcgttgggccggtgggttgtgctcggtggttcggaggtggtgccggatctgcctggtgccgtggtcttcccggatgttgcggcgttggtggcggcggtggaggcgggggctgaggttccgcagatggtggtggcgccggttcgtccggtggccgggcccggtggcactgcggaggctgcccgtcgtgccgccgtggacgttctggggttggtgcaggagtggttggctgctgatgtgttgtccggtgcgcggttgttggtgttgaccgagcgtgcggtcgatgccggcgcggaggtgccggtggaggttgcggcggcttcggtgtggggtctggtgcgggtggcgcagacggagaatccggggcggctgctgctggcggacgtggacgaggtgtccgtacccggtgcggaactggccggctggctgcgagacggggtggagctgggcgagccccagtgggcggtccgcggcggacagacccgggtgcccaggctgacccgggccactctcgcggacccgtcggcactcaacggggacacggcacctgaggcccaggcggtaccgtccgccgggcccgagatcgagaccaggcccgagaccgagaccgggcccgagaccaacattgacgcccgcccgggcacggtgctggtgaccggcgcttcgggggtgctgggtgggttggtggcccggcatctggcgaggtcgggccgggcggaacacctggtgctgctctcccgtcgcggcggcgacgccccgggaatgccggaactcctcgccgacctcgacgcatcaggtgtccgggtcaccgtggccgcctgtgatgcagccgaccgggaccagctttccagtgtccttgagggcgtgcccctgacgggtgtggtgcatgcggcgggtgtgctggatgacggggtggtcggctcgctgactgcggagcggctggagtcggtgctgcggccgaaggtggatgcggcctggaacctgcacgagctgacccgtgacctgggcctggagctggacacgtttgtgttgttctcgtccatcgcgggggtgatcggtaatgccggtcaggccaactacgcggcgggcaacaccttcctggacgccctggccgcccaccggcgccaccacggcctgcccgccgtatcgctggcctggggaccctgggagaacggcatggccggcgaactggccgaagccgacctgctgcgaatggcccgccagggactgcgcccgctctccgacgcacgcggcctcgccgcgctggacgccgccgcggaccgcgcccgaccgctgctggtggcagcagagttgcatctcggtgcgctgcgccgctccgcagaggtgccgccgctgctgtccgcgctcgtcccggcggctcgcgggaacgcgcagcccgcccgccggacggccggccagccggccgccgaggaccgtaacgcgctggcagcccgcctggccgggctgagccccgcggagagccgggaggtgctgcaggacctcgtcatcggccaggccgccctggtgctgggcatggccggacccggtgccctcgacgggaaccgtccgttcagggacgtcggcttcgactcgctgaccgccgtcgaactgcgcaaccggctcagcacggcgaccggcctgcggctgccggccacggcggtcttcgactacccgacccccgtcgccctggccgaattcgccctggacgagctgatcggctccggcgcggtaccggcggacgcggcacctccggcagcggccgacgacggccgcgaggacgacgacccggtggcgatcgtcggaatggcgtgccgcttccccggcggggccacgacccccgccgagttctggcagatgctggcctcgggcggggacgggatcggccccttcccggacaaccgtggctggccggaggacgtggtggattccgacccggacgcgatgggcaagagcctggtcggggagggcggtttcctctacgacgccgccgagttcgacgcggagttcttcggcaccagcccgcgtgaggcgctggcgatggatccgcagcagcggctgttgctggagacctcctgggaggcgctggagaacgccggcatcgacccgaccgagctgcggaccagcgcgtccggcgtgttcaccggcatgttccaccacgactacctctcggccaccgcgctgccggaagaggccgagggatacgtcggcctcggcgccaccgggtcggtggcctccggacgagtggcgtacgcgctgggcctggagggcccagcggtgaccgtggacactgcctgctcctcctcgctcgtggcactccacctggccgcccagtccctgcggtcaggtgagagcacactcgccttggtcagtggcgtcaacgtgatggccactccggtcaccttcatcgacttctcccggcagcgtgggctggccgcggacggccgttgcaaggcgtacgcggaggacgcggatggcactgcctggagcgagggcgtcggcgtgctggtcgtggaacgtctgtccgacgcccggcgcaacggccacaaggtactggcggtggtgcggggcagcgcgatcaaccaggacggcgcttccaacggcctgaccgcaccgaacgggccgtcccaacagcgagtgatccgggcggcgttggccaacgcgggcatcgggaccgacggggtggacgtggtcgagggccacggcaccgggacgcggctgggcgaccccatcgaggcacaggccctgctggccacctacggccggaaccgtccggcggaccggccgctgtggctgggctcggtgaagtccaacatcggacacgcccaggcggcggccggtgtcgccgggatcatcaagatggtgctggcactggagcacggccagatccccaagactctgcacgcggagcggccgtcttcgcaggtggactggggtgcgggggcggtgcggctggtcactgaggcggtgccgtggccggagacgggtgagcgtccgcgtcgggctggggtgtcttcgttcgggttctccgggaccaatgctcatgtgatcatcgagcaggccccgagcgtggagggcgtgtctcttccgggggcggcgcccgaggccgtggacggtgtttccggtacggagctgcctctgttgccgtgggtggtttccggccgttcggaggcggggctggccgggcaggctgggcgtttggccgcgttcgtcggacaggactcgggtgtgcggccggtggatgtgggctggtcgttggtgtcgtcgcgtgcggtgctggagcatcgggcggtggtgctgggctcaagccgggaggagttggtcaccgctctgggcgccctcgcggagggccgtgatctgccgggtgtggtgagcggtgctgcgggcggtgcgggcaaggtcggcttcgtcttcaccgggcagggtgcccagcgggttgggatgggccgacagctttacgaggtgttcccggtgttcgcggcggcgttcgacgaggtgtgcgcggggctggacgcgcaccttgagggttcgctggcggcggtggtacggggcgaaggcgacccgacctggccgggggagggccgtatcgatgcgacggtgtgggcgcagcccgcgctgttcgcggtggaggtggcgctgttccgcttgctcacctcgtggggtgtgacaccgcaggtggtggcgggtcattcgatcggtgagctggctgcggcacacgtggcgggggtgtggtccctggacgatgcctgcgcggtggtggcggcccggggccgtctgatgcaggagttgccgtctggtggggcgatggtggcggcggaagccaccgaggagcaggtccgcaaggccctcgccgaccgccccggggtggggatcgccgcggtgaacgggccgcgtgcgatcgtggtgtccggcgtcgaggacgacgtgctggcggtggcggaggagctggcgcaggccgggtcgcgcacgcgtcgccttcaggtctcgcacgctttccactcaccgctgatggagccgatgctggaccggttcgcccaggcgatcgcccacgtcacctacgggaagccgcagctaacgatggtctcggccctgaccggtcgccccgtttcggaggaggtcaccgccccctcgtactgggtccggcacgtccgtgaggcggtccgcttcgcagacgccacagaggcgatgcgcgggctcggcgtgggcaccttcatcgaactcggcccggacggtgtcctgtccggcatgggcccgcagacccgcaccgacagcgagggccaggacacgaccggctccgaggcatggctgccgttgctgcgccgtggccgcgacgaaccgcgcgccctgctgaccgcactggccaaggcgttcgtccgcggagtcccggtcgcctgggacgcgctctacaccggcaccggcgcccagcgcgtcgacctgcccggctacgccttccagcggcagcggtactggctgagcgcggcctccgcgagccgcgccgaggacctcggtctggaacgaccggggcaccccctgctaggggcgaccgtcgagctgccggcctccggtgaggtggtgctgaccgggcagctgacccggtccgcacagccctggctgggccagctgctggtggccgggcgtccggtcgtcgccccctcggcgctggtggacatggcggtgcgcgcggccgacgaggccggcggcggccgcatcgaagaactgctcgtcgaggccccgctggtgctcccgcagcgcggcggcgtccgggtacaggtgaccgtcggcacggccgacgccgagggacgccgggaggtcgccgtgttcgcgcggcggccggagggggagtgggtgcggcacgccagcggcacgctggccccggccgcagcggacgacgaggccgccgcggacgcggcgtggagcgtgcactggccgccgcccggcgccgtaccggccgacgtgcccgggctctacgaggggctggcggccaccggcctcggccacggtccggccttccagggggtgcgggctgcctggcgccggggcgaggagctgttcgccgagatcgccctgccgcaggacgtggcggtggacgggttcggtgtgcacccggccctgctcgacgccgctctccacctgacctgcctggaacaggcggagcagcagcatcggccggatgccgaggggccgttgctgcccttcgcctggagcggagtgcggctgtacgcgtccggcgccacggcggcgcgggtacgggtggtgccgtccccgtccggtgagggcgtggcggtgacgctggccgacccggtcggccggccgatcgcctcggcggacgcggtggtcccggcgcccctggctcccggcgcgctggaacaggccgacggggcgggccgggacgggctcttcctggtgcggtggtccccggtggacgaggtgccggagcccggtgagccggggcgccggtgggcggtactcggcgacgacggcggactggaggtgcccggcgcgacggcgtaccgggacgtcaccgagctgctcgccgcggtgcgggacggcgctccggtgccggacacggtggcgctgtgctgcgtgtccgacagcgacccggaccccaccacggcccaggccactgacccggaccaggccacggcccaagccacggctccagaccccgccgcagacctggcccccacccccgcgacactccccgcagccgcacgcaccgccaccgtccacgtcctggagaccctccgcgcgctgctggcggcaggcgtgctggacggatcgcagctggtcgtggtggtcgagcgcgggctggacgtggacgccggcacggacctgcggctggcgggtgccggtgtcgccggactggtccggacggcggctgccgagcatccggagcgcatcgtgctggcggacgtggcgcggccggccggctccggtcccctggtgatggccggagcggccctgggcgaaccgcaattcgcggtgcgtgacggccggctgctggtgccgcgactggcccgggcagccggggaggaactgccggtgccggacggccgggactggcgcctggcggcctgcggacccgacgccgccggcctggtacggccggtggacggcgaggtggggcagcggcccctggccgccggtgaggtgcggttggcggtgcacgcggccgcggtgaaccggcaggacgtacggcacgtgctggaggcgtcccccgagccgtcgcgaccgctcggacgggagggcgccggggtagtgctggagaccggacccggactcgcctggccggctgtcggagaacgggtgaccggcctgttcccgggcgccttcgcggccaccgcgatcgccgacgcacggctgctggtgccggtgccggacggttggtcggccgcgcaggccgccgccgcgccggtggcgttcctgaccgcgtaccggctgctggcgcaggccgcaccgcgccccggggagcgggtgctgatccaccccgccgccggccccgagggcctggccgccgtccagctggcgcgccacttgggcgccgaggtgttcgccacggcgccgccgtccgcctcgccggtcctgcaccggctcggcctggacggacagcacctggcgccgtccgcgacggcacacttcgaggagacgttccgtaccgcgaccgacggacacgggatggacgtcgtgctcaccttcctcccgacatccgccgacccggccacagcgacagcggccccagacacagcaacgacgaccccagacacagcaacgacagacgcagcactccgcctgctccaccccggcggccgttcgctgtccgcagccgccgccgagacgggccaccagcaccccgaagagctcgaagcccacctggcagagctggccgggctgttcgagaagggcgcgctgcaccccctgccggtggcctgccgggacgtacggcaggccgcgcaggtcttccggacgccggtccgggaggcgtccacggtcgccaagacggtgctgacgctgccgcccgcaccggtcggcaggcccggcacggtgctggtgaccggcgcctcgggggcgctcggcggcctgacggcacggcacctggcccgtaccggccgggccgagcggctgctgctgacctcacggcggggcgcggccgcccccggaatgccgcgcctggccgccgaactggccgcctccggacactcggtgcaggtcctggcctgcgacgtcgcggaccgccgacagctcgccgccgtgctggccggggtgccggccacggccccgctgaccggggtggtgcacaccgcgggcgtactggacgacggcgtgctcgcctcgctcaccccggagcgtgtcgagcaggtgatgcggcccaaggtggacgccgcctggcacctgcacgacctgacccgccatctcggcctggacaccttcgtcctgttctcctccgtggccggtgtgctgggcaccccgggccaggccaactacgcggccggcaacgccttcctggacgcgctggccgcccaccggcgccaccacggcctgcccgccgtatcgctggcgtggggaccgtgggaggacggcatggccggacagctcaccgacgccggacggcagcggatgttccgtcagggactgcggccgctgtccgcggccgacggtctcgcgctgttcgacgccgctctcggcaggccggacgcgctgctggtcacgacccggctggacctgcccgcgctgcgccgcaccgcgagtgccgtaccggccctggtgaccggcctggtccggcccggccggcgcaccgcgggaggaccggcgccggacgccggccggaagctggccgccgagctggcggcgctgccgcccgagcagttccaccacgccgtcctgaacctggtgcgtacccaggcggcgctggtgctcggacggccgggaccgcagtccgtcgagcccgcccggtacttccgccagctcggcttcgactcgctgaccgcggtggagttccgtaaccgcgtcagcggcgcgctcggcctgcggctgccggccgcggtcgtcttcgactaccccacgccgcgggaactggcggagtacctccaggagcggatcgccgagcaggagatcgactacgggccggtgctcggcgagctggaccggctcaggtcgctgctcagcgggatcgtccggcggggcggccgcaaggcggagatcatgagccgtttcgagaccatcatggaggagttccgcggcgccgagacggaccagggctcctccgacacggacatcaccgaggcgacggacgacgagatgttcgacctcatcgacaaggagctggggatcgtgccgggcaccgacggaggagcggcgtga

SEQ ID NO: 56 - DNA, 14025 bp, Streptomyces sp.
  source 1..14025: mol_type = genomic DNA
gtgaacgacaccgatgggccggccctgaaggacaccgccgcagccggcgcacagcctttggcgatcgtcggtatgagctgccgctaccccggcggggtccgttcacccgaggacctgtggaagctgctggccgaaggcggccaggggctctccggcttcccgcgggaccgcggctggaacctcggctcgctgcacggccaggacccggacggcaccggaaccacctacctcacccgaggcgggttcctcgacgacgccgccggtttcgacgccgccttcttcggcatcccgccgcgcgaggcccaggccatggacccccagcagcggctgctcctggaggcgtcctgggaggcgctggagaacgccgccctcctccccgcagacctccgcggcacccgcaccggcgtgttcgtgggcgccgaacccagggagtacggcccccggccgcacagcgcaccgggcagcgccaagggccacctgttcaccggcacctcgcccggcgtgatgtccggccggatctcctacctgctgggactgcacggcccgtcgctcaccgtggacacttccgcctccagctccctggtcgccatccacctcgcggcgcaggcgctgcggaccggtgactgctcgctggcgctggccggcggggtgtccgtgatgtccaccccggggaacttcatcgccttcagcggcctgcgggccctgtcggccgacggggtctgccggcccttctcggccgccgccgacggcaccgtatggtccgaaggcgtcggcctgatcgtcctggagcgcctgtccgacgccgtccgcaacggccaccccgtgctggccgtactgcgcggttcggccatcaactccgacggcaccggcgagggactgaccgcgccgaacggcagcgcccagcaggccgtcatccgccgcgccctggccgacgcgcacctgaccgccgaccaggtggacgcggtggaggcgcacggcacggggacgcggctgggtgacccgatcgaggcgcgggcgctgctggccacgtacgggcaggagcgtcccgagggccggccgctgtggctgggctcggtgaagtccaatctcggccacgcccaggcggccgccggtgcagcgggtgtcatcaagatggtgctcgccctgcgccacggcgaactccccgcaaccctgcacgcacacgagcccacccccgaggtggactggtcctccggcggggtacgcctgctgaccgctccggccccgtggccggccggtgcccgcacccgccgtgccggggtctccggcttcggcctcggtggcaccaacgtgcaccttcttctggaggaggggccgactgtatcccccgcacaggatgccgaagccgtcgaggagtccggggggcctgcggttccggtggtgaccggtgccagcgcttgggtgctgtccgggcggactgctggggcgttgtcggcgcaggcggggcggctgcgggagtggatggggacgcgtccggagttggaaccggccgatgtggcgtggtcgctggctgccggccgctcggtcttcgagcaccggggtgttgtggtgggtacggggcgtgccgagctgctcggtggcgtggggagcctggcgtcgggtgttccgggtgggtcggtggtgtccggggtggcccggtcgggtgttcggccgggtgttcgtccggtgtttgcttttgctggtcaggggtcgcagtgggtggggatggggcgggagctggcgggggtttcgccggtgttcgcggggcggttggcggagtgtgctgcggcgttggcgccgcatgtggactggtcgctcagcgatgttctggcgggtgcggagggtgctcctgcgttggaggcggcggatgtcgttcagcctgcgttgtgggcggtgatggtgtcgctggctgcggtgtgggaggcggctggtgtcgctccggatgcggtggttggtcactcgcagggggagattgccgctgccacggtggccggcatgctgtcgcttgaggacggtgcccgtgtggtggcgttgcggtcgcaggcgctgaaggtgctggcgggtgcgggcggcatgctgtccgtgagccgtccggcgtcggaggtcgaggagcgcctggtccggttcggggagagggtgtccctggcggcggtcaacggaccgtcggccactgtcgtctcgggtgagccggcggctctggaggagcttcagtccgagttcgaggcggcgggggcccgggcgcggatggtggcggtggattacgcctcgcattcggctcaggtggaccggctggaggaggagatcacctctgttctggctgggatttccccgcgtcggggacgtgttccgatggtgtccgcgatgactggcgagacgctgacgggtgaggagctggacgccgggtactggttccgcagtctgcgggcgacggtggattacgaccgtgcggtgcgcgtgctggccggtcagggccatcagatgttcgtcgaggtgacgccgcatccggtgctggtcggcgccatgcacgacacccttcaggcactcgcccaggaggccggtcctggcgcgataccgggcgccgtctgcgggtcgctgcggcgggacgacggcggtgccgagcgcctgctggcctcgctggccgaagcgttcgtcaatggggcgtcggtggactggcaggtggtgcttcccgcagggaagcgggtggagctgccgacgtacgcgttccagcatgagcggtactggctcgacgccgacgctcccacgcagccggccgaggcgccgcagcccgcggacctcaccgctgccaccgacacgaccgcagggtgggggctgcgcggacagctcgccggccggccggagcccgaacagcaccagctcgttctggacctggtcatcacccaggccgcagccgtactcggccattcggcaccggcggccctggaccccgcacgtaccttcaaggaactgggcttcgactcggtgaccggggtcgacctgcgcaaccggctgagcaccgcggccgccctccaactgcccagcacgctgatcttcgaccaccccacaccgctcgcgctcgccggcttcctgcacggcgaggcgcagggccacgccccttcggccggggccgtcggctccaccaggacgccggcacctgccggaggtgacgagcccattgccatcgtggcgatgagctgccgtctgccgggcggcctcagcacacccgagcgcctgtgggagctgctggccgagggcggcgaggcagtggccccactccccgccgaccgcggctgggacctctcccggcagcacggcacccggcccgacggggacgcggcctccgcgagtcacgatgcgtccaccaccgggctgtccacccgtggcggtggcttcctcaccggcgcaggcgacttcgacgccgccttcttcgggatcagcccgcgcgaagcactggcgatggatccgcagcagcggctgctcctcgaactctcctgggaagccttcgagcgcgcaggcatcgacccggggcgcctgcggggctcggcgaccggcgtcttcgccggcgcgtccccgtccggatacggcagccatgcgcccgaggaactcacgggccacctgcagcccggcatcacccccagcgtgatctccggccgggtggcctacacactcggactcgaaggccccgcgctgacggtggacaccgcctgctcgtcttcgctggtggcgctgcacttggccgtccggtcgctgcggtccggcgagtgcgagctggctctcgcgggtggtgtcaccgtgcatgccaccacgtcctggctgaactggttcgaccgtcaacggggactggccgccgacggccgctgcaaggcatacgccgaccaggccgacgggatgggcatgggcgaaggcgccggcctcctggtactggaacgcctgtccgacgcgcagcgcaacggtcacaaggtgttggcggtggtgcggggcagtgcggtcaatcaggacggtgcttccaatggcctgaccgcgccaaatggtccgtcgcagcagcgggtgatccgggcggcgttggctgacgcgggcctggagccggttgatgtggatgtggtggaggggcacggtacggggacgcggttgggtgatccgatcgaggcgcaggcgctgctggccacctacgggcaggaccgtccggaaggccggccgctgtggctgggctcggtgaagtccaacatcgggcacgctcagtccgccgcgggcgtggcgggcgtcctcaaactcgtcctcgccctccaacacggagaagtgccgaggacgctgcacgccgaccagccttcttcgcaggtggactgggatgcgggggcggtgcgtctggtcactgaggcggtgccgtggccggcgacgggcgagcgtccgcgtcgggctggggtgtcttcgttcggcatcagtggcaccaacgctcacgtgatcattgagcaggccccgaatgccgaaggtgcgctcctttcgggggcggctccgggcatcggcgcggacggtgattccggtgcggagctgcctctgttgccgtgggtggtctctggtcgttcggaggcggggctggccgggcaggctgggcggctggcggagtttgtcgaggcgtcggggtctggtgtgcggccggtgaatgtgggctggtcgttggtgtcgtcgcgtgcggtgttggagcatcgggcggtggtgctgggttcgggtcgggaggagttggttgctgggctgggggcgcttgcggagggtcgtgatctgccgggtgtggtgagcggtgctgcgggcggtgcgggcaaggttggttttgttttcaccgggcagggtgcgcagcgggttgggatgggtcgtcagctgtacgaatcgttcccggtgtttgcggcggcgttcgatgaggtgtgtgcggggctggatgcgcatcttgagggttcgttggcggcggtggtgcggggtgagggggacccgtcgtggccgggtgggggccgtattgatgagacggtgtgggcgcagcctgcgctgttcgcggtggaggtggcgctgttccgcttgctggcgtcgtggggtgtggtgccgcaggtggtggcgggtcactcgatcggtgagctggctgcggcgcatgtggcaggggtttggtcgctggaggatgcctgtgcggtggtggcggctcggggccgtctgatgcaggagttgccgtctggtggggcgatggtggcggtggaggccgccgaggagcaggtccgcagggccttggccgatcgtccgggggtggggattgctgcggtgaacgggccgcgtgcggtcgtgctgtccggcaccgagaccgacgtgctggcggtggcggaggagctggcgcaggccgggtctcgcacgcgtcgccttcaggtctcgcacgcgttccactcaccgctgatggagccgatgctggaccggttcgcccaggcgatcgcccacgtcacgtaccggaagccgcagctaacgatggtctcggccctgaccggttgccccgtttcggaggaggtcaccgccccctcgtactgggtccggcacgtccgcgaggcggtccgcttcgcagacgccacagagacgatgcgcgggctcggcgtgggcaccttcatcgaactcggtccggacggtgtcctgtccggtatgggcccgcagacccgcgccgacagcgagggccaggacacgaccggctccgaggcatggctgccggtgctgcggcgtggccgcgacgaaccgcgcgccgtgctgacggcgctggcgaaggtgtttgtccgtggggtcccggttgcctgggacgcgctctacaccggtaccggcgctcagcgcgtggatctgcccacgtacgccttccagcgtcaacgttactggctgccgaccgcgtccgcccctctggacgcggcgggtctgggccagtacacgacccgtcatccgttgttgggggcggctgtgaaatcgccggccagtggtggcttggtgctcacggggcggctttcgctttcggggcagccctggctggcggatcacgtggtggcgggccaggtggttgtgcccggtgcggcgttggtcgagatggcggtccgtgccggtgatgaggtggggtgctcgcgggtcgaggagttgctgagcgaggccccgttggtgctgcctgcgcagggtggggtgcgggtgcaggtcatggtggaggagccggacgcgtccgggcgccgcgtggtcagtgtctatgcgcaggacgaggacgtcccgccggagggggagtggacccggcacgcaaccggcacgctctccccggtaagggaagctggcgacggacgtgcacacactcgcctccggcagtggccgtctcccggagccgagcccgtcgagctgaccgatttctattccgcgttgtcggcccgtggcctctcctacggtccggtcttccagggtgtgcgggcggcgtggtgtctgggtgaggagctgttcgccgaggtcgctctgccgccgggtgtggtggtggacgggtttgggttgcatcctgcgttgttggatgcggcgttgcacgtcattgtgggtgtgggggagcgtgcgggtgggttggaggtgccgtttgcctggcgggacgtggagttgtatgccacgggtgctcaggtggcccgggtccgggtggtgccgggagctgatggcgagggtgtttcgctcagcctggcggatgctgacggcgcgctgatcgcctccgtcggttccctggccctgcggccgttcacagctcccgcttccggtgcaggagacggcctgtgtgggatcgactgggtgcctgccacggccaccacatccgacctgggctcggccccggagccgttgggccggtgggctgtgctcggggctgtgtcgggtgtgccggatctgcctggtgccgtggtcttcccggatgttgcggcgttggtggcggcggtggaggcgggggctgaggttccgcagatggtggtggcgccggttcgtccggtggccgggcccggtggcactgcggaggctgcccgtcgtgccgccgtggacgttctggggttggtgcaggagtggttggctgctgatgcgctgtccggtgcgcggttgttggtgttgaccgagcgtgcggtcgatgccggcgcggaggtgccggtggaggttgcggcggcttcggtgtggggtctggtgcgggtggcgcagaaggagaatccggggcggctgctgctggcggacgtggacgaggtgtccgtacctggtgtggatctggccgactggctgcgggccggggttgagctgggtgagacgcagtgggcagtgcggggcggacagattcgggtaccgcgcctggtgcgcctgccgtcccggcttctcacggaggagatccgtccgggcacggtgctggtgaccggtgcttcgggggtgctgggtgggttggtggcccggcatctggcgaggtcgggtcgggcggagcatctggtgctgctctcgcgtcgcggcggcgaggcccccgggatgcctgggctcgtcgttgatcttcaggagatgggtgtcaaggtcacggtggctgcctgtgatgcggccgatcgggatcagcttgccggtgttcttgagggtgtggtgctgacgggtgtggtgcatgcggcgggtgtgctggatgacggggtggtcggctcgctgactgcggagcggctggagtcggtgctgcggccgaaggtggatgcggcctggaacctgcatgagctgacccgtgacctgggcctggagctggacacttttgtgttgttctcgtccatcgcgggggtgatcggtaatgccggtcaggccaattacgcggcgggcaacacgttcctggacgccctggccgcgtaccggcgccaccagggcctggccgccgtatcgctggcctggggaccgtgggaaaacggcatggccggtgccctgaacgaggtggaccagcgccggatgtcccgacagggactgcgcccgctgtccgcaccggacgggctggccctgttggacaccgccgtccgcaccgaccggtccctgctggtcaccactggactggacctggccgcgctgcgtcgttccggggaggtaccgccgctgctctccggcctgctgccggacccacgcggcggccactcgcagcccgcccgccgcacggttgccgaatcggccgccgacaaccggagcccgctggctgcacggctcgtcggcctcgcaccaggcgagcggaccgagactgctctccaactggtcgcgacggaggccgcgctggtactggggatgaccggaccggagaccgtcgaacccgcccgctcgttccgggacaccggcttcgactcgctcaccgccgttgagttgcgcaaccgactgaacagggcgacgggcctgcggctgcctgccacgaccgtcttcgaccacccgaccccggccacgttggcggcgcatgtgctggccgaactgctcggtgaggacgaggccggcccgatgggtgacaccgtcccggcggccaatgttccgcgtgcgggcgacgatccggtcgtcattgtcggcatggcctgccggttccctggcggggtgtcctcggcggaggggctgtgggagctggtggagtcgcggggtgacgggatcggtgccttcccgtcggaccgtggctggccggaggatgtggtggatcctgacccggacgcggtgggtaagagcgtctccggtgagggcggcttcctgtacgacgccgctgagttcgacgcggagttcttcggcatcagcccgcgcgaggcggtcgcgatggatccgcagcagcggctgttgctggagacttcctgggaggcgttggagcacgccggcatcgacgccaccgcactgcgcggcagcccgaccggcgtattcgccgggctcttctaccacgactacctgccctggcatgcggtcccggaagaggtcgagggatacctcggtaccggtagcacgggctcggtggcttccgggcgggtggcttatgcgctgggcctggagggcccggcggtgacggtggatacggcctgctcgtcttcgctggtggcgttgcacctggctgcccagtcgctgcggtccggcgaaagcaccctggcactggcaggcggagtcacggtgatgggaacccccggtacctttatcgacttttcccgtcagcgtgggctggctgcggacggccgatgcaagtcgtatgcggagggcgcggacggtactgcctggggcgaaggcgtgggcgtgctggtcgtggaacgtctctcggatgcccggcgcaatggacatcgagtgctggcggtggtgcggggcagtgcggtcaatcaggacggtgcttcgaacggtttgacggcgccgaatggtccgtcgcagcgtcgggtgatccgggcggcgttggctgacgcgggcctggagccggttgatgtggatgtggtggaggggcacgggacggggacgcggttgggtgatccgatcgaggcgcaggcgttgctggccacgtacgggcaggaccgtccggagggccggccgttgtggctggggtcggtgaagtccaacatcgggcatgcccaggcggctgcgggtgtggctgggattgtgaagatggtggccgcgttggagcgaggggtggttccggcgacgttgtatgcggagcggccgtcttcgcaggtggactggggtgcgggggcggtgcggctggtcactgaggcggtgccgtggccggagacgggtgagcgtccgcgtcgggccggtgtctcctcgttcgggttctccgggaccaacgctcatgtgatcatcgagcaggccccgagcgtggagggcgtgtctcttccgggggcggcgcccgaggccgtggacggtgtttccggtacggagctgcctctgttgccgtgggtggtttccggccgttcggaggcggggctggccgggcaggctgggcgtttggccgcgttcgtcggacaggactcgggtgtgcggccggtggatgtgggctggtcgttggtgtcgtcgcgtgcggtgctggagcatcgggcggtggtgctgggctcaagccgggaggagttggtcaccgctctgggcgccctcgcggagggccgtgacctgccgggtgtggtgagcggcgctgcgggcggtgcgggcaagatcggcttcgtcttcaccgggcaaggcgcccagcgggttgggatgggccgacagctctacgaagcattcccggtgttcgcggcggcgttcgacgaggtgtgcgcggggctggacgcgcaccttgagggttcgctggcggcggtggtacggggcgaaggcgacccgacctggccgggggagggccgtatcgatgagacggtctgggcgcagcccgcgctgttcgccgtggaggtggcactgttccgcttgctcgcgtcgtggggtgtagcgccgcaggtggtggcgggtcactcgatcggtgagctggctgcggcacatgtggcgggggtgtggtccttggacgatgcctgcgcggtggtggcggcccggggccgtctgatgcaggagttgccgtctggtggggcgatggtggcggtggaggccgccgaggagcaggtccgcaaggccctcgccgaccgccccggggtggggatcgccgcggtgaacgggccgcgtgcggtcgtgctgtccggcaccgagaccgacgtgctggcggtggcggaggagctggcgcgggcggggtcgcgcacgcgtcgtttgcaggtctcgcacgcgttccactcaccgctgatggagccgatgctggaccggttcgcccaggcgatcgcccacgtcacctacgggaagccgcagctaacgatggtctcggccctgaccggtcgccccgtttcggaggaggtcaccgccccctcgtactgggtccggcacgtccgcgaggcggtccgcttcgcagacgccacagagacgatgcgcgggctcggcgtgggcaccttcatcgaactcggcccggacggcgtcttgtccggcatggccgcgcagagccgtaccgacagcgagggcgagtcgggttccgaagcctggctgccgttgctgcgtcgtggccgcgacgaaccgcgcgccctgctgaccgcactggcgaaggtgttcgtccgtggagtcccggtgcgctgggaagaggtctatgcgggcaccggcgcccagcgtgtagacctgcccacgtacgccttccagcgtcagcgctactggctgccgaccgcacccgcgcacttggacgcggcgggcctgggtcaatccgccaccctccacccattgttgggggcggcggtggggttgccggccagtggtggcgtggtgctcaccgggcggctttcgctttcggggcagtcctggctggcggatcacgtggtggcgggccaggcggtggtgccgggcgcggtgctggtggagatggcggtccgtgccggtgacgaggtggggtgctcgcgggtcgaggagctgttgagcgaggctccgttggtgctgcccacgcagggcggcgtgcgggtgcaggtcacggtggaggaaccggacgcgtccggacgccgcgcagtcagcatccacgcccagagcgaggggaaccccccggaggcggcgtggacccggcacgcgaccggcgttctcgccccggccgaagcggcagccgctaccgccgacaggatggcgcagtggccgccgaccggcgcggtgtctgtcgcattggacgacttctactccgggttggcgaagcacggactgagctacggtccggtcttccagggtgtgcgggcggcgtggcgtttgggtgaggagctgttcgccgaggtcgctctgccgccgggtgtggtggtggacgggtttgggttgcatcctgcgttgttggatgcggcgttgcacgtcattgtgggtgtgggggagcgtgcgggtgggttggaggtgccgtttgcctggcgggacgtggagttgtatgccacgggtgctcaggtggcccgggtccgtgtggtgccgggagctggtggcgagggtgtttcgctcagcctggcggatgctgacggcgcgctgatcgcgtccgtcggttccctgaccctgcggccgttcgacgctcccgccgtccaatccgccgagggcctgttcacggtggaatgggtacccgccgcgctgacggcggacggttccggcgcggcggagggcccgtgggccgtgctcggcgaggacgtcctgggcgccgcgagctacccggacctgccggctctggtggcggcgatcgaggcgggtgccgaggccccgcggaccgtcgtggtgcgggccacgcctgagcggcccacaccggatgtcgcggctgcggcccggcggatcacgacggacgttctggggttggtgcaccagtggttggctgctgacgtgttgtccggtgcgcggttgttggtgttgaccgagcgtgcggtcgatgccggcccgggggtgccggtggaggttgcggcggcttcggtgtggggtctggtgcgggtggcgcagacggagaatccggggcggctgctgctggcggacgtggacgaggtgtccgtacccggtgcggaactggccggctggctgcgagacgggatagagctgggcgagccgcagtgggcggtccgcggcgggcagatccaggtgcccaggctcacccgcgccacaaccgagaccggtgtcgaggcccgcccgggcacggtgttggtgaccggtgcttcgggggtgctgggtgggttggtggcccggcatctggcgaggtcgggtcgggcggagcatttggtgctgctctcccgtcgcggcggcgaggcccccggaatgcctggactcgtggtcgatcttcaggagatgggtgtcaaggtcacggtggctgcctgtgatgcggccgatcgggatcagcttgccggtgttcttgagggtgtggcgctgacgggtgtggtgcatgcggcgggtgtgctggatgacggggtggtcggttcgctgactgcggagcggctggagtcggtgctgcggccgaaggtggatgcggcctggaacctgcacgagctgacccttgacctggggctggagctggacacttttgtgttgttctcgtccatcgcgggggtgatcggtaatgccggtcaggccaattacgcggcgggcaacacgttcctggacgccctggccgcgtaccgtcaccggcagggcctgcccgccgtgtcgatcgcctggggaccctgggagaacggcatggccggccggctcaccggcgccgaacgtcaaagactgacccagcagggactgctgccagtcaccgacgcgcgcggcctcgcggtgttggacgccgccgtgggccggtccgaaccactgctggtggctgccgagctggccctggcggcgctgcaccgcaccggggaggttccgtcgctgctctccgggctggtgcccggtccgcgcgccggtaagcccgcccgtccgacagcaggcggccggcgcagcgtcctggaccgtaccgaggaacagcagacactcgccgagcggctgggccgactgggccggggcgaggccctggacaccctgcgggaactcgtcctggcgcagtccgccctggtactgggcatgtctggccccgacaggctggacgtggaccgctccttccgcgacgtcggcttcgactcgctcaccgccgtcaaactgcgcaaccggctgaacactgccaccgggctgcggctgccacccacagtggtcttcgaccatccgagcccggcggcgctgagcggcttcctcgcggaggagatgctcggctcggctcccgagcaaccggacgtgctcgcggcccactccggccttgaccgggtcgaagcctcgctcgccgggctgatggccgacgagtccgtacggatgcaggtcagcgagcggctcaagcagatgctcggcgtgctgaacgcgaccgccggaaccgacgacggtgacgacgacggcgactcggtcgccgaccggatccagtcggccagcgacgacgacatgttcgacttcatcgacaaccagctcgggctctga

SEQ ID NO: 57 - DNA, 1143 bp, Streptomyces sp.
  source 1..1143: mol_type = genomic DNA
gtggccgacaagacaaccctcgtgaagtgcctggtctgggacctggacaacaccatctgggacggcaccctgctggaggacgcgaacgtcgaaccgttccccggcatcctggacaccctcgtggagctggactcccgcggcatcctgcactccgtctccagcaagaacgaccacgacctggcctggaagcggctggaggagctgggcctggccgagtacttcgtgcacccccggatcggctggggccgcaagtccgacgcggtccgcgagatagccgagcggctgaacttcgccgagaagaccatcgcgttcatcgacgaccagcccgccgagcgcgccgaggtcacctacggcgccccggacgtccgctgctacccggccgaacaggccacctcgctcaccggactgcccgagttcagcccggcgaccgtcaccgtcgactcccgccggcgccgcgagatgtaccaggccggcgcccgccgggacgccgagaaggagaccttcaacggccccgacgaggacttcctgcgcagcctcgacctggtcatgaccatcaagcgggccgacgaggaagacctctcccgcgtcgaggaactcacgctgcgcaccagccagatgaacgcgaccggcgtgcactactcggacgccacgctgcgcgcgttgctggccgaccccgaccacgaggtactgaccgtcaccctcaccgaccgcttcggcccgcacggcgcggtgggcgtcctcctcctggagcgccacccgcagctgtggcacctcaagctgctggcgacctcctgccgggtggtcagcttcggcgccggctcggtgatcctcaactggctcatcgaccaggcggcccgggccggcgcgcacctcgccgccgacttccggcgcaccgaacgcaaccgcatgatggacatcgcctaccggttcgccggcttcgcggacgacgcctgcgcctgccttgccgcgctcgaagcttccgccggggagggcgtgcagcggctgcatctggtcgctgatcgccgggaggcgccgtcgaccatgcgtctgcttgcgcctgaactggcgtctggcagcggggagaagacctccgagggcgggggaaacacctccgataccggggagaaggctcccgtcggctga

SEQ ID NO: 58 - DNA, 1122 bp, Streptomyces sp.
  source 1..1122: mol_type = genomic DNA
atgacagaccacctggccgacgggcgcgccctgatcagcgagctcgtcggcgaccgggccggcgactgggacgtggccggcgaactgcccgtcgagacgctgcggaccctgggcgcccgcggcctgctgtgcgcacaggtccccgccgaccacggcggcctcggcctgagcagcgccgccaacggcgaactcaccgcccacgccggcagcctgtgcagctcactgcgctcggtgatgacctcacagggcatcgtggcctggaccgtccagcgcttcggcgaccgcacccagcggcgcgagctgctcgcggagctgaccggcggcaaactcaccgccgtcggcttcagcgaaccgggcgccggcagcgacctggcggccatgaccacccggatcaaggccgacggggacggctacgtcatcgacggcgagaagaagtgggtcaccggcacccgctacgccgacttcgtgctgatcatcggccggtacggcgacggggacggcgccgccgccgcgatcgtccccgtcgacgcgcccggtgtgcacatccgggcggtgccgcacccgatgggctgccgggccgccggacacgccgacatccggctggaccaggtacggctgcccgcctcccacgtgctcggcggcggcggacagcccctgtccatgctgttcaccaccgccctgacctacgggcgggtgtccatcggctggggctgcgtcggcatcctgcgggcctgcctggacgcggccggccggcacgcgcaacagcgccaccagggcgaagtggccctggccgagcaccaactggtacgcgggcacctcgccgacctgatggtcgccgaacaggtcgcgctgcgcgcctgcgagcacgccagccggcagtgggacgccgcggccccggacctgggcaccatggccgtcctggccaagcacgtcagcgcccagcaggccgcccgcagcgccgccaccgcggtccagctgctcggctcgaccggctccgccgacggccacgtggtggcccgcgcgttccgtgacgccaagctcatggaactgatcgagggcagcaacgagatctgccgcctgatcctcgccgaccacgtgctgtccaccacgaagaccggctga

SEQ ID NO: 59 - DNA, 282 bp, Streptomyces sp.
  source 1..282: mol_type = genomic DNA
atgaccgtcgaaggcgaaaacaccacgctcaccgccgagaccgtgcacaaggaactgctcgacttcctcaccgagcggatccagaccaccgtcgagccggaccaggacctgttcgcctccggcatggtctcctccatgttcgccatgcaactcgtcgtccacctggaggaggcgtacgacatcgccatcgtcggcggcgacctcaagctggacaacttccgcaccgtgcagaccatgaccgacctggtgatacggctgcagggcacggcggaggcgcactga

SEQ ID NO: 60 - DNA, 867 bp, Streptomyces sp.
  source 1..867: mol_type = genomic DNA
atgagtactgctttcgatgagcaccggctcgccgtcgtcggcgccggcgtgatgggcacgaacatctcggcactggccctcggccacggcgtcgacgtcgtcctggtcgagacggacgaggccgcccgcgaccgggcggacggcaccgtgcggcacaagctgcggcacgcccagctgatgaacgtactgcccgcggaccgcccgcagggcacgctgacgatcaccgcgaagctggacgacatcgcggacgccgacacggtcgtcgaggccgtcaccgaggtcgccgaggtcaagaaggaggtgctcggcgcggcttccaagctggtcgcgcccggcaccaccctgatctccaacacctcctgcatcccgatcgaggagatggccgcctggacggcgcgccccgaagagctcctgggcgtgcacttcatgaaccccgcctacatgatcaagatggtcgaggtcatccggggcccgcagacctctcccgccaccctggaacgcgccaccgacctgctcacggtcctgggccgggaatccctcgtcatcgaggactccgcgggcttcgtgatcaaccggctgctgcacccgctgatcaacaccgcggccatgctcgtccaggagggcgtggcctccgtcgagacggtggacaacctgctgcagggctgcctcggccacgccaccggaccgctgcgcaccggcgacctgatcggcctggagaacctggtcgactccctcaatgtgctctacgaacgccgcggggacgagagctgccgcccctgcgacctgctgctcgccaaggtgcgggacggccacctgggccggaagaccggccggggcttctacgactacggaaaggccgcatcatga

SEQ ID NO: 61 - DNA, 2832 bp, Streptomyces sp.
  source 1..2832: mol_type = genomic DNA
atgaacgaccagccggccctctcggcacgcgccccggaatccgcaccccggccgggggacgcgcatacggccaccacgaacaaggaaaaggccctctggctgctggagcagctggtcccggacaccggggtgaacaacctggggctggcactccaggtgagcggccggctgcggccggacgtgctcggcgcggccctggccgtggtggccggccggttcgaggtgctgcgtaccgtgttccgcgcgagcggcgccgagctgttcaaggagaccgttccggcgggggagttcgccgtcccggtggagccgctggagctgtccggcgggccgctggccgaggatctgaccgcgttcgtcgaccggcccttcccgctcgacggccgccccctgatccgggtcgccaccgccaccaccgccgacggcgacgtgctctgcgtagccgtgcaccacttggtgttcgacatggtgtcggtcggcgtcctgctacaggcgctcgtcccggtctacgacgccatcgcggctggtcggccgctgcccgaacaggccgtcgcgccggtcccggctctcgccgagcccgcgccgcgccccgccgacctcgaccactggcgccggaccctggacggcttcgaccccaccggcctggacctgtggtgcggcaccccgcgcgggcggcagccggtgctggccggcgaaagcgccgtgcggaccctcggcccccgggcccagcaggccgcagtcgagttgcagcgtgccgcgcgggctccgatgggcgcggtgatgctcgccgcctatgccgccctgctcgccgcccacggcgccggcccggacctggtgatcggctcaccggtggacgtccgaggggccgacgccggcgccatcggctaccacgtcaacgtcgtccccatccgtatccgggtggacctcgccgagggcttccgcccgctcgcccgccaggccagggacgccttcctcggcgcgatggcgcacgcgcacacctcggtggacgaactcgccggagagctgccgggcatcggcacgtcctggcagacccagctcttccggcacctgttcaacttcctccccgacgcgcccgtcggcgagctgcgggtggacgggatgccggcccggctgctgacggtggagaacccgcacagcaagttcgacctggaactggtcggctccccggcccgtgccgagctgatgctgcgtcactcccgtacgctggcccccgccgacgccgcggccctgctggcccgcttcgaggcgctgctcgtcacggccgccgaggacccggaccggccgctcggcgagacggcgggctggagcgagcatgaccgcgagacggtcgaccgggccaacgacaccgcactggacagtcaaccagactcgtttgaggcggcgttcacctcctgggctcgtagccggcccgccgatgtggccgtgctggacggtgaccggagcctcacctacggcgagctggacgcggccgcggacgccgttcgcgagttgctggcggacgccggggtgcggcccgcagaggtggtcgcggtcgcggcggcgccgggcgagacggctgccgcgctcctcggctgctggcgggccggtgccgtctgcctcccgctcgaccccggagaggaatcgttcctgcaggcccggcggctgacgcaggccgcggcgaagaccgtgctcaccggagcgggagtgcaactgcctgccggagtggacctgcccgccggggcggacctgccgcccgtcctgccgctgccagcgttccccggtaccggccccgagcgctccggcgcgcccgtagacccgtacggcgacgccccccgacgggacgcgcccgcctaccggctgcccgcaggcgaggagaacgaggacgaggcgcagtcggccgtcgtgctgagccacgccggtatcgccgacaccgccgcccacttcgcccgggagctcggcgtcgggcccggcaccggtgtgctgagcctggcccggcccggcaccctcgcggcactcttcgacgccgccctggcgctgagcgtcggcggccggctcgtgccggccccccaggagacccgtaccgacgccgcagccctgcgcgcggccgccgaacggcacggggcggacgtgctggccgtaccgccgggcaccgcgcctcagctgctggaggagctgggcagccaactgtccgggctgaccgtcctggtcgccggtgacgagctgtccgcgtcggccgcgaaggcgctgctcgccgcggactgccggctgcactgcggctacggagccccgcagaccaccggctgggccctcgccggccgggtcaccacccccgaggacacccgccggggccgcccgatcggcaacacccgggcgttcgtcgccgcccccgacggacgggagctgcccgtcggtgtgcgcggcgaactctgcctcgccggcaccggtacggccctggacacaccggacggcccggccctgccaaaccaccccgggtacgggcgccaccaccgcaccgggcggctcgccgaatggcagccggacggcaccctcgtctggcacccccgcgagcagcccgccgcggacccggccgccgggcccaccgcaccggacgccgcggccgacgaccccctcgtcggccagttcgtcgcgatctggcagcagctgctgagctgcgaggtcaccgcgcagacccacttcttcgacgcgggcggccactcgctgctcgccgccgtgctgacgcagcaggtcgaggacctcaccggcatcgccctgcaactgcgcgacgtcttcgagcacccgacccccgccgccctggccgccagggtgcgccgatga

SEQ ID NO: 62 - DNA, 840 bp, Streptomyces sp.
  source 1..840: mol_type = genomic DNA
gtgccgaacgagaacgcctggagcgtcatagccgagcctgcctcgaattccctgatactcggcgtcgactttcctgcggccggccgtcacgaggcgggcttcgacgacctggtgaagggaatgggcaccgcctggtccgggcacggggtgctgcagacctcgccgccgccggtgcggctggccgaccggccgtccggcgacttctacaccgaccactggatgcggtcgggcgactgggagaagtacgaggtcgtggcggtgctcggatactgcgtcggcgcggtctacgcgggccgggtcgccgagcggctggcggcggtgcagggcaggatgccccaggtggtgctgttcgacgcgcagctcgccgatctgccgctgctggccacggagatgcacaagatgatcgggcaggccgccgcggtgctcagcgagcaggaggctgaggagggccggaaacgggccgtcgaaatcgtcgccaccccgtcgatcggcatggcggaggcggccgggcagatggtcgagctgtaccgggagctggccacttcgggtttccggcggatcggtctgtcggagtcccgctgcgaagagatggtgctgcttttcgagtcgtacatgacctggctctcggcctcttcccgtatcgacccggccggtgtctggggcggtgcgatcggcctcacctcggccgactacggcgaattggagaaggccggcgccaccaccgtcgtgaacgccgccaccgcggcgatcggccggcgtatacccgtcgagacccgccacatcgatctcctgcgggatgcgtcgaccgtgcgcgcgctgctcgccaacaccgaattctga

SEQ ID NO: 63 - DNA, 15027 bp, Streptomyces sp.
  source 1..15027: mol_type = genomic DNA
atgacaggacaggccgacaagctccttgaagcgctgcgtgcctccgtgaaggagaccgagcggctgcggcagcagaaccggcagctcgtctcggcggcctcggaaccgctggcgatcgtcggcatgagctgccggtacccgggtggtgtgaccaccccggagcagctgtgggagctggtctccaccggcggcgacgccatctccggcgtccccgccgaccgaggctgggacaccgagggcctgcacgaccccggccgaccggacagctcatccggggccgagggcggattcctgcgggacgcggccgccttcgacgcggacttcttcgggatcagcccgcgcgaggcgctggccatggacccgcagcagcgcctgttgctggagacgtcctgggaggcgctggagcgggccggggtggcacccgcctccctccgcggctcgcagaccggtgtgttcgtcggcgtcggcccctccgactacggcgccaaccccgccctgcacaccgacgacctgcagtggcacctgatcaccggcggcgcgaccagcgtgatgtccggacgcatctcctacctgctcggtctgcagggaccggcggtgtcggtggacaccgcctgttcctcctcgctggtggcgctccacctggcggcgcaggccgtgcgcgcgggggagtgctcgctggcactggccggcggggccacggtgatggcctcggccatcggaatccgcggcttctcccggcaggacggcctggccgcggacggccgctgcaaggcgttttccgccgacgcggacggtatgggcatgggcgagggcgccggcatggtcgtggtggagcggctgtccgacgcccgccgtaacgggcacccggtgctggcggtgctgcggggcagtgcggtcaaccaggacggtgcgtcgaacggtctgaccgcgcccaacgggccgtcgcagcagcgggtgatccgtgctgctctggctggtgcgcgcctgacggcgtccgaggtggacgtggtggaggcgcacgggaccggtacgaagctgggcgacccgatcgaggcgcaggcgctgctggccacttacggtcaggagcgccccgagggccggccgttgtggctgggctcggtgaagtccaacatcggccacgcccagcaggctgccggtgtcgcgggtgtcatcaagatggtgctggctctccagcacggacagctgccgcgcaccctgcacgcggacgagccctccccgcacgtcgactgggcatccggcgaggtgaagctgctgaccgagccggtgccgtggcccagcggggaccgggtacggcgggccggtgtctcctccttcggcctcagtgggaccaacgcgcacgtcattctggaggaggctcctgcggcgcaggatgctgaggctgctgaggagtccggggaactggtggttccggtggtgaccggtgctggtgcttgggtgctgtctgggcggactgctggggcgttgtcggcgcaggcggggcggctgggagagtgggtgtcggcccgtccggctcttcggccggttgatgtggcgtggtctctggctgccggccgctcggtcttcgagcaccgtgcggtggtggtgggcggggaccgggagcaggcgctggcgggcctcgcgggcctggcgtccggtgcgccctcgggatcgctggtcgcgggcatggcccgatcggatgcccgtccggtgtttgcttttgctggtcagggttcgcagtgggttggtatgggtcgggagctggcgggggtttcgccggtgttcgcggggcggttggcggagtgtgctgcggcgttggcgccgtacgttgactggtcgctgaccgatgtcttggcgggtgcggagggcgctcctgcgttggaggcggcggatgtcgttcagcctgcgttgtgggcggtgatggtgtcgcttgccgctgtctgggaggcggctggtgtcgctccggatgcggtggtgggtcactctcagggggagatcgctgcggctacggtcgcggggatgctgtcgcttgaggacggtgcccgtgtggtggcgttgcggtcgcgtgcgttgaaggtgctggcgggtgccggtggcatgctgtcggtcagtcgtccggcggcggaggtcgaggaacgcctggtccggttcggggagcggctggctctggcggccttcaacgggccgtcggcgacggtggtgtccggcgagccggaggcgttggaggagctgagggcggagttcgaggcggagggtgcccgggcgcggatggtggcggtggattacgcctcgcattcggctcaggtggaccggctggaggtggagatcacctctgttctggctggggtttccccgcgtcggggacgtgttccgatggtgtccgcgatgactggcgagacgctgacgggtgaggagctggacgccgggtactggttccgcagtctgcgggcgacggtggattacgaccgtgcggtgcgcgtgctggccggtcagggccatcaggtgttcgtcgaggtcacgccgcatccggtgctggtcggcgccatgcacgacacccttcaggcactcgctcaggaagcgggcccgggcgtcatgccggcggccgtctgcgggacgcttcgccgcgacgaggacggtgcggatcgtctggtggcgtcgctggcggaagccttcgtcaatggggcgtcggtggactggcaggcggtgcttcccgcagggaagcgggtggagctgccgacgtacgcgttccagcatgagcggttctggccgaaggcagccgccgcggtttccggtggtgatccggtatcgctggggctgggtgcggtgggacatccgctgttgggtgcggcggttgaactggcgggcggtgcgggtctggtgtgtacgggccggttgtcggtgcgtacgcatccgtggctggccgatcacgccgtcagcggtgtggtgctgctgcccggcaccggcttcgtggagctggctgtgtgtgcgggggatcaggccgggtgtggtgtgctggaggagctgacgctccaggcgccgctggtcttccccgaggacggcagtgctgtgcaggtccaggtggtgttggccggagcggatgcggagggtcggcgtagcgtcgaggtcttctcccggtcggatgcggctggtcagtcgccggggtggacgcagcacgccagcggcgtgctggcccctgccggtccggcggctgcggccgaggaggacttcgcggtgtggccgccccgtaacgccgccacagtggacgtctccggcctctaccagtccgctgccgaaggaccctacgggtacggtccggcgttccaggggctgcgggcagcatggcggcggggcgaggacgtcttcgccgaggtggcgctgccggaagaggtggcgcaggacgccgccgccttcggcctgcacccggcactgctggatgccgtactgcatgccagtgccgtgcttgaggctgagggtgagccgggcgagatccggttgccgttcgcctggacgggtgtggagttgtatgccagtggtgcgtcggtgctgcgcgcccggttgcgtcgtgatgcgcagggcacgctgacggtgtcggccgccgatgcggcgggtcagccggtgatttccgtggcctcgctgatcacccgcccggtggacaccggccagctcggcagtgccgtgtcggctgtcgcggatgccttgttcaccgtcgcctggatgcccatcagcggcatcgagtccatagcccccgtagccggggaatgggcgctgatcggcgccgaccgcttcgctgtcgccgagaccctgggcaggtccggcgtcccagtgcagtgcttcccggggctggaagaggccgctgctgccgtggaggcggggcagtgcacgcctgcgttcgtcctggtctgcgccggtggcgagggcctgggcgaggagggcccggcgcgggccgcggcccgggtgactggcgagaccctggttctggtccaggactggctgggtgaggagcggctggcggagagccggctggtggttctcacgcgtggtgcggtggcggtctcagccggtgagggcgtggccgacctgtcggcagcagccctcaccggcctggtgcgctcggctcagtcggagaacccggaccggctgctcctcgtagacctgccggccaccggcactgcggagcaggtcaccgagctgccggttgccgttgccaccggggagccggagttggcgctccggaatggcgtagtgcatgcacgacggctggtgcggccctctggggagctgccggttccggagggccggtggcggctggtaccggacgcgggcggttcgctggaggggctgtcgctgctcccgtcgccggaggtcgatggaccgctggcggccggacaggtccgtatcgccgttggtgctgccgggctgaacttccgcgacgtactgatcgcgctgggcatgtacccgggcggcggtgtgctgggcagcgagatcgccggaacggtgctcgaagccgggccgggagtggaccacctcgccccgggcgaccgggtgacgggaatcggtgagggcgggttcggcccggtggttgtcaccgatgcccggcagttggtgcggattccggacggctggtcgttcgccgaggcggcctcggtgccgacggcgttcatgactgcttggtacgcgttggtggagttggccggggcgaagccggggcagcgggtgttggtgcatgctgcgaccggtggtgttgggatggcggcggtgcagatcgcccggtacctggggttggaggtcttcgcgaccgcgagtacggggaagtggccggtgctggcggcgatgggtctggacgcggagcacgtctcgtcctcccgtactgccgcattcgaggcggaattcttggccgccacggacggtgccggtgtggatatcgtggtgaattccctggcgggtgagttgaccgatgcttcgctgcggttgctgccgcggggcggggcgtttgtggagatgggtcgtaccgacgtccgcgacgcccaggcagtggccgcggaccgacccggtgtcacctaccgctcgttcgacctcggcgaggcaggccccgagggtttgggcaccatgctcggccggatcgtggaactgctgtccgccggcgaactgcggcggctgccgatgcgggcctgggaggtgcgccgggcgcgggacgcgttccggttcatgcagcaggcccggcacaccggcaagatcgtcctgacgatccccgcgcacctctccgcaacgcagcagccggaatcggcccgggccggtgcccgttcggcgttgatcacgggtggtacgggtacgttgggtggcttggtggccgggcatctggtggtgtctgggcgtgcggagcacgtgctcttgaccagccgttcgggacccggtgcggccggtgctgcggcgttggccgcgcggttggcacaggcaggcgcttcggtgcgggtggtggcgtgtgatgctgcggaccgccagaagctggccgccgtgatggcggcgatcccggccgggtatccgttgcggaccgtggtgcacaccgcgggtgtgctggacgacggtacggtcggatccctgaccgcgcagcgggtggccggggtgctgcggccgaaggcggacggtgcctggaacctgcacgagctgacccgggacctggttccggagctggaggactttgtactgttctcctcctcggcggcggcgttcggggtgccgggccagggcaattacgtggcggccaacgccttcctggacgcactggctgccgagcggcgggcgtcggggcttccgggtgtttcgttggggtgggggctgtgggccgaggtgtccgggctgaccgggcagctggccgagtctgaccgctctcggttgtcgcgtggtggtgcgttggcgctcagcgtcgacgaggggctggcgctgctggacctgtgtctggcccgggacgaggcgctgctggtgccgtcccggctggacctggccgggctgcgggcccaggcggcgcggagtgcggagattccgccgctgtggcgggccttggtcggtacgggaaccgggcggcgcagtgccgccggggccggtgacgcgaacgcggactcgctgcatcagcaactgtccgggctgtccggtcaggagcggacccggctgctgaccgatctggtccgtacgcatgcggcggctgtcctggggcatgcctcggccgaggctgtcgagccggggcgggcctttaccgacctgggcttcgactcgctcaccgccgtcgaactgcgtaaccggctgagcaccgcgaccgggctgcggctgcccgccaccctggtcttcgactaccccaacccggcagcgctcgccggccacctcaaggccaggatcatcggcgaaggcgacggcgacgggccggccgcgagcccgctgccgtccggtacgacgcagcaggcctccggggaaccgatcgcgatcgtcggcctggcctgccggttccccggcggggtgaacgaccccgaggggttctggcggctgctcgccgacggcaccgacgcggtctcccgcttcccggacgaccgcggctgggacgtggaggcgctctacgacccggaccccgacagcgtcggcacctcctacaccagggaaggcggcttcatcggcgacgccggcggcttcgacgcggacttcttcgggatcagcccgcgcgaggcgctggccatggacccccagcagcgacaggtcctggagacctcctgggaggcgctggagcgggccggcatcgacccgtccgcgctgcgcggcagccagaccggtgtcttcgtcggcggcttcgcctcggcctacggcatcggcgtggcgaccggcgacgtgggcgcggaggggaacctgaccaccggcttcgccaccagcgtgctgtccggccgggtgtcctacgccctcggcctggaaggcccggccgtgacggtggacacggcgtgctcctcgtccctggtcgcgctgcacctggcttgccaggcgatccgcgcgggggagtgctccatggcgctcgccggcggttccacgatcatctcgaccccggacgggattgtcggcttcagccgccagcgcggcctggccgcggacggccgctgcaaggcattctccgccgacgcggacggcatgggcttcgccgagggcaccggcgtcctggtcgtggaacgcctctcggacgcccgccgtaacgggcacccggtgctggcggtggtacggggcagtgcggtcaaccaggacggtgcgtcgaacggtctgaccgcgcccaacgggccgtcgcagcagcgggtgatccgtgctgctctggctggtgcgcgcctgacggcgtccgaggtggacgtggtggaggcgcacgggaccggtacgaagctgggcgacccgatcgaggcgcaggcgctgctggccacttacggtcaggagcgccccgagggccggccgctgtggctgggctcggtgaagtccaacatcggtcacactcaggccgctgccggtgtcgcgggtgtcatcaagatggtgctggccctccagcacggtcaactgccgcgcaccctgcacgcggacgagccctccccgcacgtcgactgggccgagggcgaggtgcggctgctgaccgagccggtgccgtggcagacgaacgaccggccgcgcatcgccggcatctcctccttcggcatcagcggcaccaacgtccacgtcctcttggaggaggcccccgccgaggaacccgcgcacggtaacggggaccccgccgacgctgacacagcgacttcggcaccggtgctctccatggtcggggcgggcccggctgccgagacggtgagcgcctggctggtgtccggccgttcggctgcgggcctggccgcgcaggcggaccggctgcgggattggctgacgacccgcccggagctggaatcggccgatgtggcgtggtcgctggcagccacccgctcggtcttcgagcaccgtgcggtggtggtgggcggggaccgggagcaggcgctggcgggcctcgcgggcctggcatccggcgcgccctcgggatcgctggtcgcgggcacggcccgatcggatgcccgtccggtgttcgccttcgccgggcaggggtcgcagtgggtcgggatgggccgcgagctggcggcggtgtcgccggtgttcgcggggaggctggcggagtgcgcggcggcgttggcgccgtacgttgactggtcgctgaccgatgtcctggccggcgcggaaggtgcccccgcgttggaagcggcggatgtcgtccagcccgcgctgtgggcggtgatggtctccctcgccgctgtctgggaggccgccggcgtcgctccggatgcggtggtgggccattcacagggggagatcgccgctgccacggtcgccggaatgctgtcgcttcaggacggtgcccgtgtggtggcgttgcggtcgcaggccctgaaggtgctggcgggtgcgggtgggatgctgtcggtcagccgtccggcgtcggaggtcgaagaacgcctggcccggttcggagatcggctgtccctggctgcggtcaacgggccctcggccaccgtcgtctcaggcgaaccggcggctctggaggagctgaagtccgagttcgaggcggagggcgcccgcgcccgtatggtcgcggtggactacgcctcccactgtgctcaggtggaccggctggaggaggagatcaccaccgtcctggccggaatttccccgcgccggggccgcgtcccgatggtctcggcgatgaccggcgagacccttaccggcgaggagctggacgccgggtactggtaccgcagtctgcgcgccaccgtgcattacgaccgcgccgtgcgggttctggccggtcgtgggcatcaggtgttcgtcgaggtgacgccgcatccggtgctgatgggcgcgatgaacgacaccctggaagaggtcgcccaggaggcgggcccgggcatcgtcccggcggccgtctgcgggacgcttcgccgcgaagagggcggcccggcccagctggtcacctcgctcgccgaggcgttcgtcaacggggcaccggtggactggcaggcggtgcttcccgcagggaagcgggtggagctgccgacgtacgcgttccagcatgagcggttctggccgaaggcagccgctgcggtttccggtggtgatccggtatcgctgggcctgggtgcggtgggacatccgctgttgggtgcggcggttgaactggcgggtggcgaaggtctggtgtgtacgggccggttgtcggtgcgtacgcatccgtggctggccgatcacgccgtgggcggtgtggtgttgctgccgggcaccggtttcgtggagctggcggtgcgcgctggtgaccaggccggctgcggactcctggaagaactgacgctgcacgccccgctggtcttccccgaggacggcgctgcggtccagatccaggtggtgctggcggggccggacgccgaaggccgccgtggcgtcgaggtcttctcccggtcggatgcggctggtcagtcgcagggatggacgcagcacgccagcggcgtgctggcccctgccggcccggcggctgcggccgaggaggacttcgcggtgtggccgccccgtaacgccgccgcagtggacgtctccggcctctaccagtccgctgccgaaggaccctacgggtacggtccggcgttccaggggctgcgggcggtatggcgccgtggcgaggacgtcttcgccgaggtggcgctgccggaagaggtggcgcaggacgccgccgccttcggcctgcacccggcactgctggactccgtactgcaggctgccttcctgatgggaggtgccgaagagggtgcgaccgaaaaggttcggttgccgtttgcctggacgggtgtggagttgtatgccggtggtgcgtcggtgctgcgcgcccggttgcgtcgtgatgcgcagggcacgctgacggtgtcggccgccgatgcggcgggtcagccggtgatttccgtaacctcgctgatcacccgcccggtggacaccgaccagctcggcagtgccgtgtcgccggttgcggatgccctcttcaccgtcgcctggatgcccatcagcggcaccgagtccatagcccccgtagccggggaatgggcgctgatcggcgccgaccgcttcgctgtcgccgagaccctgggccggaccggcatcccagtgcagtgcttccccggactgaaggagctggccgctgccgcagcggaccgggagatcgaccccgccttcgtcctgatctgcaccggtgatgagggccagattctggaagacccggcgcggaccgctgcccgggtgactggcgagaccctactcctggtgcaggagtggctgactgaggaccggctggcggacagccgactcgtgcttctcacgcgtggtgcggtagcggtctcagctaatgagggcgtggccgatctgccggcagcagccctggccggcctggtgcgctcggcccagtccgagaaccccgaccggctgctcctggtcgacctcccggcgaccaataccgccgaccagatcgccgtactcccgaatgcgctcccagccgcactcgccgccggggaacccgaggtggccatccgcgacaccgccgtccacggacggcggctgacccgtccatccggctccacggcggcagttgaacgacccgccaacccgtccggtgcccgttcggcgctcatcacgggcggtacgggcacgttgggcggcttggtggccgggcacctggtggtgtcgggacgtgcggagcacgtagtgctgatcagccgttcggggcccggtgcggccgatgcagcagtgctggccgcacggttggcccaggcaggcgcttcggtgcgggtggtggcatgtgatgccgcggaccgccaggagctgaccgcagtgatggcaacaatcccagcgcaggcgccgctacgcaccgtggtgcacaccgcgggtgtgctggacgacggtacggtcggatccctgaccgcgcagcgggtggccgaggtgctgcggccgaaggcggacggtgcctggaacctccacgagctgaccgagggactggacctggaccacttcatactgttctcctcctcggcggcggcgttcggagtgccgggccagggcaattacgtggcggccaacgccttcctggacgcactggccgccgagcggcgggcgtcggggcttccgggtatttcgttggggtgggggctgtgggccgaggtgtccggactgaccgggcaactggccgagtccgaccggtcccggatgtcgcgtggtggtgcgttggcgctcagcgccgacgaggggctggcgctgctggacctgtgtctggcccgggacgaggcgctgctcgtgccgtcccggctggacctggccgggctgcgggcccaggcagcgcgcagtgcccaggttccgccgctgtggcgggctctggtcggcgccggaaccgggcggcgcagcgccgccgggacggacgacgcgaacgcggactcgctgcaccggcaactggccgcactgcccgccgccgagcgcgacgagatgctgctgaacctggtacgggcccacgtcgccgccgtactggggcacgcctcggccgaggccgtcgagccgggccgggccttcaccgacctgggcttcgactcgctcaccgccgtccagctccgtaaccggctgagcaccgcgaccgggctgcgactgcccgccaccctggtcttcgactaccccaccccggcagcgctcgccggccatctgcgcggacggctcggcgacgggccgcaggcgcgcacgaagtccgccaccgccccggcggcagcggagacatcaggagccggggcgccggacgagccgatcgccatcgtgggaatgagctgccgcttccccggcggcacggcggaccccgaggacctgtggcggatgctgcgcgacggcgaggacgccatctcggccttccccaccgaccgtggctgggacctggacgggctgcacgacccggacgccgcgcggagcggcaccgcccgtacgcaggccggcgggttcgtgcaggacgccgcctccttcgacgccggattcttcgagatcagtccgcgcgaggccctcgccatggacccgcagcagcggctgttgctggagcttgcctgggaggcgttcgagcggtccgggatcgacccggccaccctgcgctccagcccgaccggcaccttcgtcggcggctacacctccggctacgagacgagcgtgctggcggagggcggcaccgagtcccaggggcacctgatgactggcatcgccaccagcgtcctgtccggccggctggcctacaccttcgggctggagggcccggcggtgacggtcgacacggcctgctcgtcggcgctggtggcgctgcacctggccgcgcagtcgctgcggtcgggggagtgctcgctggcgctggccggcggtgtgacggtcatggccaccccgggtacgttcgtggagttctcccgccagcgtgggctggccgcggacggccgctgcaaggcgttcgccgccgcggccgacggcaccggcttcgccgagggcgccggcctgctcgtcatcgagcggctttcggatgcgcagcgcaatgggcacccggtgttggcggtgctgcggggtagtgccaccaaccaggacggtgcgtcgaacggtctgaccgcgcccaacgggccgtcgcagcagcgggtgatccgggctgcgttggccaactcgcgtcttacgcctggtgacgtggatgtggtggaggcgcacggtactggtacgaagctgggtgacccgatcgaggcgcaggcgctgctggccacttacggtcaggatcgcggcgagagcgatccgttgtggctgggctcggtgaagtccaacatcggtcacactcaggccgctgccggtgtcgcgggtgtcatcaagatggtgctggctctccaacacggacagctgccgcgcaccctgcacgtggacgagcccagcccgcacatcgactggaccggcggcgaagtgcggctgctcaccgaaccggtgccgtggcaggcgaacggtcggccccgccgggccggcgtctcctccttcgggatcagcggcaccaacgcacacgtcatcctcgaagaagcacccacgccgcccgccggggccgagagccacgtaccgctcgccgggtccgggagccacgtgctgctccccggggccgagagccacgtaccgcccgccgaccccgagacccacgcggcgcccccgcccggtgccgcaaccgccccgacggccggctcggtcgtaccgtcggcggtgccgtggccgctgtccgcgcgaaccccggaggcgctgcgggcccaggcgggacggctgcgccagttcctgctggagcggccggaactcgacctggtggacgtcggattctcgctggccaccacgcgtaccgccttcgaccaccgggcggtggtgcagggcacggaccgggacgggctgctggcgggcctcgcggtggccgccgcaggggtgcagacccgggacgtggtgaccggaaccgtgagggcgggccgcaaaacggtcttcgtcttccccggcacggccgcatcgcccggagggcccggcgcggacgtccgcctggacgccgcggccgcgctgctggaggagtcgccggtcttcgccgaccggatcgccgactgcgagcaggccctggcgcccttcgtggactggaaggtgaccgaggtgctgcgggccggtgaggccggcccggcgctcaccacccccgagatccaccgcccggtgctgtgggcggtactggtggcgctcgccgcgctgtggcgctccgccggtgtgcatccggccgcggtggccgggcacggggtgggcgagatcgccgccgcccaggtggccggtgtgctgacggcggcggacgcggcccggctggccgtgctgtgtggcggggcatcggccggtccgggagccgtccaaccgcggccggggacggtgccgctctactccacccggtccatggactgggtggacggtgcggagctgaacgccgagcactggctgccgtacccggggcaggaaccgccggcccccgtacgggaactggccgaggccctggctggggccgggttgggcgcgtacgtcgaggtcggcccggagccggtgcttgccaccgccctggcccaggccctccaggccgaagggaacggcgcggaccgcgacggtgcgcccgtggtcaccgcgaccctgcgccgtggggagttgggactgcgggcgttcacctccgcgctggccgccctgcacgtacggggcgtgaccgtcgactgggccgcggtgctggccggcggacgccgggtggacctgcccacctacgccttccagcgagagcggtactggccgcagccgccggcccggaccaatgccgaagctcccgcggcgggctccctgtccccggacgagcagcggttgtggtccgcgttggcggagcaggacatgagcgcgctgagcgaggtgctgggcaccggcacgccgttgcgcgaggacatgcctctgggcgaagtactggcgtacgtcgccgcctggcgccgggagcgcaccgcaccggacggggcggccgagaccggtgccgcgggcgcggccgagggcaccgcacctgaagatgcggacggcgccggggccggctggcggcagcggctcgccgaagtcggcccggcgcagcagcaggagctgctgagcgcactggtccgcgaggagatcgcggcgatgctcggatacgggtccgtggactccgtcccgcagtacggcgacgtcttcgaactcggcatgacctcgatgtcggccgttcaactgcggctgcgaatgaacgagctgactggtcttacactgccggagggcttcgtctatgacctctatatgccggaagccatcgcggagtacctgctcggcgagctgacggtggcacttcgggaaggcactcccgaagaccggtag

SEQ ID NO: 64 - DNA, 16809 bp, Streptomyces sp.
  source 1..16809: mol_type = genomic DNA
gtggacaaccaagagaaactcttcgactacctcaagaagaccgcggcccagctccaggaaacgcgtaagcgtctgcaccggctggaggccgccgagcaggagccgctggcgatcgtcgggatggcgtgccggttccccggtggggtgggcagtccggaggagttctgggagtttcttgcttccggtggcgatggcgtggacggtttcccgaccgaccgcggctgggacctggagagtgtgtacgacccggacggtgagcgcgaaggcacctcgtatgtcgcccagggcgcgttcctgcgggaagcggccgagttcgacgcggaattcttcggaatcagcccgcgcgaagccctcgccatggacccgcagcagcgcctgttgctggagacctcctgggaggcgctggagcgtgccggcatcgacccgacgtcgctgaaggagagtgccaccggtgtgtttgccggtggcttcacctccggttacgggctcggcatatccatgagccaggacgactccggcgctgagggtcacgtgctgacggggaatgcgaccagtgttctgtccgggcgggtgtcgtatgtgctgggtcttgaggggccggcggtgacggtggacactgcctgttcgtcgtcgttggtggcgcttcacctggcggcgcaggcggtgcggtcgggggagtgttcgctggcgttggccggcggggcgaccgtgatggcgacaccggggacgttcatcgacttctcccgtcagcagggcctgtcggtcgatggccggtgccgggcgttctcggaggacgcggatggtacgggctgggccgagggtgccggcatggttgtggtggagcggctgtcggatgcccgccgcaacggccacaaggtgctggcgctgttgcggggcagtgcggtcaaccaggacggtgcgtcgaacgggctgaccgcgcccaacgggccgtcgcagcagcgggtgatccgtgcggccctggccaacgcccggttggctccggcggacgtggacgtggtcgaggcccatggcaccggtaccacactcggtgaccccatcgaggcgcaggcgctgctggccacgtacggccgggatcgtggcgagggtgacgcgctgtggctggggtcggtgaagtccaacatcggccacacccaagccgcggctggcgttgcgggcgtcatcaagatggtgctggccctccagcatgagcggttgccgcagacgctgtacgccgaggtgccctccacccacgtggactggtcggcgggtgcggtccggttgctcacggaaccggcgccctggccggccgacggagaccgggtgcggcgggccgctgtctcggcgttcggcatgagcggtaccaacgctcacctcatcctggaggaggcccccaccgcgcaagaggccgaagccgccgaggaacccaaggaaccggagacagaaccggacgggccggctgcgcgggtgctgtccggtgaaggcgccggtgcggtccgggcctggcaggtctcgggccggtcggcggcagcgccggcggcgcaggcggaccggctgcacggctggctgacggcccgtccggagctgcgaccggccgatgtcgcctggtcgctggcgaccgtgcggaccgccttcgagcaccgtgccgtcatcgtcggcagtgacgaccgcgccgcgctgctgacgagcctggaaagtctcgccgccggaacgccgaacggttcggtggtgtccggaacggcccgccccggcgtccggcccgtgttcgtcttccccggtcaggggtcgcagtggctgggtatgggacaggagctggcgcgtacctcgccggtgttcgcggcgcggttggcagagtgtgctgcggcgttggcgccgtacgttgactggtcgctgaccgatgtcctggcgggaacggggtacgccccggcgctggaggcggcggatgtcgtccagcctgcgttgtgggcggtgatggtgtcgctggccgctgtctgggaggcggctggtgtcgctccggatgcggtggttggtcactcgcagggggagattgccgctgccacggtggccgggatgctgtcgcttgaggacggtgcccgcgtggtggcgttgcggtcgcgtgcgctgaaggtgctggcgggtgcgggcggcatgctgtccgtgagccgtccggcgtcggaggtcgaagaacgcctggcccggttcggcgaccggttgtccttggcggcggtcaacggcccgtcggccaccgtcgtctcgggcgagccgggggcccttgaagagctgaaggcggagtacgaagcggaaggcgcccgggcccgcatggtcgcggtcgactacgcctcgcactccgcccaggtggaccggctggaagaggagatcaccaccgtcctggccggagtctccccgcgccggggccgattccccatggtctccgcgatgaccggcgaaaccctcaccggcgaagaactggacgccggctactggtaccgcagcctgcgcaccaccgtacatttcgaacgggcagtgcgctccctggccggccaggggcagcgggtcttcatcgaggtgtcgccgcatccggtgctgttgagcgcgatgacggactcgctggaggaggccgcacaggacgcgggcccgggcgccgtgcccgccgccctgtgcgggacgctccgccgggacgacggcggcgcggaacggctggtcacctcgctcgccgaggcgttcgtcaacggcgcgccggtggactgggcgtcggtgctgcccgccggcgagcgggtcgagctgcccacctacgtcttccagcaccgccggtactggcccaagggcccgatggcggggctgcgccaggatggcggcgacgcactggcgctgggcctcggcgcggtcggccacccactgttcggcgctgccgtcgaactggccgggggcgacggcgtggtgtgcctgggacggctctcgctgcgcacccacgcctggctggccgaccatgccgtcggcggtgtggtgctgctgcccggcaccggcttcgtggagttggcggtacgcgccggtgaccaggtcggctgcgggctcctggaagaactgacgctgcacgccccgctggtgctgcccgccgacggcagcggggtgcagctccaggtcgcggtggccgcaccggacacggagggacgtcgaggagtccgggtcttctcccgcccggaggacgccgccgaccggcagccgtggacgctgcacgccggcgggacgctcggccccaccgccgcgcccgcaccggccgacccggacttcgccctgtggcccccgcaggacgcggtgccggcggacgtctcggacgtctacccggtgcggctggcggaggtctacggccccgccttccacgggctgcgtgcggcctggcggcgcggcgcggacgttttcgccgaggtggcactgcccgaggaggccgcgcagcaggcgggtgccttcggcctgcacccggcgctgctggacgcggcgctgcacgcctcgttcctggcccagccggccgccgcgcaggagaccgggcaggtcaccatgccgttcgcctggaccgacgtggcattgcacgccggtggcgcctcggtgctgcgggtgcggctgcgccaggacgcgcagggcggactctcgctggcggccgccgacaccaccggagccccggtggtcaccgtgggatcgctggtgcggcgcccggtatcggcggagcaacttcgcgctacgcgagacgagttgtccgactcgctgttcgtccagacatgggccgagctgcctgatggcgacgccccggccggcgagtgggcgctgctcggcgccgaccgctacgggctggccgacgggctggaggcggcgctggaggcagcaggcgtgcaactgcgccccttccccggcctccccgagctggtcgccgcggcgcagaccggcaccggagccccacggaccgttgtggtctgcgccgacgccctgtccgacgaggacgtgttcggcgggagcgccgagacctcggcgggcaaggaggcgccgaacaactccccacaaccggccgacccggcgcgccacctcccgggcgcggcgcaccgggccggcgccgccgcgctccgcttcgtacagcagtggctggccgaacccgccctggaagacatcaagttggtcgtcgtcaccagaggggcggacgcgatcaccgatgaggacgtggtcgacccggccgcggcggtcgtccgcagcctgctgcggtcggcgcaggcggagaacccggaccggttcctgcttgccgacctccccgcgtgcgacggcgccccgcaggaggcggcgctgctgcccgcggcgctgggcagcgaggagccggaggtggcggtccgcggccggatccggtacggccggcgtctgacccgccccacgggtgaggtgacgcgcatccagtggccgtccgacacccgagccgacccggcagcccgctcgctgctggtcaccggcggcaccggaaccctgggcggactggtggcccggcacctggcggccaccggccgggccggctgcgtggtactgaccagccgttccggaccggccgcgaccggtgcggccgcacaggccgccgaactggcccagctgggcgtaccggtccgcatcgtcgcctgcgacgcagccgaccgggacgccctggccgccgtcctggagcagatcccggccgcgaacccgctgggctccgtcgtgcacgccgcgggcgtcgtcgacgacggtgtgatcatgtcgctgacgccggagcggctgtccgcggtgctgcggccgaaggccgatgcggcctggcacctgcatgagctgacggccggactccagctggaccggttcgtgctgttctcgtccgccgcagccgtcttcggctcggccggccaggccagctacgtggcggccaacgcgttcctggacgccgtggccgcgcaccgcaggtccaccggcctcacaggcacgtcgctgcaactcggcccgtgggctcacgaggccggcatcggccgcaacctggatgccacctccctcgcccgtatcgaccggggcttcgtaccgctgaccgccgacgagggactggtcgtcctggatacggcgctgaccagggatgaagcggtgctgatgcccgccaggccggacaccgcggcgctgcgcgcacagaccgcggccgggggcgaggtgccgccgctgtggcgggcactggcggggacgtccgcccggcgcaccgccgcctccgggactgccgccggctccggtacggaggccggcgaggcactgcgccacaggctggcctcggtcggcggcaccgagcgggaccggctcctgctggaactggtgctcgcacacgccgccgccgtgctgggacacgcctccccggacgcgatcgacgccggccgtgccttcaccgacgtcggcttcgactcgctgaccgcggtggaactgcgcaaccggctgaaggcacagaccggactgcggctgccggcgacgctggtgttcgaccaccccaccccggccgcgctcgccaccttcctgcgcggcgaactggccggagacctgcctgccaccgccgttcccaccgcaccggtagcggcgggcgccgacgaacccatcgcgatcgtcggcatggcctgccgcttcccgggcggcgcggccggcccggagcagctgtggcacctgctggactccggtacggacgcgatcggcccgttcccccgggaccgcggctgggacctggaccggctccaggacccggacgcggaacgcaccggcgtctcctaccagcaggtcggcggattcgtaccggacgccgcctccttcgacgccggcttcttcgggatcagcccgcgcgaggcgctggccatggacccccagcagcggctgctcctggagacctcctgggaggcgttcgaacgcgcgggcatcgacccggcatcggtgcgcggcagccagaccggcgccttcatcggcggctactcctcccggtacgcccacgacgcgtcgctggagggcaccggcgtcgccgaggcacacctggcgaccggcaacgcgaccagcgtgctctccggccggctctcctacgtcttcggcctggagggcccggcggtgacggtggacaccgcgtgctcctcctcgctggtggcgctgcacatggccgcacaggcactgcgcgcgggagagtgctcgctcgctctcgcgggcggcgtcacggtcaccgccaccccggacgtgttcgtcaccttcgccaccgcgggcggggtgtcgctggacggccgctgccgctccttcgccgcctcggcggacggctccggcttcgccgagggggtgggcctgctggtggtcgaacggctctccgacgcgcgccgccatgggcacaaggttctggccgtcgtacggggcagtgccgtcaaccaggacggcgcctccaacggcctgaccgcgcccaatggcccctcccagcagcgggtgattcgtgccgcgctggccaatgcccgtctcactcctggtgatgtggacgtggtggaggcgcatggctcggcgaccaagctgggcgaccccatcgaggcgcaggcgctgctggccacctacggccgggagcggccggaggggcggccgttgtggttggggtcggtgaagtccaacatcggccatacgcaggcggctgctggtgttgcgggtgtcatcaagacggtgctggctcttcagcatgagcggttgccgcgcacgctgcatgtggatgagccgacgccgcatgtggactggtcggcgggtgaggtcaggctgctggcggagtcggtgccgtggcccggtggcgagcgggtgcgccgcgccggtgtctccgggttcgggatgagtggcaccaatgtgcacgtgattctggaggaggccccggccgaggaggccgaggagacggacaccgacgggatctccgccccggtgaccaccgcaccggcgcctgcgctgctcggcgatggtggtcccacggcgtggttggtgtcgggtcggtctgcggagggtcttgccgcgcaggcgggtcggttgggggagtgggcggctgggcgtgctgcgttgcgtccggtggatgtggcgtggtcgttggcggctacgcgggcggtgttggaataccggggtgtggttgtcgctggtgaccgggcggggctgttggacggggtacgggggttggcaggcggtgagttggccgcgccgggtgtggtgtcgggggttgctcgggccacgggtcgtacggtgtttgtgttcgccggtcagggcgctcagtgggcggggatgggccgggatttgttggcttcgtcgccgttgttcgcggcgcgactcgatgagtgtgcggcggcgttggcgccgtatgtggactggtcgctgactgaggtgttggcgggtgcggagggtgctccggcgctggaggcggctgaggttgttcagcctgcgttgtgggcggtgatggtgtcgctggctgctgtgtgggaggccgctggggttactccggatgcggtggtgggccactcgcagggggagattgctgcggcgacggtggcggggatgctgtcgctggacgacgctgcccgggtggtggcggtgcgttcgcgtgcgttgtctgggctggatgtggccggggcgatggtgtcggtggtgatgccggctgacgcggtacgcgagttggtggcgggcttcgacggtcggttgtcggtagctgcggtcaatggcccggcgtcggtggtggtatccggacaggcagaggcactggcgtccttcgagcgggagctggcggcacgtaaggtgttgcgctggcgcatcccggcgacggactttgtggcgcattcggcggcggtggagccgcttgccgcccagctccgggaggagctgtcggagatagccccgcaggcgggccgcatcccgatggtctccaccgttacgggggagtggctggccggtgaggaagcggatgccgcgtactggttcgcgaacctgcgccagatggtgcgcttcgaggacgccgtccgtagcctgctggagtccgggtacggagcgttcatcgaaatctcgccgcatccggtgctgacggccgcggtgaccgaaaccgcccaggacacggacgcgaacgccacccccgtgctgagcatcggcaccctgtcgcagaccgacgcgggcgcggccggtctgctgcgcggcttcgcgcaggcccacgtggcggggctgccggtggactggcgcaaggtcctgccggccggcgaggtcgtagagcttcccacctacgccttccaacaccgccgctactggctggagacctccaccgcagccgccgccacggccgccacaaccgtttcctcggccgccgaggcgcagttctgggccgccgtcgaaagcggcgaccggaccgggctcgcccaggcactggccatcgagaacgagcaccaactcgacggcctgctgcccgcgctggcgtcctggcggcgccgtgaacaggaacgttcggccactgaaagctggcggtaccggatcacctgggcgccggtggccgacccagcaccggctcgcctggccggccgctggctgctgctcgtaccggcccaggaggccgacggcgaggcggcccggcagtgctgtgcggcgctcaccgcccgcggcgccactgtcgtgccggtccaggtgccgacaggcaccgttcagcgggactggatcgccgcgctgctcgaccggacacagcaggacgccgagccggcatccggagtgctctcgctgctggcgctggacgagacgccgctgccggaccacccggtggtgacggcgggcctggccgccaccctcgcactggtgcaggcgctcggcgacaccggcatcgcggcgccgctgtgggtggccacccgcggcgccgtcgccaccggccccgacgaggcgccgaccagcccgctgcaggcccagatctggggcttcggcaggatcgccgggctggagcacccggaccgctggggcgggctggtcgacctgcccgctgtcctggacgaacgtaccggcgggcagctggcgaccgcactggccggctctacggagaaccaggtcgccctccggccgtccggggccctgggccgcaggctcgcccatgccccgcagtccccggcggccgcgacctcgcagtccgcgacgggaaccgcgctgatcaccggcggcaccggcgcactcggcggccacgtggccgcctggctggccgcacgcggcgccgagcggctggtgctgaccagccgctccggccctgcagccgccggagccgcggcacaggccgccgcactggccgcgcgcggcacccgggtggacgtactggcctgcgacacggccgaccgttcccaactggccgccgcgataggccggatcggcaccgacggcccccggttcaccaccgtcgtgcacaccgcggcggtactggacgacggcgtggtcgacgccctctcgccggcccggctggagaccgcgctcgcggccaaggccgccggcgccgcccacctggacgagctgaccgccgacctggacctggacgctttcgtcctgttctcctccgtggtcgccaccaccggagcccccggacaggccaactacggtgcggccaacgcctacctggacgccctcgcccagcaccggcgcagtcgcggcctgcccgcactcgccgtggcctggggcccgtgggcggtcggcgtggcacagggctccgcagcggcacggcagcggctggcccgcaaccagtgggaagaggtgatggacccggacctggcggtacgggccctgggagaggcgctggacggcggcgacaccgtactgacggtgatgaacatcgactggtcggcgatcctcgcccagccgcagtcctcggccggtctgctgcaggccgccgtgatgcgggacctgcccgaggtccaccggctggccgcgctgctggccgagaccccgcaggcaccggccgagccggccctggcggaacggctggccggactgggccgccccgaacagcgccgggtactgagcgacctggtacgggccggggccgcccgggtgctcggccacacctcgcccgacgcggtcgaggaccggagcgccttcagcgacctcggcttcgactcgctgacgtccgtcgaactccgtaacgacctcagcgccgcaacgggtctcaggcttcccgcgaccctgctgttcgaccaccccaccccggccgctctcaccggctacctggagaccgaactcctgggcaccgggccgcagacacccgaggcaccggccgcgctgccggccaccatcgcggcggacgaaccgatcgcgatcgtcgggatggcctgccgctacgcgggaggcgtggccggccccgacgacctgtggcagctgctcacctccgcgggcgacggcatctccgggttccccgacgaccgtggctgggacctggaggcgctggacggcccctcgtacgtacggcacggcggcttcatcaaggacatcggcggcttcgacgccggtttcttcgggatcagcccgcgcgaggccctcgccatggacccgcagcagcggctgctgctggagacgtcctgggaggcactggagcgcagcggcatcgccccgacctccctgcggtccaccgcgaccggcctgttcatcggcggctacgcgtccggctacggctacggcgccgacttcgagggtgcggcccacctcatcaccggcaccgcgaccagtgtgctctccggccgggtgtcctacaccctcggcctggaaggcccggccgtgacggtggacaccgcctgctcgtcctcgctggtggccctgcacatggcggcccaggccctgcgctccggggagtgctcgatggcactggccggcggcgtcaccctgatggtgacgcccgaaggcttcatcggcttcagcgagcagagcggcctcgccaaggacggccgctgcaaggcgttctccgaccaggccgacggcatgggcatggccgagggcgccggcgtcctcgtcatcgagcggctttcggatgcgcagcgcaatgggcacccggtgttggcggtgctgcggggtagtgccaccaaccaggacggtgcgtcgaacgggctgaccgcgcccaacgggccgtcgcagcagcgggtgatccgggctgcgttggccaactcgcgtcttacgcctggtgacgtggatgtggtggaggcgcacggtactggtacgaagttgggtgacccgatcgaggcgcaggcgctgctggccacctacggtcaggaccgaccggagggccggccgttgcggctggggtcggtgaagtccaacatcggtcacacccaggccgctgccggtgtcgccggtgtcatcaagatggtgctggcgctccagcacgagcagctgccgcgcaccctgcacgtcgacgaactgtcctcacacgtggactggaccgcgggcgaggtggaggtgctgacggaaccggtgccgtggcccagcgggggccgggtgcggcgggccggcgtctcctccttcggcctcagcggcaccaacgcgcacgtcatcctggaagaggccccggcacccgccgtcacccaggacggtgagagccccgccgaaaccttcgaggggaccggggccgagggcacggtactcgttcccggcgtggcgccggcctggctggtgtccggccgctcggccgcgggcctggccgcgcaggcggagcggctgcgggactggctggcggcccgcccggagctggaatcggccgatgtggcctggtcgctggctgccggccgctcggtcttcgagcaccggggtgtcgtggtgggtacggggcgtgccgagctgctcggtggcgtggggagcctggcgtcgggtgttccgggtgggtcggtggtgtccggcgtggcccgctccaacgttcgtccggtgtttgcttttgcgggtcaggggtcgcagtgggtcgggatgggtcgggagctggcgggggtttcgccggtgttcgcggcgcggttggcggagtgtgcggcggcgttggcgccgtatgtggactggtcgctcagcgatgtcctggcgggtgcggagggtgctcctgcgttggaggcggcggatgttgtccagcctgctctgtgggcggtgatggtgtcgcttgccgctgtctgggaggcggctggtgtcgccccggaggcggtggttggtcactcgcagggggagattgccgctgccacggtggccgggatgctgtcgcttgaggacggtgcccgcgtggtggcgttgcggtcgcgtgcgttgaaggtgctggcgggtgcgggcggcatgctgtccgtgagccgtccggcggcggaggtcgaggaacgcctggtccggttcggcgaccggttgtccctggcggcggtcaacggcccctcggccactgtggtctcgggcgagccggcggctctggaggagcttcagtccgagttcgaagcggagggtgctcgggcccgtatggtcgcggtcgattacgcttcgcactccgctcaggtggaccggctggaggaggagatcaccaccgtcctggccggaatctccccgcgccggggccgcatcccgatggtctccgcgatgactggcgagaccctgacgggcgaggagctggacgccgggtactggttccgcagcctgcgggcgaccgtgcattacgaccgggccgtgcgggtgctggccggtcgtggtcatcaggtgttcgttgaggtgacgccgcatccggtgctgatgggggcgatgaacgacaccctggaagaggtcgcccaggaaacaggcccgggggcggagcccgccgcggtgtgtggaaccctgcggcgcgatgacggcggcgccgagcgcctgctgctctcgttggccgaggcgttcgtcaacggagccgagctggactggcaggcggtgttgccggccggacggcgggtggagctgccgacgtacgcgttccagcatgagcggttctggccgaaggtggctgcggctttcggtggtgatccggtgtcgctggggctgggtgcggtgggtcatccgctgttgggtgcggcggttgaactggcgggtggtgcgggtctggtgtgtacgggtcggttgtcggtgcgtacgcatccgtggctggctgatcacgccgtgggtggtgtggtgctgctgccgggcaccggtttcgtggagctggctgtgtgcgcgggggatcaggccgggtgtggtgtgctggaggagctgacgctccaggcgccgctggtcttccccgaggacggcagtgctgtgcaggttcaggtggtgctggcgggtccggacgccgaaggccgccgtagcgtcgaggtcttctcccgggtcgatgtggctggtcagtcgcaggggtggacgcagcacgcgagcggcgtgctggcccctgccggtccggtggctgcggccgaggaggacttcgcggtgtggccgcctcgtgacgccacgacggtggacatctctggcctgtatcaggcagcgggcgcgaactcgtacggctatggtccggcgttccaggggctgcgggcggtgtggcgccgaggccaggagatcttcgccgaggtggcgctgccggagcaggtggcggaggatgccgctgcctttggtgtgcacccggcgttgctggacgccgtactgcatgccagtgccttgctcgatgctgagggggagccgggcgagatccggttgccgttcgcctggacgggtgtggagttgtatgccagtggtgcgtcggtgctgcgcgcccggttgcgtcgtgacgcgcagggcacgctgacggtgtcggccgccgatgcggcgggtcagccggtgatttccgtagcgtcgctgatcacccgcccggtggacaccggccagctccgcaccgctgacaccggaatggccgatgcgctgttcggcgtggaatgggcagcggtcgccgcggccgaggcggccgccggggagtgggcgctgatcggcgccgaccgcttcgatgtcgctggacagctgacggctgccgggacacaggtgcgctgtttcgccgggctggaggagctggccgctgccgcagcggaccgggagatcgaccctgccttcgtcctggtctgcgccggtgacgagggccggggcgaggaggacccggcgcgggctgcggcccgggtgactggtgagatgctcggcctggtgcaggagtggctggctgaggagcggctggcggagagccggctggtggttctcacgcgtggtgcggtggcggtttcggccagtgagggggtggccgatctgccggcagccgctctggccggtctggtgcgttcggcccagtcggagaatcccgaccggctgctcctcgtcgacctgccggccaccggcaccaccgatcaggctgaggtgctcccgcttgccgttgccaccggggaaccggagctggcgctccgaaacggtgcggtgcatgcacgacggctggtgcgtccttctgcggagctgccggttccggagggccggtggcggctggagccggacgcgggcggttcgctggaaggactgtcgctgcttgcggcgccggaggccgatggagcgttggcggcaggacaggtgcgggtggcggtccgggcgaccggcctgaacttccgcgacgtactgatcgcgctgggcatgtacccgggcggtggggtactgggcagcgagatcgccggaacggtgctcgaagctggaccgggggtggaccacctcgtcccgggcgaccgggtgatggggctcggtgagggcggttttggtccggtggttgtcaccgacgcccggcagctggtgcgcgtaccggacggctggtcctacgccgaagccgcctccgttcccagcgcgttcatgactgcttggtacgcgttggtggagttggccggggcgaagccggggcagcgggtgttggtgcatgctgcgaccggtggtgttgggatggcggcggtgcagatcgcccggtacctggggttggaggtcttcgcgaccgcgagtccggggaagtggtcggtgctggcgtcgatggggctggacgcggagcacgtctcgtcctcccgtactgccgcattcgaggcggatttcctggctgccacggacggcgcgggtgtggacatcgtggtgaactcgctggcgggtgagttgaccgatgcttcgctgcggttgctgccgcggggcggggcgttcgtggagatgggccgtaccgacgtccgcgacgcccaggcagtggccgcagagcaaccgggcgtcacctaccgcccgttcgagctcagcgaggcgggaccggcagcactgggcacgatcctggagcgggtcgttgcgctgctgtccaccggcgagctgcaacagctcccgctacgggcttggggcgtccggcaggcacgggacgcgttccggttcatgcagcaggcccggcacaccggcaagatcgtcctgacgatccccgcacacctctccgcaacgcagccggaatcggcccggtccggtgcccgttcggcgttgatcacgggtggtacgggcacgttgggtggcttggtggccgggcatctggtggtgtctgggcgtgcggagcacgtggtgctgaccagtcgttcggggcccggtgcggccggtgtggcggcgttggctgcgcggttggcccaggcaggcgcttcggtgcgggtggtggcatgtgatgccgcggaccgccaggagctggctgctgtagtggcaacaatcccagcgcaggcgtcgctacgcaccgtggtgcataccgcgggtgtgctggacgacggtacggtcggttcgctgaccgcgcagcgggtggccggggtgctgcggccgaaggcggacggtgcctggaacctccacgagctgacccagggactggacctcgaccacttcgtcctgttctcctccgcggcagccaccttctccagccccggccagggcaactacgtggcggccaacgccttcctggacgcactggccgcgaagcggcgggcggccgggctgcccgccacctcgctggcctggggcatgtgggccgacgccagcgcgctgaccgggcagctcagcgagtccgaacgggcccgcatcagccgcggcggagtcatggcgctcagtgccgacgagggcttggcgctgcttgacctggcgctggcccgggacgaggcgctgctgatgccggccaagctcgacctggccgggatgcgggcgcagagcgccggcagcacggaggttccgccgctgttccgggccttggtctcggccggtagcgggcggcgtagcgccgccggggccggtgacgcgggtgcggactcgctgcatcagcggttgtccgggctgtccggtcaggagcggacccggctgctgatcgacctggtccgtacacaggcggcggctgtcctggggcatgcctcggccgaggccgtggagccggggcgggcctttaccgacctgggcttcgactcgctcaccgccgtcgaactgcgtaaccggctgagcaccgcgaccgggctgcggctgcccgccaccctggtcttcgactaccccaacactctcgaattggccgccttcctgaaggaaaagctgctgcctcagattgttgaacatggcgagccggacgctgcggaggagaaactccggacggcactcgcgaccgtaccgctgtccaggttccgggaggccgggctgatggaggcgctgctgcggctcgccggcgtccaggacgacgctctgctcgccgaagagggcgatcagggcgaggacatcgacgaactggacgccgacagcctgatccgtctcgcgctgggcggcgagacggaatga

SEQ ID NO: 65 - DNA, 23793 bp, Streptomyces sp.
  source 1..23793: mol_type = genomic DNA
gtgtccgacgaaaacaagcttcgtagttacctcaagcgagccatcgcggacgcccaggacgcccgcgcccggctgcgtgaggtgcaggagcaggcggcggagccggtggcgatcgtgggcatggggtgccgtttccccggtggcgcgggcagcccggaggacttctggaagctgttggcttccggtggtgacgcggtgggtggtttcccgaccgaccgtggctgggacctggagggcatttacgatccggacggtgagcgcgagaacacctcgtacgtcgcccagggcgccttcctacagggggcgaccgagttcgatgcggggttcttcgggatcagcccgcgcgaggcgctggccatggacccgcagcagcgcctgctgctggagtcggcctgggaggcactggagcgcgccggcatcgacccggcgtccctgaaggagagcgccaccggtgtcttcgccggcggattcgcctccggatacggcatcgggctctcctgggcagccggtgagggcggctccggtgtcgagggccacctgatgactggcaacgcgaccagcgtgctctccggccggctgtcgtacgccctgggtctggagggcccggcggtcacggtggacaccgcctgctcctcctcgctggtggcactccacctggccgttcaggcactgcggctgggagagtgctcgctggccttggccggtggtgtcacggtcctggtctccccggggacgttcgtggagttctcccgtcagcagggtctgtcggccgatggccggtgccgggcgttctcggaggatgcggacggtacgggctgggccgagggcgtcggcatgctggtggtggagcggctgtcggacgcgcggcgcaatgggcacaaggtgctggcggtgctgcggggcagtgcggtcaaccaggacggtgcctccaacggcctctcggcgcccaacggcccgtcccagcagcgcgtcatccgggcggcgttggccagtgcggggctctcggcgtccgaggtggacgtggtggaggcgcacggcaccggtacgacgttgggcgacccgatcgaggcgcaggcgctgctggccacctacggtcaggaccgtcgggaagggcggccgttgtggctggggtcggtgaagtcgaacatcggtcacactcaggctgcggctggtgttgcgggtgtgatcaagatggtgctggcgttgcagcatggtcagttgccgcggacgttgcatgctgatgtggcgtcgtcgcatgtggactggtcggcgggtgaggtgcggctgctgacggagtcggtgccgtggccggtgggtgagcgtccgcgtcgggctggtgtgtcggcgttcggtatcagcggtaccaacgctcacgtgattctggaggaggctccgccggtggagaccggcgaggccgacgggtctgctgaggccggctccgggacggccgggtcgggcgggccggccgggtcggtggcggatgcggcgccggtgctggcgggtgctggtgcgtgggtggtgtcgggtcgtacggcagaggcgttgtcggcgcaggccgggcggttgcgggagtgggtggcagcgcgtccggggctgggtgcggctgatgtggcttggtcgttggcggcgacgcggtcggcctttgagcatcgcgcggtggtgctcggttcggagcgtggtgcccttctcgatggcgtgcacagcctggcggctggtacttcctcgcctgtggtggtgtcgggcgtggctcggccggatgtccgggtgggtctgatctttgcggggcagggtgcgcagtgggtgggtatgggccgtggcctgtatgcgggcagtgcggtgttcgccgaggtctttgaccgggtgtgtgggctgctggagctggaactcggggccggcgtgcgtctgcgggatgtggttttgggtgcggagggggtggacgctgggctggcggatcggacgctgtatgcgcaggctgggctgttcgctttcgaggtggctctggctgcggtgctgaaggctgttggtgtggtgccggatgcggtggtgggccactcggtcggcgaggtggctgcggctcatgtggcgggtgtgttgtcgttgccggatgcctgtgcgctggtggcggctcgtgcccggttgatgcaggagttgccgtcgggtggggcgatggcggcggtcaacgccgccgaggcagatgtcatcgcctcgtttgacgaggtgtcgggtgaggtggccgtcgcggcggtcaacggtccggagtcggtggtggtctccggtacggccgatacggttgatgcggtggtggagctgtggcgggggcggggctgccgtgtgcggcgcctgcgggtttcgcacgctttccactccccggcgatggacccggttctggacgagcttgatggcgtggcgcaggggttggtgtatcaccggcctgaggtgatgtgggcgggtgcgctcaccggtgagttggtgtccgatccgcaggccgggtactggcctgcgcagacccgccaggccgtccggttcgcggatgcggtggccaccctcgcacgggagggtgtctcggtcttccttgaggtgggtccggacggctcactctcctccctcggcccggatgcggtggccggtgtggagggtgcggaggcggcgttcgtcccccttcagcggcgcaccgaggacggcgccaccgacctggagaccggtctggcccgtgccttcgtcaacggtgcgccggtggactggacgcgtgtgctgcccgccggtgagcaggtcgagctgcccacgtatgccttccggcaccagcggtactggcccgaggggatcgtctcgctgatgcccccgacggccggcggtgcgaaccggatcaccgcgggcacggaggcggaggccgagttctgggcggccgtcgagggagaggacctcacccggctcgccgagacgctggacgtggacggcgaccgtccgttcagcgaggtgctgccggcactggcctcctggcggcggcgcgacctggagcgctccctgactgctgactggcgctaccgcaccacctgggcaggcgttgccgagcctgacgctcgtgtgctgtccggtacgtggttggtcgctgtccccgcgggggcggctgcggtggggctggcgcagggttgcaccgaggcgctgcgcgcccgtggtgctgaggccgtggtggttgagatcccggctgggacggtggaccgggccgggcttgcggcgttgctgaccggcgcggcggatgcgtcggccgtctctggtgtgctgtcgttgctggcgctggacgagacgccgctgccggatcactcggcggtgcccgccgggctggccgccaccttggctctcgtacaggctctcggtgatgccgggttcgaggtgccgctgtgggtggtgacgtccggtgcggtggcggccggtgcggaggaggtgctggcccgtccggtgcaggcgcaggtgtggggcctgggtcgggtggtcgcgctggagcacccggagcgttggggtggtctggtcgatctgccggagtcgctcgatgagcgggccggtgcacggctggtggcggtactggctggctatggtgagaacgaggtggccctgcgaccgtccggaattctgggtcgccggctcgcgcacgtatcgggtcgcggtgcgcgggagcgctgggcgccgcggggaaccgtactggtcaccggcggcaccggtgcgattgccgggcatgtctcgcgatggttggcaggccggggtgccgaccgtgtggtgctggcgggccgttccggtccggcagcagccgatgtcgcggcacaggccgcggagctggccgccgctggcgtccacgtggacgtgatctcctgcgatgtcagcgaacgcggcccgctgaccaacctgctgagctggatcggcagcagcggcccggagctgtcctcggtgatgcataccgcgggcgtgctggacgacggtgtgctggaccggctgtcggtctcccgcctgcagtctgtgctggcggtcaaggttcacggtgcggtgctgctggacgagctgaccgtggacctggacctggacgcgttcgtactgttctccgctgcggctgccaccctgggcggtccggggcagggcaactacgcggcggccaatgccttcctggacgcgctggcggagaaccggcgcgcccgcgggctggccggcctggcggtggcctggggcccgtgggccggcggaggcatggccgactccaacgaggcggtccgctcccggatgcggaagatgcccatgccgccgatggacccgcagctcgcggtccgcgcgctggccgaggcgttgcaggggccggatgccgccgtcgctgtcatggatgtggactgggcgcagctggcatccgatgccggtgccacggggatgctgaagcggccgttggtgcgggatctgcctgagatccgtcagttgctcgcggcggccgggaacacggtcaccgtggcgcctgcggagggggagctggctcgtcagctggctgggctggaccgcgcggagcaggagcggatgctcgccgacgtggtgcgtggcgaggctgcggtggtgctggggtacggctcggctgatgcggtggaggaccgtcgggcgttcaaggatctcgggttcgattcgctgacagcggtcgagttgcgtaaccgtctcaatgcggcgaccgggttgcgtctgccgtcgaccctggtcttcgattaccccactccgctggcgctggctgccttcctgcgcgacgagctgaccgggcagcagaagggcacggccgctccggtaatcaccgtggtggcggcgcctgcggacgagccgctggcgattgtcgggatggcgtgccggttccctggtggagtgggcagtccggaggagttctgggagttccttgcttccggtggcgatggcgtgggcgatttcccggccgaccgtggctgggacttggagagtgtgtacgacccggagggcgagcgggagggcacttcgtacgtcgcgcagggtgccttcctgcgggaggcggctgagttcgacgcggggttcttcggaatcagcccgcgcgaagctctcgcgatggacccgcagcagcggttgctgctggagacgtcctgggaggcgctggagcgtgccggcatcgacccggcctcgctgaaggagagcgccaccggtgtctttgccggcggtttcacctcggggtatgggtttgggctgtcgcttgccaatcagggggactctggtgccgaggggcattcgctgacggggaatgcgaccagtgttctgtccgggcgggtgtcgtatgtgctgggtctggagggtccggcggtgacggtggacacggcgtgttcgtcgtcgttggtggcgcttcatctggcggcgcaggcggtgcggtcgggggagtgctcgctggcgttggcgggtggggcgacggtgatggcgacgccgggtacgttcatcgacttctcccgtcagcagggtctgtcggcggatggccggtgccgggcgttttctgaggatgcggatggcacgggctgggccgagggtgccggcatggttgtggtggagcggctgtcggatgcccgccgcaacggccacaaggtgctggcggtgctgcggggcagtgcggtcaatcaggatggtgcgtccaatggtctgactgcccctaacgggccgtcgcagcagcgggtgatccgtgcggccctggccaacgcccggttggctccgggggacgtggacgtggtcgaggcccatggcaccggtaccacactcggtgaccccatcgaggcgcaggcgctgctggccacgtacggccgggatcgtggtgaccgggacgcgctgtggctggggtcggtgaagtccaacatcggtcacacccaggccgcggccggtgttgccggtgtgatcaagatggtgctggcgctccagcatgagcggctgccgcagacgctgtacgccgaggtgccgtcctcgcacgtggactggtcggctggtgcggtgcggctgctcaccgagtcggcgccttggccggccgacggggatcgggtgcggcgggccgctgtttcggcgttcggcatgagcggtaccaacgctcacctcatcctggaagaggctcccacagcgcaggaggccgaagctgtcgaggggcccaaggaaccggtggttccggtggtcaccggtgccggtgcgtgggtgctgtctgggcggactgctgaggcgttgtcggcgcaggcggggcggctgcgggagtgggtttcgcttcggccggctcttcggccggttgatgtggcgtggtcgttggctgctactcggtcggtcttcgagcaccgtgcggtggtggttggcggagaccgggagcaggtgctcgcggggctcgggagcctggcgtccgctgcgtcctcgggctcgctcgtttcgggcgtggcccgctccaatgcccgtccggtgttcgccttcgctggtcagggctcgcagtgggtcgggatggggcgcgagctggcgggggtttcgccggtgttcgcggtgcggttggcggagtgtgctgcggcgttggcgccgtatgtggactggtcgctgaccgatgtcctggcgggtgcggagggtgctcctgcgttggaggcggcagatgtcgtccagcccgcactgtgggcggtgatggtttctctcgctgctgtctgggaggcggcgggggtcgctccggaggcggtggtgggtcactcgcagggggagatcgctgcggctacggtcgcggggatgctgtcgcttgaggacggcgcgcgcgtggtggcgttgcggtcgcgtgcgttgaaggtgctggcgggtgcgggtgggatgctgtccgtgagccgtccggcggcggaggttgaggagcgtctggtccggttcggggagcgggtgtccttggcggcggtcaacggaccgtcggccactgtcgtctcgggtgagccggcggctctggaggagcttcagtccgagttcgaggcggagggcgcccgcgctcggatggtcgcggtggattacgcctcgcattcggctcaggtggaccggctggaggaggagatcaccaccgtcctggccgggatctccccgcgccggggccgcgtcccgatggtctccgcgatgaccggcgagaccctcaccggcgaagaactggacgccgggtactggtaccgcagcctgcgcgccaccgtgcattacgaccgggccgtgcgggttctggtcggtcgagggcatcaggtgtttgttgaggtgacgccgcatcccgtgctgatgggcgcgatgaacgacaccctggaagaggtggtccaggacgcgggtgcagagcccgccgccgtatgcggaacgctccgccgggacgacggcggcgccgagcgcctgctgacttccctggccgaggcgttcgtcaacggggcaccggtggactggcaggcagtgctgcctgctggtgagcaggtggagctccccacctacgcgttccgacgtcgccggttctggccggagggcatgctggtcctgccgatgcccggttccgcgaccgccggggccggtgccagcacggacgcggaggcggcgttctgggcggccgttgaggatggagacctgtcggggctgggtgatgcgctggctctggagggcgggcgtccgttcaatgaggtgctgccggcgctggcgtcgtggcggcggcgtgagcgggatcgttcgaccaccgctggctggcgttatcgcactggctggtcggtcatcgccgagcctgatgttcggctgctgtccggtacgtggctgatcgccgtccccgcggggacggctgctgcggagctggcgcaggactgtgtcggggcgctgagcgcccgtggtgctgaggccgtggtggtcgaagtaccggtcgggtcggtgaaccggggcgagctggcggcgttgctgacggtcgcggcggatgcgtcggccgtgtccggtgtgctgtcgctgctggcgctggatgaggcgccgatacccgggcatccggtggtgcccagcggcctggccaccaccttgggtctggtgcaggctctcggtgacgccgggatcgaggcgccgctgtgggtggcgacctccggtgcagtcgctgccgggcggggcgaggtgctggcccgtccggtgcaggcgcaggtctggggtctgggccgggtggtcgcgctggagcacccggagcgttggggtggtctggtcgatctgccggagacgctcgatgagcgggccggtgcgcgcctggtggcggtactggccggctgcggcgagaacgaggtggcgatccgggcggccgggatcttgggccgcaggctcgcgcacgtctcgggtcgcggtgcgcgggagagctgggtgccgcggggaaccgtactggtcaccggcggcaccggtgccattgccgggcatgtctcacgatggatggccggccggggtgccgagcgcctggtgctgaccagccgttcgggtccggcggctctgggcgcggcagcgctggcggccgaactcgcggcagccggttccgccgtggagatcgtggcttgcgatgtcagcgagcgtgccggtctggccgggctgctggactggatcgatgcgagcggtccccggctctcttcggtgatgcataccgcgggtgtgctggacgacggtgtggtggaccgcctcgacacggcgcggctggagactgtgctgggcgtgaaggcgcacggtgcggtgctgctggatgagctgaccgccgggctggatctggatgcgttcgtgctgttctcctcggccgcttccaccctcggcggcccgggccagggcaactatgcggcggccaatgccttcctggacgcgctggcggagaatcggcgtggtcgtgggctggctggcctggcggtggcctggggcctgtggggcggtggtggcctcggtgagtcgagcgaggtcattcggtcccggatgcgtcggatgccgatgccgccgatggacccgcagttggctgtgcgggcgttgggcgaggcgttggacggtccggatgccgtggtgacggtgatggaggtggactgggcgcagctggcatccgatgccggcgtcacggggatgttgaagcggccgttggtgcgggatttgcctgagatccgggagctgtccgcggcctccggccgtgcgccggccctggcgccggtggagggcgaactggccgctcgggtagcggggttgtcccgctcggagcaggaccgggtgctgaccggcgtggtccgtgccgaggccgctgtcgtgctcgggcacgactcggcggacggggtgcaggcgcggcaggcgttcaaggatctcggtttcgattcgctgacctcggtcgagttgcgcaaccgtctcaacgcggcgaccgggctgcggttgccggcgaccctggtcttcgaccacccgactccggtcgcgctggccgccttcctgcgcggcgaaatgatcggcggccacccggacgccgccgacagtggaacttccgctattcagactgcggtggacggcacgccccccgctgcggacgagccgctggcgatcgtcggcatggcctgccgcttccccggtggcgcgagcagcccggaggagttctgggagctgttggcttccggtggtgacgcggtgggtggtttcccgaccgaccgtggctgggatctggagggcatttacgatccggacggtgagcgcgagaacacctcgtacgtcgcccagggcgccttcctgcagagggcagccgagttcgatgcgggtttcttcgggatcagcccgcgcgaagccctcgccatggacccccagcagcggctgatgctggagacgtcctgggaggcgttggaacgcgccggtatcgacccgtggtccctgcgtggcagctcgaccggcgtattcgccggcggcttcactaccgggtacggccttggggtctctgcagcggctggtgagggcggctccggtgtcgagggccacctgatgactgggaacgcgaccagtgtcctgtccggccgggtgtcgtatgtgctcggtctggaggggccggcggtgacggtggacacggcgtgttcgtcgtcgctggtggcgctcaacctggccgcgcagtcggtacggtccggggagtgctctatggctctggccggtggtgtgaccgtgatggctacgccgtcctccttcgtggagttttcccgtcagcagggtttgtcggctgacgggcggtgccgggcgtttgcggatgatgcggacggtacgggctgggctgagggtgccggtgttctggtggtggagcggctttcggatgcgcggcgtaatgggcatcgggtgctggctgttgtccgtggtacggcggtgaatcaggacggagcgtcgaatggtctttcggcgccgaacggtccgtctcagcagcgggtgatccgggcggcgctggccaacgcccgactgtccgccgccgaggtggacgtggtcgaggcgcatggcaccggcaccaccctcggtgaccccatcgaggctcaggccctgttggcgacctacggcaaggaccgcggggagagtgacccgttgtggctggggtcggtgaagtcgaacatcggtcacactcaggctgcggctggtgttgcgggtgtgatcaagatggtgctggcgttgcagcatggtcagttgccgcggacgttgcatgctgatgtggcgtcgtcgcatgtggactggtcggcgggtgaggtgcggctgctgacggagtcggtgccgtggccggtgggggagcgtccgcgtcgggctggtgtgtcggcgttcggtatcagcggtaccaacgcgcacgtgattctggaagaggctccgccggtggaggccggcgagcccgtcgaggccggttccggaactgccggggcggtggcgggtgctggtgcgtgggtggtgtcggggcgtacggctgaggcgttgtcggcgcaggccgggcggctgcgcgagtgggtgtcggcccgtccggaactgggtgcggctgatgttgcctggtcgctggcggcgacgcggtcggcgttcgagcaccgcgcggtggtgctcgggaccgagcgtgatgcgttgctgtccggcctgcacaagctggccgccgggacctcctcgtccgaggtggtgtcgggcatggcccggccggatgcccgggtgggtctgatcttcgcgggtcagggtgcgcagtgggcgggtatgggccgtggcctgtatgcgggcagtgcggtgttcgccgaggcctttgaccgggtgtgtgggctgctggagctggaactgggggccgaggtacgtctgcgggatgtcgtcctggagggtgacggggaagctgctgggttggcggatcagacgctgtatgcgcaggcgggtctgtttgccttcgaggtggctctggcggcggtgctgaaggctgctggtgtggtgccggatgcggtggtcggtcattcggtgggcgaggtggctgcggcccatgtggctggtgtgttgtcgctgtcggatgcctgtgcgctggtggcggctcgtgctcggttgatgcaggagttgccgtcgggtggggcgatggcggcggtcaacgccgccgaggcagatgtcatcgcctcgttcgacggggtgtcgggtgaggtggctgtcgctgcggtcaacggcccggaatccgtggtcatctccggtgcaaccgaggccgtggacgcggtggtggagctgtggcgggaccggggctgccgggtgcggcgcctgcgggtctcgcacgccttccactccccggccatggacccggtcctggacgagctccgcaccgtcgccgaagggatggagttccgccgcccggaggtgatgtgggcgggtgccctcaccggtgacctggtgtccgacccgcaggccggctactggcccgcgcagacccgccagaccgtccgcttcgccgacgcgatagccaccctcgccaccgatggcatctccgtcttcctcgaagtgggtccggacggctcgctctcctccctcggcccggatgcggtcgtggaggtggatggggccgagcccgccttcctcccgctccagcggcgtgacgacgaaggcgccacgggcctgctgaccggcctggctcgcgccttcgtcaacggtgccccggtggactggacgcgtgtgctgcccaccggcgaacacgtcgagctgcccacctacgccttccggcaccagcggttctggccggaggctccggcggcgttgtcagccaatacctcggccaccgcccgggatggagcggggagcgcggccgagtcggcgttctggtcggccgtcgagagcggtgacctggagcagctggcggacactctggcggtggacggcgaccgtcccttccaccaggtgctgcccgcactggcgtcttggcaccgtcgcgagctggaccgttcggccaccgccgactggcgctaccgaaccgtctggtcgcccgttgcagagcttgacgctcgtgtgctgtccggtacgtggttggtcgccgtccctacggagtcggctgcggcagacctggcgcaggactgtatcgaggcgctgagcgctcgtggtgccgaggccgtggtggtcgaggttccggccggaacggtggaccgggtcgaactcgccgcgttgctgactgacacggtggatgcgtcggccgtctctggggtgctgtcgctgctggcgctggacgagacaccgctgccggaccactcggcggtgcccgccggactggccgtcaccctggctctcgtacaggctctcggtgacgccgggatcgaggtgccgctgtgggtggcgacgtccggcgcggtggcggccggtgcggaaggagccttggcccgtccggtgcaggcgcaggtgtggggcctgggtcgggtggtcgcgctggagcacccggagcgttggggtggtctggtcgatctgccggagtcgcttgacgagcggtccggtgcgcggctggtggcggtactggccggctgtggtgagaacgaggttgctctgcggccgtccggaatcctgggccgtcggctcgcgcgtgttacgcgacacggttccgaggagaactggacgccccgcggcagtgtgctgatcaccggagggacgggcgcgatcgcggggcatacggcgcgctggctctgtggtgagggcgctgagcgtctggtgctgacgagccgctcgggtccggcggcggagggtgtggccgcgctggccgcggagcttgctgaggcgggcagcacggttgcggtggtggcgtgtgatgtgagtgaacgggcgccgctgtctgacttgttggattggatcggccgtagcggtccggagctgtcctcggtgatgcataccgcgggtgtgctggatgacggtgtggtggaccgcctcgacactgcgcggctggagactgtgctgggcgtgaaggtgaggggtgcggtgctgctggacgagctgaccgcggacttggacctggatgcgttcgtactgttctccgctgcggctgccactctgggtggttcggggcagggtaactacgcggcggccaatgccttcttggacgcgttggcggaggaacgccgggcccgtgggctgaacgctctggcggtggcgtgggggccgtgggctggtggtggtatggctgcgtcgagtggtgcggtgcgggcgcgggttgagcggggtgcgatgccgccgatggacccgcggcttgccgtccgtgcactggccgaggcgttgcagggaccggacgccgccgttgccgtcatggacgtggactggacacagctggcctccggccctggggccgtggagctgctgaagcggccgttggtgcgggacctgcctgagatccgtcagctgctcgcggcggccgggaacacggtcaccgtggcgcctgcggagggggagctggcccgtcagctggctgggctggaccgctcggagcaggagcggttgctgacggaggtggtacgggcggaagccgctgctgtgttgggctatggctcggctgatgcggtggaggaccgtcgggcgttcaaggacctcgggttcgattcgctgaccgcggtcgagttgcgtaaccgcctgaacgcggcgaccgggttgcggctgccgtccactctggtcttcgattaccccaccccgctggcgctggccgctttcctgcgcggtgagctgaccggtggggagttggacaccgctggtgctccggtggccacggtggcggcggcgcctgcggacgagccgttggcgattgtcgggatggcgtgccgattccccggcggggtgggcagcccgcgggagttctgggaactgctgacctccggtggcgacgcggtcggtgacttcccgtccgaccgcggctgggatctggacggcctcattgacccggagggtgagcgcgaggggacctcgtatgtcgcccagggcgcgttcctgcgggaggctgccgagttcgatgccgggttcttcggaatcagtccgcgtgaggcgctggcgatggacccgcagcagcgcctgctgctggagacgtcctgggaagcgctggagcgcgccggcattgacccggcctcgctgagggagagcgccaccggtgtctttgccggcggtttcacctcgggttatgggcttgggctgtcgctctcccatcagggggattccggtgccgagggccattcgctgacgggcaatgcgaccagtgtcctgtccggtcgggtgtcctatgtgctcggcctggaggggccggcggtgacggtggacaccgcctgttcgtcctcgttggtggcgcttcatttggcggcgcaggcggtgcggtcgggggagtgctcgctggcgttggcgggtggggcgacggtgatggcgacgccggggaccttcatcgacttctcccggcagcagggtctctcggcggacggccggtgccgggccttctccgaggacgcggatggtacgggctgggccgagggcgccggcatggtcgtggtggagcggctgagcgatgcccgccgcaacggccacaaggtgctggcggtgctgcggggcagcgcggtcaaccaggacggtgcgtccaacggtctgaccgcgcccaacggcccctcgcagcagcgcgtcatccgcgccgcgctggccaactccggcctgaccaccgccgacatcgacgtggtggaggcgcacggcaccggtaccacactcggtgaccccatcgaggcccaggcagtactggcgacgtatggccggggccgtgcggaggaccggccgttgtggctggggtcggtgaagtccaacatcggtcacactcaggccgcggccggtgtggcgggtgtgatcaagatggtgctggcgttgcagtacgggcagctgccgcggactctccatgccgaggtgccgtcgtcgcacgtggactggtcggcgggtgcggtgcggctgctgaacgagcagctggcctggccgtccgatgatgatcgggtgcggcgggccgctgtttcggcgttcggtatgagcggtaccaatgcccacctcatcctggaggaagcccctgcgccggagcccgccgaggtgatcgacggccttgaggacgcgacaacaccgaaggcggcagcgctggccgcggacgccggtatcggcgcttgggtgctgtccgggcggactgctgatgcgctggcggcgcaggcgggccggctgcgggagtggacggcgacgcgtccggagctggcaccggccgatgtggcctggtcgctggcggcgacacgttccgtcttcgagcaccgtgcggtggtcgtgggcggggaacgcgagcaggtgctggctgggcttggagggctgacgtcgggtattccggctggttcggtggtatccggtgtggcccgctcccatgcccggccggtgttcgctttcgctggtcagggctcgcagtgggtcgggatgggccgcgagctggctgaggtttcgccggtgttcgcggcgcggttggcggagtgtgcggcggcgttggccccgtacgttgactggtcgctggccgatgtcctggccggcgcggaaggtgcccctgcgttggaagcggcggatgtcgttcagcccgcgctgtgggcggtgatggtctccctcgccgctgtctgggaggccgccggtgtcgccccggaggccgtggtcggccactcgcagggggagatcgccgctgccacggtcgccggaatgctttcgcttcaggacggcgcgcgtgtggtggcgttgcggtcgcgtgcgctgaaggtgctggcgggtgcgggcggcatgctgtccgtcagtcgtccggtggcggaggtcgaggagcgtctggtccggttcggggagcggctgtccctggctgcggtcaacggcccgtcggcgacggtggtgtccggcgagcccgaggcccttgaagagctgaaggcggagttcgaggcggagggtgcccgcgctcggatggtcgcggtggactatgcctcgcactccgctcaggtggaccggctggaggaggagatcacctctgtcctggccgggatctccccgcgccggggccgtgtcccgatggtctccgcgatgactggcgagaccctgaccggcgaggagctggacgtcgggtattggtaccgcagtctgcgcgccaccgtgcattacgaccgtgccgtgcgggttctggccggtcgtgggcatcaggtgttcgtcgaggtgacaccgcacccggtgctgatgggcgcgatgaacgacaccctcgtggacctcgccccggacgcgggtggagagcctgccgccgtatgcggaacactccgccgtgacgacggcggtgccgagcgcctgctgacttccctggccgaggcgttcgtcaacggggcaacggtggactggcagacagtgctgcctgcgggcgaccaggtggagcttcccacctacgcgttccggcgtcgccggttctggccggagggcatgctggtcctgccgatgcccggttccgcgactaccgtggccggggctggcaccgaggcggaggccgggttctgggcggcggttgaggacggtgatctggcgcgggtcgcggagacgctggcgctggaggacgcgggccagctgggtgcggtgttgccggcgttggcgtcgtggcggcgtcgtgagctggaccgttcggctactgctgactggcgctaccggaccgtctggtcgcctgttgccgagcctgatgctcgtgtgctgacggggacttgggtggtgcttgcgtcggccggttgctcggaggagctgcggcatcagtgtgtgacggcgctgacggcccgtggtgccgaggccgtggtggtcgaggttccggctgggacggtggatcgggccgggctgacggcactgctgaccggcgcggcggcggacccgtcagccatctccggtgtgctgtcgctgctggccctggacgagtctgtgctgccggatcgtccggttgtgaatgctgggctggctgccaccttgggcctggtgcaggctcttggtgacgctggggttgaggcgccgctgtgggtggcgacctccggtgcagtcgctgccggccgcggcgagatgctgtcgcatccggtgcaggcgcaggtgtggggtctgggccgggtcgtcggcctggagcacccggagcgttggggtggtctggtcgatctgccggagacgctcgatgagcgggccggcgcacggctggtggcggtactggccggctgcggcgagaacgaggtggcgatccgggcggccgggatcttgggccgcaggctcgcgcacgtctcgggccgtggtgtgggggagagctggtcgccgcgggggactgtattggtcaccggtgggaccggtgcgattgccgggcatgtctcgcggtggctggccggccggggtgccgagcgtctggtgttggcgggccgttccggtccggcggcagccgatgtcgcggctcgagccgcggagctggccaccggcggcgtccacgtggacgtgatctcctgcgatgtcagcgaacgcggtccgctgaccaacctgctgggctggatcggcagcagcggtccggagctgtcctcggtgatgcataccgcgggtgtgctggatgacggtgtggtggaccgcctcgacacggcgcggctggagaccgtgctgggggtgaaggcccacggcgcggtgctgctggacgagctgaccgcgaacatggatctggacgcgttcgtgctgttctcctcggccgcctccaccgtcggcggcccgggccagggcaactatgcggcggccaatgccttcctggacgcgctggcggagaatcggcgcggtcgtgggctggccggtctggcggtggcctggggcctgtggggcggtggtggcctcggtgagtccagtgaggtcattcggtcccggatgcgtcggatgccgatgccgccgatggacccgcagttggctgtgcgggcgttgggcgaggcgttggacggtccggatgccgtggtgacggtgatggaggtggactgggcgcagctggcatccggtgcgggtgctgctgatatgcgggtccggccgttggtgcgggacctgcccgagatccgggagctgtctgcggcctccggccgtgcgccggccctggcgccggtggagggcgaactggccgctcgggtagcggggttgtcccgctcggagcaggaccgggtgctgaccggcgtggtccgcgccgaggccgccgccgtactgggacatgcctcttcggacggggtgcaggcgcggcaggccttcaaggacctgggattcgattcgctgacctcggtcgagttgcgtaaccgtctcaatgcggcgaccgggctgcggctgccggccaccctggtcttcgaccaccccaccccgaccgcgctcgccaccttcctgcgcggcgaactgaccggccaggtgccggaggccgtagctgcgacgccggtccccgctgcccccgctgcggacgagccgctggcgattgtcggcatggcctgccgtttccccggtggcgcgagcacgcccgaggggttctgggagctgttggcttccggtggtgacgcggtgggtggtttcccgaccgaccgtggctgggatctggagggcatttacgatccggacggtgagcgggagaacacctcgtacgtcgcccagggcgcgttcttgcggggggcggctgagttcgatgcggggttcttcgggatcagtccgcgtgaggcgctggcgatggacccgcagcagcggctgatgctggagacgtcctgggaggcgttggaacgggccggtatcgacccgtcgtccctgcgcggcagttcgaccggtgtgttcgccggcggtttcgcctctggttatggccttggggtttccgcagcggccggtgagggcggctccggtgtcgagggccatttgatgaccggcaacgcgaccagtgtcctgtccgggcgggtgtcgtacgtgctcggtttggaaggtccggcggtgacggtggacactgcctgttcgtcctcgctggtggcgctcaacctggcggcccaggcggtgcggtccggcgagtgctcgatggctctggccggtggcgtcacggtgatggcgacgccgggtacgttcatcgacttctcccgtcagcagggtctgtcggcggatggccggtgccgggcgttctcggaggatgcggacggtacgggctgggccgagggcgcgggcatgctggtggtggagcggctgtcggacgcgcggcgcaatgggcacaaggtgctggcggtgctgcggggcagtgcggtcaaccaggacggtgcctccaacggcctgaccgcccccaacggcccctcccagcagcgcgtcatccgggcagcgttggccagtgcgggtctctcggcgtccgaggtggatgtggtggaggcgcacggcactggcacgaagcttggtgacccgatcgaggcgcaggcgctgctggcgacgtatggccgggatcgtggtgaccgggatgcgttgtggctggggtcggtgaagtccaacatcggccacacccaggccgcggccggtgttgccggtgtcatcaagatggtgctggcactccagcacggccagctgccgcggacgttgcacgctgatgtggcgtcgtcgcatgtggactggtcggcgggtgaggtgcggctgctgacggagtcggtgccgtggccggtgggtgagcgtccgcgtcgggcggggatctcggcgttcggtatcagcggtaccaacgctcacgtgattctggaggaggcccccgcgatcgaggcgcggagcgagggcgccggtcaggagccggttgccgctcttgagggtgctggtgcgtgggtggtgtcggggcgtacggctgaggcgttgtcggcgcaggcggggcggctgcgggagtggctgacggcccgtccggaactgcgaccggccgatgttgcctggtcgttggcggcgacgcggtcggcgttcgagcaccgtgctgtggtgcttggtacggagcgtggcgccctgctcgatggcgtgcacagcctggctgctggtacgtcctcgcctgtggtggtgtcgggcgtggcccggcccgacatccgggtgggtctgatcttcgcgggccagggtgcgcagtgggcgggtatgggccgtggcctgtatgcgggcagtgcggtgttcgccgaggtgttcgaccgggtgtgcgggctgctggagttggagctggggaccgaggtacgtctccgggacgtcgttctgaacagcgacgatgcggctgctggactggcggaccagaccctgtacgcgcaggccggtctgttcgccttcgaggtggctctggcggcggtgctgaaggctgctggtgtggtgccggatgcggtggtgggccactcggtcggcgaagtggctgcggctcacgtggctggggtgctgtcgttgccggacgcttgtgcgctggtggcggctcgtgcccggctgatgcaggagctgccgccgggcggggcgatggcagcgatcaacgccgccgaggcagacgtcatcgcctcgttcgacggggtgtcgggcgaggtggccgtcgcggcggtcaacggcccggaatccgtagtcatctccggtgcggccgaggcagtcgacgcggtggtggagttgtggcgggaccggggttgccgtgtgcggcgcctgcgggtctcgcacgctttccactccccggccatggacccggtcctggacgagctccgcaccgtcgccgaagggatggagttccgccgcccggaggtgatgtgggctggcgccctcaccggtgacctggtgtccgacccgcaggccggctactggcccgcgcagacccgccagaccgtccgcttcgccgacgcagtagctgccctcgcggcggaaggcatctccgtcttcctcgaagtgggcccggacggctcgctctcctccctcggcccggatgcggtcgcagaggtggacggggccgagcccgccttcctcccgctccagcggcgtgacgacgaaggcgccacgggcctgctgaccggcctggctcgtgccttcgtccacggtgtgccggtggactggacgcgtgtgctgcccaccggcgaacacgtcgagctgcccacctacgccttccggcaccagcggttctggccggaggctccggcggcgctgtcggccggtgtttcggccagcggccgggacggggcggggagcgcggccgagtcggcgttctggtcggccgtcgagagcggtgacctggaacagctggcggacactctggcggtggacggcgaccgtcccttccaccaggtgctgcccgcactgacccagtggcggcggcgtgacctggagcgttcgacgactgccggctggcgctatcggatggtctgggagccggtcgccgagcccgacccgggtgcgctgaccggcacctggctggtcgtcgtccccgcgcagtcggccgctgcggagctggcgcagggctgcgccgatgcgctgagcgcccgtggtgccgaggtcgcggtggtcgaaatcccggccgggaccatggaccggggcgagctggccgcgttgttgggcaaggcggcggacccgtcggccgtctctggtgtgctgtcgctgctggcgctggacgagacaccgctgccggaccactcggcggtgcctgttggactggccgccaccctggctctcgtacaggcactcggcgacgccgacatccaggcgccgctgtggccggtcacctgtggtgccgtggcggccgtccccggcgaggtgccggccggcacggtgcaggcgcaggtctggggcctgggccgcgtcatcggcatggaacacccggaccgctggggcgggctggtggacctgcccgaggtgctggaccagcgggcggccgcacggctggccgcggtgctgggcggctgtggcgaggaccaggtggctgtccgggccaccgggatactcggccgccggatgcgccacgccgccgaagccggccccggcgaggggcactggacgccgcgtggcagtgcgctgatcaccggtggcaccggcgccgtcggcgggcacgtggcccgttggctggccgaggccgacgcggcccgcgtggtgctgaccggacgcagcggacccgccgccggcggtgtcgcggtgaccgctgccgaactggccgctcggggaacccgggtggacgtggtggcgtgcgacgtgagtgaccgctccgcgctggccgggctggtgtcctggatcggaacgagcggaccggcgctgtccaccgtgctgcacacggccgcggtactggacgacggggtggtggaccggctctccgccgagcggttgcagacagtgctggcggcgaaggccgcgggcgccgctcacctggacgagctgacggccggactggacctggacgccttcgtactgttctcctccgccgcatccaccctcggctccgcaggtcagggcaactacgcggccgccaactcctacctggacgcactggcggagaatcggcgggcccgcgggctgaccggtctgtcggtggcctggggccagtggggaggcggcggcatggccgactccaaggaggcgatccgggagcggatgaagaagctcccgctgtcggcgatggacccgcggtcggcggtccgggcgctgggcgagtcgctgcacggcccggaccccgtggtgacagtgatggacgtcgactgggccgtgctggcgcagagcgcggcgaacttgacggaggtgccgctggtgcgggacctgccggcggtgcgccgactggccacggccggcagccgggccgtcgagcagcccgcctccccggagggcgagctggcacgccggctggccggtctggaccgggccgggcaggagcggaccctcaccgatctggtgcgggccgaggccgccatggtgctgggccatccctcggccgaggccgtgcaggatcagcaggcgttcaaggacctcggattcgattcgctgacagcagtcgaactgcgtaaccggttgaacactgcgaccggactgcgagtcccggttacattgattttcgatcaccccactccggtggcggtggccgcctggttgcgtaccgagctcgccccggacgagccgactgcgctgccggtgctcgacgacctcgaccggctcgaagccggtctgttgagcgcggcacccgaccaggacacgaacgaacggatcacaaggcggctgcaaagcattctctccaactggatcgaaaagcagggccggtcggattccggcagcggcggggtggaaatcgagtcggccacacccgacgagatgttcgagtttctcgacagggaacttggtctgtccgactag

SEQ ID NO: 66 - DNA, 19845 bp, Streptomyces sp.
  source 1..19845: mol_type = genomic DNA
atggcagatgaagaggccctccgcgactacctcaagtgggtgaccacggacctctacaacacgcggcagcgcctgcgtgaggtggaggaacgcagccacgagcccattgcggtcgtcggcatcggctgccgttaccccggcggcgtcgacaccccggaggacttctggaacctgctcgccaccgcacgcgacgggatcgccgacatccccaccgaccgcggctgggacctcgcgcagctgcgggtccaggacccggacggcagcacgcggcttcccgagggcgggttcatctcggacgccagcgggttcgacgccgcgttcttcgggatcagcccgcgcgaggcggtggcgatggacccgcagcagcggctgctgctggaggtggcctgggaggcgctggagcgctccggcatcgacccgcacgtcctgcgcggcagccagaccggcgtcttcgccggcgcctcgctgtccggctacggcttcggcacggacgaggacctggacgtccacctgatgacgggcacctccaccagcgtcatctccggccgggtctcctacgccctgggcctggagggcccggcggtcaccgtggacaccgcctgctcctcctccctggtcgcactccacctggccgcccagtcggtacggtccggggagtgctcgctggcgctggccggcggcgtcacggtcaccaccgtcccggtcatgttcacccagttctccaagcagctgggcctggccggggacggccgctgcaaggccttctccgccgacgccgacggcatgggcatatcggagggcgccggcatggtcgtcctggagcggctctccgacgcccgccgcaacggccacccggtgctcgcggtcatccgcggttcggcgatcaaccaggacggcgcctccaacgggctggccgcccccaacggtccttcccagcagcgcgtcatccgcgccgccctggccaacgcccgcctgtccaccgccgatgtcgacctggtggaggcgcacggcaccggcaccacgctcggtgaccccatcgaggcccaggcactgctggccacctacggccagggccgcgcggaggaccagccggtcaggctgggctcggtgaagtccaacatcggccacacccaggccggcgccggcatcgccggcgtgatcaagctggtgctggcgctgcagcaccagcagatgccgcagaccctgcacgcagccgagccctccccgcacatcgactggtcggccggcaacatcgagctgctgacccaggcccacccctggccgctgggggagcgcgtccggcgcggcggcgtctcctccttcggcgtcagcggcaccaacgcccacgtcatcgtggaggaggccccggcggaggaacccgccgaggcgcagaccgggacggacggcgaggacggcacggacaccgcacctcaggacaccggcagccacaccgccgagccgctgctgccggagtccggcgcctggctggtgtcggcccgtacggctgaggcgctgtcggcgcaggccgggcggctgcgggagtgggtggcggcgcgtccggagctgggtgcggctgatgtggcctggtcgttggcggcgacgcggtcggcgttcgagcaccgcgcggtggtgctcggttcggagcgcgaggagctgctgtccgcagtggcagacctggcggccggagtcccgtcgtcggtggtggtgtcgggggtggcccggtctgatgtccgggtgggtctgatcttcgccgggcagggtgcgcagtgggttggtatgggccgtggcctgtacgcgggcagtgcggtgttcgccgaggtcttcgaccgggtgtgcggactgttggagctcgaactggggaccgcagtgcgtctgcgggatgtggttttgggtgcggagggggtggacgaagggctggcggatcagacgctgtacgcgcaggccgggctgttcgccttcgaggtggctttggcggaggtgctgaaggctgctggtgtggtgccggatgcggtggtgggccattcagtcggggaggtggctgcggcccacgtggcgggtgtgttgtcgttgccggatgcctgtgcgctggtggcggctcgtgcccggttgatgcaggagttgccgtcgggtggggcgatggcggcggtcaacgccgccgaggcagatgtcatcgcctcgtttgacgaggtgtcgggtgaggtggctgtcgcggcggtcaacggcccggaatccgtggtcatctccggtgcaaccgaggccgtggacgcggtggtggagctgtggcgggaccggggttgccgtgtgcggcgcctgcgggtttcgcacgctttccactccccggcgatggaaccggtcctggacgagctccgtaccgtcgcggagaagctggagttccgtcgtcccgaggtgatgtgggcgggtgccctcaccggtgagctggtgtccgagccgcaggccggctactggcccgcgcagacccgccagaccgtccgcttcgccgacgcgatagccaccctcgccaccgatggcatctccgtcttcctcgaagtgggtccggacggctcactctcctccctcggcccggatgcggtcgccgatgtggacggggccgagcccgccttcgtccccctccagcggcgcaccgaggacggcgccaccgacctggagaccggtctggcccgtgccttcgtccacggcgcgccggtggactggaagcgtgtcctgcccgccggtgagcaggtcgagctgcccacgtatgccttccggcaccagcggtactggcccaagggcatgatggcgctgcccggcacggtgaagccgggtgccggcgaccccgcagagctgggcctggaagcagtgggtcaccccatgctgcgcgccgttgtcgagctggccggtgaggaccagctggtgtgcaccggacggctgtccgcccgcacccagacctggctggccgaccacatggtcggcggcgtgacgttgctgcccggcaccggcttcgtggagctggtcgtcagcgcgggccaccaggcaggcagcccgctgctgaccgaactcaccctgcacgagccgctggtgctgcccgccgaaggccccggcacgcagatccaggtggttgtcggcgagggcgacgaggacggcaaccgccaggtggacgtcttcgcccgcccggaccgctcggaggaggagcacagctgggtccggcacgccaccgccgtcgtcgccccggcccgcatccccgcgccggacgcggtccggactccggccgacctcaccgcctggccgccgcgcaacgcccgggcactggacatcgacgacctctacgccgtccagctggccgacgtctacggccccgcccagcagggtctgcaggctgtttggcagcgcggcgcggacatcttcgccgaggtggcactgccgtcggacgccggtgccgcggccccgtacgccctgcacccggtgctgctcgacgcggccctgctcgccgaccgcgtggccgggcaggcccaggggcgggccacggacgacggccccgaccgtattcgcctgccccacgcctggagcggcgtggaactgcacgccgccggcgcgtccgtactgcgcgtcaggctgcgccggaaccccgacggcaccctggcagtgaccgccgccgaccccaccggagcgatggtgctctcggtggaatcggtgcggacccggccgctctccgccgacgagctccagagcgacgacgcgtggacccgcgacgccctgttcaccgtggaatggcttccgctgcaccgcagcggctcctccaactcctcgcaggccacggcccccgccccggacgggccgtgggcgctgatcggcgccgaccgcttcgccgtggccgagcagctgaccgccgccggaacaccggtgcgcggctacgccggaatcgccgaactcgtggccgctgtcggcgcgggcgagaccgtaccgagcaccgtactcgcctgcctcagcggcaccagcacacacagcggacccgccggcacggacacccacgggatcgccgaggccgcccgccaggtcaccaccgaagccctgatcctcgcgcaggactggctcgccgcacccgagctggaggcggccaagctcgtcctggtgacccggggcggcgtgctggtgcaccccggcgagcaggccgtggacccggccgcgggcgctgtctggggcctgctgcgctcggcccagatggagcaccccgaccgcttcgtgctggccgacctccccgcggacggcagcgccggcgacaccgccgcgctgcccgccgcactggcctccggcgaaccggaagtggccgtacggcagcagagcggctacgcccggcgcatggtccgccccaccgtcaccgaccgcaccgcaccggcccccgcatctgccgaggcacacccgcgcaccgtactggtgaccggcggcaccggaaccctgggcggggtcgtcgcccggcacctggccgccaccggcagagccgacgagaccctgctgctcagccgttccggcccggccgcccccggcgccgccgccctggccgcccggatagccgcgcacggcgccgacgtccgggtgatcgcctgcgacgcggccgaccgggacgccctggccggcgtcctggacacgatccccgccgaccgcccgctgcgctccgtcgtgcacaccgccggcgtcatcgacgacggcctgatcggctccctgagcgccgagcagctggccgccgtcctgcgccccaaggcggacgccgcctggcacctgcacgacctcacccggcacctggacctggaccacttcgtcctgttctcctcggtggccgccgccttcggcggcgcgggccagggcaactacgcggcggccaacgccttcctggacaccctggccggccaccggcgggccgccgggctggccggtaccgcgctggcctggggcccgtggatgccggaggccggcatcggccgcgacctcgacgaggtgatgctgcgccggatgaccggctccggcctcaccgccctcgacgagcaccagggcctgaccctgctggacctggccctcgaacggaccgaggcgctcctcgtcccggcccggctgaacaaggccgcgctgcgggcccaggcagccgccaccggcatccccgccctctggacggagctggccgacgacctcgccccgcgccgggccgccgtcacctcggccgccggcgcctccgccgcaggcacctccgatcagctgcgcgcgcacctggccgcgcaactgggcccggagcgcgaccggctcctgctcgatctcgtccgcagtcacgctgccgccgtactgggccacccctcgggcgacgccatcgaggccggccggcccttcaccgacctgggcttcgactcgctgatcgccgtcgagttccgtaaccgtctcaacgcggcgaccgggttgcggctgccgtccaccctggtcttcgaccaccccaacccgacggcgctggcccggctgctgcacgagcagctgaccgacgacctgccgcagggcgcggcccccgccgccccgcaggccgccttcgcgtacgccgcggacgagccgatcgccatcgtcggcatggcctgccgcttcccgggcggtgtgaccagccccgaacagctgtggcagatgctcgccgacggcaccgacggcctgtccaccttccccaccgaccgcggctgggacctcgaagcgctctacgaccccgacatcgagcacgccggtacctcgtacacgcaggtgggcgggttcctgcaggacgcaggcgccttcgacgccggcttcttcggcatcagcccccgcgaagccctcgccatggacccgcagcagcgcctgttgctggagacgtcctgggaggcactggaacgcgccggcatcgacccggcgaccctgcgcagcagccgcaccggcgccttcgtcggcggctacggttcctcctacctcgccgtcagcgtcaccgacgaggaagaactggagaacctcggcgcccacctgatgaccggcaacgccgccagcgtgctgtccggccgggtctcctacaccctgggcctggagggcccggcggtcacggtggacaccgcctgttcgtcgtcgctggtcgcgctccacctggccgcccaggcactgcgcaccggggagtgctcgctggccctggccggcggcgtcacgatcatggccagcccggacggcttcctggcgttctcccacgcccggggcctggccgcggacggccgctccaaggcgttctccgaccaggccgacggcatgggcatggccgagggcgccggcatgctcgtggtggaacgcctgagcgacgcccgccgcaacggacacccggtactggcggtgctgcggggcagtgccatcaaccaggacggtgcgtcgaacggtctgaccgcgcccaacgggccttcgcagcagcgcgtcatccgtgccgcgctggccaactccggcctgtccaccgccgacatcgacgtggtggaggcgcacggcaccggcaccacactcggtgaccccatcgaggcccaggcgctgctggccacctacggccaggaccgcggcgaccgcggctcgctgctgctggggtcggtgaagtccaacatcggccacactcaggctgcggcgggtgttgcgggtgtcatgaagatggtgctggctctgcagcacgagcggctgccgcagacgctgtacgccgaggtgccgtcctcgcatgtggactggtcggcgggtgaggtgcggctgctcaccgagccggtgccgtggccgtccggtggtgagcgggtgcggcgtgcaggtgtctcctccttcggcatcagcgggaccaatgcgcacgtcattctggaggaggcgcctgcgggggagggcgttgaagctgttgaagaggctggggagcccggggagccggtcgttccggtggtgactggtgctggtgcttgggtgctgtctgggcggactgctgaggcattgtcggcgcaggcggggcggttgcgggagtgggtgtcggcccgtccggctcttcggccggttgatgtggcgtggtctttggcggcgactcggtcggttttcgagcaccgtgcggtggtggtgggcggggaccgggagcaggtgctggcggggcttggggggctggcgtcgggcgagccctcgggtgcgttggttgcgggtacggcccggtcggatgttcgtccggtgtttgcttttgctggtcagggttcgcagtgggttggtatgggtcgggagctggcggaggtttcgccggtgttcgcggggcggttggcggagtgtgcgacggcgttggcgccgtatgtggactggtcgctgaccgatgttctggcgggtgctgagggggctcctgcgttggaggcggcggatgtcgttcagcctgcgttgtgggcggtgatggtgtcgctggctgcggtgtgggaggcggctggtgtcgctccggatgcggtggttggtcactcgcagggggagattgccgctgccacggtggccgggatgctgtcgcttgaggacggtgcccgtgtggtggcgttgcggtcgcaggcgctgaaggtgctggcgggtgcgggcggcatgctgtccgtgagccgtccggcgtcggaggtcgaggagcgtctggtccggttcggggagcgggtgtccttggcggcggtcaacggaccgtcggccactgtcgtctcgggtgagccggcggctctggaggagcttcagtccgagttcgaggcggcgggagcccgcgctcggatggtcgcggtggattacgcctcgcattcggctcaggtggaccggctggaggaggagatcaccaccgtcctggccgggatctctccgcgccggggccgggtcccgatggtctcggcgatgaccggtgagactgtcactggcgaagagctggacgccggctactggtatcgcagtctgcgcgccaccgtgcattacgaccgtgccgtgcgggttctggccggtcgtgggcatcaggtgttcgtcgaggtgacgccgcatccggtgctgatgggcgcgatgaacgacaccctcgtggacctcgcccaggaggccggcccgggggcagagccgggagccgtctgcgggacgctgcggcgtgacgacggcggtgcggatcgtctggtggcctcgctggcggaggcgttcgtcaacggggcgtcggtggactgggcggcggtgctgcccgtcggtgagcaggtcgagttgccgacgtacgcgttccggcgtcgccggtactggccggagggcatgctggcgctgccgacgggcggttcgcttgccgctggcggggagggcgcgagccctgatgcggagtcggcgttctgggcagctgtcgaggggggtgacctgtcggggctgggtgacgcgctggccctggagggtgggcgtccgttcaatgaggtgctgccggcgttggcgtcgtggcggcggcgtgagcgggatcgttcgaccacggctggctggcgttatcgcactggctggtcggtcatcgtcgagccggatgttcggctgctgtccggtacgtggctgatcgccgtccccgcggagtcggccgcggtggagctggcgcaggactgcgtcggggcactgagcgcccgtggtgctgaggccgtggtggtcgaagtaccggccgggtcggtggaccggtccgagctggcggcattgctcaccggcgcggcggacccgtcagccatctccggtgtgctgtcgctgctggcgctggacgagatgccgttgccggaccgtccggtcgtgaatgctgggctggctgccaccttgggcctggtgcaggctcttggtgacgctggggttgaggcgccgctgtgggtggcaacgcgtggtgcggtggcggccgggcggggtgaggcgttggcccgtccggtgcaggcgcaggtgtggggtctgggccgggtcgtcggcctggagcacccggagcgttggggtggtctggtcgatctgccggagacgctcgatgagcggtccggtgcgcggctggtggcggtactggccggctgcggcgagaacgaggtcgccgtccgacgggccggaatcctgggccgtcggctgtcgagcgttccgcacagcagcggcccggacaagggctggacgcctcgcggcaccgtgctgatcaccggtggaaccggggcgatcgccggacacgtcgcgcgccgactggccggccggggcgccgaacgtctggtgctgaccagccgctcgggcccggccgccgcgggcgcggcagcgctggcggccgaactcgcggcagccggttccgccgtggagatcgtcgcttgcgatgtcagcgaacgggtcggtctggccgggctgctggacgggatcgatgcgagcggcccccggctctcctccgtactgcacacggccgccgttctggacgacggtgtcgtggaccgactctcggtggcccggttggcgcccgttctggaggtgaaggcccacgctgcggcgctcttggacgagctgaccgcgcacctggacctggacgcgttcgtattgttctcctcggcggcctccaccctcggtggcccgggccagggcagctacgcggcggccaactcctacttggacgcgctggcggagaaccgccgggcccgcggcctggccggtctgacggtggcctggggcctgtggggcggtgccggtctggcgcagtccaacgaggccatccgctcccggatgcgccggaccgcgatgccgccgatggaccctcagctcgcggtggaggctctgatcgaggccatggacggcccggacgccgcggtgacggtgatggacttcgatccgcagcagttgggcgcggcgccgggtacggcgcacgtcctggaaatgccgctggtccgggacatgccggagatccggcggctcgccgccgcccgcgcaggcaacggcagcaccggaccggccttccgcgccgacggcgaactcgccgcccggctggccgggctgagccgatcggagcaggaccggatcctgaccgacgtggtccgcgccgaggccgccgccgtactggggcacgactctgcggacgaggtgcaggcccggcaggccttcagggacctgggcttcgactcgctgacctccgtcgagctgcgcaaccagctgaacgcggcgaccggactgcggctgcctgcgaccctggtcttcgaccacccgactccggtcgcgctggccgccttcctgcacggcgaactgaccgggcaggtgacacagaccgcagccgctgcgccggtctccgcaacccccgccgcggacgaaccgctggcgatcgtcggcatggcctgccgcttccccggcggcgcgagcacgcccgaggagttctgggaactgctggcctccggcggcgacgcggtgggcggtttccccaccgaccgcggctgggacctggacggcttcttcgacccggaccggcacggcagcggaacctcctacaccgactccggcgccttcctgcgaggggccgccgagttcgatgcgggtttcttcgggatcagtccgcgtgaggcgctggcgatggacccgcagcagcggctgatgctggaaacctcctgggaggcgttggaacgggccggtatcgacccgtcgtccctgcgtggcagcccgaccggcgtgttcgccggcggcttcggctccggctacgccatcggcatggcactcaccggccaggacggctccggcgtcgagggccacctgatgaccggcaactccaccagcgtcctctccggccgggtctcctacgccctgggcctggaaggcccggcggtgacggtggacacggcgtgttcgtcctcgctggtggcgctcaacctggcggcccaggcattgcggtccggggactgctcgatggccctggccggcggtgtgaccgtcatggccactcccttctccttcgtggagttctcccgtcagcagggtctgtcggcggatggccggtgccgggcgttctcggaggatgcggacggtacgggctgggccgagggcgccggtgttctggtggtggagcggctttcggatgcgcggcgtaatgggcatcgggtgctggctgttgtccgtggcacggcggtgaatcaggatggtgcgtcgaatggtctttcggcgccgaacgggccgtctcagcagcgagtgattcgggcggcgttggccaatgccgggttgtcttcgtccgaggtggatgtggtggaggcgcacggcactggcacgaagttgggtgacccgatcgaggcgcaggcgctgctggcgacgtatggccgggatcgtggtgatcgggatgcgttgtggctggggtcggtgaagtccaacattggccacactcaggctgcggctggtgttgcgggtgtgatcaagatggtgctggcgttgcagcatggtcagttgccgcggacattgcatgctgatgtggcgtcgtcgcatgtggactggtcggcgggtgaggtgcggctgctgacggagtcggtgccgtggccggtgggtgagcgtccgcgccgggctggtgtgtcggcgttcggtatcagcggtaccaacgctcacgtgattctggaggaggcccccgcgatcgaggcgcggagcgagggcgccggtcaggtgccggttgccgctcttgagggtgctggtgcgtgggtggtgtcggggcgtacggctgaggcgttgtcggcgcaggcggggcggttgcgggagtggctgacggcgcgtccggaactgcgaccggccgatgttgcctggtcgttggcggcgacgcggtcggcgttcgagcaccgtgccgtagtgctcgggaccgagcgtgaggaactgctgtctgacctgcacagcctggccgctggtacgtcctcgcctgtggtggtgtccggtgtggcccggcccgatgtccgggtgggcctggtgtttgctgggcagggtgcgcagtgggcgggtatgggccggggcctgtatgcgggcagtgcggtgttcgccgaggtgttcgaccgggtgtgcgggctgctggagttggagctgggggccgcggtgcgtctccgggacgtcgttctgaacagcgacgatgcggctgctggactggcggaccagaccctgtacgcgcaggcgggcctgtttgccttcgaggtggccctggcggcggtgctgaaggctgctggtgtggtgccggatgcggtggtgggccactcggtgggcgaagtggctgcggcccatgtggctggtgtgttgtcgttgccggatgcctgtgcgctggtggcggctcgtgcccggttgatgcaggagttgccgtcgggtggggcgatggcggcgatcaacgccgccgaggcagatgtcatcacctcgtttgacgaggtgtcgggtgaggtggctgtcgctgcggtcaacggcccggaatccgtggtcatctccggtgcaaccgaggccgtggacgcggtggtggagctgtggcgggaccggggctgccgggtgcggcccctgcgggtctcgcacgccttccactccccggccatggacccggtcctggacgagctccgcaccgtcgccgaagggatggagttccgccgcccggaggtgatgtgggctggcgccctcaccggtgagctggtgtccgacccgcaggccggctactggcccgcgcagacccgccaggccgtccgattcgcggatgcggtggccaccctggcacgggagggtgtctccgtcttcctcgaagtgggtccggacggctcgctctcctcgctcggccccgacgcagtcgccgacgtggagggtgcggaggcggcgttcgtcgcacttcagcggcgtgacgaggaaggtgttgcaggtctgctgtccggcctggctcgcgccttcgtcaacggtgccccggtggactggacgcgtgtgctgcccgccggtgaacacgtcgagctgcccacgtacgccttccggcaccagcggtactggcccgagggcgttctcccgctcatgccatcggcggtcgcgaccgtggccggggctggcaccgaggcggaggccggattctgggcggcggttgaggacggtgatctggcgcgggtcgcggagacgctggctttggaggatgcgggtcagctggatttggtgttgccggcgttggcgtcgtggcggcgtcgtgagctggatcgttcggctactgctgactggcgctaccggaccgtctggtcgcctgttgccgagcctgacgcccgtgtgctgacggggacttgggtggtgcttgcgccggccggttgctcggaggagctgcggcatcagtgtgtgacggcgctgacggcccgtggtgccgaggtcgtggtggtcgaggttccggctgggacggtggaccgggccgggctgacggcactgctgactggcgctgcggatgcgtcgaccgtgtccggtgtgctgtcgctgctggccctggacgagacgccggtacccgggtatccggtggtgcccgtgggcctggctgccaccctgggcctggtgcaggctctcggtgacgccggggtcgaggcaccgctgtggatcgccacgcgtggtgcggtggcggccggccgaggcgaggcgttggcccgtccggtgcaggcgcaggtctggggcctgggccgggtggtcgcgctggaacaccccgagcgctggggcggcctggttgacctgccggagacattcgacgagcggtccggtgcgcggctggtggcgatgctcgccggttgcggagagaacgaggtggccctgcggccatccggaatcctgggccgccggctggcgcacgtatcgggtcgcggtgcgcgggagcgctgggcgccgcggggaaccgtactggtcaccggcggcaccggtgccatcgccgggcatgtctcacgatggctggccggccggggtgcccagcgtctggtgctgaccagccgttcgggcccagcggctgtgggcgcggcagcgctggcggccgaactcgcggcagccggttccgctgtggagatcgtggcttgcgatgtcagcgagcgtgccggtctggccgggctgctggactggatcgatgcgagcggtccccggctctcttcggtgatgcataccgcgggtgtgctcgatgacggtgtggtggaccgcctcgacactgcgcgtctggagactgtgctgggcgtgaaggcgcagggtgcggtgctgctggatgagctgaccgcggacctggacctggacgcgtttgtgttgttctctgctgcggctgccactctgggtggttcggggcagggtaactatgcggcggccaatgccttcttggacgcgttggcggaggagcgccgggcccgtgggctgaacgcgctgtctgtggcgtgggggccgtggtcgggtggtggtatggctgcgtcgagtggtgcggtgcgggcgcgggttgagcggggtgcgatgccgccgatggacccgcggcttgcggtccgtgcgctggccgaggcgttgcgggggccggatgccgccgttgccgtcatggacgtggactggacacagctggcttccggtccgggggccgtggagctgctgaagcggccgttggtgcgggacctgcccgaaatccgcgacatggccgcggccggtggcggttcaaccgccgccggccgcgtcgagggcgagttggctcgtcagctggctgggctggaccgcgcggagcaggagcggacgctcgccgacgtggtgcgtggcgaggctgcggtggtgctgggctatggctcggccgatgcggtggaggaccgtcgggcgttcaaggacctcgggttcgattcgctgaccgcggtcgaactgcgcaaccggctcaatgcggcgaccgggctgcggctgccgtccaccctggtcttcgattaccccaccccgctggcgatggccgccttcctgcgcggtgaactgaccggtggggagttggacaccgctggtgcttcggcggccacggtggtggcggcgcctgcggacgagccgttggcgatcgtcggaatggcatgccgcttccccggcggggtgggcagcccggaggagttctgggagttcctggcttccggtggcgatggcgtgggcgatttcccgagcgaccgtggctgggacttggagagtgtttacgacccggagggtgagcgggagggtacttcgtatgtcgcgcagggcgcgttcctgcgggaggcggctgagttcgacgcggggttcttcgggatcagcccgcgcgaagccctcgcgatggacccgcagcagcggctgttgctggagacgtcctgggaggcgctggagcgtgccggcatcgacccggcgtccctgaaggagagcgccaccggtgtcttcgccggcggtttcacctccggttacgggctcggcgtttccatgagccaggacgactctggcgctgagggtcacgtgctgaccggcaatgcgaccagcgtcctgtccggccgggtgtcgtatgtgctgggtcttgaggggccggcggtgacggtggacacggcgtgttcgtcgtcgttggtggcgcttcacctggcggcgcaggcggtgcggtcgggggagtgttcgctggcgttggccggcggggcgaccgtgatggcgacgccggggaccttcatcgacttctcccggcagcagggcctgtcgatcgatggccggtgccgggcgttctcggaggacgcggacggtacgggctgggccgagggtgccggcatggtcgtggtggagcggctgagcgatgcccgccgcaacggccacaaggtgctggcggtgctgcggggcagtgcggtcaaccaggacggtgcgtcgaatggtctgaccgcccccaacgggccttcgcagcagcgcgtcattcgggcagcgttggccaacgcccggctgtccgctgccgaggtggacgtggtggaggcgcacggtaccggtacgaagttgggtgacccgatcgaggcgcaggcgctgctggcgacgtatggccgggatcgtggtgagcgggacgcgctgtggctggggtcggtgaagtccaacatcggccacacccaggccgcggccggtgtggcgggcgtcatcaagatggtgctggcactccagcacgagcagctgccgcgaactctccatgccgaggtgccgtcgtcgcacgtggactggtcggctggtgcggtgcggctgctgaacgagcagctggcctggccgtccgatgggaatcgggtacggcgggccgctgtctcggcgttcggtatgagcggtaccaacgctcacctcatcctggaggaggcccccgcaccgcaggacggcgaagccgtagaggagcacggggaaccagtggtcccggtggtcaccggtgccagcgcttgggtgctgtccgggcggaccgctgaggcgttgtcggcgcaggcgggccggctgcgggagtggacggcgacgcgcccggagctggcaccggccgatgtggcctggtcgctggcagccacccgctcggtcttcgagcaccggtccgtcgtggtgggcacgggccgtgccgagctgctcggcggcgtggaaagcctggcgacgggcgttccggctggttcggtggtgtccggtgtggcccgctcccacgcccggccggtgttcgcttttgcgggtcagggttcgcagtgggttggtatgggtcgggagctggcggaggtttcgccggtgttcgcggggcggctggcggagtgtgctgcggcgttggccccgtacgttgactggtcgctgaccgatgtcctcgcgggtgcggaggatgcccctgcgctggaggcggcggatgtcgtccagcctgcgctgtgggcggtgatggtctccctcgccgctgtctgggaggcggcgggggtcgctccggaggcggtggtgggccattcacagggggagatcgccgctgccacggtcgccggaatgctgtcgcttcaggacggtgcccgtgtggtggcgctgcggtcccgtgcccttaaggtgctggcaggagcgggcggcatgctgtccgtgagccgtccggcgtcggaggtcgaggagcgcctggcccggttcggcgaccggttgtccttggcggcggtcaacggtccctcggccactgtggtctcgggcgagccggcggctctggaggagcttcagtccgagttcgaggcggagggcgcccgggcccgcatggtcgctgtggattacgcctcgcactccgcccaggtggaccggctggaggaggagatcaccaccgttctggccgggatctctccgcgccggggccgcatcccgatggtctcggcgatgaccggcgagattctcacgggtgaagagctggacgccggctactggttccgcagtctgcgggcgacggtgcattacgaccgtgcggtgcgggtgctggcgggtcgtggtcatcaggtgttcgtcgaggtgacgccgcatccggtgctgatgggcgcgatgaacgacactctcgtggacctcgcccaggacgcgggtggagagcctgccgccgtgtgcgggacgctccgccgggacgacggcggcgcagagcgcctgctgacttccctggccgaggcgttcgtcaatggcgcaccggtggactggcagacagtgctgcctgcgggcgaccaggtggagcttcccacctacgcgttccggcgtcgtcgcttctggccggagggcatgctggtcctgccgatccccggttccgcgaccaccggttccacgtccgccgggggcggtgccagcactgacgcggaggcggcattctgggcggccgttgaggatggagacctgtccgggctgggtgacgcgctggccctggagggcgggcgtccgttcaatgaggtgctgccggcgctggcgtcgtggcgccgccgggagcgggaccgttcgaccgctgccggttggcgctatcgcactggctggtcggccatcgccgagccggacgctcgtgtgctgtccggtacgtggctgatggtggttgcggccggggtggacggcggtgcggcgcagcagtgcgcggccgcgctcagcgcccgcggtgccgaggccgtcatgatcgaggtagcggccgggtcggtggaccggtccgagctggcggcattgctcaccggcgcggcggacccgtcagccatctccggtgtgctgtcgctgctggcgctggacgagacgccgctgccggaccgtccggtcgtgaacgccggcttggccgccaccctgggcctggtccaggctctcggggacaccgggatcgaagcaccgctgtggatcgccacgcgtggtgcggtggcggccgggcggggcgaggcgttggcccgtccggtgcaggcgcaggtctggggcctgggccgggtggtcgcgctggagcaccccgagcgctggggcggcctggtcgacctgccggagacattcgatgaccgctccggcggacggctggtggcagtgctggccggctgcaaggagaacgaggtcgccatccggacggccggaaccctgggccggcggatgtcgcgggtgccgcgacgcagcccccgtacaccctggtcgccgcggggaaccgtactggtcaccggcggtaccggtgcgattgccgggcatgtctcgcagtggctggccggccggggtgccgagcgtctggtgttggcgggccgttccggtccggcagcagccgatgtcgcggcgcgggccgcggagctggccaccgctggcgttcgcgtggacgtgatctcctgcgatgtcagcgaacgcggcccgctgggcagcctgttgggctggatcggcagcaacggcccggagctgtcctcggtgatgcacaccgcgggtgtgctggatgacggtgtgctggaccgcctcaccaccccgaggctggagaccgtgctgggcgtgaaggcccacggcgcggcgctcctggacgagctgaccgcggacctggacctggacgcgttcgtactgttctcctcggcagcctccacgctcggcggcccgggccagggcaactacgcagccgccaacgcctacctggacgcgctggcggagaaccgccgggcccgcgggctggccggtctcgcggtggcctggggcctgtggggcggtggcggactgggcgaatccaacgaggccatccgctcccggatgcgccggctcccgatgccgccgatggacccccagctcgccgtccgggcgctgggcgaatcgctggacggcccggagccgacggtgacggtgatggacttcgactggcagcagctgggctcggcaccgggttcggcggacgagctggatctgccgctggtccgggacattccggagatccgccgactggccgcggagcgggccgccgcaggcagcgtctcccgcagcgacagcaggggcgaactcgccgcccggctggcgggggcgggccgctcggagcaggaccggattctgaccgacgtggtccgcgccgaggccgccgccgtactggggcacgactcggcggacggtgtgcaggcccgccaggccttcaaggacctgggcttcgactccctcaccgccgtcgaactgcgcaaccgtctcaacgcggcgaccgggctgcggctgccggccaccctggtcttcgaccaccccaccccgaccgcgctcgccggctggctgcgcaccgaactcgcccccgaagagacggcagcagtaccgctcctcgccgaactcgacaggctggaggccgccctgcgctccggaccggtggacgacccggtgcgggaggaggccaccagccgactgcgccgactgctggacaccatcggcgaacaagccgccgaccagggcaggaagcgacagacaaccgacatcgacgccgcctccgacgaagagctgttcgccctcgtcgacggactcgaatga

SEQ ID NO: 67 - DNA, 9738 bp, Streptomyces sp.
  source 1..9738: mol_type = genomic DNA
atgagcgaggagaagcttcgctatttcctcaagcgggtgaccgccaacctgcacgagaccaggcagcgcctgcaggaaatggaggcggccggcggcgagcccatcgccctggtgggcatggcctgccgcttccccggcggggtgcgcagcccccaggacctgtggcagctgctggacgcgggccgcgagtcggtggccgcactgccgcgcgaccgaggctgggacctgggtgacgacgggaccggacaggacgccgccgaggtccaggccggcaacttcctgcacgacgccaccggcttcgaccccgccttcttcgggatcagcccgcgcgaggcggtgtcgatggacccgcagcagcggttgctgctggaggtggcctgggaggcgttggaacgctccggtatcgacccgtcgtccctgcggggcagcccgacgggcgtgttcgccggcgcctccgcctccgggtacggctgggccagcggcgcccagggcgacctggacgggcacgtgatgaccggcaacgcgatgagcgtgctctccggccgcctcgcctacaccctgggcctggaaggcccggcggtcaccgtggacaccgcctgctcctcggcgctggtctcgctgcacctggcctgccaggcactgcgcgcgggagagtgctccatggccctggtcggcggtgcctacgtggcggccaccccggtactgttcaccgacttcagccgctccctggggctctccccggacggccggtgccaggcgttcggcgccggcgccgacggcatgggcgtcgccgagggcgtcggcgtcgtggtcgccgaacggctctccgacgcacggcgcaacggccaccgcgtcctcgcggtcgtacgcggcagcgccgtcaaccaggacggcgcctccaacggcctgaccgcccccaacggccccgcgcagcagcgcgtcatccgcgccgccctggcgggtgcccgcctgaccgcgtccgaggtggacgtggtggaggcgcacggcaccggtaccccgctgggcgacccgatcgaggcccaggccctgctggccacctacggccaggagcgccccgagggccgcccgctgtggctgggctcggtgaagtccaacatcggccacgcccagcaggccgcgggcatggccggaatcatcaagatggtcctggccctgcagcaccagaacctgccgcgcaccctccacgtcgaaaagcccaccgacgaggtggactggtcggcgggcgaggtcaaactgctggccgagccggtgccgtggcccagtggcgagcgggtacgccgggccggtgtctccggtttcggcatgagcggcaccaatgtgcacgtgatcctggaggaggccccggccgaagaggccgcggaggccacggatccgaccgggcaggagggaccggacgcgggcgagggtgaggcggaggccctgtccgagtcggcgcagcacggtccggaccaggaggccgtgtcggcccccgcgccggcgctgctcgccgacggcggccccacggcctggctggtgtcgggccggtctgcggagggtcttgccgcgcaggcgggacggctgcgggagtgggcggccgagcgcccagccctgcgtccgtccgacgtggcctggtcgctggcggccacccgatcggtcttcgagcaccgtggtgtggtcgtcgccgacgagcacaccgcacttctgtccgggctggacgatctggctgccgcaacggctactgccgatacggtggttggcgttgctcgggccacgggtcgtacggtgtttgttttcgctggtcagggtgctcagtgggtcggtatgggccgggagttgttggcctcgtcgccgttgttcgcggcgcggttggcggagtgtgcggcggcgttggcgccgtatgtggactggtcgctcagcgatgtcctgacgggtgtggagggcgctccggcgctggaggcggctgaggttgtccagcccgtgttgtgggcggtgatggtgtcgctggccgctgtctgggaggcggctggcatcgtgccggacgcggtggtggggcattcgcagggggagatcgccgctgccacggtggccgggatgctgtcgctggacgacgccgcccgggtggtggcggtgcgttcgcgtgcgttgtctgggctggatgtggccggggcgatggtgtcggtggtgatgcctgccggtgcggtgcgtgagctggtggcgggtttcgacggtcggttgtcggtggctgcggtcaatggcccggcgtcggtggtggtatccggagaagcggaggcgctggcgtccttcgagcgggagctggcggcacgtaaggtgctgcgctggcgtatcccggcgacggactttgtggcgcattcggcggcggtggagccgcttgccgaccagctcgaagcggacctgtccggcattgccccgcaggcgggccgcatcccgatggtctccaccgttacgggcgagtggctggccggtgaggaagcggatgccgcgtactggttcgcgaacctgcgccagatggtgcgcttcgaggacgccgtgcgcacgttgctggaatccggttacggggcatttgtggaggtctcgccgcatccggtactgaccgccgccgtgactgaaaccgcccaggacgcggactcgggcgccgctcaggtgctgagcatcggaaccctgtcgcagaccgacgcgggcccggccggtctgctgcgcgccttcgcgcaggcccacgtggcggggctgccggtggactggcgcaaggtcctgccggccggcgaggtggtggatcttcccacctatgccttccagcaccgccgttactggctggagccctccgccgctgctgctgccgcagtcgccgcaggtgggtcctcggacgccgaggcgcgcttctgggccgccgtcgaaggcggcgacctgacccacatggccgaggtgctcaacctggacgacgcgcacgcgttgggcgaggtgcttcccgccctcacctcctggcgccgccaggaacaggaccggtcggccaccgccgactggcgttaccacatcacctgggccccggtggccgacccggccgccaccgtcctcaccggccgctggctgctgctcaccggacccgagggcaccggcccggcgcaggcctgcgcccaggcgctcgccgcccgtggcgccgacaccaccatcgtggaggtgcccgagggcaccgtggaccgcgccgcgatggcggccgtcctcggccaggcactgcaagagaccacggcgccgacgggcatcgtctcgctgctcgccctggacgagacgccggtgcccgggcaccccgcggtcccgggcggcctggcccgcacgctggccgcggtgcaggcactgggcgacaccggggtggacgccccgctgtgggtactgacccgcggcgccgtcgccaccaccgcccaggagccgcccgtcaccccggcacagagcctgacctggggattcggccgggtcgtcgccctggagcaccctgaccgctggggcggactgatcgacctgcccgccgacttcgacggaccggacggcgcggacggacagatcacggcgcgcctgtgcgcggtgctggccggctgcggcgaggaccaaaccgcgatccggccggccggaatcttcggccgccggctgagcagggccggccgcccccggggcgaggccggaccgtacacgcccggcggcagcgccctggtcaccggcggcaccggcgccatcggtggacgcgtcgcccgctggctgtccggccgcggcgccgcccggctggtgctgaccagccgctccggtcccgccgctgccggagcggccgccctcgccgccgaactggccgccgcaggcagccgggtggacatcgtggcctgcgacgtcagcgaccgcgaccaggtcgccacggtgctcggccggatcgccgcgcaggggccctcgctgaccaccgtgctgcacaccgccggcgtcaaccaggccaccctggtgcaggacaccgacctcgccgagacggacgccgtactggcggccaagacggccggcgcccagcacctgcacgacctgacccgcgaactcgacctgaacctggacgcgttcgtcctgttctcctccatctcggccacctggggcagcggtctgcagcccgcctacgcggccgccaacacctacctggacggcctcgcccagcaccgccgcgcccacggcctgcccgccacctcggtggcctggggctcctggggcggtggcggcatgaccgacgaggccggcgcggcgcagggcatccgtcgcggcctgatcgtcatggacccggaccacgccgtacaggcgctggcccaggttctggactgcgacgaaccccagatgaccgtcgcccacgtggactggcagcggttcgcaccgccgttcacgctgcgccgccccagcccgttgatcgagaacctgccggaggtccaggaggtgctggccgccgacgggcagcacacccagggcagcggcgacacggcagccggcccgcaggccggagcctcctggcgccggcagctggcgggcctgtcccgcaccgagcagaagcggaccctgctcaccctcgtccagacccaggcggccgcggtactcgactacgccgcacctggcgaggtcgaggccacccgcgccttcagcgacctcggcttcgactcgctgacctcggtcgaactgcgcaaccggctcagcagcgccaccggcctgcaactgcccgcaaccctgctgttcgacgcccccacgcccagcgccgccgccgaattcctgctggccgaactggccgagctgaccggcacaccggacggcacacaggccgacgcatcgcgcaccgcccccgtcctggcggcggccgcggacgaaccgctggcgatcgtcggcatggcctgccgctacccgggcggcgcgagcggcccgcaggagctgtggaacctggtcaccgccgggaccgacgccatctccgtactgcccgaggaccgcggctgggacatcgaggaccgccagggaaacccccagggccccgtccggtccggcggcttcgtctacgacgcgacccacttcgacgccggcttcttcgggatcagcccgcgcgaggcggtgtcgatggacccgcagcagcgcctgctgctggaagtggcctgggaggcgttggaacgctccggtatcgacccgtcgtccctgcggggcagcccgacgggcgtgttcgccggtgcctccgcctccggctacggctggagttccggggtacagaaggagctcgacggccacctggtgaccggcatctccaccagcgtggtctccggccgcgtctcctacgtactgggcctggagggcccggcggtcaccgtggacaccgcgtgctcctcctccctggtcgcgctgcacatggcctgccaggccctgcgctccggggagtgctcgctggccctggccggcggagtcatggtggcggccaacccgctgctgttcgaccagttcagccggcagatgggactctccccggacggccggtgcaagccgttcggcgccggtgccgacggcatgggactgggcgagggcgccggcatgctggtcgtcgagcggctctccgacgcccgccgccatgggcacaaggtcctggccgtcgtacggggcagtgccgtcaaccaggacggcgcctccaacggcctgaccgcccccaacggcccgtcccagcaacgggtgatccgcgccgcactggccaacgcccgcctgacccccggtgacgtggacgtggtggaagcacacggcaccggaaccccgctgggcgacccgatcgaggcccaggcactgctggccacctatggccaggagcgcggcgagggcgacccgctgtggctgggctcggtgaagtccaacatcggtcacacccaggccgccgccggcgtcgccggtgtcatcaagatggtgctggccctccagcaccgcgagctgccgcgcaccctgcacgcagccgaaccctccccgcacatcgactggtcggccggcgacgtcagactgctgaccgaagcggccccgtggccggagaagaaccgggtacgccgcgccggcgtctcgtcgttcggcatgagcggcaccaacgcccacctcatcctggaggaggccccggccgagccggccgacactggcgaaacgcccgaaccggctgaggccgcgcctgccgttcttgcggcgggggaggcgtccgcgtggttggtgtcgggccggtctgcggagggtcttgccgcgcaggcgggtcggttgggggagtgggcggctgggcgtgctgcgttgcgtccggttgatgtggcgtggtcgttggcggctacgcgggcggtgttggagcaccggggtgtggtcgtggctggtgaccgggcggggctgttggatggcgtgcgagggctagcggctggtgagtcggccgcgccgggtgtggtgtcgggggttgctcgggccacgggtcgtacggtgtttgctttcgctggtcagggtgctcagtgggtcggtatgggccgggatttgttggcttcgtcgccgttgtttgcggcgcgcctggcggagtgtgaggcggccctggcaccgtatgtggactggtcgctcagcgaggtgctggcgggtaccggcgatgcccccgcgttggaggctgccgaggttgtccagcccgcgctgtgggcggtgatggtgtcgctggctgctgtctgggaggccgctggggttactccggatgcggtggtgggccactcgcagggggagatcgctgcggcgacggtggcggggatgctgtcgctggaggatgcggcgcgggtggtggcagtgcgttcgcgtgcgttgtcgtcgctggatgtggccggggcgatggtgtcggtggtgatgccagccgacgcggtacgcgagttggtggcgggcttcgacggccggttgtcggtagctgcggtcaatggcccggcgtcggtggtggtatccggacaggcagaggcactggcgacgttcgagcgggagctggcggcgcgcaaggtgttgcgctggcgtatcccggccacggacttcgtggcgcattcgccggccgtggagccgcttgccgccccgcttgaagcggagctgtcgggcattgccgcgcaggcgggtcgcatcccgatggtctccacggtcacgggggagtggctgaccggtgaggaaaccaatgcctcgtactggttcgcgaacctgcgccagatggtgcgcttcgaggacgccgtccgtagcctgctggagtccggttacggagcgttcatcgaaatctcgccgcatccggtgctgaccgccgcagtgaccgaaaccgcccaggacacggacgcgaacgccacccccgtgctgagcatcggcaccctgtcgcagactgacgcgggtgcggccggtctgctgcatgccttcgcacaggcccacgtggcggggctgccggtggactggcgcaaggtcctgccggccggcgaggtcgtagagctccccacctacgccttccaacaccgccgttactggctggagccctccgccgccgctaccgctgctgccgccgcggtcgccgccggtggttcctcggccgccgaagcacagttctgggccgccgtcgaaggcggcgacctgtcaggcgtcgcggacaccctgtccctcaccgatcagcagcaactcagcggcatactgcccgcgctggcgtcctggcggcgccgtgaacaggaacgttcggccactgaaagctggcggtaccggatcacctgggcgccggtggccgacccggcaccggctcgcctgtcgggccgctggctgctgctcgtaccggcttcggaggtcgacggcccgatggcgcgccagtgccgtgcggcgctcaccgcccgtggagcggacgtcgtggtgctggagatggccggggccgccgcacgtgaggcggtggccgagcaggtgcgcgcggcactgccgtcggacggattcgccggcatcctctccttgctggcgctggatgaaacgccgatgccgggccatccgtcactgtctgtggggctggccgccaccctcaccttggtgcagggcctcggcgacgccgacgtggacgccccgctgtgggtggccacgtcccacgcggtggctgccgcacccggcgagccgccggtccgtccgctgcagacacaggtctggggcctgggccgggtcgtcggcttggagcaccccgagcgctggggcgggctggtcgacctgcccgccgacttcgacggaccggacgggcagatcgcggcgcgtctgggcgcggtgctggccggctgcggtggtgaggaccaggtggcgatccgcgcggccgccatcctgggccggcgcctgacccacgccccgcagcccgcctcgcctgatcagccgtggacgccgggcggcagcgtcctggtcaccggcggcaccggtgccctcggcggacacgtcgcgcgctgggcggccggtcgtggcgcccggcgactggtgctgaccagccgttccggtcccgccgctgccggagcggccgctctggccgccgaactggccgcagcgggcagccgggtggacatcctggcctgcgacgtcagcgaccgtgcccaggtcgccggggcgctcggccggatcgtcgcgcagggcccctcgctgacctcggtgctgcacaccgccggtatcgggcaggccgtgacggtggccgaagccgacctggccgagacggacgcggtactggcggccaaggcgtccggagcccggcacctggacgagctcacccgcgaactcggcctggaactggacgcgttcgtcctgttctcctccatctccgcgatctggggcagcgtcgtgcagcccgcctacgccgccgccaacacctacctggacggcctcgccgagcaccgccgggcccgcggcctgcccgcgacttcggtggcctggggcccctggggcggcggcggtatgaccgacgcggagaccgccggacggctggcccgtggcggcgtggcgctgatggacaaggaccgcgcggtccaggccctggcacacctcgtggacggtggggaggggccgatgaccgtcgtcgacgtcgactggtcgcgattcgccgtgccgttcacgctgcgtcgccccagcccgctgatcgccggcctgcccgaggccgccgcggctctggcggcagaggcgaccgccccggtcaccgacccggccgccggagcctcgctgcgcgaggagctggccgggctgccgagtgccgagcagcaccgggcactggtccggctggtgcaggaacacgcggccgcggtgctcgactacgccgcacccggtgaggtagaggccacccgcgccttcagtgacctcggtttcgactcgctgacctcggtcgaactgcgtaaccggctcagcagcgccaccggcctgcaactgcccgccaccctgctgttcgaccaccccaccccgcaagcggcggccgaccacctgtggagcgagcagttccaggacggcacgggcgcggtgtccctggtggaggaggtcgaccggctcgggtcgctgctgaccggggcggcgccggacgagaagacgtacgacatggtcaccgagcggctgcaccacctcctcgcccagtggaaggagagcggcgccccggaagagagccaagcagtcgcggacaagatcggatccgcaacggacgacgagatattcgagttcatccaccgagaactcggccggtaa

SEQ ID NO: 68 - DNA, 14484 bp, Streptomyces sp.
  source 1..14484: mol_type = genomic DNA
atggccaacgaagacaagctccgcgattacctcaagctcgtgacggccaatctgcgccaggtccgccgccggctgaccgaggtggaggaacgcagccaggagccgatcgcgatcgtcggtatgggctgccgtttccccggcggggtgaacggccccgaggacctgtggcggctgctggcttcgggcaccgacgcgatcggcccgttcccggccgaccgcggctgggacctggagaacctctacgaccctgacccggaccgtccgggcacctcctacgcgcggcatggcgggttcgtctacgacgcgacccacttcgacgccgacttcttcgggatcagcccgcgcgaagcggcggccatggacccgcagcagcggttgctgctggaggttgcctgggaggcactggagcaggccggcatcgacgcgaacgcgctgcgcggcagctccaccggtgtgttcgccggctcctacggccagggctacggcaccggtccctcggtcaaaacccaggggctggaggggcacctgctgaccggcgtctccaccagtgtcacctcgggccgcctggccttcaccttcggcctggaaggcccggcggtgacggtggacaccgcgtgctcctcctcgctggtggcgctgcacatggccgcgcaggcactgcgcgcgggggagtgctcgctggcgctggccggcggttccacggtgatctccgcgccgtccgtgttcgtgggattctcccggcagcggggcatgtcggacgacggacgctgcaagtccttcagccaggacgcgaacggttcgggctgggccgaaggcgtgggcctgctggtggtcgagcgcctctccgacgcacggcgcaacggccaccacgtgctcgccgtgctgcggggcagtgccgtcaaccaggacggtgcgtccaacggcctgaccgcgcccaacggtcccgcgcagcagcgggtgatccgtgccgcgctggccaatgcccgtctcactcctggtgatgtggacgtggtggaggcgcacggttcggcgacgaagctgggcgatccgatcgaggctgaggccctgctggccacctacggccgggagcggccggaggggcggccgttgtggttggggtcggtgaagtccaatattggtcatacgcaggctgctgccggtgtggcgggtgtcatcaagacggtgctggctcttcagcatgagcggttgccgcgcacgttgcatgtggatgagccgacgccgcatgtggactggtcggcgggtgaggtcaggctgctggcggagtcggtgccgtggcccggtggtgagcgggtgcgccgcgccggtgtctccgggttcgggatgagtggcaccaatgtgcacgtgattctggaggaggccccggccgaggaagccccggccggggaggctgaggggacggacgccgacgagacccccgcctcggccccggccccggtggccaccgcccccgcggctgcgctgctcgccggtggtggtcccacggcgtggttggtgtcgggccggtctgcggagggtcttgccgcgcaggcgggtcggttgggggagtgggcggctgggcgtgctgcgttgcgtccggtggatgtggcgtggtcgttggcggctacgcgggcggtgttggagcaccgtggtgtggtcgtggctggcgaccgggcggggctgttggatggcgtgcgagggctagcggctggtgagtcggccgcgccgggtgtggtgtcgggggttgctcgggccacgggtcgtacggtgtttgtgttcgccggtcagggcgctcagtgggtcggtatgggccgggagttgttggcctcgtcgccgttgttcgcggcgcgcctggcggagtgtgaggcggcgctggcgccgtatgtgcactggtcgctcagcgatgtcctgacgggtgcggagggtgctccggcgttggaggctgctgaggttgttcagcctgcgttgtgggcggtgatggtgtcgctggctgctgtctgggaggtggcgggtgtctccccggatgcggtggtgggccactcgcagggggagatcgctgcggcgacggtggcggggatgctgtcgctggacgacgctgcccgggtggtggcggtgcgttcgcgcgcgttgtcgtcgctggatgttgctggggcgatggtgtcggtggtgatgcccgccggtgcggtacgtgagctggtggcgggcttcgacggtcggttgtcggtagctgcggtcaatggcccggcgtcggtggtggtctccggacaggcagaggcactggcgtccttcgagcgggagttggcggcacgtaaggtgctgcgctggcgtatcccggcgacggactttgtggcgcattcggcggcggtggagccgcttgccgcccagctcgaagcggagctgtcgggcattgccccgcaggcgggccgcatcccgatggtctctaccgttacgggcgagtggctgaccggtgaggaggctgacgccggttactggttcgcgaacctgcgccagatggtgcgcttcgaggacgccgtccggagcctgctggagtccgggtacggagcgttcatcgaggtctcgccgcatccggtgctgacggccgcggtgaccgaaaccgcccaggacacggacgcgaacgcgaacgctaccccggtgctgagcatcggcaccctgtcgcaggctgacgcgggtgcggccggtctgctgcgtgccttcgcgcaggcgcatgtggcggggcttccggtggactggcgcaaggtcctgccggccggcgaggtggtggagcttcccacctacgccttccagcaccgccgctactggctggagacctccaccgccgtcggcggcggggacggggcgcccaccgaggtggaggcgcggttctgggccgccgtcgaaggcggcgacctgtcggacctcgcggacacgttggccgtcgaggaccgtcggcagcttcttgaggtgctgccggtgctggcgtcctggcggcgccgcgagcgggagcgttcggccaccgagggctggcgctaccaggtcacctgggcgccggtggccgacccggcaccggctcgcctggccggccgctggctgctgctcgtaccggcccaggaggtcgacggcgaggcggcccggcagtgccgtgcggcgctgtccgcccgtggagcggacgtcgtggtgctggagacggccggggccgtcgcacgtgaggcggtggccgagcaggtgcgcgcggcactgccgtcggacgggttcgccggcgttctctccctgctcgcgctggacgagacgccgatgccgggccacccggcgctgactgtgggtctggccgccaccctcatcctggtgcagggcctcggcgacgccgacgtggacgccccgctgtgggtagccacccgtggcgccgtcgccaccggctccgacgaggcgccgaacagcccgctgcaggccctgatctggggcttcggccgggttgtcgccctggagcaccccgaccgctggggcggactgatcgacctgcccgccgacttcgacggaccggacggaccggacaacgcggacgggcaggtcgcggcacgcctgggcgcggtgctggccggctgtggcgaagaccaggtggcgatccgcgcggccgccatcctgggccgacggctgacccacgccccgcagcccgtaatgcccgaccagccgtggacgccgggcggcagtgtcctggtcaccggcggtaccacggcggtcggcgggcacgtggcccgctgggcggccgaacgcggcgcccggcggctggtgctgaccggtgctcccgagcacgagggggtcgcggcgctcgccgccggactggccgcagcgggcagccgggtggacgtcgtggcgtgcgatgtgagcgaccgcgccgagacggccggggtactggcgtggacgcgggcccggggaccggagctgtccacggtgctgcacctggcggagatcgtcgacagcgtgccggtggacgacctggacgcggcgcagctggccgaggtgctggcggccaaggccggatcggcggcgatactggacgagttgaccgccgactgcgacctggacgcgttcgtactgttctcctcggtcgccgccgtgtggggcggcggccggcagcccggcttcagcgcggccaacgcctacctggaggcgctggccgaggaccggcgcgcccgtggcctgaccgctctcgcggtggcctggggcccgtggaacgcgggcaccaccgccaccggagacgacggcgtccagctggagcggcgcgggctgcggctgctggacccccgggcggccaccggcgcgctggcacagatcctggacggcggcgagagccgcccggtggtggtggacgtcaactgggcgcagttcgcgccgccgttcacgctgcgccgccccagcccgctgatcgccggcctgcccgaggtcgcggcggcgctggccgccggcaccgccgaggaggcggaaaccgtccccgaggcgcgcggttcgctgctgcgccgactggccgggcagagcgccaccgagcagcagcgcaccctgaccgacctggtcagaggcgaagcggcagccgtactggaacactcctccgcccaggccgtcggcagcggccgggccttcagcgagctgggtttcgactcgctgaccgcggtggagctgcgcaaccggctggccgccgccaccggcctggcgctgcccgccacgctgctcttcgactaccccagccccaaggtcctggccggctacctggccgccgaactgctgggcagcccggccgccggggccgacgccgccgtcccggtcccggtgccggccggtgcggaggaggacgacccggtggtcgtcgtcggcatgggctgccgtttccccggcggggtcaagacccctgaggacctgtggcaggtgctggccggcggcaccgatgcgatcggagccttcccgaccgaccgcggatgggacctggacgccctctacgacccggacccggacgcgccgggcaccgcctatgtgcggcagggcggcttcgtccacgacgccacccgcttcgacgccggcttcttcgggatcagcccgcgcgaggccctcgccatggacccgcagcagcggctgctgctgggcacctcctgggaggcgctggagcgcagcggcatcgtccccggctcgctgcgcggcagccgtaccggcgtgttcgcgggcgccagcttctccggctacggcctcgcccagccggaggcggcccagggcctggaggcgcacctgctgaccggtaccacgaccagcgtgctctccggccgcctcgcctacaccctgggcctccagggcccggcgatgaccgtggacaccgcctgctcctcggcgctggtggcggtccacctggccgcgcaggcgctgcgctcgggtgagtgcaccctggcgctggccggcggcgtgttcgtcgcctcgaccccggagctgttcgtctgggtcagcaagcagcgcggactggccccggacggccgctcgaagtccttcgccgccgacgcggacggcatgggcatcgccgagggcgccggcatggtcgtactggaacggctctccgacgcccggcgcaacggccacccggtgctcgcggtggtgcgcggctcggcggtcaaccaggacggtgcgtccaacgggctgaccgcgcccaacgggcccgcgcagcagcgcgtcatccacgaggccttggccggctgctcgctgacctgcgccgaggtggacgcggtggaggcgcacggctcgggcaccccgctgggcgaccccatcgaggcccaggcactgctggccacctacggccaggaccgcggcgaacgcgacccgctgtggctggggtcggtgaagtccaacatcggccacacccagggtgccgccggcatcagcggcctgatcaagatggtcctggcgctccagcacgagcagttgccgaagtccctgcacgccgagcagccctcgccgcacgtcgactggaccgcgggcgcggtacggctgctgagccaggcgcagccctggccggccggcggcgcacggacgcgccgggccggtatctcctccttcggcgtcagtggcaccaacgcccacctcatcctcgaagaggcccccgaggcccccgaggcccccgaagccggcggtgtccctgagacctcgccggcccccgaggccctgcccgccgacgcgtccggtgacggcggtcagtccgcagcaccccccgcgctgctcgcggcgcccggccccgtcgcctggctggtgtccgggcggacggccgacggactgcgccggcaggcggcccagttggcggagcacctggcggcccaccccggggcgcgggcggcggacatcgcgtggtcgctggccaccacccgtacggtgcacgagtaccgggccgtggtgagcggcgcggaccgtacagaactcaccgagggactggacgcgctggccgccgggctgaacagcccccaggtgatcgccggcaccaccgcggccgtcgagggggccgatgaggccgggcccgtcttcgtcttccccggacagggcgcccagtgggccggcatgggccgcgaactgctggccacgtccccggtgttcgccgaacgcttcgcccagtgcgccgaggcgctggccccgcacgtgcactggtcgccggccgacgtgctggccggtgccgacggcgcgccgggactggaacgcgccgaggtggcgcagccgctgctgtgggcggtgatggtggcgctggccgcggtgtgggaggccgcgggcgtcacccccgccgcggtggtcggccactcccagggggagatcgccgccgccacggtggccggcatcctctccctcgcggacgcggccgccgtcgtggcggtacgcagccaggaactgtccgccctgggcaccagcggcgggatgctgtcggtggtgctgcccgtcgcggccgtacgcgaactgatggagccctggggcgaccggctggccgtcgccgcggtcaacggccccgccaccaccgtggtctccgggcagccggaggcgctggccgagttcggtgccgctctggcggcccggcgcgccatgcgctggccggtcccggccggtgacttcgtggcgcactcggccgaggtggaccagctgtccggggcgctggccgagcggctggccggcatccggccgcagaccgggaacatcccgatgtactccaccgtcgagtgccgctgggcggacggcccggagctggacgccggctactggttcgccaacctccgccacaccgtgcgcttccagcaggcggtgcaggagctggctacggccggccacgccaccttcgtcgaaatctccccgcagccggtgctcaccgccgccatcgacgagaccttccaggagctggccacggccggcaccccggtggtcgccgcgacgctggagcgggagcacggcggcccgcggcggctgctggcggccctggcgcagctgcacgtggccggcaccgccgtcgactggcgccgggtgctgcccgccggccgccgtatcgacctgcccacctacgccttcgccgaccagcgttactggctcggtgccgaccacccggccggccccgccgccctcgcccccgcccagcccggtgcggccaccgaggccgagtcccgcttctgggcggccgtcgaggacggcgacgcatcccgcctcgccgccacgctggccgtggacaccgcccagcccttcggcgaggtgctcgcctcgctggcgtcctggcggcgccgcgagcgggaccgcgacgtcaccgccggctggcgctaccgggtcggctggagcccgctgaacgacccggccccggcccgcccgtccggtacctggctggtggcggcccccgccgcaccggcccccggcaccgtggacctcatccaggcatgcaccgacgcactgaccgcgcgcggcgcccggacccaggtggtccaggtggccgcgggcaccgagcgcgccacgctggccgaggccctgcgccccgtcggcatcgcgccgtccgaagcaggtcccgccaccaccccgccgcctgtggccggtgtgctgtcgctgctaggactggacgagacgccgctggccgaccacccggcggtacccgctggcctggccggcaccctgaccctgctgcaagcactgtcggacgccgacgtcagcgcgcccctgtggatggcgaccaccggcgcggtgtccaccgcccccggcgaccggctgacccgccccgtacaggcagccacctggggcctgggccgggtcgtcgcactggaacagcccgagcgctggggcggtctgatcgacctgcccgcgaccctggacgagcgggccgccgcccggctgtgcgcggtactggccggctgcggggaggacgaggtggccatccggcccgccggcatcctcgcccgcaggctggcccgggccccgcgtcccgccaccggccggccgtggaccccgcgcggtacggtcctgatcaccggcggcaccggtgccgtcggcggacacgtggcgcgctggctggccggccgcggcgccgaccggctggtgctgaccggccgctccggcgccggagccccgggcgccgccgccctcgccgcgcacgtggccgccgccggcaccaccgtggagatccgcgcctgcgacagcgcggaccgcacccggctggccgcactcctggaccggatcgccgccgacggaccgccgctgaccgccgtcatgcacaccgccggcgtcctggacgacggcgtgctggaccggctggacaccgaccggctggccaacgccctggccgccaaggccaccggcgccgcccacctggacgccctgaccgccgaccgcgacctggacgccttcgtcctgttctcctcggcggccgccaccttcggcggtgccgggcaggccaactacgcggcggccaacgccttcctggacgccctggccgagaaccggcgctcccgcggcctgcccgccctgtccatcgcctggggcccctgggccggcgacggcgtctcccaggccaccgaggcggcccggcagcggctgcgccgcaaccgctgggaagtcctgatggaccccgagctggccgtccaggccctgggcaccgccctggccggcacggacaccgtactgaccgtcatggacctcgacctcgccgccttcgccgccgcccccgggcagcaacaggccctggacgcacccctgctgcgcgacctggccgaggtccagcagctcaagacggcccggcacgccgacggcggcgcccagccggccgcaggcgaactgacccggcaactggccggcctgtcgcgcgccgaacaggaccggctgctggtggaccgcatccgcaccgaggccgccgccgtgctcggccacgccggctacgacgaggtcgaacccggccgcgccttcagcgagctgggcttcgactcgctgacctcggtcgaactgcgcaaccgcatcgccacctggaccgcactgcgcctcccggcgacgctgatgttcgaccaccccacccccacggtcctggccgcacacctgcgcaccacgctgctcggctccccggacgccggccccgccgcagcccccgccccggacctcacgggccacgccgaccgcgacccgatcgccgtcgtcgccatgagctgccgcttccccggcgacgtgaacacgcccgaagacctctgggccctgctggccgcaggcggcgagggaatctccggctacccgcaggaccggggctgggacgccgacgccctgttcgaccccgacccctcgcacgccggcacctcgtacacccgcgaaggcggattcctgcaccaggccacccacttcgacgccggcttcttcgggatcagcccgcgcgaggcgctggccatggacccccagcagcgcctgatgctggaactgtcctgggaggcactggagtccgccggcctggaccccgccaccctgcgcggctcgcgcaccggcgtgttcatcggcggctactcctcccactacagcctggccagcatgcagctcggcggccaggacggcgcggcccaggtggaaggccacctggtcaccggaaacgcgaccagcatcatctccggccgcgtctcctacgtcctgggcctggaaggcccggcgctgacggtggacaccgcctgctcctcggctctggtggcgctgcacaccgccgcccaggcactgcgctccggagagtgcccgctggccctggtcggcggggtcgcggtgatggccaccccctgggagatggtcggcttcgcccggcagcgcggactggcggcggacggccgctccaaggcgttctccgccgatgccgacggcatgggtatgggcgagggcgccggcatggtcgtactggaacggctctccgacgcccggcgtaacggccaccgggtcctgggcgtcatccgcggctccgccgtcaaccaggacggtgcctccaacggcctgaccgcccccaacggcccctcgcagcagcgcgtcatccgcgccgccctggccaacgcgggcctgacctgcgccgaggtggacgcggtggaggcacacggcaccggcactccgctgggcgacccgatcgaggcgcaggcgctgctggccacctacggccaggagcgtccggaggggcggccgctgtggctggggtcggtgaagtccaacatcggccacacccaggccgccgccggcatggcgggcctgatcaagatgctgctggcgctgcggcacgaagagctgccgcgcaccctgcacgccgaacagccctcgccgcatgtggactggtcggcgggcgaggtgcggttgctgacggagtcggtgccgtggccctccgacggcgaccgggtgcgcagggccggtacgtccgcgttcggtatcagtgggaccaatgcccatgtgattctggaggaggctccagcggtcgaggcgcggagcgaaagcgccgatgaggtgccggttgccgctcttgagggtgctggtgcgtgggtggtgtcgggtcgtacggctgaggcgttgtcggcgcaggccgggcggttgcgggagtgggtggcggcgcgtccggagctgggtgcggctgatgtggcttggtcgttggcggcgacgcggtcggcgttcgagcaccgtgctgtggtgcttggttcggagcgtggtgcccttctcgatggcgtgcacagcctggctgctggtacgtcctcgcctgtggtggtgtcgggggtggcccggtctgatgtccgggtgggcttgattttcgcggggcagggtgcgcagtgggtgggtatgggtcgtggcctgtatgcgggcagtgcggtgttcgccgaggtctttgaccgggtgtgtgggctgctggagttggagctgggggccgaggtacgtctgcgggatgtcgtcctggagggcgatggggacgctgctgggttggcggatcagacgctgtatgcgcaggctgggctgtttgccttcgaggtggccctggcggaggcgctgaaggctgctggtgtggtgctggatgcggtggtgggtcattcggtgggggaggtggctgcggcccatgtggctggggtgctgtcgttgccggacgcttgtgcgctggtggcggctcgtgcccggttgatgcaggagttgccgtcgggtggggcgatggcggcgatcaacgccgcggagacggcagtcgtcgcgtccctcgaagaggtgtcgggtgaggcggctgtcgctgcggtcaacggtccggagtccgtagtgatttccggtgcgactgaggccgtggacgcggtggtggagctgtggcgtggccggggctgtcgggtgcggcgcctgcgggtttcgcacgctttccactccccggcgatggaaccggtcctggacgagctccgtaccgtcgcggagaagctggagttccgtcgtcccgaggtgatgtgggcgggtgctctcaccggtgagctggtgtccgatccgcaggccgggtactggcccgcgcagacccgccagaccgtccgcttcgccgatgcggtggccaccctcgcggcggaaggcatctccgtcttcctcgaagtgggtccggacggctcgctctcctccctcggcccggacgcggtcgccgatgtggacggggccgagcccgccttcgtcccgctccagcggcgcagtaccgaaggcaccgcaggtctgctgtccggcctggcccgtgccttcgtcaacggtgtgccggtggactggaagcgtgtgctgcccgccggtgagcaggtcgagctgcccacgtacgccttccggcaccagcggtactggcccaacggcatacagaccctcccgcacgcctggggcgacagcgcctccgcaaccgcgcaggaccggttctgggcggccgtcgaggacggcgacctcaccgacatcgccggcactctcgccgcagaggatgcgcagcaactggcgcaggcactgcccgcgttggcgtcctggcggcgccgcgaacaggaccggtcggccatcgccgactggttctaccgggtggactgggagccggtcaccgacccggccccggctgaactgagcggcacctggctgttggtgtcccccgcgggcccggtcgcgcaggcgctgaccgagcactgcacgcaggcactggccgccgggggcgcccgggtcgtgcccctggaagccacgcagacagccgatcccgagcggctggcggcacagatcgccgaggtgcttcccccggacggcgtcgccggcgtcgtctccctgctggctctcgacgagaccccgctgcccggccacccggcgctgacacggggcctggccggttcccagaacctgacgcaggccctggtcgccgagcacgtgctcgccccggtgtggggcgtcacctgcggcgccgtcgccgtggcgcccgagaaggcgccggcccgtccggtgcaggcgcaggcatggggactgggccgggtcgtcgtcctggaacaccccgaccacggtggcgggctgatcgacctgcccgggcagtgggacgagaacaccgcacagcgcctgcgcgccgtactcgcaggctgcggcgaggaccaggtcgccctgcgcccgtccggcatccacggacgccggctgacccgcgccgcgcagccgcgcgaacgcgccgggcagtggacgccgcgcggcaccgccctggtcaccggcggtacgggcgccatcggcgggcacgtggcacgctggctggccgatcgcggcaccgagcggctggtgctcaccagccgctcgggccccggtgccgccaccagcgccgcgctggccgccgagctcgcggcggccggcacgcacgtggacatcctggcctgcgacgccgccgaggagaccgcgttggccggcgtgctcgaccggatcagtgtcggcggcccgccgctgaccgccgtcctgcacgcggccggcctcggccagatgaccccgctggaccaggcctcggtaccggaactggccgcggtcctcgcggccaaggccgccggcgcggccaccctggaccggctcaccgccgacctggaactggacgccttcgtcctgttctcctccgtcgccgccacctggggcagcgcgatgcaggccggctacgcggcggccaacgcctacctggacgccctggcggaggaccgccgggcccgcggcctggccgccacctcggtggcctggggcccgtggggcggcggcggcatgaccgacgaggaagccaccggccacatggaacggcgcggcctgcgcctgatcgacccgcacgcggcgctcggcgccctggcccgggcactggacgccgacgacgcgacgctcaccgtcgccgacgtggactgggaacgcttcgcgccgccgttcacactgcgccgccccagccccctgatcgagagcctgcccgacgtgatccaggcactggccgcccgcgatgacgacggcgatgccccggagacgtcggccgcaggcaacgcgctggcccgacagctcgccgccctgtccccgaccgatcaggaccggctgctgatcacactggtgcagaccgaggccgccgtcgtgctggggcaccccggccccgagtcggtacaggccgaacgggccttcagcgacctgggggccgactcgctgaccgccgtggaactgcgggaccggctgggccgcgccaccgggctgcgcctgcccagcaccctgctgttcgactacccgacctcggccgcactcgccggccacctgcgggaccgcctcaccgaggagagcagccccgcccgacccgtcctggccgaactggaccggctggaggagatgctcaccggggtgcccggcggcggcgacgccgcccagatcaccagccggctggagctcgtcctctcccgctggaagcagacccaggcccagaacgccgagaccgaagtcgcggagaagctcgaatcatcgtccgacgacgaagtcttcgacttcatcggcaaagaactcggaatctactga

SEQ ID NO: 69 - DNA, 672 bp, Streptomyces sp.
  source 1..672: mol_type = genomic DNA
atgagcgatgagaaggtgccgaccgacgacggcacgcaggacgagcaggcggccatgcgggcctcggacgcggaccgggagcaggtggcggagatattgcgggacgccgccggggacggccgcctgaccttggaggaggtcggtgaacgggtggagagcgcgctgcgcgcccgtacggtcggcgaactcgcccggctgacccgtgacctgcccgtctccccggtgccggccggttccggggagcagcagcccgggcgaggccgggacgtcgtgcagatcgaccagcggtggggcaccctgcaccgctccggtgcctgggaggtcccgcgccacctggaggtacggatgcacggcggggacgtgaaactcgacttcaccgaggccgagttcagccacgccaggctggacctcgacgtgcagatcagcgtaggcgggaatctggtcctggtggtccggccgggcatcgtggtgcacaccgacgacctgaccgtgaaggtgggcgaggtcaagtaccgcaatcccgcctccgacgcgcaggcgccggtcgaacgcgaggtgtacgtcaccgggcgtctcaagggcggtgacgtggtggtccgtcacccgaggcggacgctgggccggtggctgcgcggcgaggacgccaagaacggcgcggcggagaagagctga

SEQ ID NO: 70 - DNA, 1014 bp, Streptomyces sp.
  source 1..1014: mol_type = genomic DNA
atgaacctgctcatgggagccatggcctcctcggccgtcggcaccgccgccgaactgggcatcgccgacgccctggccgacggcccggccaccgccgacgaactggccaagaccgtcgaggcggacaccgactccctcgactccgtcctgcaggccctgaccgcactgggcgtcttcacccgcgacgcggacggccgctacgccaactccccgatgtccgaaccgctcaagagcgaccaccccgattcggtccgccacctggcgaccctgctgtgcggcctctacgggcaggccgcgaccggcggcctcacccaggccgtacgcgccggcacgtccgtgatgccctgcaccttcggcgtcggactctacgagtacctcgacagccacccggacaccgcccggatcttcgacctcgccatgcaggactggacccgtccgatcgccaccctgctggccgacaagcttcccctcgacgacgtgcacaccgtcgtcgacgtcggcggtggcaacggcaccctgctccaggccctcctgagcgcccacccgcacctgaacggcatctgcgccgaccgccccgacacctgtgaccgcgccgagcgcaccctggccgaatcccagcaggccgatctcgcccgccgcctcaccttccggcccaccgacatcctccaggacgtccccaccggcggcgacctctacctcatcaagaacgtcctgcacgactggaacaccaccaccagcgtgcagatcctgcgcaacatccacaccgccatgaccgccaccccccgcacttccgccggagccccgcccaagctcctgatcatcgacccgctggccgagcacgactccggagccgcgttccgcaacgtcatcaagatggtggtcggcgagccccggacccgctcccgcagcgaggcggacatccgccgcgaagccaccgaggccggcttcgaggtcctgtccatcactccttgccccgcagacctcagcgtcatcacctgcgtccccgcctcctga

SEQ ID NO: 71 - protein, 226 aa, Streptomyces sp.
MREQKIMSRLSASNSVMYELASQNPNMSDLDRLTNLYWALCSVLRAEVPGEVVELGCNAGLTSVMLEMVLEAESPDRTLHLYDSFDGLPAPSHADQYLREGDCKATEADVCAAFERWGLRRPEIHAGWFDTTLPQQLPDRVAFAYLDGDFYESILTSLTHVWPRLSVGGSVLVDDYCDRLASPRAWDGLPGVKKACDAFFADVPVRSRVLVGAGDLAFAEFRKTEA

SEQ ID NO: 72 - protein, 222 aa, Streptomyces sp.
VLHDSLKIGSVEGLLSPDEVTRLTACVTDGLGADRRFTVSERRRSVHSLEGESLARVKEVYEPHGRVEYDELPDEITGVLEGAVARRLGELRRIFPTVRRSMDWLYVEYGAGQYVTPHVDYPFNEIAPDRPKVAAIGIVLHKAEAGGEFFVETGGGDALWNDEGEVKEGANDHSPWFNAMRRTRWHVTSRAGDAVCWGTQLVHGTEPVAAGRSGKFIGFLGS

SEQ ID NO: 73 - protein, 296 aa, Streptomyces sp.
VKTDFPYHPSQDRRGDRLTPPVAERNARRSRGVAGRIVWAHSLWIDPELERLYGGDRHAPDVNRERAVRLSDGRLDPLAMRPLDSLVSVTGPFLSRHTSTVGVDPADEAFDLVRRTHSGYLCELAIGVIRPGSPQRGVTPSGPDHRERSPFTAVVPRDHDAVSRAAGLLESSANELLSQWYEAFTHARGTTHGILLPPLRWAQGVFALSIRYEYCRRVRDEPGFTAGERQHAGLLLDEVTEALPQPGAFRDSLPGGVQGSVVNELLSCSYPLWDRVRFCLDSPFDGLRSLAEHSTA

SEQ ID NO: 74 - protein, 239 aa, Streptomyces sp.
MSHAAFPAVESATHNAEGEHGHHRARFPYVALEGLNGAGKSTLRNSLEESFWALGMNVYTIGQHSWQSPGAARVILDIKERRHTHPPERIQLAYLDDRRAQGDNILSRVLHEVPVIGDRSLVSDIVYLEALDGIPAEDTFALYRRHAVILPDLIVHVDVSAATALANLGKRERRRKPHENPEQMRSLAATYESVLWAQRLPGLPPVLRYQPEVAADGTSYTNPAASIGDRILASLGLAS

SEQ ID NO: 75 - protein, 194 aa, Streptomyces sp.
MSAPGRAIVLAGTSGAGKSSTAEELQKTLPDAFLVLGLDTFFGMVPYRWTSAGTASAEGFSYIRDTDPHDGAARLRIGYGPVGAHLLAGMRRATEALLREGNNVIVDEMPIDSGVVPAWKRDLLGHSVLWVHLTAPLPVLEAREAERFPEKFRGLSRGHFDVCVPADFDIVVNSAELDPRERADRIAAALPPRR

SEQ ID NO: 76 - protein, 408 aa, Streptomyces sp.
MSDARSATPAVPLRRNRDYNLWWSGAAASQTGSFMVTVAVPMLILTNSGSAGQAGLVTGASAVAKFVVAFPAGAVADRCSRRRLLLLSSLMQLAAVASILIAVASQHVRLVHLAAAALVEGAGYALHSATELPLLRKVVPEPQRRAALGREQGRKAAAQLTGPPLGGTLFSWVPWAPFLAASVSFLGVTTAALGIRAPLGPQRRRKEAAPPFFPEMLEGVRYIRRSAFMSYMLLWFALVNGAFAGLSFLLVVLCHDRGASASAVGTAQGVGSAGAVLGALASAWLTARLTHRQLLKLASWVLVIGSAAMAVPTLSPLPTGLAYGVSLFLTPAVNVSFTHHMVGTVPDALTGRVSMTMMTAARSLNWLFVIAVGLAAERWDPLVPLFTLSSLFLALACANHLRRVRTQR

SEQ ID NO: 77 - protein, 42 aa, Streptomyces sp.
MAVRPVIKARNTHLICENAARGGVRDARGVTVVPALPLRAQR

SEQ ID NO: 78 - protein, 1125 aa, Streptomyces sp.
LTTSLWSRRRLLSTLAGAAAVPALPSILTLNAGVAHAAGPADGNGPGDVLPTTERGQAVWAYKTGGRAVREAAAAALVGSPAAVTAFLTRELPAARAEDNRFAIIMGLSQGGKAVNETAGAALTAGDEAVAAYLRDGYKKPVEEDLRVAVFTVMNNSGVAVRREADKALRENTRSAFQKFLGETQYTAQQEDERVEVFSILNNASPEVKKYAQRALSEGTPEAIRQFLSSGQHIARARDEERATIDQLVEIVQRESRQAKLQTENAVTASERAKSAAEKAKAAALLAAEHAKAAEQDVRKSADAATSAARAASGAADAAGAAIAASRTAQSASHRAAQAAQAAAAAAAMAGHAASRAYNAAIAASKDASKASAARVAAQGARNAAAQARTAAKAADQATIASQQAAVAGTAAAGAARSAASAANAAAESAAAAGAAQSEAAKARQAAAEADAAAARATRAAATARSLANQAAEAAGVARDAANSAADHAEKAAAAAEEAAKNAGKAVEYAKRSAAYAEAAMEAATKADNAVKKALQVEKAAREAEAARIAADTELGVLEARLRAKAETEDAKRAERQRAQADRATQRVKDLIAAAEAALRAGDTAKAVASGRQAAVGLLESSGTWTREAAEFALAGTDQDTVNWIDTDRLLAQQQDDRETVLTIATSSTADVAEAAHKVLAGDSPEAVRDFLDRGVLEAAATDNRVALFRVLDENPGRAVREKAEAALKDGSAQALHRFLTEELTEAVKEDDSVEVFRLLGSGGPYMQSAAKIVLEGSARMRRGFVSHDQYNIARLDHDRATHVAAIQSSIALAAKVAAEALESAARASKAAAEARDAAADATDWANKADAYAQRATDSATEARRNADAADKSAAAAAQSAKEASNAAVTARGAARAANFSMRQASASAQQAVASAWQAQASASRAQASATQAGKAAKAAAAAASEARRIVFVKRKAEAEAAAKRAAEKAKEHQSNGTNPSQTSDNDKPWYVENGVWPEDVKSAKDWAQVTGHWSTIAGGAAVALGVGGFFFAPLGGAALAVGLVSWGLQGASALLNYIGNGPDSSEFHSALGMFAVGGILLGTGQLLSKFGKMPAFGPAVEQVGEKIANVAHDITTTVAGWLTW

SEQ ID NO: 79 - protein, 125 aa, Streptomyces sp.
MLTSTLSETQWWVAAVLSGFALAVAAQSMATTARDKLIAASLPLAGAIAIGVSSAARGYTDTLPLLLYTTTMIVLVILRLLFAKYINRQLALKRSGKPMENMSGKQTAIFLLAFMATLVLVSFVI

SEQ ID NO: 80 - protein, 167 aa, Streptomyces sp.
MRITRRLIVTGTVLAASAATAVTVAQATGNPAPATVTVAGAEPGSAVEDFSYPNADKIKKELGIDLKRGDGHIMLVDCNNDGRLMTIWSRTIGQVCFRVTGDTGYLSLSIPKVYGVKADADHKANLTLTADGSKQNIALGKNESGSAGETADPERRPHDLVEISTSK

SEQ ID NO: 81 - protein, 49 aa, Streptomyces sp.
MDVQMSTEADSDLGELEVLEMDELQVSGSSTAYNYIAKNADRSSGIPAG

SEQ ID NO: 82 - protein, 279 aa, Streptomyces sp.
MICCPPRQMDVPGQSRRIHRAISRAGANWLKKATDMPTARGLDASSASASAARRFRVAKSREPACGSDNRSTAWSSASKSSHTCDSADSRSISWVACRARRTARTGPRRGAGFFVRKVNSYLNDTVVLSMVSCVGCYWGRCIFCSYGNRSLERGAYQQASPGQLADTVCRIVKSTGIDFVTVVDENTNLLLLVRAMRLVRERGLRVRFNTRNRLEPILLDSAFCQELAELGCEGMAVGYEGVTQRLLFTVLDLRIADFGRQVGVTDWPARYAVLRRQED

SEQ ID NO: 83 - protein, 257 aa, Streptomyces sp.
MDDTTLISRFLRDGFVKLEGAVAPRVAADCARLLWRETGCDPDDPTTWTQPVHWVPGLAQGPFAAAPNSPALHHAYDLLVGAGRWEPRYSLGTFPLRFPHEQEPDDAGWHIEGSYLPEGESWYFTNLRSRGRALLMLFLFSEVGDQDAPTRIRVGSHLDVPKVLEGYGEDGASGLTLAPDLVAASDHRPLTLATGSPGDVFLCHPFLVHAAQPHHGRRPRFMAQPPLMPAAPYELERADGAYSPVEIAIRRGLGRDR

SEQ ID NO: 84 - protein, 59 aa, Streptomyces sp.
MARRQPHAPERLLVAAHPELREREALKELGLIASMTDALKRRGVPDLTARVAAELGALA

SEQ ID NO: 85 - protein, 314 aa, Streptomyces sp.
MSRAVVYESFGGPEVLELREVPEPHAGPGEVRVRVTAAGLNPMDWGIASQPEAAARFGIAVPAGFGFDFAGAVDEVGAGAEGFAVGDRVHGGALGRAVADFVVVKTPTGTTDPLWHTPEGISDEVAATLPVAGMTAAAALAAIDLRSGDTVLVGGAAGGVGVFVVQLAQLAGARVIGTAGPGTFPFLRRFGVEPVTYGPGLADRVRTLAPEGVTAATDLFGIETAETALALGVPAERISTIAAGPNPPGGARATGGFDASPDAMERIGDAILAGAITVPIAAAFPVEQIREAVTLQAGRHVHGKVVITLRPGPH

SEQ ID NO: 86 - protein, 66 aa, Streptomyces sp.
VQTLAPLLAEGSGVVLTASAVNVLGLPMLSAYAAGKAALRSMTRSLARELLPRNRGRRWHSALTPV

SEQ ID NO: 87 - protein, 288 aa, Streptomyces sp.
MHTAAVSDVPVGNRTELGDFLRSRRGRVAPQEAGLPPSAGRRRTAGLRREEVAILAGVSIEYYRRLEQGKQRRPSPAVLEAIGQVLKLTDDERRHLAELSRGRPPSPVTGARTGRASRAVREEVRRMLEIAHPYPACVLSRSSDLLTANQGALRLFPGIEDWPEPERNTARYAFVHPRARELYADWDDVARGVVAHLRAAVVVHRGAPDIREVVDELSARSTAFASLWQRHDVRARTNGRKVYHHPDAGRLTLTYEAYDVARSEGQRLIVYQAPPGTPDHEALLRLSA

SEQ ID NO: 88 - protein, 494 aa, Streptomyces sp.
MGGAEAHEEGTVGPAVSTQAEPTRRGRQLVLAVCCTSVFLSSLNNTVLNVALPAMQRDLRSPLSGLQWTTDAYLIVLAALLMLAGSAGDRLGRRRVFRTGLALFTLGSLLCAFAPGLGWLIAFRTVQAVGASMLNPVAMSIITNTFTAPRERARAIGVWGAVMGVAMAVGPLVGGVLVQGLGWRSIFWLNLPVGAAAFVCAARFVPESRAPRPRRVDPVGQALVVVLLGCLTYAIIEGPGTGWTAPRTLACALAAPAALLALVLHERRRTEPLVDLRVFRGAGFSGASAMAVLSFFVLGGFLFLNTLYLQNVRGLSALAAGLCLLPMAVMNIVTSPLSGRLTATRGPRLPLLLAGAATAAGGVLLATGGALTTDALLFTAYVLIGVGAGLANTPVTDAAMSGMPREQAGVAAAVVATSRQFGQALGVAVLGAVLTAGLHRSGTPFTRAVLPGYWIVTACGLAIVVLALATTRRVVPAASPAGRRRRQAAGARLG

SEQ ID NO: 89 - protein, 204 aa, Streptomyces sp.
MPKTVDRDEQRRQIGAALLSLASERGLDEVSVRTVATYCGRSPGAVQKYFRSKDEMLAFAAEMAGERIEARMAAVDPGLPPRAALRALVLTTLPLDAERRAEAAAQLAFAVRAAHHEPLAEVRRRVDQDIREALAHWLTSAGYGHGPDPATDCLAIADAVIALSDGLALRLLYTPQAHDDLVRALDRGLEALVPAQAPGEPDAR

SEQ ID NO: 90 - protein, 92 aa, Streptomyces sp.
MDDRLDPVTRASDQDREKALARERDRKDLREWLAEWRCWLGGGVILSAVWGVRCLRKGELGFYWPVTPLGIWAAVLVAIAVWPRSGGGGGEG

SEQ ID NO: 91 - protein, 7698 aa, Streptomyces sp.
MGRVVPGTPHHSRPLSSAQRGIWFAQQLDPDSPSYHTAEYVEIEGPLDLPAFETAVRRAVTETDSLNVRFTDDGQGPCQTIQRGGTWSYRVFDVGAEPDPRAAAEAWMRAETDRPGGLEAGRTVTLALLRAGADRAFFYLRAHHLVTDGYMCALFTHRVAELYTALVTGAPQREDSSGSLDLLLADDAAYRESERFAQDRAHWLDRLAGRPEPVNLAGRTARVSRSFLRYGTRVPDATADGLRALARRAGVGLPVPVIAATALYLGRMTGADEVALGIPVTARSGGGRHIRGPMSNELPLWVEVRPGMSGRDLLRQVSAGARQLLRHQRYRYEDIVRDVRTVGGERRLFGPTVNIMSFATELTFGEARATAHNLSVGPVTDLSFRLDDRLGGGALHLDVDANPALYDAAETAAHQRRFLRVLESFAAADPERPVGRIDFLLPGERAEVLPVRPAATDAPEPDEPGPTLPTLFEAQAARTPDAVAVVTEDGETEDGELTYAGLNHRANRLARLLLKRGAGPERLVAVALPRSADLVVALLAVVKTGAAYLPVDPAHPAERIALMLRDADPVCLVTTGEPAAGAPYGADVVRLDDDATVRALADGPGDDLTDADRAGQLDPAHPAYVIYTSGSTGTPKGVAVPHRNVVRLFRQTEAWFGFGPDDVWTLFHSYAFDFSVWELWGALLHGGRLVVVPQDVSRSPERFLDLLVRHRVTVLNQTPSAFYPLIRADHDHPELSARLALRTVVFGGEALDPGRLRAWYERHPADAPRLVNMYGITETTVHVTHAPLDERDAAADPPRSVIGEPIPDLRAYVLDNALRPVPPGVTGELYVAGPGLARGYLGRPGLSAQRFVADPFGRPGERMYRTGDRARRHADGRLDHAGRADDQVKLRGFRIEPGEVEAVLARHGAVAQAVVLLREDRPGDRRLVAYAVPAGGPRPESAALREFAGRHLPGHMVPAAVVVLDRLPLTANGKLDRAALPSPEYTPGAAHRPARDAAERVLCDLFAEILGVRRVGVDDGFFDLGGDSLLATRLLSRIRTALGAELPIRVLFDTPTVAGLATAVRESGGEARPVVRRTERPEALPLSFAQRRLWFLNRLEGREAGADYNMPFVLRLEGALDRAALEAALTDVVTRHESLRTVFPEVDGVPRQVVRDPGAATVTLPARDVDAAELAEVLARSAATGFDLAAELPLRAALFALAPDRHVLSLVVHHIAGDAWSMVPLGRDLSTAYAARLRGEEPSWAPLPVTYADYTLWQRDLLGEERDEDSLIARQVAYWARTLAGLPDELALPADRPRPGVASHRGGTVPLRLDAALHVRLAALAEAHGVTLFMVLQAGLAALLTRHGAGTDIPLGSPVAGRTDDALDDLIGCFVNTLVLRTDTSGDPGFGELLARVRETDLAAYAHQDVPFEHLVEVLNPTRSLSRHPLFQVLLTLTGRTAAPDALPGLRAEAERVESGTAKFDLTFNLTEAVDAGSGPGGVEGYVGYSADLFERGTVEALAARFVRLLEAAAADPDLPLGALDILGEAERHRLLTEWSGTGGRTGTGAAAGGARSTASLPELFERQAAATPEARAIAAPDAELSYAQADARANRLARLLISRGIGPEQVVAVALPGSAELVVALLAVLKTGAAYLPLDLAQPGARIAGLLDDATPALLLTTSHAATGLPDDTGAGTGTDTGPARLLLDAPDTVRALERQSAARPTDADRSAPQLPDHPAYVIYTSGSTGRPKGVVVTHRGLAHYVARARRVYTGLDGTVLVPTSLAYDLAVTGLYGALTSGGCAVVAGLDDDPWLTALLARTPATFMKATPSHLVLLAALPAEFAPVRQLMLGGEALPAAHLEEWRARHPEVAVVNHYGPTEATVGCLDLHLAPGEAPGGATVPVGRPMAGMRAYVLDAGLRPVPPGVAGELYVAGPQLARGYLGRPGLTAERFVACPFAPGMGVPPAEGWGRMYRTGDLARWTADGVLVFAGRADEQVKIRGHRVEPGEVEAALAGHPSVARAAVAVREDRPGDQRLAAYVVPAGDADGVDPAELRAYLARRLPEPMVPALFTTLGALPLTRNGKVDRRALPAPALAPASTGRPPRSPEEEILCGLFAEVLGVPRAGADDGFFDLGGHSLLATRLVSRVRTVLGAELPVRAVFETPTVAGLAARLKDGSGRRRPPLRPFPRRPETVPLSFAQRRLWFLHQLEGAPNYNLPFVVRLEGPLDRAALTAALGDVVARHESLRTVFPDSGGTPRQAVLDAGPAAASVELPVRHVADEDALAATVVAAAAHRFELTRELPLRAELCALGADVHVLIVVVHHIAGDGWSNGPLARDLSEAYAARRTGQAPRWEPLPVQYADYTLWQQELLGSEDDPDSLAARQIAYWSRTLDGLPEELDLPADRLRPAVPGYTGGVVTVSVDAGLHARLEELARTSGASLFMVLQAGLAALFTRLGAGTDIPLGSAIAGRTDDALDDLVGFFLNTLVLRLDTSGDPAFEELLGRVRETALAAYAHQDVPFEHLVEVLNPARSAARHPLFQTMLTLHNNARPRLDLPGLRVSPDMAEVGTAKFDLSFGLVEERGPAGEPAGLAGILEYSTDLFDHATAEELVARFVRLLGAAADGPGRRIGELDILSPSERVRILGDWAGTSQDAPALTVPELFERQATLTPDATAVVAAGTTLTFAELRARADGLARHLAERGVGPDTLVGLCVSRSAWMVPAMLGIWKAGGACVPLDPDYPAERLAFMAADSGVRVLVAEPDLADVLEGQAAETVLLGSPDADGEPFPMPAVRADHAAFIVYTSGSTGVPKGVTVRHGGIANLFANHRERVFAPAARAAGGRLRAAHTASLSFDLCVGQLLALLAGHELHVVDEDTRRDAVALGAYVRDHGIGYLSITPTLTQELLGLGLLGAEGHQPAAILHGAEATGPGLWQRLRGLSATEVHNYYAPSECTVDAIGATVSGSERPRIGQPVAGMAAYVLDAGLSPVPPGVRGELYLAGAQLARGYLNRPVLTAERFVANPFSSVPGARMYRTGDLARWTADGALEYAGRADDQVKLRGFRIELGEVEAVLARHPDVAHAVAVVREDRPGDRRLVAYTVAARGAVPDPAGLRAHAARTLPEFMVPSAVVPLDALPLTPSGKVARRALPAPGSAVPQGGREPRTAREEILCGLFAEVLGCARPGVDEGFFDLGGHSLLATRLLSRVRAVLDVELTVRDVFEAPTVAALAARLDAGGAARPALRPVERPGTLPLSFAQRRLWFLNRLDGAGAAYAMPLALRLRGDLDRDALAAALADVVTRHEVLRTVYPETAGTPEQVVLAPERAAPGLSCRAVAETGLAAAVAAAAAHPFDLAAEPPLHAELLALDPDDHVLVLVLHHIAGDGWSNLPLTRDVTAAYAARHAGEAPSWTPLPVQYADYTLWQHRLFGDPADPDSLIARQTAYWTETLAGLPEELPLPTDRPRPAVARHRGGRVPFTLDAAAHAGLADLARACDASLFMVGQAALAALLSRLGAGDDIPIGTPVAGRTDDALDDAVGCFVNTLVLRTATGGDPAFTDLVARARETALAAYDHQDVPFEHLVEHLEPVRSLARHPLFQVMLALRNTGEAPLGLPGLAVSAVPVETTAVKFDLAFTVSERTGPQGEAAGLTGELDYASDLFDAETARRIAARFVRVLEAVAADPARKLEDLPVLDAAERERVLTGWNATERPLPPVTLPELFAAQAARTPDAVAVAGEGGTLSYAELDAWADRLARALVQRGAGPERVVAVALPRSVELVVALLAVLKSGAAYLPVDPGHPAERTRAMLDEARPVCVLDDPEGVRNPRPETAITAPPVPLSPSHPAYVLYTSGSTGTPKGVVVPHAGVVNRLLWAQAEYGLDASDAVLQKTPYGFDVSVWEFFWPLITGARLVVARPDGHRDPAYLAALIREHAVTTVHFVPSMLRAFLDEPAAARCTSLRRVLCSGEALPAQLAAHGRDILGGSKLHNLYGPTEASVDVTSWACADRPGPVPIGRPVHNTRAYVLDAALRPVPPGVTGELYLAGVQLARGYLGRPGLTAERFVACPFAPGTGVPPAEGWGRMYRTGDLARWTADGVLEYAGRADDQVKLRGFRIEPGEIETALARHGDVAQVAVVLREDRPGDQRLVAYVTSAPGAVPEPRELRESVARTLPASMVPAEIVVLDALPLTPNGKLDRRALPAPDATAQAPGRAPRTLHEEILCGIFADVLGRPLPGIDDGFFDLGGHSLLATRLVSRARAALGAELSVRDVFEAPTVAGLAARIGSGGSRRPALRPAAHRPDVVPLSFAQRRLWFLHRLEGPSPTYNMPFALRLSGPLDTAALRAALDDVVARHETLRTVFPEIKGAPGQIVLGPDETAVELRTHRTDEAGLTGALAEAAGYRFELTAEPPLRADLFALADDDHVLLLVLHHIAGDGWSNVPFARDLSTAYTARCAGHAPAWAPLPVQYADYALWQHDLLGSEHDPDSLISAQVAHWTEALAGLPEELALPADRPRPAVSAHRGGRVVFALDAELHTRLAGLAREHGATLFMVVQAGLAALLTRLGAGTDIPLGSAVAGRTDAALDDLVGFFVNTLVLRLDTSGDPAFSALLDRARETDLAAYAHQDVPFDHLVELLNPARSAARHPLFQVMLTLQNNATPEFAMAGLRVTPDVAELAAAKFDLLFSLTDHYTEDGAPDRVDGVLEYSADLFDHGTAEALTARFVRLLCAVAEAPDRPIGTVDLTDAAERERLLETWNGAALTTPPVTLPEAFEAQTRRTPDAVAVTDGSTNLTYAALNARANQLARLLLGRGIGPEQLVAVALPRSAELVVALLAVLKTGAAYLPLDPAHPAERLRFLLGDARAAYGITGSALVGDLPETDTRWLTPDSDEARREAGDQPEHDVTDAERTAPLRPEHPAYVIHTSGSTGTPKGVLVEHRALAAYLAFAREAYPGTAGTTLLHSPVSFDLTVTALYAPLTAGGRVRIAELSADAPAPGPYTFLKVTPSHLALLGATPPGPETAVADLVAGGEQLTGEQLAPWRRAQPQAAVVNEYGPTETTVGCVVLRMPPGATAGDGPLPIGRPVPGTRAYVLDERLRPVPPGVTGELYLAGAQLARGYLNRPALTAERFTADPFGGPGGRMYRTGDLARWSTEGELHFAGRADEQVKVRGHRIEPGEIETALLRHPAVARAAVVAREDRTSGTQLVAYVVPADGERSGDAAARAQLDEWQEVYDSLYGGDGHQDAEPAPFGADFRGWTSSYDGRLLPVEQMRRWREATVDRIRELRPRRVLEIGVGSGLLLSRLAPDCETYWGTDIAADAVDRLRTQVPERPDLAGRVELRVQPADDLTGLPEGFFDTVVINSVAQYFPDAGYLTDVLGTALGLLAPGGAVFLGDVRDLRLLRCMHTAVQLRRATPSADPAAVRRAVEQAVLLEKELLLDPAFFAALAARNPDVAGVDLRVKRGAYVNELNRYRYDVVLHKRSADGPTDLAAAPELRWGHDVTGPDDLARRLTTERVPALRVTGVPDERLAAEAGAARGLDEGRPLADVLALLGPAEQPTAPDLGPDTFHELGERAGYQVAVTRSGDDAELLDVLFTDPAVTALSGVHRPAAEPSHPLANDPAASRQHGELVASLTASLRAELPAHMVPAAVLVLTELPLTGNGKLDRAALPAPYLGSVEGSRGPSTPRERVLCDLFAQVLGLAAAGVDDNFFHLGGHSLLATRLISRIRTALGVELPVRALFETPTVAGLAARLDDEESRDATRPALRPMSRPDTLPLSFAQQRLWFLHRLEGPNATYNMPIALRMRGAVDRTALRAALADVAARHESLRTVFPVTDGVPRQLVLDADGARPELLVRHTDETGLREAVQAAARYPFDLAADLPLRAELFDLGEREHLLVLVVHHIAGDGWSMAPLARDLATAYAARTRGRAPSWTPLPVQYADYTLWQRALLGDETDPDSPFARQIAYWSETLADLPQELALPTDRPRPAVSTYRGEMLGFDLSPELHRRLADLARDTGTSLHMVLHAGLAALLTRLGAGTDLPIGSGVAGRHDEALDDAVGLFVNTLVMRTDTSGDPSFGELLRRVRETALNAYSHQDVPFEHLVEALNPVRSPNRHPLFQVAMVLQNTPEAGLALPGVEVTPEPAASGTSRFDLFFSVTERGSSGGVRGTLEYSTDLFDRRTAETLVERWARLLEAVTADPARPIGEADVLTPAEHARTARASGADGPQVPPATVPELFARQVARHPGALAVLSDSGTLTYQELNVRANRLAHLLLSRGIGPGHVVALAFPRGAGLIVALLATLKAGAAYLPLDTGYPAERLAFMLDDARPSLLLTGVAGQEIPGSGRLSRLDLDDGDGHLAGQPDTDPGAEQAGRPDPAHPAYVIYTSGSTGVPKGVAVTHAGAAALAATQADSFGAGPGSRVLQFASPSFDAAFWELCMAMLTGAALVVAPAERLLPGPDLAAVLAEHEVTHLTVPPVALGVMTPDQLAPVTCLVVAGESCPPQTAAAFAAGRRMVNAYGPTESTVCGTMSRPLTGDGAVAPIGTPITGTRAYVLDAALRPVPTGVAGELYLAGAGLAQGYLGRPGVTAERFTADPFGAPGARMYRTGDLARWNAEGELEFAGRTDDQVKVRGHRVELGEVEAAMAAHPEVAQAVATVREDGPGGTRLLGYVVPAADGPRFTEATGAAEAAQAADGAPERALGEWRQLYDTLYGTTQTAPFGEDFTGWNSTYDGEPLPADEMRQWRDATVARIRDLRPRRVLEIGVGRGLLLSKLAGECAAYWGTDLSAAVIESLTAQVAERPALAGRVELRTQPADDLTGLPEGFFDTVVLNSVAQYFPDAAYLTDVIGAALRLLTPGGALFLGDLRNLRLLPSLRAATGTTAPERELLVDPDFFPALPATVPEIGAVDLRLKRGAYHNELSRYRYDVVLHRAPLTAPARPFADAPTLPWHPGLGGTAEIAALLTERQPDRLRLTGIPNARLAGEAATGHLVTVDPETLCATGERLGFWTAVTWSGTAGDDCFDSLFAAQDAAAPDDTAPYGSAPVTGVYVPADGRTAPPSFTNDPAAAVAPSAALGSSVRAWLRRRLPEYLVPSVVIPLDVLPLTPSGKADRTALPAPETTAGTTAGRPPATARERLLCDIFADVLGLPSVGADDDFFDLGGDSIVSIQVVSRARAAGMAIGPQDVFRHKTVAALAVAAGTSDAAPAATEAPQAGIGVVPLTPDLHALRERDADPGTGYEAMTVPAPAGLGAEALEAALQAVLDHHDMLRARLTSGGPVAADGPGWLLEVRPPGTVRAADHVRRTGTAPHEAAVAEEERAAAARLAPESGVLLQAVHFDAPPGSPGLLLLVLHRLVTDPQTWRILLRDVTDACEAAADGRQPRLAPVGTSFRRWAQHLRTQARDPRRTGELSRWTGMLAGPGSPDDAPDARATGNPDGIPAHEAGDAMLTTLLSAERTGQVLTDVPAAFHADAEDVLLTAFALAVADWQRRSSAADGTVLADVRRPGRVPLGEGIDLSRTVGRCTTTHPVRLDLGDPGGEALREDGGHAAATALKRVKEELRSLPDGGLGHGLLRHLHPGTAPELAALPVPRFAFHYLGELPQAPSGPDPAGEPACYWAEVAITAVDGPGGPRLAATWRRAHAPLTGRDLNDLATAWTRALETLAAQAARPGAGGRTPSDVPLVRLSQDKLEQLEAAWRRSQ

SEQ ID NO: 92 - protein, 2376 aa, Streptomyces sp.
VTQPELEDILPLAPLQEGLLFHALYDTAALDLYTLQSAYDLDGDLDATALREAARTLLRRHAALRAGFWHEGLDEPVQIVPREVALAWQQADLTGTAPEEREAEAERIAAEDRLRRFDPARPPLLRFTLLRLGTERHRFVVTAHHLVLDGWSVQLLLEELFQLYVRGGREDGMPPVARYRDYLAWLAGQDRAAAEDVWRRALKGIEEPTRVAPADPSREPALPGQILRDLPDGLANALDALARRERLTLSTVFLGAWAVVLGRLTGKDDVVFGTTVSGRPPQVPGVERMIGLFANTLPVRVRLDPEQPLNGMLARLQEEQTHLLACQHVGLSGIQRTLGSGELFDTVTVFENLPAGQSGLAATAHGGGPRVTASRSQGVTHYPLNFMVMPGRPLRLRLDHQTGLFDRDAAEAIMARVVRVLENLVADPGRRLGELEILAPAERARVLGEWSGTAGEPPAELMPQAFAARVATTPDAVALVCGEVSWTFAELDARADRLARRLAARGAGPDALVGLCVRRSADMIAAVLAIWKAGGVCVPLDPGYPAERLAFMLADAAVPLVLTERELAGRLGEDRAASLVLLDDDTPSETGVALRTPPLHGDHAAYIVYTSGSTGVPKGVVVSHLGLANLYADHRDRVFAPAADAVAASAGRRLRVAHTASVSFDASMGQLAGLVAGHELHVVDEDTRRDATALLAYVRERSVDYLGIAPSLMRELTALGLLDGPGHRPAVILLGAEAVDAALWEALRAHPETEVHNYYGPSECTVDVVGALVRDSARPVIGRPVANTRAYVLDARLRPVPPGVVGEVYLAGIQLARGYLGRPGLTAARFVADPFGTPGTRMYRTGDLARWTADGRLDFAGRTDDQVKLRGYRVEPGEIATVLARHATVAHAAAAVREDRPGDQRLTAYVVPAPGARAHAEELREFCAGTLPEFMVPAQIVVLDALPMTPSGKLDRRALPAPGARTTGGREPRDPVEDILCGLFAEVLGVDRVGVDDGFFTLGGHSLLATRLVSRVRAALGAELAVRDVFEAPTVAGLAARLGTPREDTRRRPPLRPAGERPAELPLSFAQRRLWFLHQMEGPTPTYHMPFALRLTGGLDVAALRAALNDVVARHEVLRTVYPDTGGTPRQHVLGPAEAPAPLPVRTVAEDALAAALADAYARPFDLTREPPLRAELFALGPDDHVLLLVLHHIAGDAWSNLPLARDLSAAYAARRTGEAPAWAPLPVQYADYTLWQRGFLGEESDPDSALSRQVAYWRTALDGLPAELELRHDRPRPEVSDHRGGTVPLHVGPATHARLAGLARAHGASLFMVVQAALAALLTREGAGTDIPIGTPVAGRTDEGLDDLVGCFVNTLVLRTDTGGDPVFTDLLDRVRRTGLAAYSHQDVPFEHLVEVLNPARSAARHPLFQVLLSLHHAADLPVELPGLRVRAEETRSRSAKFDLTFRLAERKGPDGGPGGLEGGVEYSTDLFDRSTVEGLAARFARLLDAVAADPGRRIRRLDVLSPAERERVLTGWNATERPLPPVTLPELFAAQVARTPDAVAVVGERDTLSYAELDAWAQRLARALTGRGAGPERVVAVVLPRSVELVVALLAVLKSGAAYLPIDPALPAERIRLLLDEARPVYVLDDADGVRNPRPETAGTARPVPLSPSHPAYVLYTSGSTGAPKGVVVPHAAVVNRLLGIQEEFGLEPSDICLYKAPAAFDASVWELFWPLTTGARIVVAAPDGHRDPAYLAALIREHAVTTVHFVPSMLRAFLDEPGAARCTSLRRVQTGGEPLPAELRDRATHLLGAPLNHLYGPTETTIHATTWSCGRPSAAAPDTTAARPVPIGRPVRNTRAYVLDAALGPVPPGATGELYLAGVQLARGYLARPGLTAERFIACPYGAPGEQMYRTGDLARWTANGVLEFAGRADDQVKLRGFRIELGEIEAALSRHEDVAAAAVVLREDRPGEQRLVAYVVPAPETGPHPADLRRHAALHLPEFMVPAAVVVLAALPLTSSGKLDRRALPEPGGPLTVTAKRPARTPEEEVLCGLFAEVLGLTGTGIDDDFFDLGGDSLAAIRLVRRIREAAGVELSVSDLFAAPTVSRLMDRLRLGVRGDAFDVLLPLRESGGLPPLFCVHPAGGLSWAYTGLLRHLGPDRPVYGLQARGLARPETYPTSMDELVTDYVRQLRSVRPTGPYHLLGWSFGGLAAHAIAVRLQAEGEKVAFLALLDSYPRDPEAAVPPVEERDVLATLLDFAGHGEDVTDAEQLRVEDVMEILRRGGGPLADIEERRVGALADVFVNSAELASAHAPGRFDGDLLHFTATAGRPDGAPTAAVWEPFVTGRVRRYTVDCTHHAMCRPEPLAEIGRVVAERLDGADRPRRQDGPNR

SEQ ID NO: 93 - protein, 76 aa, Streptomyces sp.
MTNPFENADSTYLVLVNDEGQHSLWPSFAEVPAGWTVAHGEDTRDACLAHVEEHWTDMRPKSLVRVMDEAGAPSAR

SEQ ID NO: 94 - protein, 371 aa, Streptomyces sp.
LTAADREERDTAVLTYQEPPAGGGAWAETVELTPQEAEQFRKVADEITGMLPHGKPAGPGQLDDPGLLAEIEVAARWLPPRLARALSRFRRQGNPHGVLLVRNVPLDADLPPTPADGAAPVWSRLGTATVGQLAVVSWLGDVIAYADEKAGRLVQDIVPVSGAEECQENSGSVLLELHTEDGFHPYKPDFVTLLCLRADHERRARTTYGAITRALPRLSAECVRVLRRPLFRIHFSSSFGGPGAGAKGSGTGTGTGVVSPPLPVLSGPDGDPEIVADFHAMEPLDETAARAFGELRDAMLAVLDGAVLEAGDLIVVDNRTAVHGRTAFRPRYDGQDRWLRRCFAVADLRPSRAARTPGSQVCAPLKPEGGP

SEQ ID NO: 95 - protein, 56 aa, Streptomyces sp.
VTADRRAVAPAAAGRAPGRQASGCPTPGRPTSTPEAAVIGTDMSAQVRALAAKGGR

SEQ ID NO: 96 - protein, 120 aa, Streptomyces sp.
MEPMEPMAGNDPHIHVQAQAGRDDAAARNLIAATFGLVSNLPSVVATGCGLRVPYAMTSSRPDRVTCLACREHARQAHLRFAVEVERLSRMLGSVITPAQGKLAADRHRDLARRFSDDEG

SEQ ID NO: 97 - protein, 129 aa, Streptomyces sp.
MRIYVTSVFVDDQAKALSFYTDKLGFVKKHDVPLGGEARWLTVVSPEDPDGTELLLEPSGHPAVQPYKTALVQDGIPATSFAVDDVRAEFERLRGLGVTFTQEPLEMGTVTTAVLDDTCGNLIQIVHTA

SEQ ID NO: 98 - protein, 97 aa, Streptomyces sp.
MAEDVFKALADPTRRRILDELAERNGQTLFEICARLVTKHGLGLSRQAISQHLAVLESAGLVRTRRQGRYKFHDLDTAPLEHIMTRWLSTDTPESTP

SEQ ID NO: 99 - protein, 230 aa, Streptomyces sp.
MHPSVRNNARMEDRIAPTPLLPVRGAARCAVAALVLAGAAWAARALWQIRLATAGMPASGPPDQGEGKHRPLTALEDGYHIVTTVGSAATVLCAITFLIWLSRVRDNARALSGQSPRYARPWLYAGWIVPVANLWVPRGVVADIHRASAPGERLPHAVNWWWGLWLVGMLSGTGLMYTVPTDDVIERAYQAVYPLLAADAAVVAAAVAGVFVVRALTAAQRRRGVDSVRA

SEQ ID NO: 100 - protein, 337 aa, Streptomyces sp.
VHQLTGAAGSAVSEATSAAYYASIAQAEESGDGTVRHRLRPEVGDGTIEVTSLRSGFDIVRYDVAFAGGHQVGFRFPGDHFELEHCVDGRMHISEAGAGAGDLRARSVALSPRGAIDGVVAHGDGERYRAVSVTGTWSGLESYLGGLGAGALPPTGPESVARERYLGRSAALRPAAGPLEEIFLHRRSSPGRLLFMESRLVAALAALIDGLASDASPADARDRAAPALADHETAALRRVPELLWRERHAPPTLAELARASSMSVRRLTTGFRALYGTSVMEYHRRHCLDRAAALLVETDWPVSRIAHEIGYASAGNFGYAFRRDRGTSPARFRRAHS

SEQ ID NO: 101 - protein, 394 aa, Streptomyces sp.
MVLGGLSAVSPFAIDMYAPGFPEIVTSFGTSKTAVQLSLTACLVGLAVGQVVLGPVSDATGRRRLLLAGSGLFSVLSLVCAAAPSIAVFDAARLLQGVAGGAGIVIGRAVVSDLFTGAPAARRLTTLSVIGSTAPVLAPVAGGLVLGVGSWRLVFVALAVTGLLLLTAIWAWVPESLPAERRRTGGLPAVLRAMGGLLRRREMAGHLLVMGCGLAALFAYITGSPFVFENGYGLSPTQYSLVFATNAVGTVLAGTVFGRLAGRVRLNLLLTVGVAVTVVALVTLVTLLAVGISTFLTTWACLFGLTSGFGLLLPASTTIVLAVGGDAPGAASGMLGGAQFVLGAAAAPLPGVLGDTTAMSTAVVVLGCVLLSATALLTLARPWEGHGEPAGSPE

SEQ ID NO: 102 - protein, 186 aa, Streptomyces sp.
MHRSIDLHHTADTFEKCAQLCRSAREATDRAVRMRAEAAEARLRAVRMRRTMERMKAGHRSDDAPGCRRLPATETRRGATARAAQLALLTAFVHARLDEEGGTADLFHETGCPGAGASECHCPVPERVRHDVAVRHGLARDCEEALHDPDHDACDWPHSELAALLRLKVLALPYELHRQWREEWRP

SEQ ID NO: 103 - protein, 252 aa, Streptomyces sp.
MNQKREIAALIGDVFGPAAVGSYLYGSAVLGGLRPASDLDVLVVSRRRATVPQRRALLDGLLGISGFTGGARPVELTVVVQDQVHPWRYPPICDFLYGEWLRDTYAAGVVPEPEPLPDLALLVTMALAGDHPLTGPPPAQVLDPVPWADVARASVHGIPGLLDEVDSDTRNVVLTLARVWTTLATGEIRTKDAAADWALGHLPPRHRPVLRHARELYLTRRYSEESWSDDLRSRVRPHVDHVLAEIRELPYA

SEQ ID NO: 104 - protein, 335 aa, Streptomyces sp.
MTEAGRSVPPRRRLPFRQFVLKVHSRCNLACTYCYVYEGPDQSWRERPARVAEETVRRTAERIARHVTTHGLRSIRVDLHGGEPLLLGPAALIDHARAIRNAVPGDCAVAVSVQTNGTLLTEDAVRRCSAAGISVGLSLDGGSARLNHRRVDRAGRSSWPAVRRAAGLLAGVPDAYGGILCTVDVEADPAEVYRSLIALAPTGLDFLLPYGNWSAPPPGTGPFAGPVGRAARPVPYGAWLARVFDLWWDTPPGRRAVPVRLFGEIVALLLGAASRSEAVGLSPLAVVVIDTDRYLHGSGFGHPSVYCADLEHLIRHIGERLQEEVRGVPGDPVRP

SEQ ID NO: 105 - protein, 220 aa, Streptomyces sp.
MNAEAEAQAQAAAAARTESPVQAQTAADLIGSEPAATGWPDVPGDTALTIKVPEADPLVRAGFPAHVTVLYPFLHESRIDATAQHDLTRLFAGHDAFTLTLDSFGRYPGVLYLDPWPHAPVTALTKDLTRRWPEAVPYRGIFGAGLAPHLTVANHEGPATQEAAYDALQAELAPSLPLTCSVREVHLIVWDGRRWRDRTAYRLGPPAPCRLTLSQRQRFY

SEQ ID NO: 106 - protein, 265 aa, Streptomyces sp.
MRIGELAGIVGVTTRAVRHYHRTGLLPEPPRQPNGYREYALRDAIELARIRRLVELGLSLDEVKDVLADDAGKDLVEILTELDADLARQEEAIRQRRARLARLFEQAERTGRLPEEAPVSPELAAVFDDMARASAQVPGPEPAMAAKERQLLALLETGSPGGDRGWFDGLVHAFRADPAAMRRAYDVYARMDELAEAAEDDPRVELTARAIVDSIPDEALRAMRLPADGAEEAGDGFSDAFFSDFAPAQAAAVYRAIELLTERAG

SEQ ID NO: 107 - protein, 307 aa, Streptomyces sp.
VNTLRKAVRWSFAALVPGELILVLCVAGGVRIAPAVRLATEPAMLTLAAAAAVLIALDHRRHRRAGLGHRAAFAAAIADTVPAPVRKLTAHEVFLSTSFLRWVTRRGPHGVRDGDLSVPYAPGQTATMYGFLFVCVVETVGFAFLIPWPVVHAVTLVLDIWGCYFVIALHASCVVRPHVVGADGSLRLRHGALLDIRIPAGRVAAVRREQKFPDGKLAVVDKNGVADMAVAGRTTVLVELTEPVRFVRALGKTAEARAFRFYAEDPAAVVTALKAGAASPCLASTRPSASGPDASASHAASQAGSPG

SEQ ID NO: 108 - protein, 338 aa, Streptomyces sp.
MHASNSPAGTLGSPTPVLSVGPVVLPVPGRAVDLQLRISAPVTGSDLPVVLLSHGQGYSHHLSSLNGYAPLANYWAAHGFAVIQPTHLSSRTLDLDPATPGAPLHWRSRAEDMRHILDRLDLIEAAVPQLPGRLDRERIAVAGHSMGGHTASLLLGARLTDPDDGTVVDLAEPRIKAGVLLAAPGRGGDALTASVAEAFPFFATTDFSRMTTPTLVVAGDQDDTAHLTVRGPAWHADPYVLSPGPKSLLTLFGAEHGLGGVSGYDVAETTDESPERVAAVQRLTWAYLRSRLYPGDPAWEAACEALASGPDALGRVEAKQGDAAPAFSAVTTAAGSSA

SEQ ID NO: 109 - protein, 63 aa, Streptomyces sp.
MNTGNTTDTPAAARQAKFGKLPERIRFEDMVTERPSPESRTPNAYTPEGTWNHFSCLALDLGL

SEQ ID NO: 110 - protein, 187 aa, Streptomyces sp.
VNTDHVFVCGHPALDFAATLRARRTVRFEMFATPERLEAWYVESGLVDSVTPARPSDVERAKSVREAAYELITARRLGEPFGARALDVLNKAARKPSAAPQLTSAGRRTDATPEEALSAVARQAVELLSGPDVPLLKECGNPECTRVYIDRSRGARRQWCGMESCGNKIKAAAYRARKRGAPATTAR

SEQ ID NO: 111 - protein, 585 aa, Streptomyces sp.
MPRPALLAHDLVRTLGTRRVLDGVSLTVSPGRRVGIVGENGVGKSTLLRLLAGVDEPDSGQVTRPDDLGFLHQEMPFDGATTIARVLADALREARAALAELDRLTRLLAGTPDDHPGHAELLDAYGKQLEHAQESEAWDADRRAAIVLDGLGLGGFGHGRTLGSLSGGQRGRLALAALLVRRPAALLLDEPTNHLDDEAAAFVEEQVRVQPGVVVAASHDRAFLDAVCTDLIDLDPAVHGPVRFGGNYSAYRAEKQAERERWEQRYAAEQEELEALRRSAGVTARRVAPDRGPRDNEKMGYGHRAGRVQQQVSRRVRNAARRLDELERTQVGRPPKPLRLHAGALTGALVDGAPADSGQVHGATADSAPVDSPLVALCDARVPGRLDVARLDIPAGDRLLVTGDNGAGKSTLLSLLAGRLDVEREGAVRRRPGLTVGLLAQDTVFEHPDRSARDTYTHVLGAERAEAVPLGSLGLLGVTDLDQPVGRLSAGQRRRLALALLVAHPPHLLLLDEPTNHLSPRLCDELEDALGAGPGAIVVASHDRWLRGRWTGRRLRLRAGRLAPPDDVYGIRSTGGRLRPGTSDR

SEQ ID NO: 112 - protein, 544 aa, Streptomyces sp.
MSDAFVVCSSLSFSWPDDTPVFQDLSFTVGTGRTGLVAPNGSGKSTLLKLIAGELRPAAGTVTVSGTLGHLPQSLPLAAGLTVAEVLGVAEVIRALDAVESGDVDEKHFATIGDDWDIEERTRAQLDRLGLTELTLDRGLSTLSGGQIVSLGLAAQLLKRPDVLLLDEPTNNLDLDARHKLYDVLEDWNGCLLLVSHDRALLDRMDRIAELEPGALRLYGGNFTAYEEAVQAEQEVAEKNVRNAEQELKREKRELQQARERADRRASNASRNLKNAGLPRIFAGNMKRGAQESAGRAGQMHASRVSEAKARLDEAGRAVRDEQRITLDLPDTDVPAGRNLFLGEGLRVSHGERPLFADGGVDLTIRGPERIALTGPNGAGKTTLLRLLQGGLEPDAGQIKRADSRIAYLSQRLDVLDLSRTVAENFAAFAPQRTAAERMNLLARFLFRGSRAHLPVQVLSGGERLRATLACVLCAEPAPQLLLLDEPTNNLDLVSAGQLESALASYQGAFVVVSHDERFLARIGIDRWLRLADGELRETAVPEV

SEQ ID NO: 113 - protein, 320 aa, Streptomyces sp.
MHAIRMDRHGGPEAMNWTALPDPAPGKGEVLVRLAVAGVNFMDVGARTSGGPGWAAPAILGDEGAGRVTALGEGVEGFAPGDRVAWFYHPGSYAELLAVPPESLVKLPDEVSDETAAAVMMQGLTASHLTTETYPVGPGDTAVVHAAAGGVGLLLTQMIKARGGRVIGLVSREEKVAAAREAGADHVLVSRGGGFEDRVRELTDGMGAQVVYDGGGSATFRSSQRALRPHGVHAYYGPFMGVPSVAPTDLPDSIFLTYPVVHHHVPTRAALVQRTGEVFDLIRDGRLTPRIGGRYALSDAARAHADLESRRTMGKLLLVP

SEQ ID NO: 114 - protein, 199 aa, Streptomyces sp.
MPLTRKGAATRQRIVEGAASEMRRLGIFALTLDDVMRRTATSKSQLFHYFPGGKDELLPAVARHEASGVISDQQPELASLNSWSAWRSWRDKIVERYREQGPNCPLNVVMGQLGPTTEGAQAVVRELYARWEGELAAGIRGMQSQGEIDAALDAGRGARALLAGIQGGVLIHLATGSLEYLKAALDQGIEFLRRPAARA

SEQ ID NO: 115 - protein, 122 aa, Streptomyces sp.
MDGLTESNNGVPEPHSGWTFVTNHARVLAAIADDRHARIRDIAAHCRLTERAVQKIISDLERDGYLSHTREGRSNTYRIDPGKVLRHPAEAGLTVASLLSLLVQDEQDRSTQPDGRRHPSTA

SEQ ID NO: 116 - protein, 245 aa, Streptomyces sp.
MRHSMRAAKGYVMSAVIEVQHLRKSYGDLVAVDDVSFSVEEGEVFGILGPSGSGKSTIVECVKGLRGRDGGDINVLGIDPHEDRAEVTRKLDAQLRAGCSPEQMRLAEALSLYSSFYRDPADWGRLLAALGLAPEDGPDEAAGGAAELSTALPLIGNPQVAVFDELTTELDPRSRQDTWELIESVRASGLTILMVTRFLEEAVRLCDRIAVIDQGRIKASGTPAGLADHLRAERSVGPDLRLSPR

SEQ ID NO: 117 - protein, 334 aa, Streptomyces sp.
VSLAQTVGTAAGVAGRGGGEPEIVSVPIIQVLPGDSPRLTGEDKGHVLRLAAIEGALPPILVDRRTMCVIDGLHRLMAASLKGQKDIDVRFFDGSREEAFLKSVEANVEHGLPLSQADRTAAALRILKSHPTMADRAIAKSTGLGARTVAQLRRSSDSVPQSDFRVGRDGKVRPLDGAQGRLRVAELMRDNPDASLRQVARSAGVSPATALDVRRRLERGDQPVPSQRSRSRGEAGPAKPEPVGPVPVPVAAPPTAATLDKLMRDPSLRQSELGRRLLRMFRENVLGASEMNDICASVPSHCIELIVELSRRNSQIWDGFAQELNRRARIVNPD

SEQ ID NO: 118 - protein, 107 aa, Streptomyces sp.
MGHKFTLVLSREITDEEGELLKEAGFSGATFATDTLPTDADVTVTKVDFDDTAAPSLAEAIETALETVKKVPDLTVPGLTVPAVPQPPAAEDGDVLVGEVVAETAGK

SEQ ID NO: 119 - protein, 248 aa, Streptomyces sp.
MTQPSPGDEVWCRRFHPADKPTARLVCLPHAGGSAPFFLPVAAELSPDVDVVAIQYPGRQDRRAEAPVDDLSVLVDRIHQVLRRQPDLPLTLFGHSMGAVIGFELARRLEADGRPPVRLFASGRRAPGTHRDESTFTSDAAIMAEVRRLSGTAAALLDDEEMMRAALPALRADYRAIEAYRCGPDAVVDTPITVLTGDNDPKTTLDEAKAWDRHTRGGCDLRVFAGGHFFISERGAEVMQVLKEHFGK

SEQ ID NO: 120 - protein, 151 aa, Streptomyces sp.
MADTTATTTAPETYEDPRVQDTFKTVHKFIGLYGVISVIVLGTVAAMAITGNEASPFMWIRGSILLLVWPLFHRMATRSAQGDARQYDRLATVTVILPIAIIVVDLIPGLCPPWYAAMQGVSALALVGAAVLTRKAEVRNVFPKLAKKKKA

SEQ ID NO: 121 - protein, 441 aa, Streptomyces sp.
MRIPPRGGPLRWCAEVGLGLYRSVVLVGLTCVVPLVAVPVGVGGARLAGSWTAGLEAPLAVRVLATLGQGLAVAFWIAAALFIVYPLTSRALARAARRCAEDWLEVRHEVQYRTVPPVTRMATGSWWDGEEYHKTEREARRRAAVHARFHDPQLHWDGLWAVVASVTVLPVSALPLLALGLGIYLAVTPALLGYGVALIVAGLAAAPFAWRILRPVATRLLGPVPRTRLGRRVEELEAIRADLTQAQAAELERIERGLHDGAQARMVAMGMSMGAAEQLVDSNPDAAKAVLAEARATSATALAELRTLVRGINPPVLAERGLVDAVQALALDAAVVVDVLTRVPSRPERPVESAVYFAVAELLANVAKHAEAERVTVELTYATRTLTATVQDDGVGGADPASGSGLSGIERRVHAFGGRLEIDSPAGGPTRITVAVPCELS

SEQ ID NO: 122 - protein, 205 aa, Streptomyces sp.
MVQLVEAFGHTVVAAVESGPATLEALLEHRPDVAIVDVRLPPTFSDEGLQAALAARRAVPGLPVLILSQHVEQLYARELLADGHGGIGYQLKDRVLDAEQFLDALTRVAGGGTALDPTVVSKLLARPTRADPLAALTERERSVLELMAEGLSNAAIAGRLFLSEGAISKYTTTIFAKLGVVTDDDTNRRVRAVLAYLEATGRSEQ

SEQ ID NO: 123 - protein, 1058 aa, Streptomyces sp.
VNDFAQDHTPGAADGGAPAPGTAETAVSPAEEALWRLEELAPGTPVNNVSVAFAVAGRVDAERLQAALAAVAERHQALRTGYRDAGSRLVKRVLAAAECPLVLEETELPGEPTPADLAPVTAAPFPLDGTPPVRAALFRSPDTDVVCVTAHRLVLDEISGPLLRDHLTAAYEENIGTVGTAETGPAAEAPSLAFWRTELAGANTDAQELSCTAPDPDRPTLAAAQLDRTLPASAEAGLRRLADDLGVPPSAILLAAYYVLLETHGAGPDLLVGLPVDLRPVPAARALGRYTGEVPLRLRTEHKESVRDLVLRSAERLAAATAHAAAAEAELAGLLPPAAPGRPPLPFRHTFEHRTVTCPAAFTFGGLDARPFPVTSGTARFDLHLTATAPQDPHGATALRLRYRTELFDPALAELLLARYETVLRAFTADPDRAVGDVSWWSGRDHEIIGAANDTAGPVEPATVLDAVRLQVRRAPDAVVVVDGERTVGYGQLWNAAHAMATRLKDIGVRPGDVVAIALPRGAELVAAVLGTWLAGAAYVPVDAAHPRERIRYQLSDSAATVLVADEHTAALAGPDGPAVLHAPPADHAPQVPATDAPVGDPAACAYLMYTSGSTGRPKGTLIGHAALANLAAHFAAQLAAAPGDTMLWTTTFAFDMSGVELFVPLVSGGRLVAAPDRARSDGRVLRELLERHDVRFVQATPTAWRLVADRAADRLRGRSVLIGGEPVPLPLARRLRAAGCELHHAYGPTETTIWSTSRVIDQDPGARLDVGRPIRNTRVHVSDAHGRDLPVGVRGELCISGTGVALGYHGRPELDAERFGVHPEYGRFYRTGDVACWRADGLIDLFGRIDRQVKLRGNRIELGEIEATLTAHPQVAAAAVIMVGDPSDDGVLIGCLEPSGDVLDTADVHEHARARLSRSMLPGDLVTVDPLPVNANGKVDLRELERIVTARRTAPGPTGTSPSDAAGRPGTAAGADALTDQLVQVWRTVLRRADADADTDFFAAGGHSMLAAVAMQEFQNLSGRSLGLSEIFEQSTPRQLAARIRATDTAAEGAPQS

SEQ ID NO: 124 - protein, 4038 aa, Streptomyces sp.
MANEEKLRDYLKRVTTDLAQTRERLREIEDRRPEPIAIVGMACRFPGGVSSAEGLWELVESRGDGIGPFPSDRGWPEDVVDPDPDAVGKSVSGEGGFLYDAAEFDAEFFGINPREALAMDPQQRLLLETSWEALEHAGIDATALRGSPTGVFTGLFYHDYLPTHPMPEEIEGYVGVGATASVASGRVAYVLGLEGPAVTVDTACSSSLVALHLAAQSLRSGESTLALAGGVKVMATPNTFVDFSRQRAVALDGRCKSYAEGADGTAWGEGVGVLVVERLSDARRNGHKVLAVVRGSAINQDGASNGLTAPSGPAQQRVIRAALADAGLEPVDVDVVEGHGTGTRLGDPIEAQALLATYGQDRPEGRPLWLGSVKSNIGHAQAAAGVASIVKMVAAMERGVVPATLYAERPSSQVDWDAGAVRLATEAVPWPETGERPRRAGVSSFGFSGTNAHVIIEQAPAADEEVPAPGAAPEAADGVDGTELPLLPWVVSGRSEAGLAGQAGRLAEVVEASGPGVRPVDVGWSLASSRAVLEHRAVVLGSSREELVTALGALAEGRDLPGVVSGAAGGAGKVGFVFTGQGAQRVGMGRQLYESFPVFAAAFDEVCAGLDAHLEGSLAAVVRGEGDPSWPGGGRIDETVWAQPALFAVEVALFRLLASWGVVPQVVAGHSIGELAAAHVAGVWSLEDACAVVAARGRLMQELPSGGAMVAVEAAEERVREVIAGRPGVGIAAVNGPRAVVVSGVEDDVLAVAEELAQAGSRTRRLQVSHAFHSPLMEPMLDRFAQAIAEVTYHTPTVTMVSALTGGLVSEEVTDPSYWVRHVREAVRFADAAESMRGLRVGTFIELGPDGVLSGMGPQTRSDSEGEGDSGSEAWLPVLRRGRDEPRSLLTALAKVFVRGVPVAWDALYTGTGAQRVDLPTYAFQRHRYWLKVAVDSRAEGLGLAPSGHPLLGAAVGLPASGGVVLTGRLSLSGQSWLADHVVAGQVVVPGAALVEMVVRAGDEVGCSRVEELLIEAPLVLPARGGVRVQVMVEEPDASGRRVVSVYAQDEDVPPEGEWTRHATGTLSPAEAVAEGSADGFRQWPPSGAQTLDPDLFYPELAARGLSYGPAFQGVRAAWRRGEELFAEIALPQDVTANGFGLHPALLDAALHVAAANTEGDGLALPFAWNNVMVHASDAVAARVRVTSAASGEGLTVMLADASGQLIASVDRLDLRTLSPQDLAAGGQAADDTLFAVEWMPASATSDAGPDSDPAALGRWVVLGGSEVVPDLPGAVVFPDVAALVAAVEAGAEVPQMVVAPVRPVAGPGGTAEAARRAAVDVLGLVQEWLAADVLSGARLLVLTERAVDAGAEVPVEVAAASVWGLVRVAQTENPGRLLLADVDEVSVPGAELAGWLRDGVELGEPQWAVRGGQTRVPRLTRATLADPSALNGDTAPEAQAVPSAGPEIETRPETETGPETNIDARPGTVLVTGASGVLGGLVARHLARSGRAEHLVLLSRRGGDAPGMPELLADLDASGVRVTVAACDAADRDQLSSVLEGVPLTGVVHAAGVLDDGVVGSLTAERLESVLRPKVDAAWNLHELTRDLGLELDTFVLFSSIAGVIGNAGQANYAAGNTFLDALAAHRRHHGLPAVSLAWGPWENGMAGELAEADLLRMARQGLRPLSDARGLAALDAAADRARPLLVAAELHLGALRRSAEVPPLLSALVPAARGNAQPARRTAGQPAAEDRNALAARLAGLSPAESREVLQDLVIGQAALVLGMAGPGALDGNRPFRDVGFDSLTAVELRNRLSTATGLRLPATAVFDYPTPVALAEFALDELIGSGAVPADAAPPAAADDGREDDDPVAIVGMACRFPGGATTPAEFWQMLASGGDGIGPFPDNRGWPEDVVDSDPDAMGKSLVGEGGFLYDAAEFDAEFFGTSPREALAMDPQQRLLLETSWEALENAGIDPTELRTSASGVFTGMFHHDYLSATALPEEAEGYVGLGATGSVASGRVAYALGLEGPAVTVDTACSSSLVALHLAAQSLRSGESTLALVSGVNVMATPVTFIDFSRQRGLAADGRCKAYAEDADGTAWSEGVGVLVVERLSDARRNGHKVLAVVRGSAINQDGASNGLTAPNGPSQQRVIRAALANAGIGTDGVDVVEGHGTGTRLGDPIEAQALLATYGRNRPADRPLWLGSVKSNIGHAQAAAGVAGIIKMVLALEHGQIPKTLHAERPSSQVDWGAGAVRLVTEAVPWPETGERPRRAGVSSFGFSGTNAHVIIEQAPSVEGVSLPGAAPEAVDGVSGTELPLLPWVVSGRSEAGLAGQAGRLAAFVGQDSGVRPVDVGWSLVSSRAVLEHRAVVLGSSREELVTALGALAEGRDLPGVVSGAAGGAGKVGFVFTGQGAQRVGMGRQLYEVFPVFAAAFDEVCAGLDAHLEGSLAAVVRGEGDPTWPGEGRIDATVWAQPALFAVEVALFRLLTSWGVTPQVVAGHSIGELAAAHVAGVWSLDDACAVVAARGRLMQELPSGGAMVAAEATEEQVRKALADRPGVGIAAVNGPRAIVVSGVEDDVLAVAEELAQAGSRTRRLQVSHAFHSPLMEPMLDRFAQAIAHVTYGKPQLTMVSALTGRPVSEEVTAPSYWVRHVREAVRFADATEAMRGLGVGTFIELGPDGVLSGMGPQTRTDSEGQDTTGSEAWLPLLRRGRDEPRALLTALAKAFVRGVPVAWDALYTGTGAQRVDLPGYAFQRQRYWLSAASASRAEDLGLERPGHPLLGATVELPASGEVVLTGQLTRSAQPWLGQLLVAGRPVVAPSALVDMAVRAADEAGGGRIEELLVEAPLVLPQRGGVRVQVTVGTADAEGRREVAVFARRPEGEWVRHASGTLAPAAADDEAAADAAWSVHWPPPGAVPADVPGLYEGLAATGLGHGPAFQGVRAAWRRGEELFAEIALPQDVAVDGFGVHPALLDAALHLTCLEQAEQQHRPDAEGPLLPFAWSGVRLYASGATAARVRVVPSPSGEGVAVTLADPVGRPIASADAVVPAPLAPGALEQADGAGRDGLFLVRWSPVDEVPEPGEPGRRWAVLGDDGGLEVPGATAYRDVTELLAAVRDGAPVPDTVALCCVSDSDPDPTTAQATDPDQATAQATAPDPAADLAPTPATLPAAARTATVHVLETLRALLAAGVLDGSQLVVVVERGLDVDAGTDLRLAGAGVAGLVRTAAAEHPERIVLADVARPAGSGPLVMAGAALGEPQFAVRDGRLLVPRLARAAGEELPVPDGRDWRLAACGPDAAGLVRPVDGEVGQRPLAAGEVRLAVHAAAVNRQDVRHVLEASPEPSRPLGREGAGVVLETGPGLAWPAVGERVTGLFPGAFAATAIADARLLVPVPDGWSAAQAAAAPVAFLTAYRLLAQAAPRPGERVLIHPAAGPEGLAAVQLARHLGAEVFATAPPSASPVLHRLGLDGQHLAPSATAHFEETFRTATDGHGMDVVLTFLPTSADPATATAAPDTATTTPDTATTDAALRLLHPGGRSLSAAAAETGHQHPEELEAHLAELAGLFEKGALHPLPVACRDVRQAAQVFRTPVREASTVAKTVLTLPPAPVGRPGTVLVTGASGALGGLTARHLARTGRAERLLLTSRRGAAAPGMPRLAAELAASGHSVQVLACDVADRRQLAAVLAGVPATAPLTGVVHTAGVLDDGVLASLTPERVEQVMRPKVDAAWHLHDLTRHLGLDTFVLFSSVAGVLGTPGQANYAAGNAFLDALAAHRRHHGLPAVSLAWGPWEDGMAGQLTDAGRQRMFRQGLRPLSAADGLALFDAALGRPDALLVTTRLDLPALRRTASAVPALVTGLVRPGRRTAGGPAPDAGRKLAAELAALPPEQFHHAVLNLVRTQAALVLGRPGPQSVEPARYFRQLGFDSLTAVEFRNRVSGALGLRLPAAVVFDYPTPRELAEYLQERIAEQEIDYGPVLGELDRLRSLLSGIVRRGGRKAEIMSRFETIMEEFRGAETDQGSSDTDITEATDDEMFDLIDKELGIVPGTDGGAA

SEQ ID NO: 125 - protein, 4674 aa, Streptomyces sp.
VNDTDGPALKDTAAAGAQPLAIVGMSCRYPGGVRSPEDLWKLLAEGGQGLSGFPRDRGWNLGSLHGQDPDGTGTTYLTRGGFLDDAAGFDAAFFGIPPREAQAMDPQQRLLLEASWEALENAALLPADLRGTRTGVFVGAEPREYGPRPHSAPGSAKGHLFTGTSPGVMSGRISYLLGLHGPSLTVDTSASSSLVAIHLAAQALRTGDCSLALAGGVSVMSTPGNFIAFSGLRALSADGVCRPFSAAADGTVWSEGVGLIVLERLSDAVRNGHPVLAVLRGSAINSDGTGEGLTAPNGSAQQAVIRRALADAHLTADQVDAVEAHGTGTRLGDPIEARALLATYGQERPEGRPLWLGSVKSNLGHAQAAAGAAGVIKMVLALRHGELPATLHAHEPTPEVDWSSGGVRLLTAPAPWPAGARTRRAGVSGFGLGGTNVHLLLEEGPTVSPAQDAEAVEESGGPAVPVVTGASAWVLSGRTAGALSAQAGRLREWMGTRPELEPADVAWSLAAGRSVFEHRGVVVGTGRAELLGGVGSLASGVPGGSVVSGVARSGVRPGVRPVFAFAGQGSQWVGMGRELAGVSPVFAGRLAECAAALAPHVDWSLSDVLAGAEGAPALEAADVVQPALWAVMVSLAAVWEAAGVAPDAVVGHSQGEIAAATVAGMLSLEDGARVVALRSQALKVLAGAGGMLSVSRPASEVEERLVRFGERVSLAAVNGPSATVVSGEPAALEELQSEFEAAGARARMVAVDYASHSAQVDRLEEEITSVLAGISPRRGRVPMVSAMTGETLTGEELDAGYWFRSLRATVDYDRAVRVLAGQGHQMFVEVTPHPVLVGAMHDTLQALAQEAGPGAIPGAVCGSLRRDDGGAERLLASLAEAFVNGASVDWQVVLPAGKRVELPTYAFQHERYWLDADAPTQPAEAPQPADLTAATDTTAGWGLRGQLAGRPEPEQHQLVLDLVITQAAAVLGHSAPAALDPARTFKELGFDSVTGVDLRNRLSTAAALQLPSTLIFDHPTPLALAGFLHGEAQGHAPSAGAVGSTRTPAPAGGDEPIAIVAMSCRLPGGLSTPERLWELLAEGGEAVAPLPADRGWDLSRQHGTRPDGDAASASHDASTTGLSTRGGGFLTGAGDFDAAFFGISPREALAMDPQQRLLLELSWEAFERAGIDPGRLRGSATGVFAGASPSGYGSHAPEELTGHLQPGITPSVISGRVAYTLGLEGPALTVDTACSSSLVALHLAVRSLRSGECELALAGGVTVHATTSWLNWFDRQRGLAADGRCKAYADQADGMGMGEGAGLLVLERLSDAQRNGHKVLAVVRGSAVNQDGASNGLTAPNGPSQQRVIRAALADAGLEPVDVDVVEGHGTGTRLGDPIEAQALLATYGQDRPEGRPLWLGSVKSNIGHAQSAAGVAGVLKLVLALQHGEVPRTLHADQPSSQVDWDAGAVRLVTEAVPWPATGERPRRAGVSSFGISGTNAHVIIEQAPNAEGALLSGAAPGIGADGDSGAELPLLPWVVSGRSEAGLAGQAGRLAEFVEASGSGVRPVNVGWSLVSSRAVLEHRAVVLGSGREELVAGLGALAEGRDLPGVVSGAAGGAGKVGFVFTGQGAQRVGMGRQLYESFPVFAAAFDEVCAGLDAHLEGSLAAVVRGEGDPSWPGGGRIDETVWAQPALFAVEVALFRLLASWGVVPQVVAGHSIGELAAAHVAGVWSLEDACAVVAARGRLMQELPSGGAMVAVEAAEEQVRRALADRPGVGIAAVNGPRAVVLSGTETDVLAVAEELAQAGSRTRRLQVSHAFHSPLMEPMLDRFAQAIAHVTYRKPQLTMVSALTGCPVSEEVTAPSYWVRHVREAVRFADATETMRGLGVGTFIELGPDGVLSGMGPQTRADSEGQDTTGSEAWLPVLRRGRDEPRAVLTALAKVFVRGVPVAWDALYTGTGAQRVDLPTYAFQRQRYWLPTASAPLDAAGLGQYTTRHPLLGAAVKSPASGGLVLTGRLSLSGQPWLADHVVAGQVVVPGAALVEMAVRAGDEVGCSRVEELLSEAPLVLPAQGGVRVQVMVEEPDASGRRVVSVYAQDEDVPPEGEWTRHATGTLSPVREAGDGRAHTRLRQWPSPGAEPVELTDFYSALSARGLSYGPVFQGVRAAWCLGEELFAEVALPPGVVVDGFGLHPALLDAALHVIVGVGERAGGLEVPFAWRDVELYATGAQVARVRVVPGADGEGVSLSLADADGALIASVGSLALRPFTAPASGAGDGLCGIDWVPATATTSDLGSAPEPLGRWAVLGAVSGVPDLPGAVVFPDVAALVAAVEAGAEVPQMVVAPVRPVAGPGGTAEAARRAAVDVLGLVQEWLAADALSGARLLVLTERAVDAGAEVPVEVAAASVWGLVRVAQKENPGRLLLADVDEVSVPGVDLADWLRAGVELGETQWAVRGGQIRVPRLVRLPSRLLTEEIRPGTVLVTGASGVLGGLVARHLARSGRAEHLVLLSRRGGEAPGMPGLVVDLQEMGVKVTVAACDAADRDQLAGVLEGVVLTGVVHAAGVLDDGVVGSLTAERLESVLRPKVDAAWNLHELTRDLGLELDTFVLFSSIAGVIGNAGQANYAAGNTFLDALAAYRRHQGLAAVSLAWGPWENGMAGALNEVDQRRMSRQGLRPLSAPDGLALLDTAVRTDRSLLVTTGLDLAALRRSGEVPPLLSGLLPDPRGGHSQPARRTVAESAADNRSPLAARLVGLAPGERTETALQLVATEAALVLGMTGPETVEPARSFRDTGFDSLTAVELRNRLNRATGLRLPATTVFDHPTPATLAAHVLAELLGEDEAGPMGDTVPAANVPRAGDDPVVIVGMACRFPGGVSSAEGLWELVESRGDGIGAFPSDRGWPEDVVDPDPDAVGKSVSGEGGFLYDAAEFDAEFFGISPREAVAMDPQQRLLLETSWEALEHAGIDATALRGSPTGVFAGLFYHDYLPWHAVPEEVEGYLGTGSTGSVASGRVAYALGLEGPAVTVDTACSSSLVALHLAAQSLRSGESTLALAGGVTVMGTPGTFIDFSRQRGLAADGRCKSYAEGADGTAWGEGVGVLVVERLSDARRNGHRVLAVVRGSAVNQDGASNGLTAPNGPSQRRVIRAALADAGLEPVDVDVVEGHGTGTRLGDPIEAQALLATYGQDRPEGRPLWLGSVKSNIGHAQAAAGVAGIVKMVAALERGVVPATLYAERPSSQVDWGAGAVRLVTEAVPWPETGERPRRAGVSSFGFSGTNAHVIIEQAPSVEGVSLPGAAPEAVDGVSGTELPLLPWVVSGRSEAGLAGQAGRLAAFVGQDSGVRPVDVGWSLVSSRAVLEHRAVVLGSSREELVTALGALAEGRDLPGVVSGAAGGAGKIGFVFTGQGAQRVGMGRQLYEAFPVFAAAFDEVCAGLDAHLEGSLAAVVRGEGDPTWPGEGRIDETVWAQPALFAVEVALFRLLASWGVAPQVVAGHSIGELAAAHVAGVWSLDDACAVVAARGRLMQELPSGGAMVAVEAAEEQVRKALADRPGVGIAAVNGPRAVVLSGTETDVLAVAEELARAGSRTRRLQVSHAFHSPLMEPMLDRFAQAIAHVTYGKPQLTMVSALTGRPVSEEVTAPSYWVRHVREAVRFADATETMRGLGVGTFIELGPDGVLSGMAAQSRTDSEGESGSEAWLPLLRRGRDEPRALLTALAKVFVRGVPVRWEEVYAGTGAQRVDLPTYAFQRQRYWLPTAPAHLDAAGLGQSATLHPLLGAAVGLPASGGVVLTGRLSLSGQSWLADHVVAGQAVVPGAVLVEMAVRAGDEVGCSRVEELLSEAPLVLPTQGGVRVQVTVEEPDASGRRAVSIHAQSEGNPPEAAWTRHATGVLAPAEAAAATADRMAQWPPTGAVSVALDDFYSGLAKHGLSYGPVFQGVRAAWRLGEELFAEVALPPGVVVDGFGLHPALLDAALHVIVGVGERAGGLEVPFAWRDVELYATGAQVARVRVVPGAGGEGVSLSLADADGALIASVGSLTLRPFDAPAVQSAEGLFTVEWVPAALTADGSGAAEGPWAVLGEDVLGAASYPDLPALVAAIEAGAEAPRTVVVRATPERPTPDVAAAARRITTDVLGLVHQWLAADVLSGARLLVLTERAVDAGPGVPVEVAAASVWGLVRVAQTENPGRLLLADVDEVSVPGAELAGWLRDGIELGEPQWAVRGGQIQVPRLTRATTETGVEARPGTVLVTGASGVLGGLVARHLARSGRAEHLVLLSRRGGEAPGMPGLVVDLQEMGVKVTVAACDAADRDQLAGVLEGVALTGVVHAAGVLDDGVVGSLTAERLESVLRPKVDAAWNLHELTLDLGLELDTFVLFSSIAGVIGNAGQANYAAGNTFLDALAAYRHRQGLPAVSIAWGPWENGMAGRLTGAERQRLTQQGLLPVTDARGLAVLDAAVGRSEPLLVAAELALAALHRTGEVPSLLSGLVPGPRAGKPARPTAGGRRSVLDRTEEQQTLAERLGRLGRGEALDTLRELVLAQSALVLGMSGPDRLDVDRSFRDVGFDSLTAVKLRNRLNTATGLRLPPTVVFDHPSPAALSGFLAEEMLGSAPEQPDVLAAHSGLDRVEASLAGLMADESVRMQVSERLKQMLGVLNATAGTDDGDDDGDSVADRIQSASDDDMFDFIDNQLGL

SEQ ID NO: 126 - protein, 380 aa, Streptomyces sp.
VADKTTLVKCLVWDLDNTIWDGTLLEDANVEPFPGILDTLVELDSRGILHSVSSKNDHDLAWKRLEELGLAEYFVHPRIGWGRKSDAVREIAERLNFAEKTIAFIDDQPAERAEVTYGAPDVRCYPAEQATSLTGLPEFSPATVTVDSRRRREMYQAGARRDAEKETFNGPDEDFLRSLDLVMTIKRADEEDLSRVEELTLRTSQMNATGVHYSDATLRALLADPDHEVLTVTLTDRFGPHGAVGVLLLERHPQLWHLKLLATSCRVVSFGAGSVILNWLIDQAARAGAHLAADFRRTERNRMMDIAYRFAGFADDACACLAALEASAGEGVQRLHLVADRREAPSTMRLLAPELASGSGEKTSEGGGNTSDTGEKAPVG

SEQ ID NO: 127 - protein, 373 aa, Streptomyces sp.
MTDHLADGRALISELVGDRAGDWDVAGELPVETLRTLGARGLLCAQVPADHGGLGLSSAANGELTAHAGSLCSSLRSVMTSQGIVAWTVQRFGDRTQRRELLAELTGGKLTAVGFSEPGAGSDLAAMTTRIKADGDGYVIDGEKKWVTGTRYADFVLIIGRYGDGDGAAAAIVPVDAPGVHIRAVPHPMGCRAAGHADIRLDQVRLPASHVLGGGGQPLSMLFTTALTYGRVSIGWGCVGILRACLDAAGRHAQQRHQGEVALAEHQLVRGHLADLMVAEQVALRACEHASRQWDAAAPDLGTMAVLAKHVSAQQAARSAATAVQLLGSTGSADGHVVARAFRDAKLMELIEGSNEICRLILADHVLSTTKTG

SEQ ID NO: 128 - protein, 93 aa, Streptomyces sp.
MTVEGENTTLTAETVHKELLDFLTERIQTTVEPDQDLFASGMVSSMFAMQLVVHLEEAYDIAIVGGDLKLDNFRTVQTMTDLVIRLQGTAEAH

SEQ ID NO: 129 - protein, 288 aa, Streptomyces sp.
MSTAFDEHRLAVVGAGVMGTNISALALGHGVDVVLVETDEAARDRADGTVRHKLRHAQLMNVLPADRPQGTLTITAKLDDIADADTVVEAVTEVAEVKKEVLGAASKLVAPGTTLISNTSCIPIEEMAAWTARPEELLGVHFMNPAYMIKMVEVIRGPQTSPATLERATDLLTVLGRESLVIEDSAGFVINRLLHPLINTAAMLVQEGVASVETVDNLLQGCLGHATGPLRTGDLIGLENLVDSLNVLYERRGDESCRPCDLLLAKVRDGHLGRKTGRGFYDYGKAAS

SEQ ID NO: 130 - protein, 943 aa, Streptomyces sp.
MNDQPALSARAPESAPRPGDAHTATTNKEKALWLLEQLVPDTGVNNLGLALQVSGRLRPDVLGAALAVVAGRFEVLRTVFRASGAELFKETVPAGEFAVPVEPLELSGGPLAEDLTAFVDRPFPLDGRPLIRVATATTADGDVLCVAVHHLVFDMVSVGVLLQALVPVYDAIAAGRPLPEQAVAPVPALAEPAPRPADLDHWRRTLDGFDPTGLDLWCGTPRGRQPVLAGESAVRTLGPRAQQAAVELQRAARAPMGAVMLAAYAALLAAHGAGPDLVIGSPVDVRGADAGAIGYHVNVVPIRIRVDLAEGFRPLARQARDAFLGAMAHAHTSVDELAGELPGIGTSWQTQLFRHLFNFLPDAPVGELRVDGMPARLLTVENPHSKFDLELVGSPARAELMLRHSRTLAPADAAALLARFEALLVTAAEDPDRPLGETAGWSEHDRETVDRANDTALDSQPDSFEAAFTSWARSRPADVAVLDGDRSLTYGELDAAADAVRELLADAGVRPAEVVAVAAAPGETAAALLGCWRAGAVCLPLDPGEESFLQARRLTQAAAKTVLTGAGVQLPAGVDLPAGADLPPVLPLPAFPGTGPERSGAPVDPYGDAPRRDAPAYRLPAGEENEDEAQSAVVLSHAGIADTAAHFARELGVGPGTGVLSLARPGTLAALFDAALALSVGGRLVPAPQETRTDAAALRAAAERHGADVLAVPPGTAPQLLEELGSQLSGLTVLVAGDELSASAAKALLAADCRLHCGYGAPQTTGWALAGRVTTPEDTRRGRPIGNTRAFVAAPDGRELPVGVRGELCLAGTGTALDTPDGPALPNHPGYGRHHRTGRLAEWQPDGTLVWHPREQPAADPAAGPTAPDAAADDPLVGQFVAIWQQLLSCEVTAQTHFFDAGGHSLLAAVLTQQVEDLTGIALQLRDVFEHPTPAALAARVRR

SEQ ID NO: 131 - protein, 279 aa, Streptomyces sp.
VPNENAWSVIAEPASNSLILGVDFPAAGRHEAGFDDLVKGMGTAWSGHGVLQTSPPPVRLADRPSGDFYTDHWMRSGDWEKYEVVAVLGYCVGAVYAGRVAERLAAVQGRMPQVVLFDAQLADLPLLATEMHKMIGQAAAVLSEQEAEEGRKRAVEIVATPSIGMAEAAGQMVELYRELATSGFRRIGLSESRCEEMVLLFESYMTWLSASSRIDPAGVWGGAIGLTSADYGELEKAGATTVVNAATAAIGRRIPVETRHIDLLRDASTVRALLANTEF

SEQ ID NO: 132 - protein, 5008 aa, Streptomyces sp.
MTGQADKLLEALRASVKETERLRQQNRQLVSAASEPLAIVGMSCRYPGGVTTPEQLWELVSTGGDAISGVPADRGWDTEGLHDPGRPDSSSGAEGGFLRDAAAFDADFFGISPREALAMDPQQRLLLETSWEALERAGVAPASLRGSQTGVFVGVGPSDYGANPALHTDDLQWHLITGGATSVMSGRISYLLGLQGPAVSVDTACSSSLVALHLAAQAVRAGECSLALAGGATVMASAIGIRGFSRQDGLAADGRCKAFSADADGMGMGEGAGMVVVERLSDARRNGHPVLAVLRGSAVNQDGASNGLTAPNGPSQQRVIRAALAGARLTASEVDVVEAHGTGTKLGDPIEAQALLATYGQERPEGRPLWLGSVKSNIGHAQQAAGVAGVIKMVLALQHGQLPRTLHADEPSPHVDWASGEVKLLTEPVPWPSGDRVRRAGVSSFGLSGTNAHVILEEAPAAQDAEAAEESGELVVPVVTGAGAWVLSGRTAGALSAQAGRLGEWVSARPALRPVDVAWSLAAGRSVFEHRAVVVGGDREQALAGLAGLASGAPSGSLVAGMARSDARPVFAFAGQGSQWVGMGRELAGVSPVFAGRLAECAAALAPYVDWSLTDVLAGAEGAPALEAADVVQPALWAVMVSLAAVWEAAGVAPDAVVGHSQGEIAAATVAGMLSLEDGARVVALRSRALKVLAGAGGMLSVSRPAAEVEERLVRFGERLALAAFNGPSATVVSGEPEALEELRAEFEAEGARARMVAVDYASHSAQVDRLEVEITSVLAGVSPRRGRVPMVSAMTGETLTGEELDAGYWFRSLRATVDYDRAVRVLAGQGHQVFVEVTPHPVLVGAMHDTLQALAQEAGPGVMPAAVCGTLRRDEDGADRLVASLAEAFVNGASVDWQAVLPAGKRVELPTYAFQHERFWPKAAAAVSGGDPVSLGLGAVGHPLLGAAVELAGGAGLVCTGRLSVRTHPWLADHAVSGVVLLPGTGFVELAVCAGDQAGCGVLEELTLQAPLVFPEDGSAVQVQVVLAGADAEGRRSVEVFSRSDAAGQSPGWTQHASGVLAPAGPAAAAEEDFAVWPPRNAATVDVSGLYQSAAEGPYGYGPAFQGLRAAWRRGEDVFAEVALPEEVAQDAAAFGLHPALLDAVLHASAVLEAEGEPGEIRLPFAWTGVELYASGASVLRARLRRDAQGTLTVSAADAAGQPVISVASLITRPVDTGQLGSAVSAVADALFTVAWMPISGIESIAPVAGEWALIGADRFAVAETLGRSGVPVQCFPGLEEAAAAVEAGQCTPAFVLVCAGGEGLGEEGPARAAARVTGETLVLVQDWLGEERLAESRLVVLTRGAVAVSAGEGVADLSAAALTGLVRSAQSENPDRLLLVDLPATGTAEQVTELPVAVATGEPELALRNGVVHARRLVRPSGELPVPEGRWRLVPDAGGSLEGLSLLPSPEVDGPLAAGQVRIAVGAAGLNFRDVLIALGMYPGGGVLGSEIAGTVLEAGPGVDHLAPGDRVTGIGEGGFGPVVVTDARQLVRIPDGWSFAEAASVPTAFMTAWYALVELAGAKPGQRVLVHAATGGVGMAAVQIARYLGLEVFATASTGKWPVLAAMGLDAEHVSSSRTAAFEAEFLAATDGAGVDIVVNSLAGELTDASLRLLPRGGAFVEMGRTDVRDAQAVAADRPGVTYRSFDLGEAGPEGLGTMLGRIVELLSAGELRRLPMRAWEVRRARDAFRFMQQARHTGKIVLTIPAHLSATQQPESARAGARSALITGGTGTLGGLVAGHLVVSGRAEHVLLTSRSGPGAAGAAALAARLAQAGASVRVVACDAADRQKLAAVMAAIPAGYPLRTVVHTAGVLDDGTVGSLTAQRVAGVLRPKADGAWNLHELTRDLVPELEDFVLFSSSAAAFGVPGQGNYVAANAFLDALAAERRASGLPGVSLGWGLWAEVSGLTGQLAESDRSRLSRGGALALSVDEGLALLDLCLARDEALLVPSRLDLAGLRAQAARSAEIPPLWRALVGTGTGRRSAAGAGDANADSLHQQLSGLSGQERTRLLTDLVRTHAAAVLGHASAEAVEPGRAFTDLGFDSLTAVELRNRLSTATGLRLPATLVFDYPNPAALAGHLKARIIGEGDGDGPAASPLPSGTTQQASGEPIAIVGLACRFPGGVNDPEGFWRLLADGTDAVSRFPDDRGWDVEALYDPDPDSVGTSYTREGGFIGDAGGFDADFFGISPREALAMDPQQRQVLETSWEALERAGIDPSALRGSQTGVFVGGFASAYGIGVATGDVGAEGNLTTGFATSVLSGRVSYALGLEGPAVTVDTACSSSLVALHLACQAIRAGECSMALAGGSTIISTPDGIVGFSRQRGLAADGRCKAFSADADGMGFAEGTGVLVVERLSDARRNGHPVLAVVRGSAVNQDGASNGLTAPNGPSQQRVIRAALAGARLTASEVDVVEAHGTGTKLGDPIEAQALLATYGQERPEGRPLWLGSVKSNIGHTQAAAGVAGVIKMVLALQHGQLPRTLHADEPSPHVDWAEGEVRLLTEPVPWQTNDRPRIAGISSFGISGTNVHVLLEEAPAEEPAHGNGDPADADTATSAPVLSMVGAGPAAETVSAWLVSGRSAAGLAAQADRLRDWLTTRPELESADVAWSLAATRSVFEHRAVVVGGDREQALAGLAGLASGAPSGSLVAGTARSDARPVFAFAGQGSQWVGMGRELAAVSPVFAGRLAECAAALAPYVDWSLTDVLAGAEGAPALEAADVVQPALWAVMVSLAAVWEAAGVAPDAVVGHSQGEIAAATVAGMLSLQDGARVVALRSQALKVLAGAGGMLSVSRPASEVEERLARFGDRLSLAAVNGPSATVVSGEPAALEELKSEFEAEGARARMVAVDYASHCAQVDRLEEEITTVLAGISPRRGRVPMVSAMTGETLTGEELDAGYWYRSLRATVHYDRAVRVLAGRGHQVFVEVTPHPVLMGAMNDTLEEVAQEAGPGIVPAAVCGTLRREEGGPAQLVTSLAEAFVNGAPVDWQAVLPAGKRVELPTYAFQHERFWPKAAAAVSGGDPVSLGLGAVGHPLLGAAVELAGGEGLVCTGRLSVRTHPWLADHAVGGVVLLPGTGFVELAVRAGDQAGCGLLEELTLHAPLVFPEDGAAVQIQVVLAGPDAEGRRGVEVFSRSDAAGQSQGWTQHASGVLAPAGPAAAAEEDFAVWPPRNAAAVDVSGLYQSAAEGPYGYGPAFQGLRAVWRRGEDVFAEVALPEEVAQDAAAFGLHPALLDSVLQAAFLMGGAEEGATEKVRLPFAWTGVELYAGGASVLRARLRRDAQGTLTVSAADAAGQPVISVTSLITRPVDTDQLGSAVSPVADALFTVAWMPISGTESIAPVAGEWALIGADRFAVAETLGRTGIPVQCFPGLKELAAAAADREIDPAFVLICTGDEGQILEDPARTAARVTGETLLLVQEWLTEDRLADSRLVLLTRGAVAVSANEGVADLPAAALAGLVRSAQSENPDRLLLVDLPATNTADQIAVLPNALPAALAAGEPEVAIRDTAVHGRRLTRPSGSTAAVERPANPSGARSALITGGTGTLGGLVAGHLVVSGRAEHVVLISRSGPGAADAAVLAARLAQAGASVRVVACDAADRQELTAVMATIPAQAPLRTVVHTAGVLDDGTVGSLTAQRVAEVLRPKADGAWNLHELTEGLDLDHFILFSSSAAAFGVPGQGNYVAANAFLDALAAERRASGLPGISLGWGLWAEVSGLTGQLAESDRSRMSRGGALALSADEGLALLDLCLARDEALLVPSRLDLAGLRAQAARSAQVPPLWRALVGAGTGRRSAAGTDDANADSLHRQLAALPAAERDEMLLNLVRAHVAAVLGHASAEAVEPGRAFTDLGFDSLTAVQLRNRLSTATGLRLPATLVFDYPTPAALAGHLRGRLGDGPQARTKSATAPAAAETSGAGAPDEPIAIVGMSCRFPGGTADPEDLWRMLRDGEDAISAFPTDRGWDLDGLHDPDAARSGTARTQAGGFVQDAASFDAGFFEISPREALAMDPQQRLLLELAWEAFERSGIDPATLRSSPTGTFVGGYTSGYETSVLAEGGTESQGHLMTGIATSVLSGRLAYTFGLEGPAVTVDTACSSALVALHLAAQSLRSGECSLALAGGVTVMATPGTFVEFSRQRGLAADGRCKAFAAAADGTGFAEGAGLLVIERLSDAQRNGHPVLAVLRGSATNQDGASNGLTAPNGPSQQRVIRAALANSRLTPGDVDVVEAHGTGTKLGDPIEAQALLATYGQDRGESDPLWLGSVKSNIGHTQAAAGVAGVIKMVLALQHGQLPRTLHVDEPSPHIDWTGGEVRLLTEPVPWQANGRPRRAGVSSFGISGTNAHVILEEAPTPPAGAESHVPLAGSGSHVLLPGAESHVPPADPETHAAPPPGAATAPTAGSVVPSAVPWPLSARTPEALRAQAGRLRQFLLERPELDLVDVGFSLATTRTAFDHRAVVQGTDRDGLLAGLAVAAAGVQTRDVVTGTVRAGRKTVFVFPGTAASPGGPGADVRLDAAAALLEESPVFADRIADCEQALAPFVDWKVTEVLRAGEAGPALTTPEIHRPVLWAVLVALAALWRSAGVHPAAVAGHGVGEIAAAQVAGVLTAADAARLAVLCGGASAGPGAVQPRPGTVPLYSTRSMDWVDGAELNAEHWLPYPGQEPPAPVRELAEALAGAGLGAYVEVGPEPVLATALAQALQAEGNGADRDGAPVVTATLRRGELGLRAFTSALAALHVRGVTVDWAAVLAGGRRVDLPTYAFQRERYWPQPPARTNAEAPAAGSLSPDEQRLWSALAEQDMSALSEVLGTGTPLREDMPLGEVLAYVAAWRRERTAPDGAAETGAAGAAEGTAPEDADGAGAGWRQRLAEVGPAQQQELLSALVREEIAAMLGYGSVDSVPQYGDVFELGMTSMSAVQLRLRMNELTGLTLPEGFVYDLYMPEAIAEYLLGELTVALREGTPEDR

SEQ ID NO: 133 - protein, 5602 aa, Streptomyces sp.
VDNQEKLFDYLKKTAAQLQETRKRLHRLEAAEQEPLAIVGMACRFPGGVGSPEEFWEFLASGGDGVDGFPTDRGWDLESVYDPDGEREGTSYVAQGAFLREAAEFDAEFFGISPREALAMDPQQRLLLETSWEALERAGIDPTSLKESATGVFAGGFTSGYGLGISMSQDDSGAEGHVLTGNATSVLSGRVSYVLGLEGPAVTVDTACSSSLVALHLAAQAVRSGECSLALAGGATVMATPGTFIDFSRQQGLSVDGRCRAFSEDADGTGWAEGAGMVVVERLSDARRNGHKVLALLRGSAVNQDGASNGLTAPNGPSQQRVIRAALANARLAPADVDVVEAHGTGTTLGDPIEAQALLATYGRDRGEGDALWLGSVKSNIGHTQAAAGVAGVIKMVLALQHERLPQTLYAEVPSTHVDWSAGAVRLLTEPAPWPADGDRVRRAAVSAFGMSGTNAHLILEEAPTAQEAEAAEEPKEPETEPDGPAARVLSGEGAGAVRAWQVSGRSAAAPAAQADRLHGWLTARPELRPADVAWSLATVRTAFEHRAVIVGSDDRAALLTSLESLAAGTPNGSVVSGTARPGVRPVFVFPGQGSQWLGMGQELARTSPVFAARLAECAAALAPYVDWSLTDVLAGTGYAPALEAADVVQPALWAVMVSLAAVWEAAGVAPDAVVGHSQGEIAAATVAGMLSLEDGARVVALRSRALKVLAGAGGMLSVSRPASEVEERLARFGDRLSLAAVNGPSATVVSGEPGALEELKAEYEAEGARARMVAVDYASHSAQVDRLEEEITTVLAGVSPRRGRFPMVSAMTGETLTGEELDAGYWYRSLRTTVHFERAVRSLAGQGQRVFIEVSPHPVLLSAMTDSLEEAAQDAGPGAVPAALCGTLRRDDGGAERLVTSLAEAFVNGAPVDWASVLPAGERVELPTYVFQHRRYWPKGPMAGLRQDGGDALALGLGAVGHPLFGAAVELAGGDGVVCLGRLSLRTHAWLADHAVGGVVLLPGTGFVELAVRAGDQVGCGLLEELTLHAPLVLPADGSGVQLQVAVAAPDTEGRRGVRVFSRPEDAADRQPWTLHAGGTLGPTAAPAPADPDFALWPPQDAVPADVSDVYPVRLAEVYGPAFHGLRAAWRRGADVFAEVALPEEAAQQAGAFGLHPALLDAALHASFLAQPAAAQETGQVTMPFAWTDVALHAGGASVLRVRLRQDAQGGLSLAAADTTGAPVVTVGSLVRRPVSAEQLRATRDELSDSLFVQTWAELPDGDAPAGEWALLGADRYGLADGLEAALEAAGVQLRPFPGLPELVAAAQTGTGAPRTVVVCADALSDEDVFGGSAETSAGKEAPNNSPQPADPARHLPGAAHRAGAAALRFVQQWLAEPALEDIKLVVVTRGADAITDEDVVDPAAAVVRSLLRSAQAENPDRFLLADLPACDGAPQEAALLPAALGSEEPEVAVRGRIRYGRRLTRPTGEVTRIQWPSDTRADPAARSLLVTGGTGTLGGLVARHLAATGRAGCVVLTSRSGPAATGAAAQAAELAQLGVPVRIVACDAADRDALAAVLEQIPAANPLGSVVHAAGVVDDGVIMSLTPERLSAVLRPKADAAWHLHELTAGLQLDRFVLFSSAAAVFGSAGQASYVAANAFLDAVAAHRRSTGLTGTSLQLGPWAHEAGIGRNLDATSLARIDRGFVPLTADEGLVVLDTALTRDEAVLMPARPDTAALRAQTAAGGEVPPLWRALAGTSARRTAASGTAAGSGTEAGEALRHRLASVGGTERDRLLLELVLAHAAAVLGHASPDAIDAGRAFTDVGFDSLTAVELRNRLKAQTGLRLPATLVFDHPTPAALATFLRGELAGDLPATAVPTAPVAAGADEPIAIVGMACRFPGGAAGPEQLWHLLDSGTDAIGPFPRDRGWDLDRLQDPDAERTGVSYQQVGGFVPDAASFDAGFFGISPREALAMDPQQRLLLETSWEAFERAGIDPASVRGSQTGAFIGGYSSRYAHDASLEGTGVAEAHLATGNATSVLSGRLSYVFGLEGPAVTVDTACSSSLVALHMAAQALRAGECSLALAGGVTVTATPDVFVTFATAGGVSLDGRCRSFAASADGSGFAEGVGLLVVERLSDARRHGHKVLAVVRGSAVNQDGASNGLTAPNGPSQQRVIRAALANARLTPGDVDVVEAHGSATKLGDPIEAQALLATYGRERPEGRPLWLGSVKSNIGHTQAAAGVAGVIKTVLALQHERLPRTLHVDEPTPHVDWSAGEVRLLAESVPWPGGERVRRAGVSGFGMSGTNVHVILEEAPAEEAEETDTDGISAPVTTAPAPALLGDGGPTAWLVSGRSAEGLAAQAGRLGEWAAGRAALRPVDVAWSLAATRAVLEYRGVVVAGDRAGLLDGVRGLAGGELAAPGVVSGVARATGRTVFVFAGQGAQWAGMGRDLLASSPLFAARLDECAAALAPYVDWSLTEVLAGAEGAPALEAAEVVQPALWAVMVSLAAVWEAAGVTPDAVVGHSQGEIAAATVAGMLSLDDAARVVAVRSRALSGLDVAGAMVSVVMPADAVRELVAGFDGRLSVAAVNGPASVVVSGQAEALASFERELAARKVLRWRIPATDFVAHSAAVEPLAAQLREELSEIAPQAGRIPMVSTVTGEWLAGEEADAAYWFANLRQMVRFEDAVRSLLESGYGAFIEISPHPVLTAAVTETAQDTDANATPVLSIGTLSQTDAGAAGLLRGFAQAHVAGLPVDWRKVLPAGEVVELPTYAFQHRRYWLETSTAAAATAATTVSSAAEAQFWAAVESGDRTGLAQALAIENEHQLDGLLPALASWRRREQERSATESWRYRITWAPVADPAPARLAGRWLLLVPAQEADGEAARQCCAALTARGATVVPVQVPTGTVQRDWIAALLDRTQQDAEPASGVLSLLALDETPLPDHPVVTAGLAATLALVQALGDTGIAAPLWVATRGAVATGPDEAPTSPLQAQIWGFGRIAGLEHPDRWGGLVDLPAVLDERTGGQLATALAGSTENQVALRPSGALGRRLAHAPQSPAAATSQSATGTALITGGTGALGGHVAAWLAARGAERLVLTSRSGPAAAGAAAQAAALAARGTRVDVLACDTADRSQLAAAIGRIGTDGPRFTTVVHTAAVLDDGVVDALSPARLETALAAKAAGAAHLDELTADLDLDAFVLFSSVVATTGAPGQANYGAANAYLDALAQHRRSRGLPALAVAWGPWAVGVAQGSAAARQRLARNQWEEVMDPDLAVRALGEALDGGDTVLTVMNIDWSAILAQPQSSAGLLQAAVMRDLPEVHRLAALLAETPQAPAEPALAERLAGLGRPEQRRVLSDLVRAGAARVLGHTSPDAVEDRSAFSDLGFDSLTSVELRNDLSAATGLRLPATLLFDHPTPAALTGYLETELLGTGPQTPEAPAALPATIAADEPIAIVGMACRYAGGVAGPDDLWQLLTSAGDGISGFPDDRGWDLEALDGPSYVRHGGFIKDIGGFDAGFFGISPREALAMDPQQRLLLETSWEALERSGIAPTSLRSTATGLFIGGYASGYGYGADFEGAAHLITGTATSVLSGRVSYTLGLEGPAVTVDTACSSSLVALHMAAQALRSGECSMALAGGVTLMVTPEGFIGFSEQSGLAKDGRCKAFSDQADGMGMAEGAGVLVIERLSDAQRNGHPVLAVLRGSATNQDGASNGLTAPNGPSQQRVIRAALANSRLTPGDVDVVEAHGTGTKLGDPIEAQALLATYGQDRPEGRPLRLGSVKSNIGHTQAAAGVAGVIKMVLALQHEQLPRTLHVDELSSHVDWTAGEVEVLTEPVPWPSGGRVRRAGVSSFGLSGTNAHVILEEAPAPAVTQDGESPAETFEGTGAEGTVLVPGVAPAWLVSGRSAAGLAAQAERLRDWLAARPELESADVAWSLAAGRSVFEHRGVVVGTGRAELLGGVGSLASGVPGGSVVSGVARSNVRPVFAFAGQGSQWVGMGRELAGVSPVFAARLAECAAALAPYVDWSLSDVLAGAEGAPALEAADVVQPALWAVMVSLAAVWEAAGVAPEAVVGHSQGEIAAATVAGMLSLEDGARVVALRSRALKVLAGAGGMLSVSRPAAEVEERLVRFGDRLSLAAVNGPSATVVSGEPAALEELQSEFEAEGARARMVAVDYASHSAQVDRLEEEITTVLAGISPRRGRIPMVSAMTGETLTGEELDAGYWFRSLRATVHYDRAVRVLAGRGHQVFVEVTPHPVLMGAMNDTLEEVAQETGPGAEPAAVCGTLRRDDGGAERLLLSLAEAFVNGAELDWQAVLPAGRRVELPTYAFQHERFWPKVAAAFGGDPVSLGLGAVGHPLLGAAVELAGGAGLVCTGRLSVRTHPWLADHAVGGVVLLPGTGFVELAVCAGDQAGCGVLEELTLQAPLVFPEDGSAVQVQVVLAGPDAEGRRSVEVFSRVDVAGQSQGWTQHASGVLAPAGPVAAAEEDFAVWPPRDATTVDISGLYQAAGANSYGYGPAFQGLRAVWRRGQEIFAEVALPEQVAEDAAAFGVHPALLDAVLHASALLDAEGEPGEIRLPFAWTGVELYASGASVLRARLRRDAQGTLTVSAADAAGQPVISVASLITRPVDTGQLRTADTGMADALFGVEWAAVAAAEAAAGEWALIGADRFDVAGQLTAAGTQVRCFAGLEELAAAAADREIDPAFVLVCAGDEGRGEEDPARAAARVTGEMLGLVQEWLAEERLAESRLVVLTRGAVAVSASEGVADLPAAALAGLVRSAQSENPDRLLLVDLPATGTTDQAEVLPLAVATGEPELALRNGAVHARRLVRPSAELPVPEGRWRLEPDAGGSLEGLSLLAAPEADGALAAGQVRVAVRATGLNFRDVLIALGMYPGGGVLGSEIAGTVLEAGPGVDHLVPGDRVMGLGEGGFGPVVVTDARQLVRVPDGWSYAEAASVPSAFMTAWYALVELAGAKPGQRVLVHAATGGVGMAAVQIARYLGLEVFATASPGKWSVLASMGLDAEHVSSSRTAAFEADFLAATDGAGVDIVVNSLAGELTDASLRLLPRGGAFVEMGRTDVRDAQAVAAEQPGVTYRPFELSEAGPAALGTILERVVALLSTGELQQLPLRAWGVRQARDAFRFMQQARHTGKIVLTIPAHLSATQPESARSGARSALITGGTGTLGGLVAGHLVVSGRAEHVVLTSRSGPGAAGVAALAARLAQAGASVRVVACDAADRQELAAVVATIPAQASLRTVVHTAGVLDDGTVGSLTAQRVAGVLRPKADGAWNLHELTQGLDLDHFVLFSSAAATFSSPGQGNYVAANAFLDALAAKRRAAGLPATSLAWGMWADASALTGQLSESERARISRGGVMALSADEGLALLDLALARDEALLMPAKLDLAGMRAQSAGSTEVPPLFRALVSAGSGRRSAAGAGDAGADSLHQRLSGLSGQERTRLLIDLVRTQAAAVLGHASAEAVEPGRAFTDLGFDSLTAVELRNRLSTATGLRLPATLVFDYPNTLELAAFLKEKLLPQIVEHGEPDAAEEKLRTALATVPLSRFREAGLMEALLRLAGVQDDALLAEEGDQGEDIDELDADSLIRLALGGETE

SEQ ID NO: 134 - protein, 7930 aa, Streptomyces sp.
VSDENKLRSYLKRAIADAQDARARLREVQEQAAEPVAIVGMGCRFPGGAGSPEDFWKLLASGGDAVGGFPTDRGWDLEGIYDPDGERENTSYVAQGAFLQGATEFDAGFFGISPREALAMDPQQRLLLESAWEALERAGIDPASLKESATGVFAGGFASGYGIGLSWAAGEGGSGVEGHLMTGNATSVLSGRLSYALGLEGPAVTVDTACSSSLVALHLAVQALRLGECSLALAGGVTVLVSPGTFVEFSRQQGLSADGRCRAFSEDADGTGWAEGVGMLVVERLSDARRNGHKVLAVLRGSAVNQDGASNGLSAPNGPSQQRVIRAALASAGLSASEVDVVEAHGTGTTLGDPIEAQALLATYGQDRREGRPLWLGSVKSNIGHTQAAAGVAGVIKMVLALQHGQLPRTLHADVASSHVDWSAGEVRLLTESVPWPVGERPRRAGVSAFGISGTNAHVILEEAPPVETGEADGSAEAGSGTAGSGGPAGSVADAAPVLAGAGAWVVSGRTAEALSAQAGRLREWVAARPGLGAADVAWSLAATRSAFEHRAVVLGSERGALLDGVHSLAAGTSSPVVVSGVARPDVRVGLIFAGQGAQWVGMGRGLYAGSAVFAEVFDRVCGLLELELGAGVRLRDVVLGAEGVDAGLADRTLYAQAGLFAFEVALAAVLKAVGVVPDAVVGHSVGEVAAAHVAGVLSLPDACALVAARARLMQELPSGGAMAAVNAAEADVIASFDEVSGEVAVAAVNGPESVVVSGTADTVDAVVELWRGRGCRVRRLRVSHAFHSPAMDPVLDELDGVAQGLVYHRPEVMWAGALTGELVSDPQAGYWPAQTRQAVRFADAVATLAREGVSVFLEVGPDGSLSSLGPDAVAGVEGAEAAFVPLQRRTEDGATDLETGLARAFVNGAPVDWTRVLPAGEQVELPTYAFRHQRYWPEGIVSLMPPTAGGANRITAGTEAEAEFWAAVEGEDLTRLAETLDVDGDRPFSEVLPALASWRRRDLERSLTADWRYRTTWAGVAEPDARVLSGTWLVAVPAGAAAVGLAQGCTEALRARGAEAVVVEIPAGTVDRAGLAALLTGAADASAVSGVLSLLALDETPLPDHSAVPAGLAATLALVQALGDAGFEVPLWVVTSGAVAAGAEEVLARPVQAQVWGLGRVVALEHPERWGGLVDLPESLDERAGARLVAVLAGYGENEVALRPSGILGRRLAHVSGRGARERWAPRGTVLVTGGTGAIAGHVSRWLAGRGADRVVLAGRSGPAAADVAAQAAELAAAGVHVDVISCDVSERGPLTNLLSWIGSSGPELSSVMHTAGVLDDGVLDRLSVSRLQSVLAVKVHGAVLLDELTVDLDLDAFVLFSAAAATLGGPGQGNYAAANAFLDALAENRRARGLAGLAVAWGPWAGGGMADSNEAVRSRMRKMPMPPMDPQLAVRALAEALQGPDAAVAVMDVDWAQLASDAGATGMLKRPLVRDLPEIRQLLAAAGNTVTVAPAEGELARQLAGLDRAEQERMLADVVRGEAAVVLGYGSADAVEDRRAFKDLGFDSLTAVELRNRLNAATGLRLPSTLVFDYPTPLALAAFLRDELTGQQKGTAAPVITVVAAPADEPLAIVGMACRFPGGVGSPEEFWEFLASGGDGVGDFPADRGWDLESVYDPEGEREGTSYVAQGAFLREAAEFDAGFFGISPREALAMDPQQRLLLETSWEALERAGIDPASLKESATGVFAGGFTSGYGFGLSLANQGDSGAEGHSLTGNATSVLSGRVSYVLGLEGPAVTVDTACSSSLVALHLAAQAVRSGECSLALAGGATVMATPGTFIDFSRQQGLSADGRCRAFSEDADGTGWAEGAGMVVVERLSDARRNGHKVLAVLRGSAVNQDGASNGLTAPNGPSQQRVIRAALANARLAPGDVDVVEAHGTGTTLGDPIEAQALLATYGRDRGDRDALWLGSVKSNIGHTQAAAGVAGVIKMVLALQHERLPQTLYAEVPSSHVDWSAGAVRLLTESAPWPADGDRVRRAAVSAFGMSGTNAHLILEEAPTAQEAEAVEGPKEPVVPVVTGAGAWVLSGRTAEALSAQAGRLREWVSLRPALRPVDVAWSLAATRSVFEHRAVVVGGDREQVLAGLGSLASAASSGSLVSGVARSNARPVFAFAGQGSQWVGMGRELAGVSPVFAVRLAECAAALAPYVDWSLTDVLAGAEGAPALEAADVVQPALWAVMVSLAAVWEAAGVAPEAVVGHSQGEIAAATVAGMLSLEDGARVVALRSRALKVLAGAGGMLSVSRPAAEVEERLVRFGERVSLAAVNGPSATVVSGEPAALEELQSEFEAEGARARMVAVDYASHSAQVDRLEEEITTVLAGISPRRGRVPMVSAMTGETLTGEELDAGYWYRSLRATVHYDRAVRVLVGRGHQVFVEVTPHPVLMGAMNDTLEEVVQDAGAEPAAVCGTLRRDDGGAERLLTSLAEAFVNGAPVDWQAVLPAGEQVELPTYAFRRRRFWPEGMLVLPMPGSATAGAGASTDAEAAFWAAVEDGDLSGLGDALALEGGRPFNEVLPALASWRRRERDRSTTAGWRYRTGWSVIAEPDVRLLSGTWLIAVPAGTAAAELAQDCVGALSARGAEAVVVEVPVGSVNRGELAALLTVAADASAVSGVLSLLALDEAPIPGHPVVPSGLATTLGLVQALGDAGIEAPLWVATSGAVAAGRGEVLARPVQAQVWGLGRVVALEHPERWGGLVDLPETLDERAGARLVAVLAGCGENEVAIRAAGILGRRLAHVSGRGARESWVPRGTVLVTGGTGAIAGHVSRWMAGRGAERLVLTSRSGPAALGAAALAAELAAAGSAVEIVACDVSERAGLAGLLDWIDASGPRLSSVMHTAGVLDDGVVDRLDTARLETVLGVKAHGAVLLDELTAGLDLDAFVLFSSAASTLGGPGQGNYAAANAFLDALAENRRGRGLAGLAVAWGLWGGGGLGESSEVIRSRMRRMPMPPMDPQLAVRALGEALDGPDAVVTVMEVDWAQLASDAGVTGMLKRPLVRDLPEIRELSAASGRAPALAPVEGELAARVAGLSRSEQDRVLTGVVRAEAAVVLGHDSADGVQARQAFKDLGFDSLTSVELRNRLNAATGLRLPATLVFDHPTPVALAAFLRGEMIGGHPDAADSGTSAIQTAVDGTPPAADEPLAIVGMACRFPGGASSPEEFWELLASGGDAVGGFPTDRGWDLEGIYDPDGERENTSYVAQGAFLQRAAEFDAGFFGISPREALAMDPQQRLMLETSWEALERAGIDPWSLRGSSTGVFAGGFTTGYGLGVSAAAGEGGSGVEGHLMTGNATSVLSGRVSYVLGLEGPAVTVDTACSSSLVALNLAAQSVRSGECSMALAGGVTVMATPSSFVEFSRQQGLSADGRCRAFADDADGTGWAEGAGVLVVERLSDARRNGHRVLAVVRGTAVNQDGASNGLSAPNGPSQQRVIRAALANARLSAAEVDVVEAHGTGTTLGDPIEAQALLATYGKDRGESDPLWLGSVKSNIGHTQAAAGVAGVIKMVLALQHGQLPRTLHADVASSHVDWSAGEVRLLTESVPWPVGERPRRAGVSAFGISGTNAHVILEEAPPVEAGEPVEAGSGTAGAVAGAGAWVVSGRTAEALSAQAGRLREWVSARPELGAADVAWSLAATRSAFEHRAVVLGTERDALLSGLHKLAAGTSSSEVVSGMARPDARVGLIFAGQGAQWAGMGRGLYAGSAVFAEAFDRVCGLLELELGAEVRLRDVVLEGDGEAAGLADQTLYAQAGLFAFEVALAAVLKAAGVVPDAVVGHSVGEVAAAHVAGVLSLSDACALVAARARLMQELPSGGAMAAVNAAEADVIASFDGVSGEVAVAAVNGPESVVISGATEAVDAVVELWRDRGCRVRRLRVSHAFHSPAMDPVLDELRTVAEGMEFRRPEVMWAGALTGDLVSDPQAGYWPAQTRQTVRFADAIATLATDGISVFLEVGPDGSLSSLGPDAVVEVDGAEPAFLPLQRRDDEGATGLLTGLARAFVNGAPVDWTRVLPTGEHVELPTYAFRHQRFWPEAPAALSANTSATARDGAGSAAESAFWSAVESGDLEQLADTLAVDGDRPFHQVLPALASWHRRELDRSATADWRYRTVWSPVAELDARVLSGTWLVAVPTESAAADLAQDCIEALSARGAEAVVVEVPAGTVDRVELAALLTDTVDASAVSGVLSLLALDETPLPDHSAVPAGLAVTLALVQALGDAGIEVPLWVATSGAVAAGAEGALARPVQAQVWGLGRVVALEHPERWGGLVDLPESLDERSGARLVAVLAGCGENEVALRPSGILGRRLARVTRHGSEENWTPRGSVLITGGTGAIAGHTARWLCGEGAERLVLTSRSGPAAEGVAALAAELAEAGSTVAVVACDVSERAPLSDLLDWIGRSGPELSSVMHTAGVLDDGVVDRLDTARLETVLGVKVRGAVLLDELTADLDLDAFVLFSAAAATLGGSGQGNYAAANAFLDALAEERRARGLNALAVAWGPWAGGGMAASSGAVRARVERGAMPPMDPRLAVRALAEALQGPDAAVAVMDVDWTQLASGPGAVELLKRPLVRDLPEIRQLLAAAGNTVTVAPAEGELARQLAGLDRSEQERLLTEVVRAEAAAVLGYGSADAVEDRRAFKDLGFDSLTAVELRNRLNAATGLRLPSTLVFDYPTPLALAAFLRGELTGGELDTAGAPVATVAAAPADEPLAIVGMACRFPGGVGSPREFWELLTSGGDAVGDFPSDRGWDLDGLIDPEGEREGTSYVAQGAFLREAAEFDAGFFGISPREALAMDPQQRLLLETSWEALERAGIDPASLRESATGVFAGGFTSGYGLGLSLSHQGDSGAEGHSLTGNATSVLSGRVSYVLGLEGPAVTVDTACSSSLVALHLAAQAVRSGECSLALAGGATVMATPGTFIDFSRQQGLSADGRCRAFSEDADGTGWAEGAGMVVVERLSDARRNGHKVLAVLRGSAVNQDGASNGLTAPNGPSQQRVIRAALANSGLTTADIDVVEAHGTGTTLGDPIEAQAVLATYGRGRAEDRPLWLGSVKSNIGHTQAAAGVAGVIKMVLALQYGQLPRTLHAEVPSSHVDWSAGAVRLLNEQLAWPSDDDRVRRAAVSAFGMSGTNAHLILEEAPAPEPAEVIDGLEDATTPKAAALAADAGIGAWVLSGRTADALAAQAGRLREWTATRPELAPADVAWSLAATRSVFEHRAVVVGGEREQVLAGLGGLTSGIPAGSVVSGVARSHARPVFAFAGQGSQWVGMGRELAEVSPVFAARLAECAAALAPYVDWSLADVLAGAEGAPALEAADVVQPALWAVMVSLAAVWEAAGVAPEAVVGHSQGEIAAATVAGMLSLQDGARVVALRSRALKVLAGAGGMLSVSRPVAEVEERLVRFGERLSLAAVNGPSATVVSGEPEALEELKAEFEAEGARARMVAVDYASHSAQVDRLEEEITSVLAGISPRRGRVPMVSAMTGETLTGEELDVGYWYRSLRATVHYDRAVRVLAGRGHQVFVEVTPHPVLMGAMNDTLVDLAPDAGGEPAAVCGTLRRDDGGAERLLTSLAEAFVNGATVDWQTVLPAGDQVELPTYAFRRRRFWPEGMLVLPMPGSATTVAGAGTEAEAGFWAAVEDGDLARVAETLALEDAGQLGAVLPALASWRRRELDRSATADWRYRTVWSPVAEPDARVLTGTWVVLASAGCSEELRHQCVTALTARGAEAVVVEVPAGTVDRAGLTALLTGAAADPSAISGVLSLLALDESVLPDRPVVNAGLAATLGLVQALGDAGVEAPLWVATSGAVAAGRGEMLSHPVQAQVWGLGRVVGLEHPERWGGLVDLPETLDERAGARLVAVLAGCGENEVAIRAAGILGRRLAHVSGRGVGESWSPRGTVLVTGGTGAIAGHVSRWLAGRGAERLVLAGRSGPAAADVAARAAELATGGVHVDVISCDVSERGPLTNLLGWIGSSGPELSSVMHTAGVLDDGVVDRLDTARLETVLGVKAHGAVLLDELTANMDLDAFVLFSSAASTVGGPGQGNYAAANAFLDALAENRRGRGLAGLAVAWGLWGGGGLGESSEVIRSRMRRMPMPPMDPQLAVRALGEALDGPDAVVTVMEVDWAQLASGAGAADMRVRPLVRDLPEIRELSAASGRAPALAPVEGELAARVAGLSRSEQDRVLTGVVRAEAAAVLGHASSDGVQARQAFKDLGFDSLTSVELRNRLNAATGLRLPATLVFDHPTPTALATFLRGELTGQVPEAVAATPVPAAPAADEPLAIVGMACRFPGGASTPEGFWELLASGGDAVGGFPTDRGWDLEGIYDPDGERENTSYVAQGAFLRGAAEFDAGFFGISPREALAMDPQQRLMLETSWEALERAGIDPSSLRGSSTGVFAGGFASGYGLGVSAAAGEGGSGVEGHLMTGNATSVLSGRVSYVLGLEGPAVTVDTACSSSLVALNLAAQAVRSGECSMALAGGVTVMATPGTFIDFSRQQGLSADGRCRAFSEDADGTGWAEGAGMLVVERLSDARRNGHKVLAVLRGSAVNQDGASNGLTAPNGPSQQRVIRAALASAGLSASEVDVVEAHGTGTKLGDPIEAQALLATYGRDRGDRDALWLGSVKSNIGHTQAAAGVAGVIKMVLALQHGQLPRTLHADVASSHVDWSAGEVRLLTESVPWPVGERPRRAGISAFGISGTNAHVILEEAPAIEARSEGAGQEPVAALEGAGAWVVSGRTAEALSAQAGRLREWLTARPELRPADVAWSLAATRSAFEHRAVVLGTERGALLDGVHSLAAGTSSPVVVSGVARPDIRVGLIFAGQGAQWAGMGRGLYAGSAVFAEVFDRVCGLLELELGTEVRLRDVVLNSDDAAAGLADQTLYAQAGLFAFEVALAAVLKAAGVVPDAVVGHSVGEVAAAHVAGVLSLPDACALVAARARLMQELPPGGAMAAINAAEADVIASFDGVSGEVAVAAVNGPESVVISGAAEAVDAVVELWRDRGCRVRRLRVSHAFHSPAMDPVLDELRTVAEGMEFRRPEVMWAGALTGDLVSDPQAGYWPAQTRQTVRFADAVAALAAEGISVFLEVGPDGSLSSLGPDAVAEVDGAEPAFLPLQRRDDEGATGLLTGLARAFVHGVPVDWTRVLPTGEHVELPTYAFRHQRFWPEAPAALSAGVSASGRDGAGSAAESAFWSAVESGDLEQLADTLAVDGDRPFHQVLPALTQWRRRDLERSTTAGWRYRMVWEPVAEPDPGALTGTWLVVVPAQSAAAELAQGCADALSARGAEVAVVEIPAGTMDRGELAALLGKAADPSAVSGVLSLLALDETPLPDHSAVPVGLAATLALVQALGDADIQAPLWPVTCGAVAAVPGEVPAGTVQAQVWGLGRVIGMEHPDRWGGLVDLPEVLDQRAAARLAAVLGGCGEDQVAVRATGILGRRMRHAAEAGPGEGHWTPRGSALITGGTGAVGGHVARWLAEADAARVVLTGRSGPAAGGVAVTAAELAARGTRVDVVACDVSDRSALAGLVSWIGTSGPALSTVLHTAAVLDDGVVDRLSAERLQTVLAAKAAGAAHLDELTAGLDLDAFVLFSSAASTLGSAGQGNYAAANSYLDALAENRRARGLTGLSVAWGQWGGGGMADSKEAIRERMKKLPLSAMDPRSAVRALGESLHGPDPVVTVMDVDWAVLAQSAANLTEVPLVRDLPAVRRLATAGSRAVEQPASPEGELARRLAGLDRAGQERTLTDLVRAEAAMVLGHPSAEAVQDQQAFKDLGFDSLTAVELRNRLNTATGLRVPVTLIFDHPTPVAVAAWLRTELAPDEPTALPVLDDLDRLEAGLLSAAPDQDTNERITRRLQSILSNWIEKQGRSDSGSGGVEIESATPDEMFEFLDRELGLSD

SEQ ID NO: 135 - protein, 6614 aa, Streptomyces sp.
MADEEALRDYLKWVTTDLYNTRQRLREVEERSHEPIAVVGIGCRYPGGVDTPEDFWNLLATARDGIADIPTDRGWDLAQLRVQDPDGSTRLPEGGFISDASGFDAAFFGISPREAVAMDPQQRLLLEVAWEALERSGIDPHVLRGSQTGVFAGASLSGYGFGTDEDLDVHLMTGTSTSVISGRVSYALGLEGPAVTVDTACSSSLVALHLAAQSVRSGECSLALAGGVTVTTVPVMFTQFSKQLGLAGDGRCKAFSADADGMGISEGAGMVVLERLSDARRNGHPVLAVIRGSAINQDGASNGLAAPNGPSQQRVIRAALANARLSTADVDLVEAHGTGTTLGDPIEAQALLATYGQGRAEDQPVRLGSVKSNIGHTQAGAGIAGVIKLVLALQHQQMPQTLHAAEPSPHIDWSAGNIELLTQAHPWPLGERVRRGGVSSFGVSGTNAHVIVEEAPAEEPAEAQTGTDGEDGTDTAPQDTGSHTAEPLLPESGAWLVSARTAEALSAQAGRLREWVAARPELGAADVAWSLAATRSAFEHRAVVLGSEREELLSAVADLAAGVPSSVVVSGVARSDVRVGLIFAGQGAQWVGMGRGLYAGSAVFAEVFDRVCGLLELELGTAVRLRDVVLGAEGVDEGLADQTLYAQAGLFAFEVALAEVLKAAGVVPDAVVGHSVGEVAAAHVAGVLSLPDACALVAARARLMQELPSGGAMAAVNAAEADVIASFDEVSGEVAVAAVNGPESVVISGATEAVDAVVELWRDRGCRVRRLRVSHAFHSPAMEPVLDELRTVAEKLEFRRPEVMWAGALTGELVSEPQAGYWPAQTRQTVRFADAIATLATDGISVFLEVGPDGSLSSLGPDAVADVDGAEPAFVPLQRRTEDGATDLETGLARAFVHGAPVDWKRVLPAGEQVELPTYAFRHQRYWPKGMMALPGTVKPGAGDPAELGLEAVGHPMLRAVVELAGEDQLVCTGRLSARTQTWLADHMVGGVTLLPGTGFVELVVSAGHQAGSPLLTELTLHEPLVLPAEGPGTQIQVVVGEGDEDGNRQVDVFARPDRSEEEHSWVRHATAVVAPARIPAPDAVRTPADLTAWPPRNARALDIDDLYAVQLADVYGPAQQGLQAVWQRGADIFAEVALPSDAGAAAPYALHPVLLDAALLADRVAGQAQGRATDDGPDRIRLPHAWSGVELHAAGASVLRVRLRRNPDGTLAVTAADPTGAMVLSVESVRTRPLSADELQSDDAWTRDALFTVEWLPLHRSGSSNSSQATAPAPDGPWALIGADRFAVAEQLTAAGTPVRGYAGIAELVAAVGAGETVPSTVLACLSGTSTHSGPAGTDTHGIAEAARQVTTEALILAQDWLAAPELEAAKLVLVTRGGVLVHPGEQAVDPAAGAVWGLLRSAQMEHPDRFVLADLPADGSAGDTAALPAALASGEPEVAVRQQSGYARRMVRPTVTDRTAPAPASAEAHPRTVLVTGGTGTLGGVVARHLAATGRADETLLLSRSGPAAPGAAALAARIAAHGADVRVIACDAADRDALAGVLDTIPADRPLRSVVHTAGVIDDGLIGSLSAEQLAAVLRPKADAAWHLHDLTRHLDLDHFVLFSSVAAAFGGAGQGNYAAANAFLDTLAGHRRAAGLAGTALAWGPWMPEAGIGRDLDEVMLRRMTGSGLTALDEHQGLTLLDLALERTEALLVPARLNKAALRAQAAATGIPALWTELADDLAPRRAAVTSAAGASAAGTSDQLRAHLAAQLGPERDRLLLDLVRSHAAAVLGHPSGDAIEAGRPFTDLGFDSLIAVEFRNRLNAATGLRLPSTLVFDHPNPTALARLLHEQLTDDLPQGAAPAAPQAAFAYAADEPIAIVGMACRFPGGVTSPEQLWQMLADGTDGLSTFPTDRGWDLEALYDPDIEHAGTSYTQVGGFLQDAGAFDAGFFGISPREALAMDPQQRLLLETSWEALERAGIDPATLRSSRTGAFVGGYGSSYLAVSVTDEEELENLGAHLMTGNAASVLSGRVSYTLGLEGPAVTVDTACSSSLVALHLAAQALRTGECSLALAGGVTIMASPDGFLAFSHARGLAADGRSKAFSDQADGMGMAEGAGMLVVERLSDARRNGHPVLAVLRGSAINQDGASNGLTAPNGPSQQRVIRAALANSGLSTADIDVVEAHGTGTTLGDPIEAQALLATYGQDRGDRGSLLLGSVKSNIGHTQAAAGVAGVMKMVLALQHERLPQTLYAEVPSSHVDWSAGEVRLLTEPVPWPSGGERVRRAGVSSFGISGTNAHVILEEAPAGEGVEAVEEAGEPGEPVVPVVTGAGAWVLSGRTAEALSAQAGRLREWVSARPALRPVDVAWSLAATRSVFEHRAVVVGGDREQVLAGLGGLASGEPSGALVAGTARSDVRPVFAFAGQGSQWVGMGRELAEVSPVFAGRLAECATALAPYVDWSLTDVLAGAEGAPALEAADVVQPALWAVMVSLAAVWEAAGVAPDAVVGHSQGEIAAATVAGMLSLEDGARVVALRSQALKVLAGAGGMLSVSRPASEVEERLVRFGERVSLAAVNGPSATVVSGEPAALEELQSEFEAAGARARMVAVDYASHSAQVDRLEEEITTVLAGISPRRGRVPMVSAMTGETVTGEELDAGYWYRSLRATVHYDRAVRVLAGRGHQVFVEVTPHPVLMGAMNDTLVDLAQEAGPGAEPGAVCGTLRRDDGGADRLVASLAEAFVNGASVDWAAVLPVGEQVELPTYAFRRRRYWPEGMLALPTGGSLAAGGEGASPDAESAFWAAVEGGDLSGLGDALALEGGRPFNEVLPALASWRRRERDRSTTAGWRYRTGWSVIVEPDVRLLSGTWLIAVPAESAAVELAQDCVGALSARGAEAVVVEVPAGSVDRSELAALLTGAADPSAISGVLSLLALDEMPLPDRPVVNAGLAATLGLVQALGDAGVEAPLWVATRGAVAAGRGEALARPVQAQVWGLGRVVGLEHPERWGGLVDLPETLDERSGARLVAVLAGCGENEVAVRRAGILGRRLSSVPHSSGPDKGWTPRGTVLITGGTGAIAGHVARRLAGRGAERLVLTSRSGPAAAGAAALAAELAAAGSAVEIVACDVSERVGLAGLLDGIDASGPRLSSVLHTAAVLDDGVVDRLSVARLAPVLEVKAHAAALLDELTAHLDLDAFVLFSSAASTLGGPGQGSYAAANSYLDALAENRRARGLAGLTVAWGLWGGAGLAQSNEAIRSRMRRTAMPPMDPQLAVEALIEAMDGPDAAVTVMDFDPQQLGAAPGTAHVLEMPLVRDMPEIRRLAAARAGNGSTGPAFRADGELAARLAGLSRSEQDRILTDVVRAEAAAVLGHDSADEVQARQAFRDLGFDSLTSVELRNQLNAATGLRLPATLVFDHPTPVALAAFLHGELTGQVTQTAAAAPVSATPAADEPLAIVGMACRFPGGASTPEEFWELLASGGDAVGGFPTDRGWDLDGFFDPDRHGSGTSYTDSGAFLRGAAEFDAGFFGISPREALAMDPQQRLMLETSWEALERAGIDPSSLRGSPTGVFAGGFGSGYAIGMALTGQDGSGVEGHLMTGNSTSVLSGRVSYALGLEGPAVTVDTACSSSLVALNLAAQALRSGDCSMALAGGVTVMATPFSFVEFSRQQGLSADGRCRAFSEDADGTGWAEGAGVLVVERLSDARRNGHRVLAVVRGTAVNQDGASNGLSAPNGPSQQRVIRAALANAGLSSSEVDVVEAHGTGTKLGDPIEAQALLATYGRDRGDRDALWLGSVKSNIGHTQAAAGVAGVIKMVLALQHGQLPRTLHADVASSHVDWSAGEVRLLTESVPWPVGERPRRAGVSAFGISGTNAHVILEEAPAIEARSEGAGQVPVAALEGAGAWVVSGRTAEALSAQAGRLREWLTARPELRPADVAWSLAATRSAFEHRAVVLGTEREELLSDLHSLAAGTSSPVVVSGVARPDVRVGLVFAGQGAQWAGMGRGLYAGSAVFAEVFDRVCGLLELELGAAVRLRDVVLNSDDAAAGLADQTLYAQAGLFAFEVALAAVLKAAGVVPDAVVGHSVGEVAAAHVAGVLSLPDACALVAARARLMQELPSGGAMAAINAAEADVITSFDEVSGEVAVAAVNGPESVVISGATEAVDAVVELWRDRGCRVRPLRVSHAFHSPAMDPVLDELRTVAEGMEFRRPEVMWAGALTGELVSDPQAGYWPAQTRQAVRFADAVATLAREGVSVFLEVGPDGSLSSLGPDAVADVEGAEAAFVALQRRDEEGVAGLLSGLARAFVNGAPVDWTRVLPAGEHVELPTYAFRHQRYWPEGVLPLMPSAVATVAGAGTEAEAGFWAAVEDGDLARVAETLALEDAGQLDLVLPALASWRRRELDRSATADWRYRTVWSPVAEPDARVLTGTWVVLAPAGCSEELRHQCVTALTARGAEVVVVEVPAGTVDRAGLTALLTGAADASTVSGVLSLLALDETPVPGYPVVPVGLAATLGLVQALGDAGVEAPLWIATRGAVAAGRGEALARPVQAQVWGLGRVVALEHPERWGGLVDLPETFDERSGARLVAMLAGCGENEVALRPSGILGRRLAHVSGRGARERWAPRGTVLVTGGTGAIAGHVSRWLAGRGAQRLVLTSRSGPAAVGAAALAAELAAAGSAVEIVACDVSERAGLAGLLDWIDASGPRLSSVMHTAGVLDDGVVDRLDTARLETVLGVKAQGAVLLDELTADLDLDAFVLFSAAAATLGGSGQGNYAAANAFLDALAEERRARGLNALSVAWGPWSGGGMAASSGAVRARVERGAMPPMDPRLAVRALAEALRGPDAAVAVMDVDWTQLASGPGAVELLKRPLVRDLPEIRDMAAAGGGSTAAGRVEGELARQLAGLDRAEQERTLADVVRGEAAVVLGYGSADAVEDRRAFKDLGFDSLTAVELRNRLNAATGLRLPSTLVFDYPTPLAMAAFLRGELTGGELDTAGASAATVVAAPADEPLAIVGMACRFPGGVGSPEEFWEFLASGGDGVGDFPSDRGWDLESVYDPEGEREGTSYVAQGAFLREAAEFDAGFFGISPREALAMDPQQRLLLETSWEALERAGIDPASLKESATGVFAGGFTSGYGLGVSMSQDDSGAEGHVLTGNATSVLSGRVSYVLGLEGPAVTVDTACSSSLVALHLAAQAVRSGECSLALAGGATVMATPGTFIDFSRQQGLSIDGRCRAFSEDADGTGWAEGAGMVVVERLSDARRNGHKVLAVLRGSAVNQDGASNGLTAPNGPSQQRVIRAALANARLSAAEVDVVEAHGTGTKLGDPIEAQALLATYGRDRGERDALWLGSVKSNIGHTQAAAGVAGVIKMVLALQHEQLPRTLHAEVPSSHVDWSAGAVRLLNEQLAWPSDGNRVRRAAVSAFGMSGTNAHLILEEAPAPQDGEAVEEHGEPVVPVVTGASAWVLSGRTAEALSAQAGRLREWTATRPELAPADVAWSLAATRSVFEHRSVVVGTGRAELLGGVESLATGVPAGSVVSGVARSHARPVFAFAGQGSQWVGMGRELAEVSPVFAGRLAECAAALAPYVDWSLTDVLAGAEDAPALEAADVVQPALWAVMVSLAAVWEAAGVAPEAVVGHSQGEIAAATVAGMLSLQDGARVVALRSRALKVLAGAGGMLSVSRPASEVEERLARFGDRLSLAAVNGPSATVVSGEPAALEELQSEFEAEGARARMVAVDYASHSAQVDRLEEEITTVLAGISPRRGRIPMVSAMTGEILTGEELDAGYWFRSLRATVHYDRAVRVLAGRGHQVFVEVTPHPVLMGAMNDTLVDLAQDAGGEPAAVCGTLRRDDGGAERLLTSLAEAFVNGAPVDWQTVLPAGDQVELPTYAFRRRRFWPEGMLVLPIPGSATTGSTSAGGGASTDAEAAFWAAVEDGDLSGLGDALALEGGRPFNEVLPALASWRRRERDRSTAAGWRYRTGWSAIAEPDARVLSGTWLMVVAAGVDGGAAQQCAAALSARGAEAVMIEVAAGSVDRSELAALLTGAADPSAISGVLSLLALDETPLPDRPVVNAGLAATLGLVQALGDTGIEAPLWIATRGAVAAGRGEALARPVQAQVWGLGRVVALEHPERWGGLVDLPETFDDRSGGRLVAVLAGCKENEVAIRTAGTLGRRMSRVPRRSPRTPWSPRGTVLVTGGTGAIAGHVSQWLAGRGAERLVLAGRSGPAAADVAARAAELATAGVRVDVISCDVSERGPLGSLLGWIGSNGPELSSVMHTAGVLDDGVLDRLTTPRLETVLGVKAHGAALLDELTADLDLDAFVLFSSAASTLGGPGQGNYAAANAYLDALAENRRARGLAGLAVAWGLWGGGGLGESNEAIRSRMRRLPMPPMDPQLAVRALGESLDGPEPTVTVMDFDWQQLGSAPGSADELDLPLVRDIPEIRRLAAERAAAGSVSRSDSRGELAARLAGAGRSEQDRILTDVVRAEAAAVLGHDSADGVQARQAFKDLGFDSLTAVELRNRLNAATGLRLPATLVFDHPTPTALAGWLRTELAPEETAAVPLLAELDRLEAALRSGPVDDPVREEATSRLRRLLDTIGEQAADQGRKRQTTDIDAASDEELFALVDGLE

SEQ ID NO: 136 - protein, 3245 aa, Streptomyces sp.
MSEEKLRYFLKRVTANLHETRQRLQEMEAAGGEPIALVGMACRFPGGVRSPQDLWQLLDAGRESVAALPRDRGWDLGDDGTGQDAAEVQAGNFLHDATGFDPAFFGISPREAVSMDPQQRLLLEVAWEALERSGIDPSSLRGSPTGVFAGASASGYGWASGAQGDLDGHVMTGNAMSVLSGRLAYTLGLEGPAVTVDTACSSALVSLHLACQALRAGECSMALVGGAYVAATPVLFTDFSRSLGLSPDGRCQAFGAGADGMGVAEGVGVVVAERLSDARRNGHRVLAVVRGSAVNQDGASNGLTAPNGPAQQRVIRAALAGARLTASEVDVVEAHGTGTPLGDPIEAQALLATYGQERPEGRPLWLGSVKSNIGHAQQAAGMAGIIKMVLALQHQNLPRTLHVEKPTDEVDWSAGEVKLLAEPVPWPSGERVRRAGVSGFGMSGTNVHVILEEAPAEEAAEATDPTGQEGPDAGEGEAEALSESAQHGPDQEAVSAPAPALLADGGPTAWLVSGRSAEGLAAQAGRLREWAAERPALRPSDVAWSLAATRSVFEHRGVVVADEHTALLSGLDDLAAATATADTVVGVARATGRTVFVFAGQGAQWVGMGRELLASSPLFAARLAECAAALAPYVDWSLSDVLTGVEGAPALEAAEVVQPVLWAVMVSLAAVWEAAGIVPDAVVGHSQGEIAAATVAGMLSLDDAARVVAVRSRALSGLDVAGAMVSVVMPAGAVRELVAGFDGRLSVAAVNGPASVVVSGEAEALASFERELAARKVLRWRIPATDFVAHSAAVEPLADQLEADLSGIAPQAGRIPMVSTVTGEWLAGEEADAAYWFANLRQMVRFEDAVRTLLESGYGAFVEVSPHPVLTAAVTETAQDADSGAAQVLSIGTLSQTDAGPAGLLRAFAQAHVAGLPVDWRKVLPAGEVVDLPTYAFQHRRYWLEPSAAAAAAVAAGGSSDAEARFWAAVEGGDLTHMAEVLNLDDAHALGEVLPALTSWRRQEQDRSATADWRYHITWAPVADPAATVLTGRWLLLTGPEGTGPAQACAQALAARGADTTIVEVPEGTVDRAAMAAVLGQALQETTAPTGIVSLLALDETPVPGHPAVPGGLARTLAAVQALGDTGVDAPLWVLTRGAVATTAQEPPVTPAQSLTWGFGRVVALEHPDRWGGLIDLPADFDGPDGADGQITARLCAVLAGCGEDQTAIRPAGIFGRRLSRAGRPRGEAGPYTPGGSALVTGGTGAIGGRVARWLSGRGAARLVLTSRSGPAAAGAAALAAELAAAGSRVDIVACDVSDRDQVATVLGRIAAQGPSLTTVLHTAGVNQATLVQDTDLAETDAVLAAKTAGAQHLHDLTRELDLNLDAFVLFSSISATWGSGLQPAYAAANTYLDGLAQHRRAHGLPATSVAWGSWGGGGMTDEAGAAQGIRRGLIVMDPDHAVQALAQVLDCDEPQMTVAHVDWQRFAPPFTLRRPSPLIENLPEVQEVLAADGQHTQGSGDTAAGPQAGASWRRQLAGLSRTEQKRTLLTLVQTQAAAVLDYAAPGEVEATRAFSDLGFDSLTSVELRNRLSSATGLQLPATLLFDAPTPSAAAEFLLAELAELTGTPDGTQADASRTAPVLAAAADEPLAIVGMACRYPGGASGPQELWNLVTAGTDAISVLPEDRGWDIEDRQGNPQGPVRSGGFVYDATHFDAGFFGISPREAVSMDPQQRLLLEVAWEALERSGIDPSSLRGSPTGVFAGASASGYGWSSGVQKELDGHLVTGISTSVVSGRVSYVLGLEGPAVTVDTACSSSLVALHMACQALRSGECSLALAGGVMVAANPLLFDQFSRQMGLSPDGRCKPFGAGADGMGLGEGAGMLVVERLSDARRHGHKVLAVVRGSAVNQDGASNGLTAPNGPSQQRVIRAALANARLTPGDVDVVEAHGTGTPLGDPIEAQALLATYGQERGEGDPLWLGSVKSNIGHTQAAAGVAGVIKMVLALQHRELPRTLHAAEPSPHIDWSAGDVRLLTEAAPWPEKNRVRRAGVSSFGMSGTNAHLILEEAPAEPADTGETPEPAEAAPAVLAAGEASAWLVSGRSAEGLAAQAGRLGEWAAGRAALRPVDVAWSLAATRAVLEHRGVVVAGDRAGLLDGVRGLAAGESAAPGVVSGVARATGRTVFAFAGQGAQWVGMGRDLLASSPLFAARLAECEAALAPYVDWSLSEVLAGTGDAPALEAAEVVQPALWAVMVSLAAVWEAAGVTPDAVVGHSQGEIAAATVAGMLSLEDAARVVAVRSRALSSLDVAGAMVSVVMPADAVRELVAGFDGRLSVAAVNGPASVVVSGQAEALATFERELAARKVLRWRIPATDFVAHSPAVEPLAAPLEAELSGIAAQAGRIPMVSTVTGEWLTGEETNASYWFANLRQMVRFEDAVRSLLESGYGAFIEISPHPVLTAAVTETAQDTDANATPVLSIGTLSQTDAGAAGLLHAFAQAHVAGLPVDWRKVLPAGEVVELPTYAFQHRRYWLEPSAAATAAAAAVAAGGSSAAEAQFWAAVEGGDLSGVADTLSLTDQQQLSGILPALASWRRREQERSATESWRYRITWAPVADPAPARLSGRWLLLVPASEVDGPMARQCRAALTARGADVVVLEMAGAAAREAVAEQVRAALPSDGFAGILSLLALDETPMPGHPSLSVGLAATLTLVQGLGDADVDAPLWVATSHAVAAAPGEPPVRPLQTQVWGLGRVVGLEHPERWGGLVDLPADFDGPDGQIAARLGAVLAGCGGEDQVAIRAAAILGRRLTHAPQPASPDQPWTPGGSVLVTGGTGALGGHVARWAAGRGARRLVLTSRSGPAAAGAAALAAELAAAGSRVDILACDVSDRAQVAGALGRIVAQGPSLTSVLHTAGIGQAVTVAEADLAETDAVLAAKASGARHLDELTRELGLELDAFVLFSSISAIWGSVVQPAYAAANTYLDGLAEHRRARGLPATSVAWGPWGGGGMTDAETAGRLARGGVALMDKDRAVQALAHLVDGGEGPMTVVDVDWSRFAVPFTLRRPSPLIAGLPEAAAALAAEATAPVTDPAAGASLREELAGLPSAEQHRALVRLVQEHAAAVLDYAAPGEVEATRAFSDLGFDSLTSVELRNRLSSATGLQLPATLLFDHPTPQAAADHLWSEQFQDGTGAVSLVEEVDRLGSLLTGAAPDEKTYDMVTERLHHLLAQWKESGAPEESQAVADKIGSATDDEIFEFIHRELGR

SEQ ID NO: 137 - protein, 4827 aa, Streptomyces sp.
MANEDKLRDYLKLVTANLRQVRRRLTEVEERSQEPIAIVGMGCRFPGGVNGPEDLWRLLASGTDAIGPFPADRGWDLENLYDPDPDRPGTSYARHGGFVYDATHFDADFFGISPREAAAMDPQQRLLLEVAWEALEQAGIDANALRGSSTGVFAGSYGQGYGTGPSVKTQGLEGHLLTGVSTSVTSGRLAFTFGLEGPAVTVDTACSSSLVALHMAAQALRAGECSLALAGGSTVISAPSVFVGFSRQRGMSDDGRCKSFSQDANGSGWAEGVGLLVVERLSDARRNGHHVLAVLRGSAVNQDGASNGLTAPNGPAQQRVIRAALANARLTPGDVDVVEAHGSATKLGDPIEAEALLATYGRERPEGRPLWLGSVKSNIGHTQAAAGVAGVIKTVLALQHERLPRTLHVDEPTPHVDWSAGEVRLLAESVPWPGGERVRRAGVSGFGMSGTNVHVILEEAPAEEAPAGEAEGTDADETPASAPAPVATAPAAALLAGGGPTAWLVSGRSAEGLAAQAGRLGEWAAGRAALRPVDVAWSLAATRAVLEHRGVVVAGDRAGLLDGVRGLAAGESAAPGVVSGVARATGRTVFVFAGQGAQWVGMGRELLASSPLFAARLAECEAALAPYVHWSLSDVLTGAEGAPALEAAEVVQPALWAVMVSLAAVWEVAGVSPDAVVGHSQGEIAAATVAGMLSLDDAARVVAVRSRALSSLDVAGAMVSVVMPAGAVRELVAGFDGRLSVAAVNGPASVVVSGQAEALASFERELAARKVLRWRIPATDFVAHSAAVEPLAAQLEAELSGIAPQAGRIPMVSTVTGEWLTGEEADAGYWFANLRQMVRFEDAVRSLLESGYGAFIEVSPHPVLTAAVTETAQDTDANANATPVLSIGTLSQADAGAAGLLRAFAQAHVAGLPVDWRKVLPAGEVVELPTYAFQHRRYWLETSTAVGGGDGAPTEVEARFWAAVEGGDLSDLADTLAVEDRRQLLEVLPVLASWRRRERERSATEGWRYQVTWAPVADPAPARLAGRWLLLVPAQEVDGEAARQCRAALSARGADVVVLETAGAVAREAVAEQVRAALPSDGFAGVLSLLALDETPMPGHPALTVGLAATLILVQGLGDADVDAPLWVATRGAVATGSDEAPNSPLQALIWGFGRVVALEHPDRWGGLIDLPADFDGPDGPDNADGQVAARLGAVLAGCGEDQVAIRAAAILGRRLTHAPQPVMPDQPWTPGGSVLVTGGTTAVGGHVARWAAERGARRLVLTGAPEHEGVAALAAGLAAAGSRVDVVACDVSDRAETAGVLAWTRARGPELSTVLHLAEIVDSVPVDDLDAAQLAEVLAAKAGSAAILDELTADCDLDAFVLFSSVAAVWGGGRQPGFSAANAYLEALAEDRRARGLTALAVAWGPWNAGTTATGDDGVQLERRGLRLLDPRAATGALAQILDGGESRPVVVDVNWAQFAPPFTLRRPSPLIAGLPEVAAALAAGTAEEAETVPEARGSLLRRLAGQSATEQQRTLTDLVRGEAAAVLEHSSAQAVGSGRAFSELGFDSLTAVELRNRLAAATGLALPATLLFDYPSPKVLAGYLAAELLGSPAAGADAAVPVPVPAGAEEDDPVVVVGMGCRFPGGVKTPEDLWQVLAGGTDAIGAFPTDRGWDLDALYDPDPDAPGTAYVRQGGFVHDATRFDAGFFGISPREALAMDPQQRLLLGTSWEALERSGIVPGSLRGSRTGVFAGASFSGYGLAQPEAAQGLEAHLLTGTTTSVLSGRLAYTLGLQGPAMTVDTACSSALVAVHLAAQALRSGECTLALAGGVFVASTPELFVWVSKQRGLAPDGRSKSFAADADGMGIAEGAGMVVLERLSDARRNGHPVLAVVRGSAVNQDGASNGLTAPNGPAQQRVIHEALAGCSLTCAEVDAVEAHGSGTPLGDPIEAQALLATYGQDRGERDPLWLGSVKSNIGHTQGAAGISGLIKMVLALQHEQLPKSLHAEQPSPHVDWTAGAVRLLSQAQPWPAGGARTRRAGISSFGVSGTNAHLILEEAPEAPEAPEAGGVPETSPAPEALPADASGDGGQSAAPPALLAAPGPVAWLVSGRTADGLRRQAAQLAEHLAAHPGARAADIAWSLATTRTVHEYRAVVSGADRTELTEGLDALAAGLNSPQVIAGTTAAVEGADEAGPVFVFPGQGAQWAGMGRELLATSPVFAERFAQCAEALAPHVHWSPADVLAGADGAPGLERAEVAQPLLWAVMVALAAVWEAAGVTPAAVVGHSQGEIAAATVAGILSLADAAAVVAVRSQELSALGTSGGMLSVVLPVAAVRELMEPWGDRLAVAAVNGPATTVVSGQPEALAEFGAALAARRAMRWPVPAGDFVAHSAEVDQLSGALAERLAGIRPQTGNIPMYSTVECRWADGPELDAGYWFANLRHTVRFQQAVQELATAGHATFVEISPQPVLTAAIDETFQELATAGTPVVAATLEREHGGPRRLLAALAQLHVAGTAVDWRRVLPAGRRIDLPTYAFADQRYWLGADHPAGPAALAPAQPGAATEAESRFWAAVEDGDASRLAATLAVDTAQPFGEVLASLASWRRRERDRDVTAGWRYRVGWSPLNDPAPARPSGTWLVAAPAAPAPGTVDLIQACTDALTARGARTQVVQVAAGTERATLAEALRPVGIAPSEAGPATTPPPVAGVLSLLGLDETPLADHPAVPAGLAGTLTLLQALSDADVSAPLWMATTGAVSTAPGDRLTRPVQAATWGLGRVVALEQPERWGGLIDLPATLDERAAARLCAVLAGCGEDEVAIRPAGILARRLARAPRPATGRPWTPRGTVLITGGTGAVGGHVARWLAGRGADRLVLTGRSGAGAPGAAALAAHVAAAGTTVEIRACDSADRTRLAALLDRIAADGPPLTAVMHTAGVLDDGVLDRLDTDRLANALAAKATGAAHLDALTADRDLDAFVLFSSAAATFGGAGQANYAAANAFLDALAENRRSRGLPALSIAWGPWAGDGVSQATEAARQRLRRNRWEVLMDPELAVQALGTALAGTDTVLTVMDLDLAAFAAAPGQQQALDAPLLRDLAEVQQLKTARHADGGAQPAAGELTRQLAGLSRAEQDRLLVDRIRTEAAAVLGHAGYDEVEPGRAFSELGFDSLTSVELRNRIATWTALRLPATLMFDHPTPTVLAAHLRTTLLGSPDAGPAAAPAPDLTGHADRDPIAVVAMSCRFPGDVNTPEDLWALLAAGGEGISGYPQDRGWDADALFDPDPSHAGTSYTREGGFLHQATHFDAGFFGISPREALAMDPQQRLMLELSWEALESAGLDPATLRGSRTGVFIGGYSSHYSLASMQLGGQDGAAQVEGHLVTGNATSIISGRVSYVLGLEGPALTVDTACSSALVALHTAAQALRSGECPLALVGGVAVMATPWEMVGFARQRGLAADGRSKAFSADADGMGMGEGAGMVVLERLSDARRNGHRVLGVIRGSAVNQDGASNGLTAPNGPSQQRVIRAALANAGLTCAEVDAVEAHGTGTPLGDPIEAQALLATYGQERPEGRPLWLGSVKSNIGHTQAAAGMAGLIKMLLALRHEELPRTLHAEQPSPHVDWSAGEVRLLTESVPWPSDGDRVRRAGTSAFGISGTNAHVILEEAPAVEARSESADEVPVAALEGAGAWVVSGRTAEALSAQAGRLREWVAARPELGAADVAWSLAATRSAFEHRAVVLGSERGALLDGVHSLAAGTSSPVVVSGVARSDVRVGLIFAGQGAQWVGMGRGLYAGSAVFAEVFDRVCGLLELELGAEVRLRDVVLEGDGDAAGLADQTLYAQAGLFAFEVALAEALKAAGVVLDAVVGHSVGEVAAAHVAGVLSLPDACALVAARARLMQELPSGGAMAAINAAETAVVASLEEVSGEAAVAAVNGPESVVISGATEAVDAVVELWRGRGCRVRRLRVSHAFHSPAMEPVLDELRTVAEKLEFRRPEVMWAGALTGELVSDPQAGYWPAQTRQTVRFADAVATLAAEGISVFLEVGPDGSLSSLGPDAVADVDGAEPAFVPLQRRSTEGTAGLLSGLARAFVNGVPVDWKRVLPAGEQVELPTYAFRHQRYWPNGIQTLPHAWGDSASATAQDRFWAAVEDGDLTDIAGTLAAEDAQQLAQALPALASWRRREQDRSAIADWFYRVDWEPVTDPAPAELSGTWLLVSPAGPVAQALTEHCTQALAAGGARVVPLEATQTADPERLAAQIAEVLPPDGVAGVVSLLALDETPLPGHPALTRGLAGSQNLTQALVAEHVLAPVWGVTCGAVAVAPEKAPARPVQAQAWGLGRVVVLEHPDHGGGLIDLPGQWDENTAQRLRAVLAGCGEDQVALRPSGIHGRRLTRAAQPRERAGQWTPRGTALVTGGTGAIGGHVARWLADRGTERLVLTSRSGPGAATSAALAAELAAAGTHVDILACDAAEETALAGVLDRISVGGPPLTAVLHAAGLGQMTPLDQASVPELAAVLAAKAAGAATLDRLTADLELDAFVLFSSVAATWGSAMQAGYAAANAYLDALAEDRRARGLAATSVAWGPWGGGGMTDEEATGHMERRGLRLIDPHAALGALARALDADDATLTVADVDWERFAPPFTLRRPSPLIESLPDVIQALAARDDDGDAPETSAAGNALARQLAALSPTDQDRLLITLVQTEAAVVLGHPGPESVQAERAFSDLGADSLTAVELRDRLGRATGLRLPSTLLFDYPTSAALAGHLRDRLTEESSPARPVLAELDRLEEMLTGVPGGGDAAQITSRLELVLSRWKQTQAQNAETEVAEKLESSSDDEVFDFIGKELGIY

SEQ ID NO: 138 - protein, 223 aa, Streptomyces sp.
MSDEKVPTDDGTQDEQAAMRASDADREQVAEILRDAAGDGRLTLEEVGERVESALRARTVGELARLTRDLPVSPVPAGSGEQQPGRGRDVVQIDQRWGTLHRSGAWEVPRHLEVRMHGGDVKLDFTEAEFSHARLDLDVQISVGGNLVLVVRPGIVVHTDDLTVKVGEVKYRNPASDAQAPVEREVYVTGRLKGGDVVVRHPRRTLGRWLRGEDAKNGAAEKS

SEQ ID NO: 139 - protein, 337 aa, Streptomyces sp.
MNLLMGAMASSAVGTAAELGIADALADGPATADELAKTVEADTDSLDSVLQALTALGVFTRDADGRYANSPMSEPLKSDHPDSVRHLATLLCGLYGQAATGGLTQAVRAGTSVMPCTFGVGLYEYLDSHPDTARIFDLAMQDWTRPIATLLADKLPLDDVHTVVDVGGGNGTLLQALLSAHPHLNGICADRPDTCDRAERTLAESQQADLARRLTFRPTDILQDVPTGGDLYLIKNVLHDWNTTTSVQILRNIHTAMTATPRTSAGAPPKLLIIDPLAEHDSGAAFRNVIKMVVGEPRTRSRSEADIRREATEAGFEVLSITPCPADLSVITCVPAS

(19) United States
(12) Patent Application Publication
(10) Pub. No.: US 2026/0363251 A1
(43) Pub. Date: Nov. 19, 2026

(54) MEANS AND METHODS FOR CONTROLLING PATHOGENS AND PESTS IN PLANTS

(71) Applicant: SYNGENTA CROP PROTECTION AG, Basel (CH)

(72) Inventors: Stephane BIERI, Stein (CH); Lauren RAY, Bracknell, Berkshire (GB); Philip SIDEBOTTOM, Bracknell, Berkshire (GB); Dimitrios PAPASOTIRIOU, Bracknell, Berkshire (GB); Dianne IRWIN, Bracknell, Berkshire (GB); Dimitrios DRAKOPOULOS, Stein (CH); Peter VAN DE VONDERVOORT, Echt (NL); Leon COULIER, Echt (NL); Rosa DOMINGUEZ-ESPINOSA, Bracknell, Berkshire (GB); William Guy WHITTINGHAM, Bracknell, Berkshire (GB)

(73) Assignee: SYNGENTA CROP PROTECTION AG, Basel (CH)

(21) Appl. No.: 19/118,519

(22) PCT Filed: Oct. 5, 2023
(86) PCT No.: PCT/EP2023/077621

§ 371 Date: Apr. 4, 2025

(30) Foreign Application Priority Data
Oct. 7, 2022 (EP) 22200224.8
Oct. 7, 2022 (EP) 22200225.5

(51) Int. Cl. A01N 63/50; A01N 43/36; A01N 43/40; A01P 3/00; C12N 1/205; C12R 1/465

(52) U.S. Cl. CPC A01N 63/50; A01N 43/36; A01N 43/40; A01P 3/00; C12N 1/205; C12R 2001/465

(57) ABSTRACT

The present invention relates to a microbial strain and a composition comprising a strain of Streptomyces, a process of producing the microbial strain or composition and a method of using of the microbial strain and compositions to prevent or control pests in plants.

The present invention relates to novel microbial strains which have pesticidal activity. The invention also relates to compositions comprising a Streptomyces, to a process for the preparation of the microbial strain or the compositions and to methods of using Streptomyces chrestomyceticus or the compositions in agriculture or horticulture for preventing or controlling phytopathogenic infestation of plants, harvested food crops, seeds or non-living materials.

### BACKGROUND

Pesticides are widely used in agriculture to protect plants against damage caused by phytopathogenic microorganisms. Pesticides may be from chemical origin or biological origin. Due to some negative effects of chemical pesticides on the environment, there is a growing need for pesticides from biological origin, for instance microbial origin. Known microorganisms that produce antibiotics against fungi are actinomycetes, for instance Streptomyces sp. A very well-known species is Streptomyces natalensis that produces the antifungal compound natamycin, which is used in food and crop protection. In U.S. Pat. No. 5,356,624 a Streptomyces rimosus strain is disclosed that was found active against several wood-degrading fungi. In WO2022/038180 new Streptomyces sp. are disclosed that produce several known antifungal compounds or metabolites such as streptimidone, natamycin (pimaricin), or albofungin. The extracts of these bacterial strains were found active against well-known plant pests such as Fusarium graminearum, Zymoseptoria tritici, and Puccinia striiformis.

Due to development of resistance by phytopathogenic microorganisms against pesticides, regulations by governments and societal pressure there is a continuous need for improved products such as microbial strains or compounds from biological origin that have activity against phytopathogenic microorganisms.

### SUMMARY

The present invention relates to an isolated microbial strain which comprises a genome sequence which has at least 99.8%, 99.9% identity or 100% identity to the whole genome of Streptomyces sp. Saigon413 deposited with the Westerdijk Institute under accession number CBS149411.

In a second aspect the present invention relates to an isolated microbial strain, wherein the strain comprises a nucleotide sequence which has at least 99.8% identity to SEQ ID NO: 1.

In a third aspect the present invention relates to a composition which comprises a strain of Streptomyces, malonomicin and at least one of the compounds selected from the group consisting of cyclothiazomycin C, and streptimidone, a compound according to compound I, comprising a molecular formula according to C₅₃H₉₀N₂O₄₄, further characterised by the NMR spectra listed in Table 2 and Table 3, preferably characterized by a structural Formula I,

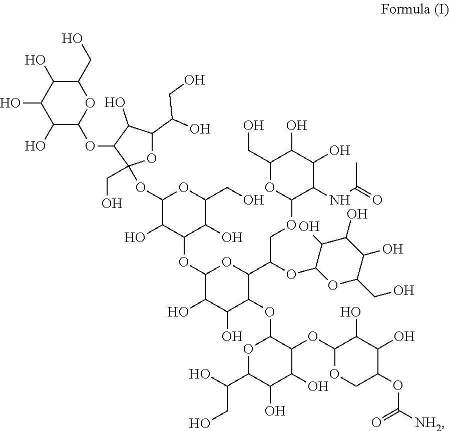 [SMILES: CC(=O)NC1C(OCC(OC2OC(CO)C(O)C(O)C2O)C2OC(OC3C(O)C(CO)OC(OC4(CO)OC(C(O)CO)C(O)C4OC4OC(CO)C(O)C(O)C4O)C3O)C(O)C(O)C2OC2OC(C(O)CO)C(O)C(O)C2OC2OCC(OC(N)=O)C(O)C2O)OC(CO)C(O)C1O]

a lipopeptide according to Formula II, or a salt thereof wherein R1=CH₃ or C₂H₅

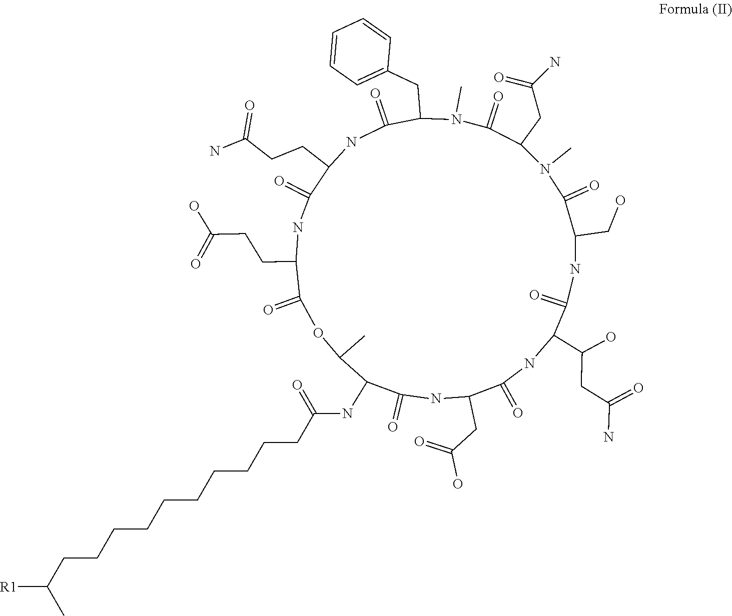

and a polyene compound characterized by a molecular formula according to C₆₇H₁₁₅NO₂₅, wherein the polyene is further characterized by the spectrum of light absorption as shown in FIG. 10.

In a fourth aspect the present invention relates to a process for producing the microbial strain as disclosed herein or a composition as disclosed herein comprising cultivating the microbial strain or the microbial strain of Streptomyces in a suitable fermentation medium under suitable fermentation conditions, and optionally comprising a step of recovering the microbial strain or composition. In a fifth aspect the present invention relates to a method for controlling or preventing infestation of a plant, plant propagation material and/or harvested food crops by a phytopathogenic microorganism, by treating the plant, plant propagation material and/or harvested food crops, by applying an effective amount of Streptomyces chrestomyceticus, an isolated microbial strain as disclosed herein, or a composition as disclosed herein to the plant, to a part thereof or a locus thereof, the plant propagation material and/or harvested food crops.

In a sixth aspect the present invention relates to a plant or a plant propagation material treated with the microbial strain, or a composition according to the present invention.

In a seventh aspect the present invention relates to the use of a microbial strain according the present invention, or a S. chrestomyceticus as disclosed herein or a composition according to the present invention as a pesticide, preferably as a fungicide.

In an eighth aspect the present invention relates to the use of a microbial strain according the present invention, or a Streptomyces which has at least 95% identity to the whole genome of Streptomyces chrestomyceticus NRRL B-3672, or at least 95% identity to the whole genome of Streptomyces sp. Saigon413 deposited with the Westerdijk Institute under accession number

CBS149411, for producing malonomicin and at least one of the compounds selected from the group consisting of cyclothiazomycin C, streptimidone, an oligosaccharide compound according to compound I, comprising a molecular formula according to C₅₃H₉₀N₂O₄₄, further characterised by the NMR spectra listed in Table 2 and Table 3, preferably characterized by a structural Formula I, a lipopeptide according to Formula II, or a salt thereof wherein R1=CH₃ or C₂H₅, and a polyene compound characterized by a molecular formula according to C₆₇H₁₁₅NO₂₅, wherein the polyene is further characterized by the spectrum of light absorption as shown in FIG. 10.

Surprisingly, it has been found that the strain of Streptomyces such as an isolated microbial strain as disclosed herein, for instance Streptomyces sp. Saigon 413, and/or its metabolites are effective in treating phytopathogenic microbial diseases on crops.

### DETAILED DESCRIPTION

The present invention relates to an isolated microbial strain which comprises a genome sequence which has at least 99.8%, 99.9% identity or 100% identity to the whole genome of Streptomyces sp. Saigon413 deposited with the Westerdijk Institute under accession number CBS149411.

The present invention also relates to an isolated microbial strain wherein the strain comprises a nucleotide sequence which has at least 99.9%, 99.91%, 99,92%, 99.93%, 99.94%, 99.95%, 99.96%, 99.97%, 99.98%, 99.99% or which has 100% identity to SEQ ID NO: 1.

An isolated microbial strain according to the present invention preferably is a microbial strain comprising a nucleotide sequence which has at least 99.9%, 99.91%, 99,92%, 99.93%, 99.94%, 99.95%, 99.96%, 99.97%, 99.98%, 99.99% or which has 100% identity to SEQ ID NO: 1, and wherein the microbial strain comprises a genome sequence which has at least 99.8%, 99.9% identity or 100% identity to the whole genome of Streptomyces sp. Saigon413 deposited with the Westerdijk Institute under accession number CBS149411.

For example, the microbial strain may comprise a nucleotide sequence which has at least 99.9% identity to SEQ ID NO: 1, and wherein the microbial strain comprises a genome sequence which has at least 99.8%, 99.9% identity or 100% identity to the whole genome of Streptomyces sp. Saigon413 deposited with the Westerdijk Institute under accession number CBS149411. By way of another example, the microbial strain may comprise a nucleotide sequence which has at least 99.91% identity to SEQ ID NO: 1, and wherein the microbial strain comprises a genome sequence which has at least 99.8%, 99.9% identity or 100% identity to the whole genome of Streptomyces sp. Saigon413 deposited with the Westerdijk Institute under accession number CBS149411. By way of another example, the microbial strain may comprise a nucleotide sequence which has at least 99.92% identity to SEQ ID NO: 1, and wherein the microbial strain comprises a genome sequence which has at least 99.8%, 99.9% identity or 100% identity to the whole genome of Streptomyces sp. Saigon413 deposited with the Westerdijk Institute under accession number CBS149411. By way of another example, the microbial strain may comprise a nucleotide sequence which has at least 99.93% identity to SEQ ID NO: 1, and wherein the microbial strain comprises a genome sequence which has at least 99.8%, 99.9% identity or 100% identity to the whole genome of Streptomyces sp. Saigon413 deposited with the Westerdijk Institute under accession number CBS149411. By way of another example, the microbial strain may comprise a nucleotide sequence which has at least 99.94% identity to SEQ ID NO: 1, and wherein the microbial strain comprises a genome sequence which has at least 99.8%, 99.9% identity or 100% identity to the whole genome of Streptomyces sp. Saigon413 deposited with the Westerdijk Institute under accession number CBS149411. By way of another example, the microbial strain may comprise a nucleotide sequence which has at least 99.95% identity to SEQ ID NO: 1, and wherein the microbial strain comprises a genome sequence which has at least 99.8%, 99.9% identity or 100% identity to the whole genome of Streptomyces sp. Saigon413 deposited with the Westerdijk Institute under accession number CBS149411. By way of another example, the microbial strain may comprise a nucleotide sequence which has at least 99.96% identity to SEQ ID NO: 1, and wherein the microbial strain comprises a genome sequence which has at least 99.8%, 99.9% identity or 100% identity to the whole genome of Streptomyces sp. Saigon413 deposited with the Westerdijk Institute under accession number CBS149411. By way of another example, the microbial strain may comprise a nucleotide sequence which has at least 99.97% identity to SEQ ID NO: 1, and wherein the microbial strain comprises a genome sequence which has at least 99.8%, 99.9% identity or 100% identity to the whole genome of Streptomyces sp. Saigon413 deposited with the Westerdijk Institute under accession number CBS149411. By way of another example, the microbial strain may comprise a nucleotide sequence which has at least 99.98% identity to SEQ ID NO: 1, and wherein the microbial strain comprises a genome sequence which has at least 99.8%, 99.9% identity or 100% identity to the whole genome of Streptomyces sp. Saigon413 deposited with the Westerdijk Institute under accession number CBS149411. By way of another example, the microbial strain may comprise a nucleotide sequence which has at least 99.99% identity to SEQ ID NO: 1, and wherein the microbial strain comprises a genome sequence which has at least 99.8%, 99.9% identity or 100% identity to the whole genome of Streptomyces sp. Saigon413 deposited with the Westerdijk Institute under accession number CBS149411. By way of another example, the microbial strain may comprise a nucleotide sequence which has at least 100% identity to SEQ ID NO: 1, and wherein the microbial strain comprises a genome sequence which has at least 99.8%, 99.9% identity or 100% identity to the whole genome of Streptomyces sp. Saigon413 deposited with the Westerdijk Institute under accession number CBS149411.

Suitably, the microbial strain may comprise a nucleotide sequence which has at least 99.99% identity to SEQ ID NO: 1, and wherein the microbial strain comprises a genome sequence which has at least 99.9% identity to the whole genome of Streptomyces sp. Saigon413 deposited with the Westerdijk Institute under accession number CBS149411. The wording ‘isolated’ with reference to the microbial strain means that the microbial strain has been isolated from it's native environment.

Surprisingly, it was found that a microbial strain according to the present invention exhibited pesticidal activity against phytopathogenic microorganisms which are found on various crops. Surprisingly, the microbial strain as disclosed herein produces one or more novel metabolites or a combination of metabolites with pesticidal, preferably fungicidal activity. Surprisingly, the microbial strain as disclosed herein produces or is able to produce at least one, at least two, at least three, at least four, at least five, or all of the compound(s) selected from malonomicin, cyclothiazomycin C, and streptimidone, an oligosaccharide compound according to compound I, comprising a molecular formula according to C₅₃H₉₀N₂O₄₄, further characterised by the NMR spectra listed in Table 1 and Table 2, preferably characterized by a structural Formula I, a lipopeptide according to Formula II, or a salt thereof wherein R1=CH₃ or C₂H₅, and a polyene compound characterized by a molecular formula according to C₆₇H₁₁₅NO₂₅, wherein the polyene is further characterized by the spectrum of light absorption as shown in FIG. 10.

Preferably the microbial strain as disclosed herein produces or is able to produce malonomicin and at least one, at least two, at least three, at least four or at least five of the compounds selected from cyclothazomycin C, streptimidone, an oligosaccharide compound according to compound I, comprising a molecular formula according to C₅₃H₉₀N₂O₄₄, further characterised by the NMR spectra listed in Table 1 and Table 2, preferably characterized by a structural Formula I as disclosed herein, a lipopeptide according to Formula II as disclosed herein, or a salt thereof wherein R1=CH₃ or C₂H₅ and a polyene compound characterized by a molecular formula according to C₆₇H₁₁₅NO₂₅, wherein the polyene is further characterized by the spectrum of light absorption as shown in FIG. 10.

Preferably the microbial strain as disclosed herein produces or is able to produce at least one, at least two, at least three of the compounds selected from malonomicin, cyclothazomycin C and streptimidone, and at least one, at least two or at least three of the compounds selected from an oligosaccharide compound according to compound I, comprising a molecular formula according to C₅₃H₉₀N₂O₄₄, further characterised by the NMR spectra listed in Table 1 and Table 2, preferably characterized by a structural Formula I as disclosed herein, a lipopeptide according to Formula II as disclosed herein, or a salt thereof wherein R1=CH₃ or C₂H₅ and a polyene compound characterized by a molecular formula according to C₆₇H₁₁₅NO₂₅, wherein the polyene is further characterized by the spectrum of light absorption as shown in FIG. 10.

The term “microbial strain, compound, metabolite or composition having pesticidal activity” or “pesticide” as used herein means a microbial strain, compound, metabolite, or composition that controls, modifies, or prevents the growth of pests. The term “pesticidally effective amount” where used means the quantity of such a microbial strain, compound, metabolite or composition or combination of such compounds that is capable of producing an effect on the growth of pests. Controlling or modifying effects include all deviation from natural development, such as killing, retardation and the like, and prevention includes barrier or other defensive formation in or on a plant to prevent pest infection.

The term “microbial strain, compound, metabolite or composition having fungicidal activity” or “fungicide” as used herein means a microbial strain, compound, metabolite, or composition that controls, modifies, or prevents the growth of fungi. The term “fungicidally effective amount” where used means the quantity of such a microbial strain, compound, metabolite or composition or combination of such compounds that is capable of producing an effect on the growth of fungi. Controlling or modifying effects include all deviation from natural development, such as killing, retardation and the like, and prevention includes barrier or other defensive formation in or on a plant to prevent fungal infection.

Preferably, the microbial strain as disclosed herein produces or is able to produce at least one compound selected from malonomicin, cyclothiazomycin C, streptimidone, and at least one compound selected from an oligosaccharide compound according to compound I, comprising a molecular formula according to C₅₃H₉₀N₂O₄₄, further characterised by the NMR spectra listed in Table 1 and Table 2, preferably characterized by a structural Formula I, a lipopeptide according to Formula II, or a salt thereof wherein R₁=CH₃ or C₂H₅, and a polyene compound characterized by a molecular formula according to C₆₇H₁₁₅NO₂₅, wherein the polyene is further characterized by the spectrum of light absorption as shown in FIG. 10.

Suitably, the microbial strain as disclosed herein produces or is able to produce malonomicin, cyclothiazomycin C, streptimidone, an oligosaccharide compound according to compound I, comprising a molecular formula according to C₅₃H₉₀N₂O₄₄, further characterised by the NMR spectra listed in Table 1 and Table 2, preferably characterized by a structural Formula I, a lipopeptide according to Formula II, or a salt thereof wherein R₁═CH₃, or C₂H₅ and a polyene compound characterized by a molecular formula according to C₆₇H₁₁₅O₂₅N, wherein the polyene is further characterized by the spectrum of light absorption as shown in FIG. 10.

As used herein, the terms “percent identity,” and “percent identical” refer to the relatedness of two or more nucleotide or amino acid sequences, which may be calculated by (i) comparing two optimally aligned sequences over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100 percent to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present invention, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100 percent.

In one embodiment the microbial strain according to the present invention is a Streptomyces chrestomyceticus, which is Streptomyces sp. Saigon413 deposited with the Westerdijk Institute under accession number CBS149411.

The present invention also relates to a composition which comprises a Streptomyces sp., preferably a Streptomyces chrestomyceticus, and malonomicin and at least one of the compounds selected from the group consisting of, cyclothiazomycin C, and streptimidone, an oligosaccharide compound according to compound I, comprising a molecular formula according to C₅₃H₉₀N₂O₄₄, further characterised by the NMR spectra listed in Table 2 and Table 3, preferably characterized by a structural Formula I,

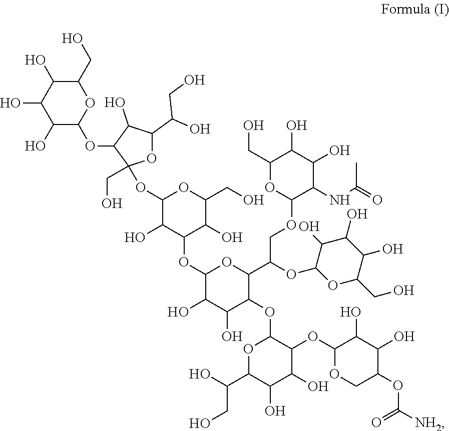 [SMILES: CC(=O)NC1C(OCC(OC2OC(CO)C(O)C(O)C2O)C2OC(OC3C(O)C(CO)OC(OC4(CO)OC(C(O)CO)C(O)C4OC4OC(CO)C(O)C(O)C4O)C3O)C(O)C(O)C2OC2OC(C(O)CO)C(O)C(O)C2OC2OCC(OC(N)=O)C(O)C2O)OC(CO)C(O)C1O]

a lipopeptide according to Formula II, or a salt thereof wherein R₁═CH₃ or C₂H₅,

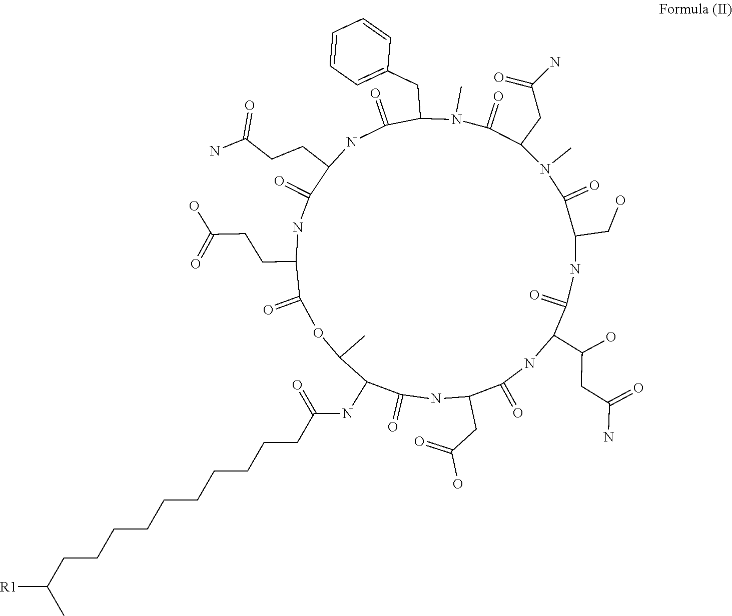

and a polyene compound characterized by a molecular formula according to C₆₇H₁₁₅NO₂₅, wherein the polyene is further characterized by the spectrum of light absorption as shown in FIG. 10. The polyene having the molecular formula of C₆₇H₁₁₅NO₂₅ has a molecular mass of 1333.7758 g.

Suitably, the present disclosure relates to a composition which comprises a strain of Streptomyces, preferably a Streptomyces chrestomyceticus, and at least one of the compounds selected from the group consisting of malonomicin, cyclothiazomycin C, and streptimidone, and at least one of the compounds selected from the group consisting of an oligosaccharide compound according to compound I, comprising a molecular formula according to C₅₃H₉₀N₂O₄₄, further characterised by the NMR spectra listed in Table 2 and Table 3, preferably characterized by a structural Formula I, a lipopeptide according to Formula II, or a salt thereof wherein R₁═CH₃ or C₂H₅, and a polyene compound characterized by a molecular formula according to C₆₇H₁₁₅NO₂₅, wherein the polyene is further characterized by the spectrum of light absorption as shown in FIG. 10.

It was found that a composition according to the present invention has a surprising level of biological activity against phytopathogenic microorganisms, in particular phytopathogenic fungi such as Zymoseptoria, Puccinia, Mycorsphearella, Pyricularia, Rhizoctonia, Blumeria, Alternaria, Colletotrichum, Ramularia, Parastagonospora, Rhynchosporium, Oculimacula, Fusarium, Gaeumannomyces sp., Botrytis, or Sclerotinia, the phytopathogenic bacteria Xanthomonas and the phytopathogenic oomycetes such as Aphanomyces sp.

Cyclothiazomycin C is a known compound. The structure of cyclothazomycin C is disclosed on p. 3 of WO2015191789 and can be produced as disclosed in Example 4 of WO2015/191789.

Malonomicin (sometimes spelt ‘malonomycin’) is {[(2S)-2-amino-3-hydroxypropanoyl]amino} {2-[(5S)-5-(aminomethyl)-4-hydroxy-2-oxo-2,5-dihydro-1H-pyrrol-3-yl]-2-oxoethyl}malonic acid can be produced as disclosed in Example 1 of WO2006/078939. Malonomicin may also be prepared according to the method disclosed in Example I A and B in EP 1860939, or according to Law et al, 2018 (Nature Catalys is | VOL 1 | DECEMBER 2018 | 977-984).

Streptimidone is a known compound of Formula III

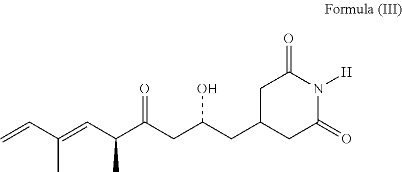 [SMILES: C=C/C(C)=C/[C@H](C)C(=O)C[C@H](O)CC1CC(=O)NC(=O)C1]

Streptimidone can be synthesised following the method disclosed in Kondo, H., Oritani, T., and Kiyota, H. Synthesis and antifungal activity of the four stereoisomers of streptimidone, a glutarimide antibiotic from Streptomyces rimosus forma paromomycinus. Eur. J. Org. Chem. (20), 3459-3462 (2000).

The composition according to the present invention preferably comprises malonomicin, cyclothiazomycin C, and streptimidone, and at least one, at least two, or three of the compounds selected from from the group consisting of an oligosaccharide compound according to compound I, comprising a molecular formula according to C₅₃H₉₀N₂O₄₄, further characterised by the NMR spectra listed in Table 1 and Table 2, preferably characterized by a structural Formula I, a lipopeptide according to Formula II, or a salt thereof wherein R1=CH₃ or C₂H₅, and a polyene compound characterized by a molecular formula according to C₆₇H₁₁₅NO₂₅, wherein the polyene is further characterized by the spectrum of light absorption as shown in FIG. 10.

The composition as disclosed herein is a non-naturally occurring composition.

Preferably, the composition according to the present invention comprises a Streptomyces, preferably a Streptomyces chrestomyceticus, or a microbial strain as disclosed herein which produces or is able to produce at least one, at least two, at least three, at least four, at least five, or all of the compound(s) selected from malonomicin, cyclothiazomycin C, and streptimidone, an oligosaccharide compound according to compound I, comprising a molecular formula according to C₅₃H₉₀N₂O₄₄, further characterised by the NMR spectra listed in Table 1 and Table 2, preferably characterized by a structural Formula I, a lipopeptide according to Formula II, or a salt thereof wherein R1=CH₃ or C₂H₅, and a polyene compound characterized by a molecular formula according to C₆₇H₁₁₅NO₂₅, wherein the polyene is further characterized by the spectrum of light absorption as shown in FIG. 10.

A lipopeptide according to Formula II as disclosed herein comprises a lipopeptide according to Formula II (a)

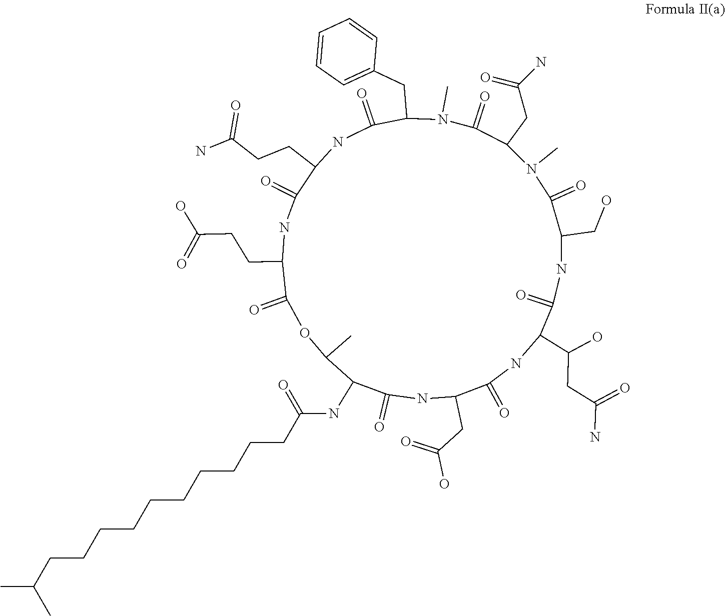 [SMILES: CC(C)CCCCCCCCCCC(=O)NC1C(=O)NC(CC(=O)O)C(=O)NC(C(O)CC(N)=O)C(=O)NC(CO)C(=O)N(C)C(CC(N)=O)C(=O)N(C)C(Cc2ccccc2)C(=O)NC(CCC(N)=O)C(=O)NC(CCC(=O)O)C(=O)OC1C]

The lipopeptide according to Formula II (a) comprises a molecular formula C₅₅H₈₅N₁₁O₁₉ and an exact mass of 1203.602 g. The lipopeptide according to Formula I (a) has a solubility in DMSO of above 10,000 ppm.

A lipopeptide according to Formula II as disclosed herein comprises a lipopeptide according to Formula II (b)

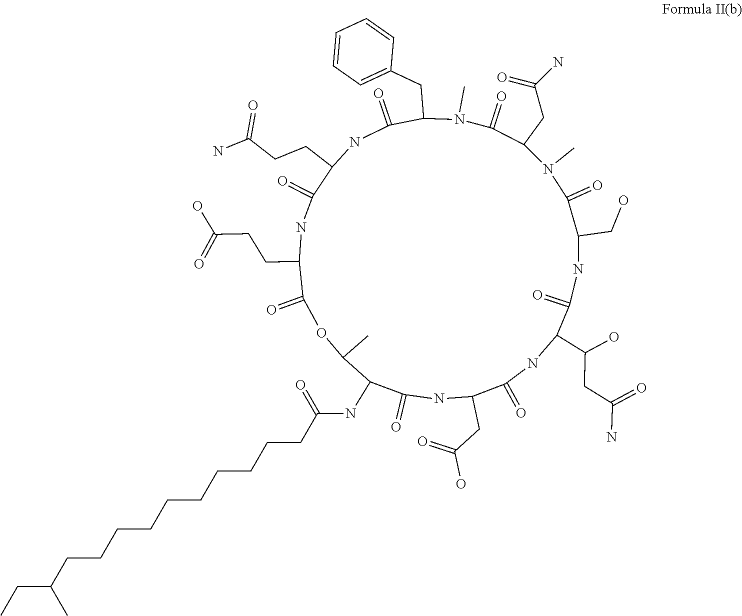 [SMILES: CCC(C)CCCCCCCCCCC(=O)NC1C(=O)NC(CC(=O)O)C(=O)NC(C(O)CC(N)=O)C(=O)NC(CO)C(=O)N(C)C(CC(N)=O)C(=O)N(C)C(Cc2ccccc2)C(=O)NC(CCC(N)=O)C(=O)NC(CCC(=O)O)C(=O)OC1C]

The lipopeptide of Formula II (b) comprises or has a molecular formula C₅₆H₈₇N₁₁O₁₉ and an exact mass of 1217.618 g. The lipopeptide according to Formula I (b) has a solubility in DMSO of above 10,000 ppm.

Preferably, the composition comprises a strain of Streptomyces, wherein the strain is a Streptomyces chrestomyceticus. Preferably, the composition comprises a strain of Streptomyces, suitably a Streptomyces chrestomyceticus, which comprises a genome sequence which has at least 91%, 92%, 93, 94%, preferably at least 95%, 96%, 97%, 98%, or at least 99% identity to the whole genome of Streptomyces chrestomyceticus NRRL-3672 or to the whole genome of Streptomyces sp. Saigon413 deposited with the Westerdijk Institute under accession number CBS149411. In one embodiment the composition comprises a Streptomyces chrestomyceticus which comprises a genome sequence which has 100% identity to the whole genome of Streptomyces sp. Saigon413 deposited with the Westerdijk Institute under accession number CBS149411. In one embodiment the composition comprises a Streptomyces chrestomyceticus which is Streptomyces sp. Saigon413 deposited with the Westerdijk Institute under accession number CBS149411

The microbial strain according to the present invention, or a strain of Streptomyces as disclosed herein, preferably comprises at least one nucleotide sequence that encodes a protein that has at least 80%, 85%, 90%, 95%, 96%, 97%, 98%, 99% identity or has 100% identity to an amino acid sequence according to SEQ ID NO: 71 to 115, preferably at least one nucleotide sequence that encodes a protein that has at least 80%, 85%, 90%, 95%, 96%, 97%, 98%, 99% identity or has 100% identity to an amino acid sequence according to SEQ ID NO: 91 and/or SEQ ID NO: 92.

In one embodiment, the microbial strain according to the present invention, or the strain of Streptomyces as disclosed herein, comprises at least one nucleotide sequence which has at least 80%, 85%, 90%, 95%, 96%, 97%, 98%, 99% identity or has 100% identity to at least one of the nucleotide sequence(s) of SEQ ID NO: 2 to 46. The nucleotide sequences according to SEQ ID NO: 2 to 46 comprise a gene cluster as shown in FIG. 11 for the production of a lipopeptide according to Formula II.

Preferably, the microbial strain according to the present invention, and/or a strain of Streptomyces, such as Streptomyces chrestomyceticus, as disclosed herein comprises at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty one, at least twenty two, at least twenty three, at least twenty four, at least twenty five, at least twenty six, at least twenty seven, at least twenty eight, at least twenty nine, at least thirty, at least thirty one, at least thirty two, at least thirty three, at least thirty four, at least thirty five, at least thirty six, at least thirty seven, at least thirty eight, at least thirty nine, at least forty, at least forty one, at least forty two, at least forty three, at least forty four, at least forty five of the nucleotide sequences of SEQ ID NO: 2, SEQ ID NO: 3, SEQ ID NO: 4, SEQ ID NO: 5, SEQ ID NO: 6, SEQ ID NO: 7 SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16; SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, or SEQ ID NO: 46 or which nucleotide sequence(s) has/have at least 80%, 85%, 90%, 95%, 96%, 97%, 98%, 99% identity thereto. Preferably, the microbial strain according to the present invention comprises a nucleotide sequence which has at least 80%, 85%, 90%, 95%, 96%, 97%, 98%, 99% identity or 100% identity to at least one of the nucleotide sequences according to SEQ ID NO: 22 and/or 23. Suitably, the microbial strain according to the present invention comprises a nucleotide sequence which has at least 80%, 85%, 90%, 95%, 96%, 97%, 98%, 99% identity or 100% identity to the nucleotide sequences according to SEQ ID NO: 22. Suitably, the microbial strain according to the present invention comprises a nucleotide sequence which has at least 80%, 85%, 90%, 95%, 96%, 97%, 98%, 99% identity or 100% identity to the nucleotide sequences according to SEQ ID NO: 23.

In another embodiment, the microbial strain according to the present invention, and/or a strain of Streptomyces, such as Streptomyces chrestomyceticus, as disclosed herein comprises at least one nucleotide sequence which encodes a protein which has at least 80%, 85%, 90%, 95%, 96%, 97%, 98%, 99% identity or which has 100% identity to at least one of the amino acid sequences according to SEQ ID NO: 116-139.

Preferably the microbial strain according to the present invention, and/or a strain of Streptomyces, such as Streptomyces chrestomyceticus, comprises or contains at least one, preferably at least two, preferably at least three, preferably at least four, preferably at least five, preferably at least at least six, preferably at least seven, preferably at least eight, preferably at least nine, preferably at least ten, preferably at least eleven, preferably at least twelve, preferably at least thirteen, preferably at least fourteen, preferably at least fifteen, preferably at least sixteen, preferably at least seventeen, preferably at least eighteen, preferably at least nineteen, preferably at least twenty, preferably at least twenty one, preferably at least twenty two, preferably at least twenty three, preferably all twenty four of the nucleotide sequences which encode a protein that has/have at least 80% identity, preferably at least 85%, preferably at least 90%, preferably at least 91%, 92%, 93%, 94%, 95%, 96%, 97%, 98%, preferably at least 99% identity, preferably 100% identity to the amino sequences according to SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, and/or SEQ ID NO: 139.

Preferably, the microbial strain according to the present invention, and/or a strain of Streptomyces, such as Streptomyces chrestomyceticus, as disclosed herein comprises at least one nucleotide sequence which encodes a protein which has at least 80%, 85%, 90%, 95%, 96%, 97%, 98%, 99% identity or which has 100% identity to at least one of the amino acid sequences according to SEQ ID NO: 136 and/or SEQ ID NO: 137. Suitably, the microbial strain according to the present invention, and/or a strain of Streptomyces, such as Streptomyces chrestomyceticus, as disclosed herein comprises a nucleotide sequence which encodes a protein which has at least 80%, 85%, 90%, 95%, 96%, 97%, 98%, 99% identity or which has 100% identity to the amino acid sequence according to SEQ ID NO: 136. Suitably, the microbial strain according to the present invention, and/or a strain of Streptomyces, such as Streptomyces chrestomyceticus, as disclosed herein comprises a nucleotide sequence which encodes a protein which has at least 80%, 85%, 90%, 95%, 96%, 97%, 98%, 99% identity or which has 100% identity to the amino acid sequence according to SEQ ID NO: 137.

In another embodiment, the microbial strain according to the present invention, and/or a strain of Streptomyces, such as Streptomyces chrestomyceticus, as disclosed herein comprises at least one nucleotide sequence which has at least 80%, 85%, 90%, 95%, 96%, 97%, 98%, 99% identity or 100% identity to at least one of the nucleotide sequence(s) of SEQ ID NO: 47 to 70. The nucleotide sequences according to SEQ ID NO: 47 to 70 comprise a gene cluster as shown in FIG. 12 for the production of a polyene which has a molecular formula according to C₆₇H₁₁₅NO₂₅, wherein the polyene is further characterized by the spectrum of light absorption as shown in FIG. 10. Preferably, the microbial strain according to the present invention and/or a strain of Streptomyces, such as Streptomyces chrestomyceticus, as disclosed herein comprises a nucleotide sequence which has at least 80%, 85%, 90%, 95%, 96%, 97%, 98%, 99% identity or 100% identity to one of the nucleotide sequences according to SEQ ID NO: 67 and/or 68. Suitably, the microbial strain according to the present invention comprises a nucleotide sequence which has at least 80%, 85%, 90%, 95%, 96%, 97%, 98%, 99% identity or 100% identity to the nucleotide sequences according to SEQ ID NO: 67. Suitably, the microbial strain according to the present invention comprises a nucleotide sequence which has at least 80%, 85%, 90%, 95%, 96%, 97%, 98%, 99% identity or 100% identity to the nucleotide sequences according to SEQ ID NO: 68.

Preferably, the microbial strain according to the present invention, and/or a strain of Streptomyces, such as Streptomyces chrestomyceticus, comprises at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty one, at least twenty two, at least twenty three, at least twenty four of the nucleotide sequences of SEQ ID NO: 47, SEQ ID NO: 48, SEQ ID NO: 49, SEQ ID NO: 50, SEQ ID NO: 51, SEQ ID NO: 52 SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61; SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, or SEQ ID NO: 70 or which nucleotide sequence(s) has/have at least 80%, 85%, 90%, 95%, 96%, 97%, 98%, 99% identity thereto.

The microbial strain or a strain of Streptomyces, or Streptomyces chrestomyceticus as disclosed herein may be a naturally occurring microorganism or a recombinant microorganism. Recombinant microorganisms can be produced by methods known to a person skilled in the art. A recombinant microorganisms may be produced by transforming the microorganism with at least one of the nucleotide sequences encoding a protein according the amino acids of SEQ ID NO: 71 to 115, and/or nucleotide sequences encoding a protein according the amino acids of SEQ ID NO: 116 to 139, preferably the nucleotide sequences according to SEQ ID NO: 2 to 46, preferably SEQ ID NO: 22 and or SEQ ID NO: 23 and/or the nucleotide sequences according to SEQ ID NO: 47 to 70, preferably SEQ ID NO: 67 and SEQ ID NO: 68.

Preferably the composition according to the present invention comprises an auxiliary, preferably an agricultural acceptable auxiliary. A composition as disclosed herein preferably is an agricultural-acceptable composition.

Suitable auxiliaries are known in the art and include for example solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers include, for example water, organic solvents, oils of vegetable or animal origin, cyclic and aromatic hydrocarbons, alcohols, esters, fatty acids, a glycol or any other suitable liquid carrier known in the art. The solvent or liquid carrier may be water or DMSO.

Suitable solid carriers include, for example ammonium salts, talc, titanium dioxide, pyrophyllite clay, silica, attapulgite clay, kieselguhr, chalk, diatomaceous earth, lime, calcium carbonate, bentonite clay, fuller's earth, cotton seed hulls, wheat flour, soybean flour, pumice, wood flour, walnut shell flour and lignin.

The amount of carrier may typically range from 0.9% to 99.99% by weight of the composition.

An adjuvant may be a surface-active agent, crystallisation inhibitor, viscosity modifier, suspending agents, spray droplet modifiers, pigments, antioxidants, foaming agents, anti-foaming agents, light-blocking agents, compatibilizing agents, sequestering agents, neutralising agents and buffers, corrosion inhibitors, dyes, odorants, spreading agents, penetration aids, micronutrients, emollients, lubricants and sticking agents. An adjuvant may for instance comprise an alkyl polyglucoside and/or polyoxyethylene (6) C9-C11 alcohol, or a methylcellulose.

A dispersant includes, but is not limited to, surfactants and wetting agents. In general, the dispersant(s) will have low toxicity for the microorganism(s) in the inoculant composition and for the plant part(s) to which the inoculant composition is to be applied.

A surfactant can be an ionic (cationic or anionic) or non-ionic surfactant, such as ionic or non-ionic emulsifier, foam formers known in the art, for instance acids such as polyacrylic acids, esters, ethers and the like.

A suitable surfactant comprises a polysorbate, for instance polysobate 20 such as Tween® 20. A composition as disclosed herein comprises an amount of surfactant of from 0.0005 wt/wt % to 0.5 wt/wt %, preferably from 0.001 to 0.1 wt/wt %, preferably from 0.002 to 0.08 wt/wt %, preferably from 0.004 to 0.06 wt/wt %, preferably from 0.005 to 0.04 wt/wt %, preferably from 0.006 to 0.025% wt/wt % of polysorbate 20.

In one embodiment, the composition as disclosed herein is a fermentation broth, preferably a spray-dried fermentation broth or a freeze-dried fermentation broth, or a formulation. Spray-drying or freeze-drying of a fermentation broth is known in the art.

The composition as disclosed herein comprises a cell count of the Streptomyces chrestomyceticus, from 1*10² to 1*10¹³ cfu/g dry weight, for instance from 1*10³ to 1*10¹² cfu/g dry weight, from 2*10³ to 2*10¹¹ cfu/g dry weight, from 5*10³ to 5*10¹¹ cfu/g dry weight, for instance from 1*10⁴ to 1*10¹⁰ cfu/g dry weight, from 2*10⁴ to 2*10¹⁰ cfu/g dry weight, such as from 1*10⁵ to 1*10⁹ cfu/g dry weight, from 2*10⁵ to 2*10⁹ cfu/g dry weight, from 5*10⁵ to 5*10⁹ cfu/g dry weight, from 1*10⁶ to 1*10⁸ cfu/g dry weight, such as from 2*10⁶ to 2*10⁸ cfu/g dry weight.

A composition as disclosed herein includes a formulation. Hence, a formulation comprises a composition as disclosed herein. A formulation may be any suitable composition for formulating a microbial strain such as Streptomyces sp., such as Streptomyces chrestomyceticus, as disclosed herein for instance Streptomyces sp. Saigon413 deposited with the Westerdijk Institute under accession number CBS149411.

Formulations of microbial strains are known in the art for instance as disclosed in Croda Crop Care, the Nouryon formulator toolbox and in: Formulation of Microbial Biopesticides: Beneficial microorganisms, nematodes and seed treatments (412 p., 6 Dec. 2012) eds. Burges H. D., Springer, ISBN 978-94-011-4926-6. Preferably, a composition as disclosed herein is a formulation, wherein a microbial strain as disclosed herein, such as Streptomyces chrestomyceticus is formulated as an oil dispersion (OD), a non-aqueous dispersion (NAD) or a flowable formulation.

Known formulations in the art are for instance emulsifiable concentratres, coatable pastes, sprayable or dilutable solutions or suspensions, powders, dusts, granulates and encapsulations.

Suspension concentrates are aqueous formulations in which finely divided solid particles of the active ingredient are suspended. Such formulations include anti-settling agents and dispersing agents and may further include a wetting agent to enhance pesticidal activity as well an anti-foam and a crystal growth inhibitor. In use, these concentrates are diluted in water and normally applied as a spray to the area to be treated. The amount of active ingredient may range from 0.5% to 95% of the concentrate.

Wettable powders are in the form of finely divided particles which disperse readily in water or other liquid carriers. The particles contain an active ingredient retained in a solid matrix. Typical solid matrices include fuller's earth, kaolin clays, silicas and other readily wet organic or inorganic solids. Wettable powders normally contain from 5% to 95% of an active ingredient plus a small amount of wetting, dispersing or emulsifying agent.

Emulsifiable concentrates are homogeneous liquid compositions dispersible in water or other liquid and may consist entirely of an active ingredient a liquid or solid emulsifying agent, or may also contain a liquid carrier, such as xylene, heavy aromatic naphthas, isophorone and other non-volatile organic solvents. In use, these concentrates are dispersed in water or other liquid and normally applied as a spray to the area to be treated. The amount of active ingredient may range from 0.5% to 95% of the concentrate.

Granular formulations include both extrudates and relatively coarse particles and are usually applied without dilution to the area in which treatment is required. Typical carriers for granular formulations include sand, fuller's earth, attapulgite clay, bentonite clays, montmorillonite clay, vermiculite, perlite, calcium carbonate, brick, pumice, pyrophyllite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or which can be coated with the active compound. Granular formulations normally contain 5% to 25% of active ingredients which may include surface-active agents such as heavy aromatic naphthas, kerosene and other petroleum fractions, or vegetable oils; and/or stickers such as dextrins, glue or synthetic resins.

An oil dispersion is a solid active ingredient dispersed in oil, which is a water immiscible carrier, An oil dispersion typically comprises (in wt %) active ingredient (5-60%) non-aqueous dispersants (1-10% of solids) aqueous dispersants (1-10% of solids) emulsifier (2-10% of iol) rheology modifier (0.2-5%), and an oil carrier to make up 100%. Suitable oils in an oil dispersion can be mineral oils, paraffinic oils, vegetable oils or methylated oils.

Suitable adjuvants, dispersants, emulsifiers and rheology modifiers in an oil dispersion depend on the type of oil and are known in the art. Suitable emulsifiers can be alcohol ethoxylates/alcoxylates, such as C16/18 ethoxylates, or C16/C18 alcoxylate, or block co-polymers. An adjuvant can be an alkyl polyglucoside. Suitable rheology modifiers can be clay, hydrogenated castor oil and derivatives, fumed silicam polyamides, polyesters or agrilan ODS.

An oil dispersion may comprise an adjuvant, wherein the adjuvant comprises alkyl polyglucoside and/or polyoxyethylene (6) C9-C11 alcohol. It was found that in a curative treatment of Zymoseptoria tritici and Puccinia recondita a composition according to the present invention formulated as an oil dispersion and an adjuvant, such as an adjuvant comprising alkyl polyglucoside and/or polyoxyethylene (6) C9-C11 alcohol resulted in improved reduction of the fungal disease

A non-aqueous dispersion is a liquid formulation wherein a solid ingredient is dispersed in a water-miscible carrier. The solid ingredient is uniformly suspended in the carrier but not dissolved.

A flowable formulation contains solid particles in a liquid which is usually water. A flowable concentration also comprises a suspension concentrate. A flowable formulation commonly comprises (in wt %) an active ingredient (5-60%), dispersants/wetting agent (1-10%), rheology modifiers (0.1-0.1%), biocides (0.1%), antifreezers (5-10%), antifoam (0.2%), adjuvants (up to 25%) and water to make up 100%.

As disclosed herein the isolated microbial strain or composition of the present invention can be advantageously in the form of a soluble concentrate (SL), or a flowable concentrate for seed treatment (FS), or a suspension concentrate (SC), and can be more preferably a seed treatment slurry to be applied onto seeds. Slurries for seed treatment applications are well-known in the art. For example, a slurry can contain the active ingredient(s), such as the isolated microbial strain or composition of the present invention, in the form of a commercially available product or not, mixed with water and optionally with at least one polymer, to optimize adhesion around the plant propagation material.

Each active ingredient can be applied to the plant propagation material from different compositions respectively, or the active ingredients can be gathered in the same composition (ready-mix composition). The composition may contain from about 0.001% to about 99% by weight of the active ingredient(s) over the total weight of the composition. Suitably, the composition contains from about 0.001% to about 60% by weight active ingredient(s) over the total weight of the composition.

A solid carrier can be a natural or synthetic solid material that is insoluble in water. This carrier is generally inert and acceptable in agriculture, especially on treated seed or other propagation material. It can be chosen, for example, from clay, diatomaceous earth, natural or synthetic silicates, titanium dioxide, magnesium silicate, aluminum silicate, talc, pyrophyllite clay, silica, attapulgite clay, kieselguhr, chalk, limestone, calcium carbonate, calcium montmorillonite, bentonite clay, Fuller's earth, cottonseed husks, wheat flour, soybean flour, pumice, wood flour, ground walnut shells, lignin, and the like.

Accordingly, the isolated microbial strain and/or the composition(s) according to the present invention can preferably adhere to the propagation material, such as a seed.

A composition according to the present invention comprises a dressing composition, which relates to a liquid composition useful for covering and/or wetting a plant propagation material, and more preferably a seed, at least in part or in totality.

The composition according to the present invention is particularly suited for dressing applications on plant propagation material, especially on seeds.

Typically, a tank-mix formulation for seed treatment application comprises 0.25% to 80% by weight, especially 1 to 75% by weight, of active ingredient(s), such as the isolated microbial strain or composition as disclosed herein, and 99.75% to 20% by weight, especially 99% to 25% by weight, of a solid or liquid auxiliary (including, for example, a solvent such as water), where the auxiliaries can be a surfactant in an amount of 0 to 40% by weight, especially 0.5 to 30% by weight, based on the total weight of the tank-mix formulation.

Typically, a pre-mix formulation for seed treatment application comprises 0.5 to 99.9% by weight, especially 1 to 95% by weight, of active ingredient(s), such as the isolated microbial strain or composition as disclosed herein, and 99.5 to 0.1% by weight, especially 99% to 5% % by weight, of a solid or liquid adjuvant (including, for example, a solvent such as water), where the auxiliaries can be a surfactant in an amount of 0 to 50% by weight, especially 0.5 to 40% by weight, based on the total weight of the pre-mix formulation.

Whereas commercial products will preferably be formulated as concentrates (e.g., pre-mix composition (formulation)), the end user will normally employ dilute formulations (e.g., tank mix composition).

Preferably, a composition, such as a formulation of Streptomyces chrestomyceticus as disclosed herein, for instance Streptomyces sp. Saigon413 deposited with the Westerdijk Institute under accession number CBS149411, comprises from 10 to 60 wt/wt % dry weight, preferably from 20 to 50 wt/wt % dry weight of spray-dried or freeze-dried fermentation broth of Streptomyces chrestomyceticus, such as Streptomyces sp. Saigon413 deposited with the Westerdijk Institute under accession number CBS149411.

In one embodiment, the isolated microbial strain and/or a composition of the present invention further comprises at least one additional active ingredient next to the strain of Streptomyces. such as Streptomyces chrestomyceticus, or isolated microbial strain and the composition as disclosed herein and used in the methods of the invention and applied simultaneously or sequentially with the microbial strain and/or compositions of the invention. An active ingredient as defined herein has fungicidal and/or insecticidal and/or herbicidal activity or has activity as plant growth regulator. The isolated microbial strain, or the composition of the present invention may be admixed with one or more additional ingredients having pesticidal activity such as fungicides, insecticides, herbicides, bactericides, acaricides, nematicides and/or the additional ingredient comprises plant growth regulators where appropriate. Pesticidal agents are referred to herein using their common name are known, for example, from “The Pesticide Manual”, 19th Ed., British Crop Protection Council, 2021.

An additional ingredient having pesticidal activity, for instance fungicidal activity may result in an unexpected synergistic activity. The additional ingredients having pesticidal activity and/or which is a plant growth regulator may be combined with the microbial strain or the composition of the invention and used in a method of the invention and applied simultaneously or sequentially with the composition of the invention. When applied simultaneously, these further ingredients may be formulated together with the compositions of the invention or mixed in, for example, a spray tank. As an alternative to directly admixing these further ingredients having pesticidal activity, the components may be used in separate fungicidal, insecticidal or herbicidal applications as part of a programme of fungal, insect or herbal control spread over part or all of a growing season.

The at least one additional ingredient having pesticidal activity and/or which is a plant growth regulator may be any suitable known fungicide, insecticide, herbicide and/or plant growth regulator. The at least one additional ingredient having pesticidal activity and/or plant growth regulator may be from chemical origin or biological origin, for instance from plant or microbial origin.

In addition, the compositions of the invention may also be applied with one or more systemically acquired resistance inducers (“SAR” inducer). SAR inducers are known and described in, for example, U.S. Pat. No. 6,919,298 and include, for example, salicylates and the commercial SAR inducer acibenzolar-S-methyl.

The isolated microbial strain or the composition according to the present invention may induce priming of plant resistance. Priming is a mechanism which leads to a physiological state that enables plants to respond more rapidly and/or more robustly after exposure to biotic or abiotic stress as described for instance in review article: P. Aranega-Bou et. al. Priming of plant resistance by natural compounds. Hexanoic acid as a model. Front. Plant. Sci. 1, October 2014.

A composition of the invention and at least one additional active ingredient is preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight. These mixtures can be used in a method for controlling pests, which comprises applying a composition comprising a mixture as described above to the pests or their environment, with the exception of a method for treatment of the human or animal body by surgery or therapy and diagnostic methods practised on the human or animal body.

A composition comprising a mixture of the isolated microbial strain or the composition of the invention, and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e., one after the other with a reasonably short period, such as a few hours or days.

In another aspect the present invention also relates to a process for producing the microbial strain according to the present invention as disclosed herein above, or the composition according to the present invention as disclosed herein above, comprising cultivating the microbial strain or a strain of Streptomyces, the Streptomyces chrestomyceticus in a suitable fermentation medium under suitable fermentation conditions, and optionally comprising a step of recovering the microbial strain or composition. Usually a fermentation broth is produced during cultivation of or when cultivating the microbial strain or strain of Streptomyces, for instance a Streptomyces chrestocmyceticus strain. Suitable fermentation conditions for cultivating Streptomyces sp. are known to a person skilled in art.

Cultivating a microbial strain as disclosed herein, or a strain of Streptomyces, for instance Streptomyces chrestomyceticus strain as disclosed herein comprises cultivating the microbial strain under aerobic conditions at a temperature of from 15 degrees Celsius to 45 degrees Celsius, preferably a temperature of from 20 to 35 degree Celsius, preferably a temperature of between 25 to 32 degrees Celsius, in the presence of a carbon source and an nitrogen source. A suitable carbon source may be molasses, such as beet or cane molasses, polysaccharides, flour, starch, sugar, or glucose. A suitable nitrogen source may be casein hydrolysate, tryptone, ammonium sulphate, ammonia, yeast extract, peptone or urea. The process for producing a microbial strain according to the present invention or the S. chrestomyceticus may be performed in a batch, fed-batch or continuous culture.

Preferably, the process according to the present invention comprises cultivating a microbial strain or a strain of Streptomyces, suitably Streptomyces chrestomyceticus, as disclosed herein, wherein the microbial strain or the strain of Streptomyces, suitably Streptomyces chrestomyceticus, produces malonomicin and at least one, at least two, at least three, at least four, or at least 5 of the compounds selected from the group consisting of the compounds selected from cyclothiazomycin C, streptimidone, an oligosaccharide compound according to compound I comprising a molecular formula according to C₅₃H₉₀N₂O₄₄, further characterised by the NMR spectra listed in Table 1 and Table 2, preferably wherein compound I is further characterized by a structural Formula I, and a lipopeptide according to Formula II, or a salt thereof wherein R₁═CH₃ or C₂H₅, and a polyene compound characterized by a molecular formula according to C₆₇H₁₁₅NO₂₅, wherein the polyene is further characterized by the spectrum of light absorption as shown in FIG. 10

Suitably, the process according to the present invention comprises cultivating a microbial strain or a strain of Streptomyces, for instance a Streptomyces chrestomyceticus, wherein the microbial strain or the strain of Streptomyces produces at least one, at least two, or at least three of the compounds selected from the group consisting of malonomicin, cyclothiazomycin C, streptimidone, and at least one, at least two or at least three of the compounds selected from an oligosaccharide compound according to compound I comprising a molecular formula according to C₅₃H₉₀N₂O₄₄, further characterised by the NMR spectra listed in Table 1 and Table 2, preferably wherein compound I is further characterized by a structural Formula I, and a lipopeptide according to Formula II, or a salt thereof wherein R₁═CH₃ or C₂H₅, and a polyene compound characterized by a molecular formula according to C₆₇H₁₁₅NO₂₅, wherein the polyene is further characterized by the spectrum of light absorption as shown in FIG. 10. Preferably, the microbial strain or the strain of Streptomyces, for instance Streptomyces chrestomyceticus, produces the compounds selected from the group consisting of malonomicin, cyclothiazomycin C, streptimidone and a compound according to compound I comprising a molecular formula according to C₅₃H₉₀N₂O₄₄, further characterised by the NMR spectra listed in Table 1 and Table 2, preferably wherein the compound I is further characterized by a structural Formula I, and a lipopeptide according to Formula II, or a salt thereof wherein R₁═CH₃ or C₂H₅, and a polyene compound characterized by a molecular formula according to C₆₇H₁₁₅NO₂₅, wherein the polyene is further characterized by the spectrum of light absorption as shown in FIG. 10.

The process according to the present invention may further comprise a step of recovering the microbial strain or composition according to the present invention. Recovering a microbial strain or the composition according to the present invention may comprise centrifugating or filtering the fermentation broth. Preferably, recovering comprises a step of drying the fermentation broth, such as by spray drying of freeze drying. Spray drying or freeze drying are methods known to a person skilled in the art. The composition comprises compounds malonomicin, cyclothiazomycin C, streptimidone, an oligosaccharide compound according to compound I comprising a molecular formula according to C₅₃H₉₀N₂O₄₄, further characterised by the NMR spectra listed in Table 1 and Table 2, preferably wherein compound I is further characterized by a structural Formula I, a lipopeptide according to Formula II, or a salt thereof wherein R₁═CH₃ or C₂H₅, and/or a polyene compound characterized by a molecular formula according to C₆₇H₁₁₅NO₂₅, wherein the polyene is further characterized by the spectrum of light absorption as shown in FIG. 10 which may be recovered by suitable methods known in the art, for instance via crystallization or chromatography, eg HPLC.

The embodiments of the microbial strain of the present invention and the strain of Streptomyces and, for instance a Streptomyces chrestomyceticus, as disclosed herein above are also applicable for the microbial strain, the strain of Streptomyces and the Streptomyces chrestomyceticus in the process and method of the present invention.

The process according to the present invention may further comprise a step of formulating the microbial strain or composition according to the present invention into a suitable formulation or composition as defined herein above.

In another aspect the present invention relates to a method for controlling or preventing infestation of a plant, plant propagation material and/or harvested food, non-food and feed crops by a phytopathogenic microorganism, by treating the plant, plant propagation material and/or harvested food crops, by applying an effective amount of Streptomyces chrestomyceticus, the microbial strain, or a composition of the present invention to the plant, to a part thereof or a locus thereof, the plant propagation material and/or harvested food crops. Surprisingly, it was found that Streptomyces chrestomyceticus, such as a microbial strain according to the present invention, or the composition according to the present invention was very effective in treating phytopathonic microorganisms on plants. The Streptomyces chrestomyceticus, such as a microbial strain according to the present invention, or the composition according to the present invention was surprisingly active as a pesticide and against fungi, bacteria and oomycetes.

The method according to the present does not include a method for treatment of the human or animal body by surgery or therapy and diagnostic methods practised on the human or animal body.

The term “plant” refers to all physical parts of a plant, including seeds, seedlings, saplings, roots, tubers, stems, stalks, foliage, and fruits. Germinated plants and young plants which are to be transplanted after germination or after emergence from the soil, may also be mentioned. These young plants can be protected before transplantation by a total or partial treatment by immersion.

The term “plant propagation material” is understood to denote generative parts of the plant, such as seeds, which can be used for the multiplication of the latter, and vegetative material, such as cuttings or tubers, (for example potatoes), roots, fruits, bulbs, rhizomes or parts of plants. The plant propagation material can be treated with the isolated microbial strain or composition of the invention before the material is sown or planted. Alternatively, the plant propagation material may be treated with the isolated microbial strain or composition of the invention during sowing or planting. Additionally, the the isolated microbial strain or composition the invention may be applied to the previously treated propagation material before or during its planting. The the isolated microbial strain or composition of the invention may be applied during the sowing of the seed. The the isolated microbial strain or also be used to plant propagation material derived from plants grown in a green house and/or during transplantation. More preferably the plant propagation material is plant seeds.

The term plants involve “useful plants” or “crops”. The wording “Useful plants” and “crops” are used interchangeably herein. “Useful plants” and “crops” comprise perennial and annual crops, such as berry plants for example blackberries, blueberries, cranberries, raspberries and strawberries; cereals for example barley, maize (corn), millet, oats, rice, rye, sorghum triticale and wheat; fibre plants for example cotton, flax, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes. The term “plants” also includes wood crops, such as pine trees, or woody plants.

The term “useful plants” is to be understood as also including useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering.

The term “useful plants” is to be understood as also including useful plants which have been so transformed by the use of recombinant DNA techniques that they are capable of synthesising one or more selectively acting toxins, such as are known, for example, from toxin-producing bacteria, especially those of the genus Bacillus.

The term “locus” as used herein means fields in or on which plants are growing, or where seeds of cultivated plants are sown, or where seed will be placed into the soil. It includes soil, seeds, and seedlings, as well as established vegetation.

Any suitable plant, plant propagation material or food or feed crop may be treated in a method according according to the present invention as defined herein. Preferably the plant, plant propagation material or food crop comprises or is wheat, barley, rice, corn, soya, sugar beet banana, tomato, cucumber, and/or groundnut.

Phytopathogenic microorganisms that are affected by the isolated microbial strain or the composition according to the present invention are fungi and fungal vectors of disease as well as phytopathogenic bacteria and viruses. Phytopathogenic microorganisms in a method according to the present invention include the following fungi and fungal vectors of disease and phytopathogenic bacteria:

Absidia corymbifera, Albugo candida, Alternaria spp. including A. solani, Aphanomyces spp, Ascochyta spp, Aspergillus spp. including A. flavus, A. fumigatus, A. nidulans, A. niger, A. terrus, Aureobasidium spp. including A. pullulans, Bacillus subtilis, Blastomyces dermatitidis, Blumeria graminis, Blumeriella jaapii, Botryosphaeria spp. including B. dothidea, B. obtusa, Botrytis spp. including B. cinerea, Bremia lactucae, Cadophora gregata, Candida spp. including C. albicans, C. glabrata, C. krusei, C. lusitaniae, C. parapsilosis, C. tropicalis, Cephaloascus fragrans, Ceratocystis spp, Cercospora spp. including C. arachidicola, C. beticola, C. kikuchii, C. sojina, Cercosporidium personatum, Cladosporium spp, Clarireedia homoeocarpa, Clavibacter spp, Claviceps purpurea, Coccidioides immitis, Cochliobolus spp, Colletotrichum spp. including C. dematium, C. lindemuthianum, C. musae, C. orbiculare, C. truncatum, Corynespora cassiicola, Cryptococcus neoformans, Diaporthe spp, Dickeya zeae, Didymella spp, Drechslera spp, Elsinoe spp, Epidermophyton spp, Eremothecium gossypiim, Erwinia spp. including E. amylovora, E. carotovora, Erysiphe spp. including E. cichoracearum, E. necator, Eutypa lata, Fusarium spp. including F. culmorum, F. graminearum, F. langsethiae, F. moniliforme, F. oxysporum, F. poae, F. proliferatum, F. pseudograminearum, F. sacchari, F. sambucinum, F. subglutinans, F. solani, F. sporotrichioides, F. tricinctum, F. virguliforme, Gaeumannomyces graminis, Gibberella spp. including G. avenacea, G. fujikuroi, G. intricans, G. moniliformis, G. zeae, Gloeodes pomigena, Gloeosporium musarum, Glomerella cingulate, Golovinomyces cichoracearum, Gymnosporangium juniperi-virginianae, Guignardia bidwellii, Gymnosporangium juniperi-virginianae, Helminthosporium spp, Hemileia spp, Histoplasma spp. including H. capsulatum, Hyaloperonospora parasitica, Kabatiella zeae, Laetisaria fuciformis, Leptographium lundbergii, Leveillula taurica, Lophodermium seditiosum, Microdochium majus, Microdochium nivale, Microsporum spp, Monilinia spp. including M. fructicola, Monographella spp including M. nivalis, Mucor spp, Mycosphaerella spp. including M. arachidis, M. fijiensis, M. graminicola, M. pomi, Nakataea oryzae, Neopseudocercosporella spp, Oculimacula spp, Oncobasidium theobromaeon, Ophiostoma spp, Pantoea stewartia, Paracoccidioides spp, Parastagonospora nodorum, Pectobacterium spp, Penicillium spp. including P. digitatum, P. italicum, Petriellidium spp, Peronosclerospora spp. including P. maydis, P. philippinensis and P. sorghi, Peronospora spp including P. destructor, Phaeosphaeria nodorum, Phakopsora pachyrhizi, Phellinus igniarus, Phialophora spp, Phlyctema vagabunda, Phoma spp, Phomopsis viticola, Phyllachora pomigena, Phyllosticta spp, Physoderma maydis, Phytophthora spp. including P. capsica, P. infestans, Plasmodiophora brassicae, Plasmopara spp. including P. halstedii, P. viticola, Plenodomus spp, Pleospora spp., Podosphaera spp. including P. leucotricha, Polymyxa graminis, Polymyxa betae, Pseudocercospora fijiensis, Pseudocercosporella herpotrichoides, Pseudomonas spp. including P. syringae, Pseudoperonospora spp. including P. cubensis, P. humuli, Pseudopeziza tracheiphila, Pseudopyrenochaeta lycopersici, Puccinia spp. including P. hordei, P. recondita, P. striiformis, P. triticina, Pyrenopeziza spp, Pyrenophora spp, Pyricularia spp. including P. oryzae, Pythium spp. including P. ultimum, Ralstonia solanacearum, Ramularia spp, Rathayibacter spp, Remotididymella destructiva, Rhizoctonia spp, Rhizomucor pusillus, Rhizopus arrhizus, Rhynchosporium spp, Robbsia andropogonis, Sarocladium oryzae, Scedosporium spp. including S. apiospermum and S. prolificans, Schizothyrium pomi, Sclerophthora macrospora, Sclerotinia spp. including S. sclerotiorum, Sclerotium spp, Septoria spp, including S. nodorum, S. tritici, Setosphaeria turcica, Sphaerotheca macularis, Sphaerotheca fusca (Sphaerotheca fuliginea), Spiroplasma kunkelii, Sporothorix spp, Stagonospora nodorum, Stagonosporopsis cucurbitacearum, Stemphylium spp, Stenocarpella macrospora, Stereum hirsutum, Streptomyces spp, Thanatephorus cucumeris, Thielaviopsis basicola, Tilletia spp, Tranzschelia discolor, Trichoderma spp. including T. harzianum, T. pseudokoningii, T. viride, Trichophyton spp, Typhula spp, Uncinula necator, Urocystis spp, Uromyces spp, Ustilago spp, Venturia spp. including V. inaequalis, Verticillium spp, Wilsonomyces carpophilus, or Xanthomonas spp, including X. oryzae and X. campestris, Xylella spp, Zymoseptoria tritici.

Phytopathogenic microorganisms that were found to be surprisingly affected by the Streptomyces chrestomyceticus, microbial strain or composition according to the present invention are fungi, for instance fungi belonging to the genus of Zymoseptoria, Puccinia, Mycorsphearella, Pyricularia, Rhizoctoonia, Blumeria, Alternaria, Colletotrichum, Ramularia, Parastagonospora, Rhynchosporium, Oculimacula, Fusarium, Gaeumannomyces, Botrytis, or Sclerotinia, preferably a fungus belonging tot he species Zymoseptoria tritici, Puccinia recondita, Puccinia striiformis, Mycorsphearella fijiensis, Mycorsphearella arachidis Pyricularia oryzae, Rhizoctonia solani, Blumeria graminis f.sp. tritici, Alternaria solani, Colletotrichum lagenarium, Ramularia collo-cygni, Parastagonospora nodorum, Rhynchosporium secalis, Oculimacula yallandae, Fusarium avenaceum, F. graminearum, F. culmorum, F. virguliforme, F. subglutinans, F. pseudograminearum, F. verticillioides, F. fujikuroi, F subglutinans, F. oxysporum, for instance, F. oxysporum f. sp. cubense, F. oxysporum f. sp. melonis, F. oxysporum f. sp. vasinfectum, F. oxysporum f. sp. lycopesici, Gaeumannomyces graminis Botrytis cinerea, or Sclerotinia sclerotiorum, or bacteria, such as a bacterium belonging to the genus Xanthomonas, preferably Xanthomonas oryzae pv. oryzae or oomycetes, such as an oomycete belonging to Aphanomyces, preferably Aphanomyces cochlioides.

Controlling or preventing means reducing infestation by phytopathogenic microorganisms especially fungi, to such a level that an improvement is demonstrated.

Suitably, the Streptomyces chrestomyceticus or the isolated microbial strain or a composition of the invention are applied either preventative, meaning prior to disease development or curative, meaning after disease development. It was found surprisingly found that both preventive and curative application of a composition according to the present invention resulted in reduction of infestation by phytopathogenic microorganisms.

A preferred method of controlling or preventing an infestation of a plant by phytopathogenic microorganisms, especially fungi, or insects comprises foliar application of the Streptomyces chrestomyceticus as disclosed herein or isolated microbial strain or composition according to the present invention. The frequency of application and the rate of application will depend on the risk of infestation by the corresponding pathogen or insect. However, the Streptomyces chrestomyceticus as disclosed herein or isolated microbial strain or composition according to the present invention can also penetrate the plant through the roots via the soil (systemic action) by drenching the locus of the plant with a liquid formulation, or by applying the the Streptomyces chrestomyceticus as disclosed herein or isolated microbial strain or composition according to the present invention in solid form to the soil, e.g. in granular form (soil application). In crops of water rice such granulates can be applied to the flooded rice field.

Phytopathogenic microorganisms that were found to be surpisingly affected, for instance in foliar application, by the Streptomyces chrestomyceticus, microbial strain or composition according to the present invention are fungi belonging to Zymoseptoria, Puccinia, Mycorsphearella, Pyricularia, Rhizoctoonia, Blumeria, Alternaria, Colletotrichum, Ramularia, Parastagonospora, Rhynchosporium, Oculimacula, Fusarium, Gaeumannomyces sp., Botrytis, or Sclerotinia, preferably a fungus belonging to Zymoseptoria tritici, Puccinia recondita, Puccinia striiformis, Mycorsphearella fijiensis, Mycorsphearella arachidis Pyricularia oryzae, Rhizoctoonia solani, Xanthomonas oryzae pv. oryzae, Blumeria graminis f.sp. tritici, Alternaria solani, Colletotrichum lagenarium, Ramularia collo-cygni, Parastagonospora nodorum, Rhynchosporium secalis, Oculimacula yallandae, Fusarium avenaceum, F. graminearum, or bacteria belonging to Xanthomonas, preferably Xanthomons oryzae.

In one embodiment there is disclosed a method for controlling or preventing infestation of a plant, plant propagation material and/or harvested food crops by a phytopathogenic microorganism, by treating the plant, plant propagation material and/or harvested food crops, wherein an effective amount of Streptomyces chrestomyceticus, the microbial strain of the present invention, or a composition as disclosed herein is applied to the plant, to a part thereof or a locus thereof, the plant propagation material and/or harvested food crops, wherein the phytopathogenic microorganism are fungi belonging to Zymoseptoria, Puccinia, Mycorsphearella, Pyricularia, Rhizoctoonia, Xanthomonas, Blumeria, Alternaria, Colletotrichum, Ramularia, Parastagonospora, Rhynchosporium, Oculimacula, Fusarium, Gaeumannomyces sp., Botrytis, or Sclerotinia, preferably a fungus belonging to Zymoseptoria tritici, Puccinia recondita, Puccinia striiformis, Mycorsphearella fijiensis, Mycorsphearella arachidis Pyricularia oryzae, Rhizoctoonia solani, Xanthomonas oryzae pv. oryzae, Blumeria graminis f.sp. tritici, Alternaria solani, Colletotrichum lagenarium, Ramularia collo-cygni, Parastagonospora nodorum, Rhynchosporium secalis, Oculimacula yallandae, Fusarium avenaceum, or F. graminearum, and wherein the plant comprises wheat, barley, rice, corn, soya, sugar beet, banana, tomato, cucumber, and/or groundnut.

In one embodiment the method of controlling or preventing an infestation of a plant by phytopathogenic microorganisms, comprises the application of the Streptomyces chrestomyceticus as disclosed herein or isolated microbial strain or composition according to the present invention, wherein the plant is wheat and the phytopathogenic microorganisms are fungi belonging to Zymoseptoria, Puccinia, Blumaria, Parastagonospora Oculimacula, preferably Zymoseptoria tritici, Puccinia recondita, Puccinia striiformi, Blumeria graminins f.sp. tritici Parastagonospora nodorum, Oculimacula yallandae, Fusarium avenaceum, or F. graminearum.

In one embodiment the method of controlling or preventing an infestation of a plant by phytopathogenic microorganisms, comprises the application of the Streptomyces chrestomyceticus as disclosed herein or isolated microbial strain or composition according to the present invention, wherein the plant is barley and the phytopathogenic microorganisms are fungi belonging to Ramularia sp, Rhynchosporium sp, preferably Ramularia collo-cygni or Rhynchosporium secalis.

In one embodiment the method of controlling or preventing an infestation of a plant by phytopathogenic microorganisms, comprises the application of the Streptomyces chrestomyceticus as disclosed herein or isolated microbial strain or composition according to the present invention, wherein the plant is banana and the phytopathogenic microorganisms is a fungus belonging to Mycorsphearella, preferably Mycorsphearella fijiensis.

In one embodiment the method of controlling or preventing an infestation of a plant by phytopathogenic microorganisms, comprises the application of the Streptomyces chrestomyceticus as disclosed herein or isolated microbial strain or composition according to the present invention, wherein the plant is rice and the phytopathogenic microorganisms is a fungus belonging to Pyricularia, Rhizoctoonia, preferably Pyricularia oryzae, Rhizoctoonia solani, or a bacteria belonging to Xanthomonas, preferably Xanthomonas oryzae pv. oryzae.

In one embodiment the method of controlling or preventing an infestation of a plant by phytopathogenic microorganisms, comprises the application of the Streptomyces chrestomyceticus as disclosed herein or isolated microbial strain or composition according to the present invention, wherein the plant is tomato and the phytopathogenic microorganisms is a fungus belonging to Alternaria sp. preferably Altemaria solani.

In one embodiment the method of controlling or preventing an infestation of a plant by phytopathogenic microorganisms, comprises the application of the Streptomyces chrestomyceticus as disclosed herein or isolated microbial strain or composition according to the present invention, wherein the plant is cucumber and the phytopathogenic microorganisms is a fungus belonging to Colletotrichum sp. preferably Colletotrichum lagenarium.

In one embodiment the method of controlling or preventing an infestation of a plant by phytopathogenic microorganisms, comprises the application of the Streptomyces chrestomyceticus as disclosed herein or isolated microbial strain or composition according to the present invention, wherein the plant is ground nut and the phytopathogenic microorganisms is a fungus belonging to Mycorsphearella sp. preferably, Mycorsphearella arachidis.

It was also surprisingly found that the Streptomyces chrestomyceticus as disclosed herein or isolated microbial strain or composition according to the present invention is mobile throughout the stem or leaves and showed fungicidal activity on other parts of the plants than where it was applied.

It is also possible to use isolated microbial strain and/or composition according to the present invention as dressing agent for the treatment of plant propagation material, e.g., seed, such as fruits, tubers or grains, or plant cuttings, for the protection against fungal infections as well as against phytopathogenic pests occurring in the soil. The propagation material can be treated with the isolated microbial strain and/or composition according to the present invention before planting: seed, for example, can be dressed before being sown. The isolated microbial strain and/or composition according to the present invention can also be applied to grains (coating), either by impregnating the seeds in a liquid formulation or by coating them with a solid formulation. The isolated microbial strain and/or composition according to the present invention can also be applied to the planting site when the propagation material is being planted, for example, to the seed furrow during sowing. Disclosed herein are such methods of treating plant propagation material and the plant propagation material so treated.

The Streptomyces chrestomyceticus as disclosed herein or isolated microbial strain or composition according to the present invention may also be applied to seeds by impregnating the seeds or tubers either with a liquid formulation or coating them with a solid formulation. Surprisingly, it was found that the Streptomyces chrestomyceticus as disclosed herein or isolated microbial strain or composition according to the present invention was effective in reducing fungal and oomycete infections on seeds.

In one embodiment the method of controlling or preventing an infestation of plants by phytopathogenic microorganisms which comprises treating seeds, wherein an effective amount of Streptomyces chrestomyceticus, the microbial strain of the present invention, or a composition as disclosed herein is applied to the seeds, wherein the phytopathegenic microorganisms are selected from Fusarium avenaceum, F. graminearum, F. culmorum, F. virguliforme, F. subglutinans, F. pseudograminearum, F. verticillioides, F. fujikuroi, F subglutinans, F. oxysporum, for instance, F. oxysporum f. sp. cubense, F. oxysporum f. sp. melonis, F. oxysporum f. sp.vasinfectum, F. oxysporum f. sp. lycopesici, Gaeumannomyces graminis Botrytis cinerea, or Sclerotinia sclerotiorum and oomycetes, such as an oomycete belonging to Aphanomyces, preferably Aphanomyces cochlioides.

Preferably, the seeds are from barley, wheat, corn, sugar beat or soya. The wording soya, soy or soybean are used interchangeable herein.

The seed treatment can occur to an unsown seed, and the term “unsown seed” is meant to include seed at any period between the harvest of the seed and the sowing of the seed in the ground for the purpose of germination and growth of the plant. Treatment to an unsown seed is not meant to include those practices in which the microbial strain or composition of the presen is applied to the soil but would include any application practice that would target the seed during the sowing/planting process.

In one aspect, the present invention relates to a plant, or a plant propagation material treated with the isolated microbial strain or composition according to the present invention.

The treated plant propagation material of the present invention can be treated in the same manner as conventional plant propagation material. The treated propagation material can be stored, handled, sown and tilled in the same manner as any other pesticide treated material.

Although it is believed that the present method can be applied to a seed in any physiological state, it is preferred that the seed be in a sufficiently durable state that it incurs no damage during the treatment process. Typically, the seed would be a seed that had been harvested from the field; removed from the plant; and separated from any cob, stalk, outer husk, and surrounding pulp or other non-seed plant material. The seed would preferably also be biologically stable to the extent that the treatment would cause no biological damage to the seed. It is believed that the treatment can be applied to the seed at any time between harvest of the seed and sowing of the seed or during the sowing process (seed directed applications).

The techniques of seed treatment application are well known to those skilled in the art, and they may be used readily in the context of the present invention.

One method of applying the composition according to the invention consists in spraying or wetting the plant propagation material with an aqueous liquid formulation or mixing the plant material with such liquid formulation. Also, before the application, the composition of the invention may be diluted with water by simple mixing at ambient temperature in order to prepare an on-farm seed treatment formulation.

In one embodiment, the method comprises applying an effective amount of the Streptomyces chrestomyceticus, or the isolated microbial strain, or of the composition according to the present invention as disclosed herein above, wherein the effective amount comprises from 2*10² to 5*10¹⁷, from 3*10² to 5*10¹⁶, from 5*10² to 5*10¹⁵, from 2*10² to 5*10¹⁴, from 2*10² to 5*10¹³, preferably 5*10² to 5*10¹², 1*10³ to 5*10¹¹, from 5*10³ to 1*10¹¹, from 1*10⁴ to 5*10¹⁰, from 5*10⁴ to 1*10¹⁰, from 1*10⁵ to 5*10⁹, from 5*10⁵ to 1*10⁹, from 1*10⁶ to 5*10⁸, from 5*10⁶ to 1*10⁸ colony forming unit (cfu) of the Streptomyces chrestomyceticus, or of the isolated microbial strain or the composition per hectare.

An effective amount of Streptomyces chrestomyceticus or the isolated microbial strain, or of the composition according to the present invention as disclosed herein above, comprises from 0.1 g to 10 kg/per hectare (ha), such as from 0.5 g to 5 kg, such as from 1 g to 1 kg/ha, such as from 5 g to 500 g/ha, such as from 10 g to 200 g/ha, such as from 50 to 100 g/ha. The weight in g and kg is dry weight of the Streptomyces chrestomyceticus, microbial strain or composition.

In one embodiment the method according to the present invention comprises treating plant propagation material wherein the plant propagation material is seed and the effective amount comprises 5×10² to 5×10¹⁵, 2×10³ to 5×10¹⁴, from 5×10³ to 5×10¹³, from 2×10⁵ to 5×10¹², preferably 5*10² to 5*10¹², 1*10³ to 5*10¹¹, from 5*10³ to 1*10¹¹, from 1*10⁴ to 5*10¹⁰, from 5*10⁴ to 1*10¹⁰, from 1*10⁵ to 5*10⁹, from 5*10⁵ to 1*10⁹, from 1*10⁶ to 5*10⁸, from 5*10⁶ to 1*10⁸ colony forming unit (cfu) of the Streptomyces chrestomyceticus or the isolated microbial strain or the composition according to the present invention per kg of seed.

When the plant propagation material is seed, an effective amount of Streptomyces chrestomyceticus or the isolated microbial strain, or of the composition according to the present invention as disclosed herein above, may also comprise from 0.0001 g to 100 g/per kg of seeds, such as from 0.0005 g to 80 g/kg seeds, such as from 0.001 g to 50 g/kg seeds, such as from 0.005 g to 10 g/kg seeds, The weight in g dry weight of the Streptomyces chrestomyceticus or the microbial strain, or composition per kg dry weight of seeds.

In another aspect, the present invention relates to the use of Streptomyces chrestomyceticus or the isolated microbial strain or the composition according to the present invention as a pesticide, preferably as a fungicide.

In another aspect the present invention relates to the use of a microbial strain of the present invention or a Streptomyces which has at least 91% identity to the whole genome of Streptomyces chrestomyceticus NRRL B-3672, or at least 91% identity to the whole genome of Streptomyces sp. Saigon413 deposited with the Westerdijk Institute under accession number CBS149411 as disclosed herein for producing malonomicin and at least one, at least two, at least three, at least four or at least five of the compounds selected from the group consisting of cyclothiazomycin C, streptimidone, an oligosaccharide compound according to compound I, comprising a molecular formula according to C₅₃H₉₀N₂O₄₄, further characterised by the NMR spectra listed in Table 2 and Table 3, preferably characterized by a structural Formula I, a lipopeptide according to Formula II, or a salt thereof wherein R1=CH₃ or C₂H₅, and a polyene compound characterized by a molecular formula according to C₆₇H₁₁₅NO₂₅, wherein the polyene is further characterized by the spectrum of light absorption as shown in FIG. 10.

The features related to the isolated microbial strain, composition according to the present invention, the strain of Streptomyces, and Streptomyces chrestomyceticus are as disclosed herein above and applicable to the use thereof.

### FIGURES

FIG. 1. 1D ¹H NMR spectrum of the compound I in D₂O at 600 MHz

FIG. 2. 1D ¹³C NMR spectrum of the compound I in D₂O at 600 MHz

FIG. 3. 2D Dept Edited 1H-13C HSQC NMR Spectrum of the compound I in D₂O.at 600 MHz showing the positive (CH) signals

FIG. 4. 2D Dept Edited 1H-13C HSQC NMR Spectrum of the compound I in D₂O.at 600 MHz showing the negative (CH₂) signals

FIG. 5. Spectrum of light absorption (UV-VIS) 200-400 nm of a lipopeptide according to Formula II (a), Formula II (b) or Lipopeptin A

FIG. 6. LC-ESI-MS/MS spectrum of precursor 1204.6 m/z (M+H)⁺ for a lipopeptide of Formula II (a) depicting fragment peaks consistent with amino acids: aspartic acid, hydroxy-glutamine, serine, methyl-asparagine, methyl-phenylalanine

FIG. 7. LC-ESI-MS/MS/MS spectrum of precursor 294.2 m/z for a lipoeptide of Formula II (a) depicting peaks consistent with the molecule C14H25-OH2-C₄H₅ON

FIG. 8. The upfield region of the 1D 1H NMR spectrum of a lipopeptide according to Formula II (a) in CD₃OD at 600 MHz

FIG. 9. The downfield region of the 1D 1H NMR spectrum of a lipopeptide according to Formula II (a) in CD₃OD at 600 MHz

FIG. 10. Spectrum of light absorption (UV-VIS) 200-500 nm of polyene compound

FIG. 11. Graphical presentation of the lipopeptide gene cluster

FIG. 12. Graphical presentation of the polyene gene cluster

### EXAMPLES

### Example 1

### Example 1.1. Source and Fermentation

### Fermentation of Streptomyces sp.

Streptomyces species were ordered from culture collections disclosed in Table 1. Streptomyces sp. Saigon413 was deposited at the Westerdijk institute under accession number CBS149411. The deposit was made by Syngenta Ltd., Jealott's Hill Research International Centre, Bracknell, Berkshire, RG42 6EY, UK under the terms of the Budapest Treaty on the International Recognition of the Deposit of Microorganisms for the Purposes of Patent Procedure. Streptomyces species were cultivated in Erlenmeyer flasks with a liquid medium consisting of (g/I) casein hydrolysate 10, glucose 40, K₂HPO₄ 1.25, soytone 2, tryptone, 8 and incubated at 28 °C in an incubator shaking 150 rpm with 25 mm throw for 4 days.

### Large Scale Fermentations

For large scale production, standard procedures were applied for cultivating Streptomyces sp., such as Streptomyces chrestomyceticus NRRL 3672 and Streptomyces chrestomyeticus CBS149411 to high cell density using fed-batch fermentation. After harvesting, the broth was spray dried or freeze dried according to methods known to a person skilled in the art.

The final product (TGAI) after spray- or freeze drying had a cell count of 1*10⁶ to 1*10¹³ CFU/g dry mass. TGAI: technical grade active ingredient (unformulated product).

### Example 1.2. Formulations

For the preparation of different formulations of the spray-dried or freeze-dried product (microorganism), general formulation technology was used as disclosed on Croda Crop Care, the Nouryon formulator toolbox and in: Formulation of Microbial Biopesticides: Beneficial microorganisms, nematodes and seed treatments (412 p., 6 Dec. 2012) eds. Burges H. D., Springer, ISBN 978-94-011-4926-6.

For foliar treatments (Example 3) of diseases on different crops, the whole fermentation broth of Streptomyces chrestomyceticus NRRL B-3672 and Streptomyces sp. Saigon 413 was tested as well as spray dried fermentation broth Streptomyces Saigon 413 TGAI at 10⁵ to 10¹⁰ cfu/g TGAI, that was formulated as oil dispersion (OD) and non-aqueous dispersion (NAD).

In some cases a surfactant polyethylene glycol sorbitan monolaurate (Tween® 20) was used.

An OD40% w/w was prepared of 40% dried fermentation broth in a vegetable oil containing an emulsifier, such as a surfactant. A UV protectant such as lauryl galleate can be added.

A NAD35% or NAD40% formulation was prepared containing 35% or 40% w/w % resp. of dried material, in an alcohol, for instance methyl-diproxitol, and a rheology modifier (organic clay AEROSIL KLUCEL G IND-cellulose 200)

For seed treatments (Example 4) spray-dried or freeze-dried S. chrestomyceticus CBS149411 (Streptomyces Saigon 413) TGAI at 10⁷ to 10¹⁰ cfu/g TGAI was used as such or formulated as a flowable concentrate (FS) or as an oil dispersion (OD).

FS300 is a flowable concentrate which contains 30% w/w % of spray dried TGAI in water, a surfactant, such as silicon-based antifoam and a dispersant, such as styrene/methacrylic acid.

An OD400 formulation contains 40% w/w of spray dried TGAI.

### Example 1.3. Isolation of 16S rDNA, Genomic DNA and Species Identification

Genomic DNA was isolated from Streptomyces sp. Saigon413 using the method described in Kutchma et al. (1998) Biotechniques 24 (3): 452-457. The 16S rRNA gene was amplified using universal 16S primers and sequenced using Sanger sequencing. The 16S rRNA of Streptomyces sp. Saigon413 is shown in SEQ ID NO: 1.

The species of strain Streptomyces sp. Saigon413 was identified by comparing the 16 S rRNA sequence according to SEQ ID NO:1 with publicly available 16S rRNA sequences that were extracted using whole genome sequence assembly of genomes from Streptomyces species (based on The Genome Taxonomy Database GTDB (Parks, D. H., et al. (2021). GTDB: Nucleic Acids Research, 50: D₇₈₅-D₇₉₄) using barrnap v0.9. Based on this comparative analysis Streptomyces sp. Saigon413 was identified as a Streptomyces chrestomyceticus species. The sequence identity between the 16S rRNA sequence of Streptomyces sp. Saigon413 and S. chrestomyceticus NRRL-3672 was 99.87%, which was determined using Muscle v3.8.31 and R package Seqinr v4.2-16.

Whole genome sequencing, using the genomic DNA from Streptomyces sp. Saigon413, was completed using both Pacific Biosciences and Illumina sequencing technologies. The genome was assembled using HFAP4 and polished with Pilon using the Illumina reads. Genomic DNA was also extracted from Streptomyces rimosus CBS 492.64, Streptomyces rimosus CBS 570.66, Streptomyces rimosus CBS 569.66, Streptomyces chrestomyceticus DSM 41224, Streptomyces rimosus subsp. rimosus DSM 40673, and Streptomyces rimosus subsp. rimosus DSM 41057 using a method described in Kieser et. al., (2000) Practical Streptomyces Genetics. Whole genome sequencing for these strains was completed using Nanopore Sequencing technology and the genomes were assembled with Flye (Kolmogorov, M., et. al., (2019), Nature Biotechnology, 37, 540).

Following assembly of the genome from Streptomyces sp. Saigon 413 and publicly available genomes, the average nucleotide identity (ANI) was calculated between Streptomyces sp. Saigon413 and closely related Streptomyces strains using fastANI (Jain, C., et. al. (2018), Nature Communications, 9, 5114) (Table 1). The highest percentage identity (ANI) of the genome of of Streptomyces sp. Saigon413 was 96.9% with the genome of and S. chrestomyceticus NRRL B-3672.

Using the 16SRNA sequence identity and ANI score (%), it was also found that the strains CBS 596.66, CBS570.66 and DSM 41429 were Streptomyce chrestomyceticus strains and not a Streptomyces rimosis or Streptomyces paromomycinus strain as indicated by the depository institute. In Table 1, the percentage identity of the whole genome and the 16SRNA sequence of Streptomyces sp. Saigon 413 and Streptomyces chrestomyceticus NRRL B-3672 is shown.

TABLE 1
Sequence identity of the 16SRNA and whole genome of Streptomyces sp. Saigon 413 with
publicly available 16SRNA and genome of other closely related Streptomyces species,.

| | | Species as | | ANI (%) | ANI (%) | 16S RNA |
|---|---|---|---|---|---|---|
| | | indicated by | Species as | vs. NRRL | vs. | sequence |
| Strain ID | Collection | the source | indicated by ANI | B-3672 | CBS149411 | identity (%) |
| CBS | CBS | | Streptomyces | 96.90 | 100 | 100 |
| 149411 | | | chrestomyceticus | | | |
| | | | Streptomyces sp. | | | |
| | | | Saigon 413 | | | |
| CBS | CBS | Streptomyces | Streptomyces sp. | 90.02 | 90.04 | 99.48 |
| 492.64 | | rimosus | | | | |
| CBS | CBS | Streptomyces | Streptomyces | 96.63 | 99.75 | 99.94 |
| 569.66 | | rimosus | chrestomyceticus | | | |
| CBS | CBS | Streptomyces | Streptomyces | 96.65 | 99.74 | 99.94 |
| 570.66 | | rimosus | chrestomyceticus | | | |
| CBS | CBS | Streptomyces | Streptomyces | 89.91 | 89.92 | 98.82 |
| 612.68 | | albofaciens | albofaciens | | | |
| CBS | CBS | Streptomyces | Streptomyces | 89.88 | 89.81 | 98.89 |
| 938.68 | | rimosus subsp. | rimosus | | | |
| | | rimosus | | | | |
| DSM | DSMZ | Streptomyces | Streptomyces | 89.86 | 89.78 | 99.02 |
| 40673 | | rimosus subsp. | rimosus | | | |
| | | rimosus | | | | |
| DSM | DSMZ | Streptomyces | Streptomyces | 90.00 | 89.96 | 98.89 |
| 41057 | | rimosus subsp. | rimosus | | | |
| | | rimosus | | | | |
| DSM | DSMZ | Streptomyces | Streptomyces | 99.97 | 96.96 | 99.87 |
| 41224 | | chrestomyceticus | chrestomyceticus | | | |
| DSM | DSMZ | Streptomyces | Streptomyces | 95.88 | 95.95 | 99.87 |
| 41429 | | paromomycinus | chrestomyceticus | | | |
| DSM | DSMZ | Streptomyces | Streptomyces | 89.79 | 89.84 | 99.02 |
| 41561 | | rimosus subsp. | rimosus | | | |
| | | rimosus | | | | |
| NRRL B- | ARS | Streptomyces | Streptomyces | 89.82 | 89.75 | 98.89 |
| 2626 | | rimosus subsp. | rimosus | | | |
| | | rimosus | | | | |
| NRRL B- | ARS | Streptomyces | Streptomyces | 100 | 96.9 | 99.87 |
| 3672 | | chrestomyceticus | chrestomyceticus | | | |

CBS Westerdijk fungal diversity institute: Uppsalalaan 8, 3584 CT, Utrecht, The Netherlands https//wi.knaw.nl/
DSMZ German Collection of Microorganisms and Cell Cultures: Inhoffenstraße 7B, 38124 Braunschweig, GERMANY - www.dsmz.de
ARS ARS Culture Collection (NRRL), 1815 N. University Street, Peoria, IL 61604, USA - https://nrrl.ncaur.usda.gov

### Example 1.4. Analysis of Streptimidone, Cyclothiazomycin C and Malonomicin

The structure of cyclothiazomycin C is disclosed on p. 3 of WO2015191789 and can be extracted and analysed according to the method discosed in Wang et al. (2010) Appl. Environmental Microbiology, Vol. 76, No. 7 p.2336. Malonomicin can be extracted and analysed according to the method disclosed in Example I (B) of WO2006/078939. Streptimidone can be extracted and isolated according to the method as disclosed in Lee et al. J. of Antibiotics (2020) 73: p. 184-188, including the supplementary information.

### Example 1.5. Purification of the Oligosaccharide Compound I

Whole broth of Streptomyces species shown in Table 1, such as Streptomyces chrestomyceticus CBS149411 (Streptomyces sp. Saigon413), was centrifuged to produce an aqueous extract and a pellet. The aqueous extract was freeze dried. The material was resuspended in a minimal volume of water and partitioned with ethyl acetate to remove lipophilic components. The aqueous suspension was retained and freeze dried and resuspended in a minimal volume of water before being applied to an activated charcoal column.

The column was washed with water and eluted with water:acetone (50:50).

The compound I was further purified by Hydrophilic Interaction Liquid Chromatography (HILIC) using Mass guided fractionation and ELSD detector. Using for example Waters XBridge Amide, 5 micron, 30×100 mm using a gradient of acetonitrile and 10 mM Ammonium Acetate.

### Characterisation of Oligosaccharide Compound I

The compound I was determined in the purified fermentation broth according to the methods disclosed below.

### Molecular Composition and Total Molecular Mass

The molecular composition and total molecular mass was C₅₃H₉₀N₂O₄₄, and 1458.487 g, respectively which were determined using MS-MS and NMR spectroscopy as disclosed below.

### Solubility

The solubility of the oligosaccharide compound I in water, pH 7.01, was >10′000 ppm, and in DMSO it was >9772 ppm.

### MS-MS Spectrometry and Liquid Chromatography

Spectra were recorded on an Orbitrap ID-X Tribrid Mass Spectrometer from Thermo Scientific equipped with an OptaMax NG Heated Electrospray Source (Spray Voltage: Static, Polarity Ion (V): 3400 (Positive ion mode) & 2400 (Negative ion mode), Sheath Gas (Arb): 40, Aux Gas (Arb): 5, Sweep Gas (Arb): 1, Ion Transfer Tube Temperature: 350 °C, Vaporizer Temperature: 350 °C). The Scan Parameters were as follows;
Experiment 1: MS OT (Orbitrap Resolution: 60,000, Scan Range (m/z): 200 to 2000, RF Lens (%): 60, AGC Target: Standard, Maximum Injection Time Mode: Auto, Microscans: 1, Data Type: Profile, Polarity: Both),
Experiment 2: tMS2 OT CID (MSn Level (n): 2, Isolation Window (m/z): 1.6, Activation Type: CID, CID Collision Energy (%): 30, Detector Type: Orbitrap, Orbitrap Resolution: 30,000, RF Lens (%): 60, Polarity: Negative). The mass spectrometer was connected to a Vanquish Flex UHPLC from Thermo Scientific using a Vanquish Split Sampler FT, Vanquish Binary Pump F, Vanquish Column Compartment H, Vanquish Diode Array Detector FG and Vanquish Charged Aerosol Detector. Liquid Chromatography Conditions included: Thermo Scientific Hypercarb™ Porous Graphitic Carbon column 5 μm 4.6×50 mm, P.N. 35005-054630. Temp: 40 °C, DAD wavelength range: 250 to 260 nm, Solvent gradient: Solvent A: H₂O with 0.1% formic acid, Solvent B: CH₃CN with 0.1% formic acid, gradient: 0 min 1% B, 99% A; 4.00 min 50% B, 50% A; 4.25 min 100% B; 4.50 min 100% B; 4.95 min 1% B, 99% A; 6.00 min 1% B, 99% A, Flow rate: 1.0 ml/min, Injection volume: 2 μL, Total run time: 6.0 min.

### NMR Spectroscopy

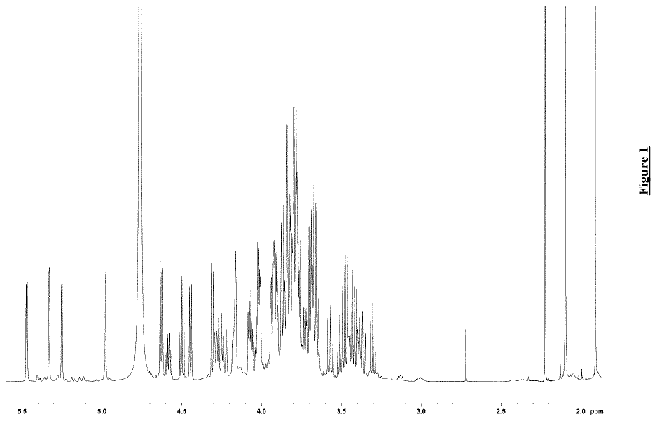

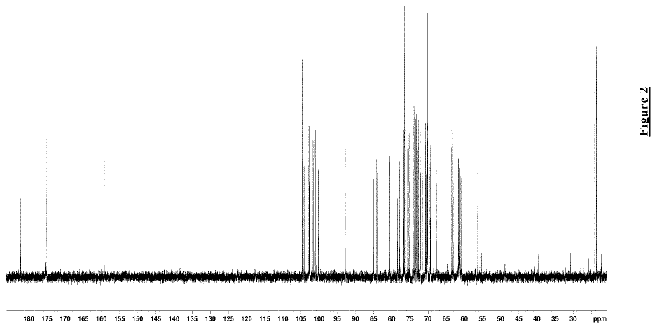

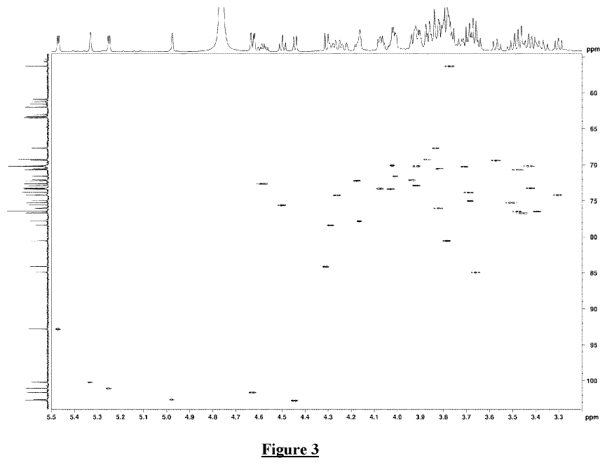

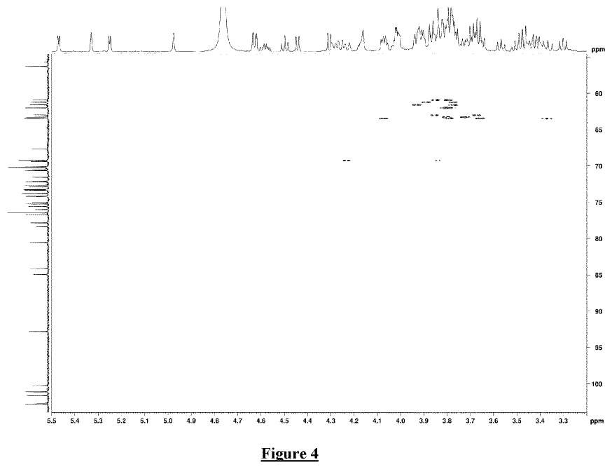

NMR spectra were recorded on a Bruker AVIII 600 NMR spectrometer, equipped with a 5 mm Bruker (¹H/¹⁹F)/¹³C/¹⁵N TCI cryoprobe fitted with Z gradients, using standard Bruker pulse sequences. Samples were dissolved in D₂O, and the spectra were recorded at 300° K and referenced to acetone at 2.225 ppm for ¹H and 31.07 ppm for ¹³C. FIGS. 1 to 4 show NMR spectra of the compound of the present invention.

The one bond ¹H-¹³C correlation spectrum contains peaks corresponding to 1 methyl (CH₃) and 40 methine (CH) groups (listed in Table 1) and 9 methylene (CH₂) groups (listed in Table 2).

In addition, the 1D ¹³C spectrum contains signals from 3 quaternary carbons at 104.7, 159.3 and 175.2 ppm (±0.1).

TABLE 2
Methyl and methine signals in the one bond ¹H-¹³C correlation
spectrum of the oligosaccharide compound I together with multiplicity
information for protons resolved in the 1D ¹H spectrum.

| d ¹³C ppm (±0.1) | d ¹H ppm (±0.05) | ¹H Multiplicity |
|---|---|---|
| 104.2 | 4.632 | d (8.0, 1H) |
| 102.8 | 4.45 | d (7.5, 1H) |
| 102.6 | 4.98 | d (2.0, 1H) |
| 101.6 | 4.628 | d (8.5, 1H) |
| 101.1 | 5.25 | d (4.0, 1H) |
| 100.2 | 5.33 | d (2.0, 1H) |
| 92.8 | 5.47 | d (4.0, 1H) |
| 84.9 | 3.66 | |
| 84.1 | 4.31 | d (8.0, 1H) |
| 80.5 | 3.79 | |
| 78.4 | 4.29 | |
| 77.8 | 4.16 | |
| 76.7 | 3.46 | |
| 76.5 | 3.48 | |
| 76.5 | 3.39 | |
| 76.0 | 3.82 | |
| 75.6 | 4.50 | t (8.0, 1H) |
| 75.2 | 3.51 | |
| 75.0 | 3.68 | |
| 74.21 | 4.26 | |
| 74.18 | 3.30 | dd (8.0, 9.0, 1H) |
| 73.8 | 3.69 | |
| 73.4 | 4.03 | |
| 73.3 | 4.07 | |
| 73.2 | 3.42 | |
| 72.9 | 3.92 | |
| 72.6 | 4.58 | ddd (5.5, 9.5, 10.0, 1H) |
| 72.2 | 4.17 | |
| 72.1 | 3.94 | |
| 71.6 | 4.01 | |
| 70.7 | 3.48 | |
| 70.5 | 3.81 | |
| 70.3 | 3.71 | |
| 70.2 | 3.92 | |
| 70.2 | 3.43 | |
| 70.1 | 4.02 | |
| 69.4 | 3.57 | dd (9.0, 10.0, 1H) |
| 69.2 | 3.87 | |
| 67.7 | 3.84 | |
| 56.3 | 3.77 | |
| 23.6 | 2.10 | s (3H) |

TABLE 3
Methylene signals in the one bond ¹H-¹³C correlation spectrum
of the compound I together with multiplicity information for
protons resolved in the 1D ¹H spectrum.

| d ¹³C ppm (±0.1) | d ¹H ppm (±0.05) | ¹H Multiplicity |
|---|---|---|
| 69.2 | 4.23 | |
| 69.2 | 3.85 | |
| 63.5 | 4.07 | |
| 63.5 | 3.37 | dd (10.5, 11.5, 1H) |
| 63.4 | 3.79 | |
| 63.4 | 3.66 | |
| 63.3 | 3.81 | |
| 63.3 | 3.72 | |
| 63.0 | 3.85 | |
| 63.0 | 3.67 | |
| 62.0 | 3.82 | |
| 62.0 | 3.78 | |
| 61.6 | 3.93 | |
| 61.6 | 3.77 | |
| 61.2 | 3.89 | |
| 61.2 | 3.77 | |
| 60.9 | 3.85 | |
| 60.9 | 3.79 | |

### Example 1.6. Lipopeptide Compound of Formula II

Purification of a liopeptide according to Formula II (Formula II (a) and Formula II (b)) The mycelia from fermentation broth from Streptomyces sp. listed in Table 1, such as Streptomyces sp. Saigon 413, was separated via centrifugation and the supernatant was treated with butanol. The butanol was removed and the extract partitioned between water and ethyl acetate. The lipopeptides were purified from the ethyl acetate fraction by preparative reverse phase (C₁₈) HPLC. The lipopeptides were relatively aploar and elute in the higher organic fraction in a gradient system with 0.1% formic acid and acetonirile (0.1% formic acid). A gradient of 60% Aqueous to 40% Aqueous with the above solvents allowed separation of a lipopeptide compound according to Formula II (a) and Formula II (b).

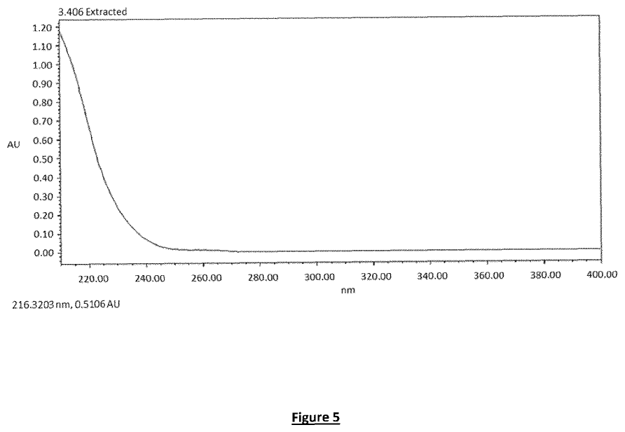

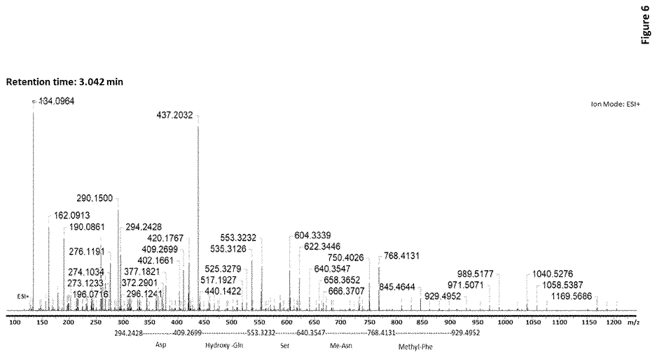

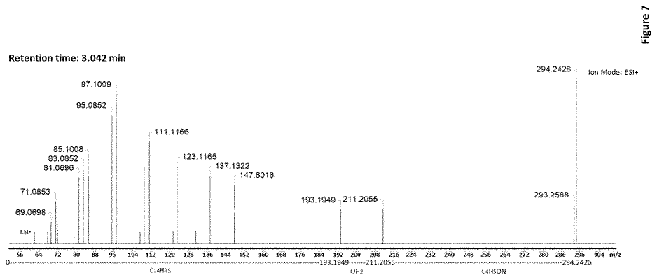

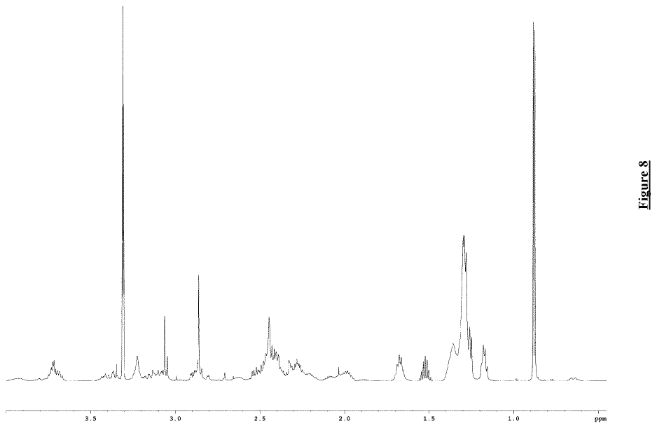

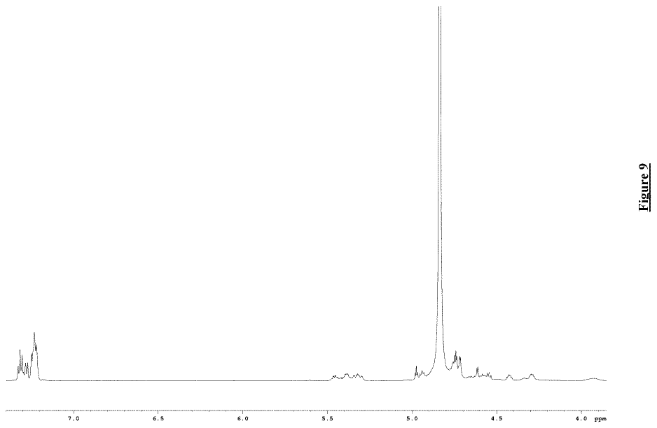

Compounds were detected by UV-VIS (FIG. 5), mass spectrometry (FIGS. 6 and 7) and NMR spectrometry (FIGS. 8 and 9).

### Lipopeptin A

Lipopeptin A was purchased from Fundación MEDINA, Centro de Excelencia en Investigación de Medicamentos Innovadores en Andalucía, Avda. del Conocimiento 34, Edificio Centro de Desarrollo Farmacéutico y Alimentario, Parque Tecnológico de Ciencias de la Salud, 18016 Granada (ESPAÑA).

### Characterisation of a Lipopeptide of Formula II

### Liquid Chromatography and High-Resolution Mass Spectrometry

Spectra were recorded on an Orbitrap ID-X Tribrid Mass Spectrometer from Thermo Scientific equipped with an OptaMax NG Heated Electrospray Source (Spray Voltage: Static, Polarity Ion (V): 3400 (Positive ion mode) & 2400 (Negative ion mode), Sheath Gas (Arb): 40, Aux Gas (Arb): 5, Sweep Gas (Arb): 1, Ion Transfer Tube Temperature: 350 °C, Vaporizer Temperature: 350 °C). The Scan Parameters were as follows;
Experiment 1: MS OT (Orbitrap Resolution: 50,000, Scan Range (m/z): 200 to 2000, RF Lens (%): 60, AGC Target: Standard, Maximum Injection Time Mode: Auto, Microscans: 1, Data Type: Profile, Polarity: Both),
Experiment 2: tMS2 OT CID (MSn Level (n): 2, Isolation Window (m/z): 1.0, Activation Type: CID, CID Collision Energy (%): 30, Detector Type: Orbitrap, Orbitrap Resolution: 30,000, RF Lens (%): 60, Polarity: Positive),
Experiment 3: tMS2 OT HCD (MSn Level (n): 2, Isolation Window (m/z): 1.0, Activation Type: HCD, HCD Collision Energy (%): 30, Detector Type: Orbitrap, Orbitrap Resolution: 30,000, RF Lens (%): 60, Polarity: Positive), Experiment 4: tMS3 OT HCD (MSn Level (n): 3, Isolation Window (m/z): 1.6, Activation Type: HCD, HCD Collision Energy (%): 30, MS2 Isolation Window (m/z): 2, MS2 Activation Type: HCD, MS2 HCD Collision Energy (%): 30, Detector Type: Orbitrap, Orbitrap Resolution: 30,000, RF Lens (%): 60, Polarity: Positive). The mass spectrometer was connected to a Vanquish Flex UHPLC from Thermo Scientific using a Vanquish Split Sampler FT, Vanquish Binary Pump F, Vanquish Column Compartment H, Vanquish Diode Array Detector FG and Vanquish Charged Aerosol Detector. Liquid Chromatography Conditions included: Waters ACQUITY UPLC C18 column 1.7 μm 3.0×50 mm, P.N. 186004660. Temp: 40 °C, DAD wavelength range: 250 to 260 nm, Solvent gradient: Solvent A: H2O with 0.1% formic acid, Solvent B: CH₃CN with 0.1% formic acid, gradient: 0 min 10% B, 90% A; 4.00 min 90% B, 10% A; 4.25 min 90% B, 10% A; 4.50 min 10% B, 90% A; 5.00 min 10% B, 90% A, Flow rate: 1.0 ml/min, Injection volume: 2 μL, Total run time: 5.0 min. A purified fermentation broth as described above was injected.

FIGS. 6 and 7 show LC-ESI-MS/MS/MS spectra of a compound of Formula II (a).

### NMR Spectroscopy

NMR spectra were recorded on a Bruker AVIII 600 NMR spectrometer, equipped with a 5 mm Bruker (¹H/¹⁹F)/¹³C/¹⁵N TCI cryoprobe fitted with Z gradients, using standard Bruker pulse sequences. Samples were dissolved in CD₃OD, and the spectra were recorded at 300° K and referenced to the residual solvent signal at 3.31 ppm for 1H. FIGS. 8 and 9 each cover half of the 1H NMR spectrum of a compound according to Formula II (a).

### Molecular Composition and Mass

The molecular composition and mass of a lipopeptide according to Formula II (a) and Formula II (b) was determined using the results of liquid chromatography and high-resolution mass spectrometry as disclosed above. The lipopeptide compounds of Formula II (a) and II (b) have the following composition. Formula II (a) Lipopeptide 1204: Molecular composition C55H85N11O19 and exact mass of 1203.602. Formula II (b) Lipopeptide 1218: Molecular composition C56H87N11O19 and exact mass of 1217.618. The reference Lipopeptin A has the following composition C54H84N10O19 and exact mass 1176.591421.

### Solubility

The solubility of a compound of Formula II (a), Formula II (b) and Lipopeptin A was determined in water and DMSO:

| Compound | solvent (pH) | solubility (ppm) |
|---|---|---|
| Formula II (a) Lipopeptide 1204 | water (2.08) | 21.4 |
| Formula II (a) Lipopeptide 1204 | water (5.55) | >10′000 |
| Formula II (a) Lipopeptide 1204 | DMSO | >10′000 |
| Formula II (b) Lipopeptide 1218 | DMSO | >10′000 |
| Lipopeptin A | DMSO | >10′000 |

Identification of the Biosynthetic Gene Cluster Producing a Lipopeptide According to Formula II in Streptomyces sp. Saigon413

To identify genes involved in the production of the lipopeptide according to Formula II, the assembled genome (see Example 1.4) was run through AntiSMASH (version 5.1.1, Blin et. al., Nucleic Acids Res (2019) doi: 10.1093/nar/gkz310), a commonly used tool to assist with the identification of biosynthetic gene clusters responsible for the production of secondary metabolites.

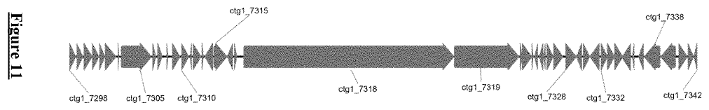

With the identification of lipopeptide compound being a lipopeptide family (see above) and the AntiSMASH output we were able to deduce that the lipopeptide compound is produced by a non-ribosomal peptide synthetase (NRPS gene cluster). The identification of a NRPS gene cluster responsible for the biosynthesis of the lipoeptide compound was based upon the structural analysis of the compound and the amino acids that are incorporated into the depsipeptide core of the lipopeptide compound. Within Streptomyces sp. Saigon413, only one NRPS biosynthetic gene cluster (FIG. 11) enabling the incorporation of amino acid precursors including asparagine, aspartic acid, glutamic acid, phenylalanine, serine and threonine, was identified and therefore was associated with the production of the lipopeptide compounds of Formula II (a) and II (b).

The NRPS biosynthetic gene cluster contains 45 coding sequences including two coding sequences for NRPS genes, a coding sequence for a regulator, and coding sequences responsible for the biosynthesis of a precursor incorporated into lipopeptide of Formula II (FIG. 11 and Table 4).

TABLE 4
Coding sequences present in NRPS biosynthetic gene cluster responsible for
the production of the lipopeptide according to Formula II. Annotations
provided are based upon pBLAST search using the non-redundant protein sequences
on the National Centre for Biotechnology Information database.

| | SEQ ID | | |
|---|---|---|---|
| Gene ID | DNA | Translated Protein | Annotation |
| CDS_1 (ctg1_7298) | SEQ ID NO: 2 | SEQ ID NO: 71 | O-methyltransferase |
| CDS_2 (ctg1_7299) | SEQ ID NO: 3 | SEQ ID NO: 72 | Oxygenase |
| CDS_3 (ctg1_7300) | SEQ ID NO: 4 | SEQ ID NO: 73 | Hypothetical protein |
| CDS_4 (ctg1_7301) | SEQ ID NO: 5 | SEQ ID NO: 74 | Deoxynucleoside kinase |
| CDS_5 (ctg1_7302) | SEQ ID NO: 6 | SEQ ID NO: 75 | Chloramphenicol |
| | | | phosphotransferase family protein |
| CDS_6 (ctg1_7303) | SEQ ID NO: 7 | SEQ ID NO: 76 | MFS transporter |
| CDS_7 (ctg1_7304) | SEQ ID NO: 8 | SEQ ID NO: 77 | Unknown protein |
| CDS_8 (ctg1_7305) | SEQ ID NO: 9 | SEQ ID NO: 78 | ALF repeat-containing protein |
| CDS_9 (ctg1_7306) | SEQ ID NO: 10 | SEQ ID NO: 79 | Hypothetical protein |
| CDS_10 (ctg1_7307) | SEQ ID NO: 11 | SEQ ID NO: 80 | Hypothetical protein |
| CDS_11 (ctg1_7308) | SEQ ID NO: 12 | SEQ ID NO: 81 | Hypothetical protein |
| CDS_12 (ctg1_7309) | SEQ ID NO: 13 | SEQ ID NO: 82 | Radical SAM enzyme |
| CDS_13 (ctg1_7310) | SEQ ID NO: 14 | SEQ ID NO: 83 | Phytanoyl-CoA dioxygenase |
| CDS_14 (ctg1_7311) | SEQ ID NO: 15 | SEQ ID NO: 84 | Transcriptional regulator |
| CDS_15 (ctg1_7312) | SEQ ID NO: 16 | SEQ ID NO: 85 | NADP-dependent oxidoreductase |
| CDS_16 (ctg1_7313) | SEQ ID NO: 17 | SEQ ID NO: 86 | Short-chain dehydrogenase/ |
| | | | reductase |
| CDS_17 (ctg1_7314) | SEQ ID NO: 18 | SEQ ID NO: 87 | Transcriptional regulator |
| CDS_18 (ctg1_7315) | SEQ ID NO: 19 | SEQ ID NO: 88 | Drug resistance transporter |
| CDS_19 (ctg1_7316) | SEQ ID NO: 20 | SEQ ID NO: 89 | Transcriptional regulator (TetR |
| | | | family) |
| CDS_20 (ctg1_7317) | SEQ ID NO: 21 | SEQ ID NO: 90 | Hypothetical protein |
| CDS_21 (ctg1_7318) | SEQ ID NO: 22 | SEQ ID NO: 91 | Non-ribosomal peptide |
| | | | synthetase |
| CDS_22 (ctg1_7319) | SEQ ID NO: 23 | SEQ ID NO: 92 | Non-ribosomal peptide |
| | | | synthetase |
| CDS_23 (ctg1_7320) | SEQ ID NO: 24 | SEQ ID NO: 93 | MbtH-like protein |
| CDS_24 (ctg1_7321) | SEQ ID NO: 25 | SEQ ID NO: 94 | TauD/TfdA family dioxygenase |
| CDS_25 (ctg1_7322) | SEQ ID NO: 26 | SEQ ID NO: 95 | SAM-dependent methyltransferase |
| CDS_26 (ctg1_7323) | SEQ ID NO: 27 | SEQ ID NO: 96 | Hypothetical protein |
| CDS_27 (ctg1_7324) | SEQ ID NO: 28 | SEQ ID NO: 97 | VOC family protein |
| CDS_28 (ctg1_7325) | SEQ ID NO: 29 | SEQ ID NO: 98 | Transcriptional regulator (ArsR |
| | | | family) |
| CDS_29 (ctg1_7326) | SEQ ID NO: 30 | SEQ ID NO: 99 | Domain of unknown function (DUF) |
| CDS_30 (ctg1_7327) | SEQ ID NO: 31 | SEQ ID NO: 100 | Transcriptional regulator (AraC |
| | | | family) |
| CDS_31 (ctg1_7328) | SEQ ID NO: 32 | SEQ ID NO: 101 | Transporter |
| CDS_32 (ctg1_7329) | SEQ ID NO: 33 | SEQ ID NO: 102 | Domain of unknown function (DUF) |
| CDS_33 (ctg1_7330) | SEQ ID NO: 34 | SEQ ID NO: 103 | Aminoglycoside adenylyltransferase |
| CDS_34 (ctg1_7331) | SEQ ID NO: 35 | SEQ ID NO: 104 | FxbS family cyclophane- |
| | | | forming radical SAM/SPASM |
| | | | peptide maturase |
| CDS_35 (ctg1_7332) | SEQ ID NO: 36 | SEQ ID NO: 105 | 2′-5′ RNA ligase family protein |
| CDS_36 (ctg1_7333) | SEQ ID NO: 37 | SEQ ID NO: 106 | Transcriptional regulator (MerR |
| | | | family) |
| CDS_37 (ctg1_7334) | SEQ ID NO: 38 | SEQ ID NO: 107 | Hypothetical protein |
| CDS_38 (ctg1_7335) | SEQ ID NO: 39 | SEQ ID NO: 108 | Chorophyllase |
| CDS_39 (ctg1_7336) | SEQ ID NO: 40 | SEQ ID NO: 109 | Hypothetical protein |
| CDS_40 (ctg1_7337) | SEQ ID NO: 41 | SEQ ID NO: 110 | ABATE domain-containing protein |
| | | | (putative stress-induced |
| | | | transcription regulator) |
| CDS_41 (ctg1_7338) | SEQ ID NO: 42 | SEQ ID NO: 111 | ABC transporter (ATP-binding |
| | | | cassette protein) |
| CDS_42 (ctg1_7339) | SEQ ID NO: 43 | SEQ ID NO: 112 | ABC transporter (ATP-binding |
| | | | cassette protein) |
| CDS_43 (ctg1_7340) | SEQ ID NO: 44 | SEQ ID NO: 113 | Quinone oxidoreductase |
| CDS_44 (ctg1_7341) | SEQ ID NO: 45 | SEQ ID NO: 114 | Transcriptional regulator (TetR |
| | | | family) |
| CDS_45 (ctg1_7342) | SEQ ID NO: 46 | SEQ ID NO: 115 | Helix-turn-helix domain |
| | | | containing protein |

Deletion of Genomic Region Including CDS_21 (SEQ ID NO: 22) and CDS_22 (SEQ ID NO: 23) from Streptomyces sp. Saigon413 and Phenotypic Analysis

To confirm the identified biosynthetic gene cluster was associated with the production of [insert compound ID here], a region containing two non-ribosomal peptide synthetase genes encoded by ctg_7318 and ctg_7319 (SEQ ID NO: 22 and 23) was deleted from Streptomyces sp. Saigon413. To generate Streptomyces sp. Saigon41347318-7319, plasmid pBCon2192 was used. Plasmid pBCon2192 was prepared from pRAR017 and contained regions of homology to either side of the region to be deleted from the strain (facilitating primary and secondary crossovers).

Plasmid pBCon2192 was used to transform E. coli ET12567/pUZ8002 using a standard electroporation method, and then introduced into Streptomyces sp. Saigon413 by mycelial conjugation (T. Kieser et. al., Practical Streptomyces Genetics, 2000, John Innes Foundation, Norwich). Thiostrepton resistant colonies were patched on ISP-4 agar media supplemented with 40 μg/ml thiostrepton and 25 μg/ml nalidixic acid. These patches were initially incubated for 6 days at 28 °C allowing plasmid replication. After 6 days at 28 °C, strains were re-patched on ISP-4 agar media supplemented with 40 μg/ml thiostrepton and incubated at 37 °C for further 6 days to force primary integration. After 6 days at 37 °C, the obtained strains were transferred onto ISP-4 solid agar media without selection and incubated for 15 days at 28 °C to allow a second crossover.

After 15 days of growth, strains were collected in 20% glycerol. 100 μl of the cell suspension was used to inoculate fresh plates, as well as to make serial dilutions up to 10-10. 100 μl of 10⁻⁸ to 10-10 were then plated onto ISP-4 agar plate. Plates were incubated at 28 °C until single colonies were observed.

Single colonies were double patched on non-selective and thiostrepton selective ISP-4 agar media. Sensitive patches (representing secondary recombination) were then screened via PCR using gDNA isolated with FastSpin kit for soil (MP Biomedicals) to identify correct colonies.

To confirm the region containing both SEQ ID NO: 22 and SEQ ID NO: 23 has been removed from the strain, a primer pair binding to the outside of the deleted region was used. Sanger sequencing of the PCR product and alignment to the Streptomyces sp. Saigon413 genome, confirmed deletion of the genomic region containing SEQ ID NO: 22 and SEQ ID NO: 23. Additionally, full genome analysis using Illumina-PCR-free sequencing confirmed that no other alterations had been made to the genome.

Cultivation of Streptomyces sp. Saigon41347318-7319 and analysis of extracts from the strain confirmed that the lipopeptide compound according to Formula II (a) was no longer produced by the strain, confirming SEQ ID O: 22 and SEQ ID NO: 23 are essential for production of the lipopeptide compound according to Formula II (a)

### Example 1.7. Polyene Compound

Purification of polyene compound

A spray dried sample from a culture of S. chrestomycetiucs CBS149411 (Streptomyces sp. Saigon 413) was washed with water. The solid residue was extracted twice with isopropanol and the isopropanol was removed. The resulting solid was purified by preparative reverse phase (C₁₈) HPLC using an acetonitrile:water gradient. Further purification was conducted by preparative reverse phase HPLC using a Zorbax C8 column and eluting with an acetonitrile:water gradient.

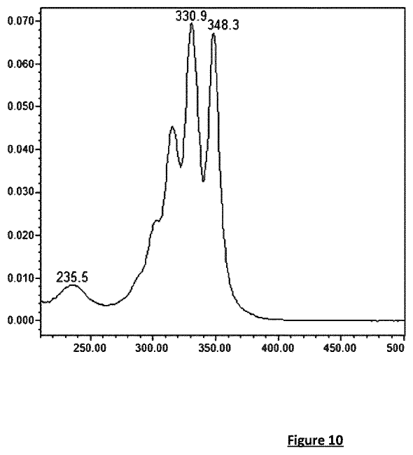

The compound was detected by UV-VIS (FIG. 10).

### Characterisation of Polyene Compound

### Liquid Chromatography and High-Resolution Mass Spectrometry

Spectra were recorded on an Orbitrap ID-X Tribrid Mass Spectrometer from Thermo Scientific equipped with an OptaMax NG Heated Electrospray Source (Spray Voltage: Static, Polarity Ion (V): 3400 (Positive ion mode) & 2400 (Negative ion mode), Sheath Gas (Arb): 40, Aux Gas (Arb): 5, Sweep Gas (Arb): 1, Ion Transfer Tube Temperature: 350 °C, Vaporizer Temperature: 350 °C). The Scan Parameters were as follows;
Experiment 1: MS OT (Orbitrap Resolution: 50,000, Scan Range (m/z): 200 to 2000, RF Lens (%): 60, AGC Target: Standard, Maximum Injection Time Mode: Auto, Microscans: 1, Data Type: Profile, Polarity: Both),
Experiment 2: tMS2 OT CID (MSn Level (n): 2, Isolation Window (m/z): 1.0, Activation Type: CID, CID Collision Energy (%): 30, Detector Type: Orbitrap, Orbitrap Resolution: 30,000, RF Lens (%): 60, Polarity: Positive),
Experiment 3: tMS2 OT HCD (MSn Level (n): 2, Isolation Window (m/z): 1.0, Activation Type: HCD, HCD Collision Energy (%): 30, Detector Type: Orbitrap, Orbitrap Resolution: 30,000, RF Lens (%): 60, Polarity: Positive), Experiment 4: tMS3 OT HCD (MSn Level (n): 3, Isolation Window (m/z): 1.6, Activation Type: HCD, HCD Collision Energy (%): 30, MS2 Isolation Window (m/z): 2, MS2 Activation Type: HCD, MS2 HCD Collision Energy (%): 30, Detector Type: Orbitrap, Orbitrap Resolution: 30,000, RF Lens (%): 60, Polarity: Positive).

The mass spectrometer was connected to a Vanquish Flex UHPLC from Thermo Scientific using a Vanquish Split Sampler FT, Vanquish Binary Pump F, Vanquish Column Compartment H, Vanquish Diode Array Detector FG and Vanquish Charged Aerosol Detector.

Liquid Chromatography Conditions included: Waters ACQUITY UPLC C₁₈ column 1.7 μm 3.0×50 mm, P.N. 186004660. Temp: 40 °C, DAD wavelength range: 250 to 260 nm, Solvent gradient: Solvent A: H2O with 0.1% formic acid, Solvent B: CH₃CN with 0.1% formic acid, gradient: 0 min 10% B, 90% A; 4.00 min 90% B, 10% A; 4.25 min 90% B, 10% A; 4.50 min 10% B, 90% A; 5.00 min 10% B, 90% A, Flow rate: 1.0 ml/min, Injection volume: 2 μL, Total run time: 5.0 min.

A purified fermentation broth as described under section 1.8.1 was injected.

The key peaks observed were:
Negative ion: C67H114NO25 [M−H]⁻ Expected: 1332.7685, Observed: 1332.7679
Positive ion: C67H114NO24 [M−H2O+H]⁺ Expected: 1316.7725, Observed: 1316.7709
Positive ion: C67H115NO24 [M−H2O+2H]²⁺ Expected: 658.8899, Observed: 658.8895
Positive ion: C67H113NO23 [M−2 (H2O)+2H]²⁺ Expected: 649.8846, Observed: 649.8843
Positive ion: C67H111NO22 [M−3 (H2O)+2H]²⁺ Expected: 640.8793, Observed: 640.8790

### Molecular Composition and Mass

The molecular composition and mass of the polyene compound was determined using the results of liquid chromatography and high-resolution mass spectrometry as disclosed above. The polyene compound has a molecular composition C₆₇H₁₁₅NO₂₅ and exact mass of 1333.7758.

### Solubility

The solubility of a compounds was determined in DMSO:

| Compound | solvent (pH) | solubility (ppm) |
|---|---|---|
| Polyene compound | DMSO | >10′000 |
| Philipin complex | DMSO | >10′000 |
| Amphotericin B | DMSO | >10′000 |

Identification of the Biosynthetic Gene Cluster Producing Polyene Compound in Streptomyces Sp. Saigon413

To identify genes involved in the production of the polyene compound identified under 1.8.2 above, the assembled genome (see Example 1.4 above) was run through AntiSMASH (version 5.1.1, Blin et. al., Nucleic Acids Res (2019) doi: 10.1093/nar/gkz310), a commonly used tool to assist with the identification of biosynthetic gene clusters responsible for the production of secondary metabolites.

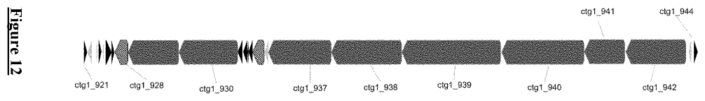

With the identification of the polyene compound (see above), and the AntiSMASH output we were able to deduce that the polyene of compound is produced by a modular type I polyketide synthase (PKS) gene cluster. The identification of a modular type I PKS gene cluster being responsible for the biosynthesis of the polyene compound was based upon the analysis of biosynthetic gene clusters that have been linked to the production of characterised polyenes, such as filipin by Streptomyces filipinensis, amphotericin by Streptomyces nodosus and thailandins A and B by Actinokineospora bangkokensis 44EHW. Within Streptomyces sp. Saigon413, one large modular type I PKS biosynthetic gene cluster (FIG. 12) was identified, and therefore was associated with the production of the polyene compound according to the present invention.

The modular type I PKS biosynthetic gene cluster contains 24 coding sequences including eight coding sequences for modular type I PKS genes, a coding sequence for a regulator, and coding sequences responsible for the biosynthesis of a precursor incorporated into polyene compound (FIG. 12 and Table 5).

TABLE 5
Coding sequences present in type I PKS biosynthetic gene cluster responsible
for the production of the polyene compound. Annotations provided are
based upon pBLAST search using the non-redundant protein sequences
on the National Centre for Biotechnology Information database.

| | SEQ ID | | |
|---|---|---|---|
| Gene ID | DNA | Protein | Annotation |
| CDS_11 (ctg1_921) | SEQ ID NO: 47 | SEQ ID NO: 116 | ABC transporter |
| CDS_12 (ctg1_922) | SEQ ID NO: 48 | SEQ ID NO: 117 | Unknown function |
| CDS_13 (ctg1_923) | SEQ ID NO: 49 | SEQ ID NO: 118 | Unknown function |
| CDS_14 (ctg1_924) | SEQ ID NO: 50 | SEQ ID NO: 119 | Thioesterase |
| CDS_15 (ctg1_925) | SEQ ID NO: 51 | SEQ ID NO: 120 | Unknown function |
| CDS_16 (ctg1_926) | SEQ ID NO: 52 | SEQ ID NO: 121 | Histidine Kinase |
| CDS_17 (ctg1_927) | SEQ ID NO: 53 | SEQ ID NO: 122 | LuxR-like regulator |
| CDS_18 (ctg1_928) | SEQ ID NO: 54 | SEQ ID NO: 123 | Non-ribosomal peptide |
| | | | synthetase |
| CDS_19 (ctg1_929) | SEQ ID NO: 55 | SEQ ID NO: 124 | Polyketide synthase |
| CDS_20 (ctg1_930) | SEQ ID NO: 56 | SEQ ID NO: 125 | Polyketide synthase |
| CDS_21 (ctg1_931) | SEQ ID NO: 57 | SEQ ID NO: 126 | FkbH-like protein |
| CDS_22 (ctg1_932) | SEQ ID NO: 58 | SEQ ID NO: 127 | Acyl-CoA dehydrogenase |
| CDS_23 (ctg1_933) | SEQ ID NO: 59 | SEQ ID NO: 128 | Acyl-carrier protein |
| CDS_24 (ctg1_934) | SEQ ID NO: 60 | SEQ ID NO: 129 | 3-hydroxybutyryl-CoA |
| | | | dehydrogenase |
| CDS_25 (ctg1_935) | SEQ ID NO: 61 | SEQ ID NO: 130 | Non-ribosomal peptide |
| | | | synthetase |
| CDS_26 (ctg1_936) | SEQ ID NO: 62 | SEQ ID NO: 131 | Unknown function |
| CDS_27 (ctg1_937) | SEQ ID NO: 63 | SEQ ID NO: 132 | Polyketide synthase |
| CDS_28 (ctg1_938) | SEQ ID NO: 64 | SEQ ID NO: 133 | Polyketide synthase |
| CDS_29 (ctg1_939) | SEQ ID NO: 65 | SEQ ID NO: 134 | Polyketide synthase |
| CDS_30 (ctg1_940) | SEQ ID NO: 66 | SEQ ID NO: 135 | Polyketide synthase |
| CDS_31 (ctg1_941) | SEQ ID NO: 67 | SEQ ID NO: 136 | Polyketide synthase |
| CDS_32 (ctg1_942) | SEQ ID NO: 68 | SEQ ID NO: 137 | Polyketide synthase |
| CDS_33 (ctg1_943) | SEQ ID NO: 69 | SEQ ID NO: 138 | Unknown function |
| CDS_43 (ctg1_944) | SEQ ID NO: 70 | SEQ ID NO: 139 | O-methyltransferase |

Deletion of Genomic Region Including CDS_31 (SEQ ID NO: 67) and CDS_32 (SEQ ID NO: 68) from Streptomyces sp. Saigon413 and Phenotypic Analysis

To confirm the identified biosynthetic gene cluster was associated with the production of the polyene compound, a region containing two polyketide synthase genes encoded by CDS_31 and CDS_32 (SEQ ID NO: 67 and SEQ ID NO: 68) was deleted from Streptomyces sp. Saigon413. To generate Streptomyces sp. Saigon413Δ941-942, plasmid p073-031 was used. Plasmid p073-031 was prepared from pRAR017 and contained regions of homology to either side of the region to be deleted from the strain (facilitating primary and secondary crossovers).

Plasmid p073-031 was used to transform E. coli ET12567/pUZ8002 using a standard electroporation method, and then introduced into Streptomyces sp. Saigon413 by mycelial conjugation (T. Kieser et. al., Practical Streptomyces Genetics, 2000, John Innes Foundation, Norwich). Thiostrepton resistant colonies were patched on ISP-4 agar media supplemented with 40 μg/ml thiostrepton and 25 μg/ml nalidixic acid. These patches were initially incubated for 6 days at 28 °C allowing plasmid replication. After 6 days at 28 °C, strains were re-patched on ISP-4 agar media supplemented with 40 μg/ml thiostrepton and incubated at 37 °C for further 6 days to force primary integration. After 6 days at 37 °C, the obtained strains were transferred onto ISP-4 solid agar media without selection and incubated for 15 days at 28 °C to allow a second crossover.

After 15 days of growth, strains were collected in 20% glycerol. 100 μl of the cell suspension was used to inoculate fresh plates, as well as to make serial dilutions up to 10⁻¹⁰. 100 μl of 10⁻⁸ to 10⁻¹⁰ were then plated onto ISP-4 agar plate. Plates were incubated at 28 °C until single colonies were observed.

Single colonies were double patched on non-selective and thiostrepton selective ISP-4 agar media. Sensitive patches (representing secondary recombination) were then screened via PCR using gDNA isolated with FastSpin kit for soil (MP Biomedicals).

To confirm the region containing both SEQ ID NO: 67 and SEQ ID NO: 68 has been removed from the strain, a primer pair binding to the outside of the deleted region was used. Sanger sequencing of the PCR product and alignment to the Streptomyces sp. Saigon413 genome, confirmed deletion of the genomic region containing SEQ ID NO: 67 and SEQ ID NO: 68. Additionally, full genome analysis using Illumina-PCR-free sequencing confirmed that no other alterations had been made to the genome.

Cultivation of Streptomyces sp. Saigon4134941-942 and analysis of extracts from the strain confirmed that the polyene compound was no longer produced by the strain. Confirming SEQ ID NO: 67 and SEQ ID NO: 68 are essential for production of the polyene compound disclosed herein.

### Example 2

2.1. Determination of Metabolites in Fermentation Broths of Several Streptomyces Strains. Of Malonomicin, CtmC and Streptimidone

The presence of the following metabolites was measured in the fermentation broth of Streptomyces sp. Saigon 413 and other Streptomyces species: malonomicin, cyclothiazomycine C, and streptimidone, an oligosaccharide compound according to compound I, a lipopeptide of Formula II, and a polyene compound. These metabolites were determined by isolating and purifying according to the methods described in section 1.4 to 1.7.

Table 6A shows that S. chrestomyceticus strains produce at least the metabolites malonomicin, CtmC, streptimidone, and at least an oligosaccharide compound I of molecular formula C₅₃H₉₀N₂O₄₄, a lipopeptide according to Formula II, and a polyene C₆₇H₁₁₅NO₂₅ in the fermentation broth of Streptomyces sp. Saigon 413 and several other Streptomyces strains

TABLE 6A
Presence of the metabolites malonomicin, CtmC, streptimidone, an oligosaccharide of compound
I, lipopeptide according to Formula II, and polyene C₆₇H₁₁₅NO₂₅ in the fermentation broth of
Streptomyces sp. Saigon 413 and several other Streptomyces strains

| | | | | | Oligosaccharide | | |
|---|---|---|---|---|---|---|---|
| | | | | | Compound I | Lipopeptide | Polyene |
| Strain ID | Species | Malonomicin | CtmC | Streptimidone | C₅₃H₉₀N₂O₄₄, | Formula II(a) | C₆₇H₁₁₅NO₂₅ |
| CBS 149411 | S. chrestomyceticus | + | + | + | + | + | + |
| | Streptomyces sp. | | | | | | |
| | Saigon 413 | | | | | | |
| CBS 492.64 | Streptomyces sp. | n.d. | n.d. | n.d. | n.d. | + | n.d. |
| | (S. rimosis) | | | | | | |
| CBS 569.66 | S. chrestomyceticus | + | + | + | + | + | + |
| | (S. rimosis) | | | | | | |
| CBS 570.66 | S. chrestomyceticus | + | + | + | + | + | + |
| | (S. rimosis) | | | | | | |
| CBS 612.68 | S. albofaciens | n.d. | n.d. | n.d. | + | n.d | n.d. |
| CBS 938.68 | S. rimosus | n.d. | n.d. | n.d. | + | n.d. | n.d. |
| DSM 40673 | S. rimosus | n.d. | n.d. | n.d. | + | n.d. | n.d. |
| DSM 41057 | S. rimosus | n.d. | n.d. | n.d. | n.d. | n.d. | n.d. |
| DSM 41224 | S. chrestomyceticus | + | n.d. | n.d. | n.d. | n.d. | + |
| DSM 41429 | S. chrestomyceticus | + | + | + | + | + | + |
| | (S. paromomycinus) | | | | | | |
| DSM-41561 | S. rimosus | n.d. | n.d. | n.d | n.d. | n.d. | n.d. |
| NRRL B-2626 | S. rimosus | n.d. | n.d. | n.d. | n.d. | n.d. | n.d. |
| NRRL B-3672 | S. chrestomyceticus | + | + | + | + | + | + |

n.d.: no detected;
+: metabolite present in fermentation broth

2.2. Efficacy of Several Streptomyces Strains Against Zymoseptoria Tritici and Puccinia recondita on Wheat

The strains listed in Table 6A were tested for their ability to control Zymoseptoria tritici and Puccinia recondita on wheat. The strains were fermented in erlenmeyer flasks as described above. End of fermentation, broth samples were frozen at −80 °C until use in glasshouse trials. At the day of application, samples were defrosted, mixed, and diluted in water supplemented with Tween20 (at 0.025% v/v) in a range of dilutions, from 10% (fermentation broth in water, v/v) to 0.3%. The spray volume was 400 liter per hectare, application was one day before infection. A positive statement (yes) reported in Table 6B indicates that the Streptomyces fermentation broth controls or reduces disease severity on the treated leaf at any of the multiple dilution ratios tested.

For infection with Zymoseptoria tritici (EPPO code: SEPTTR), the test plants of wheat cultivar Riband were inoculated by spraying a spore suspension on them one day after application (1.5Mio spores per ml in water supplemented with 0.01% Tween20). After an incubation period of 4 days at 22 °C/21 °C (day/night) and 95% rh, the inoculated test plants were kept at 22 °C/21 °C. (day/night) and 70% rh in a greenhouse. Efficacy was assessed visually when an appropriate level of disease appeared on untreated check plants (16-19 days after application).

For infection with Puccinia recondita (EPPO code: PUCCRE), the test plants of wheat cultivar Arina were inoculated by spraying them with a spore suspension one day after application (spore suspension at 80,000 spores per ml in water supplemented with Tween20 at 0.1%). After an incubation period of 1 day at 20 °C and 95% rh, the inoculated test plants were kept at 20 °C and 60% rh in a greenhouse. The percentage leaf area covered by disease was assessed visually when an appropriate level of disease appeared on untreated check plants (9-12 days after infection).

### Conclusion

The results in Table 6B show that fermentation broths from Streptomyces chrestomyceticus strains have the ability to control the development of the fungal pathogens Puccinia recondita and Zymoseptoria tritici on wheat. Streptomyces sp.CBS492.64 had the ability to control only Puccinia recondite, whereas S. albofaciens and S. rimosus did not control any the tested diseases.

TABLE 6B
The ability of several Streptomyces strains to
control Puccinia recondita or Zymoseptoria tritici
infection on wheat plants in the glasshouse.

| | | Control of | Control of |
|---|---|---|---|
| | | Puccinia | Zymoseptoria |
| | | recondita on | tritici on |
| Strain ID | Species | wheat plant | wheat plant |
| CBS 149411 | S. chrestomyceticus | Yes | Yes |
| | Streptomyces sp. | | |
| | Saigon 413 | | |
| CBS 492.64 | Streptomyces sp. | Yes | No |
| | (S. rimosis) | | |
| CBS 569.66 | S. chrestomyceticus | Yes | Yes |
| | (S. rimosis) | | |
| CBS 570.66 | S. chrestomyceticus | Yes | Yes |
| | (S. rimosis) | | |
| CBS 612.68 | S. albofaciens | No | No |
| CBS 938.68 | S. rimosus | No | No |
| DSM 40673 | S. rimosus | No | No |
| DSM 41057 | S. rimosus | No | No |
| DSM 41224 | S. chrestomyceticus | Yes | Yes |
| DSM 41429 | S. chrestomyceticus | Yes | Yes |
| | (S. paromomycinus) | | |
| DSM-41561 | S. rimosus | No | No |
| NRRL B-2626 | S. rimosus | No | No |
| NRRL B-3672 | S. chrestomyceticus | Yes | Yes |

### Example 3. Foliar Treatments with S. chrestomyceticus (Streptomyces sp. Saigon 413) Compositions to Control Fungal Infections

### 3.1. Treatment of Wheat Infected with Zymoseptoria Tritici and Puccinia Recondite with S. chrestomyceticus Fermentation Broth

Streptomyces isolate CBS 149411 (deposited at Westerdijk fungal diversity institute: Uppsalalaan 8, 3584 CT, Utrecht, The Netherlands) and NRRL B-3672 (type strain for Streptomyces chrestomycetius, deposited at ARS Culture Collection (NRRL), 1815 N. University Street, Peoria, IL 61604, USA) were fermented in a 200 L fermenter in an identical way as disclosed above. End of fermentation broth was stored frozen at −20 °C until used for assays. Wheat seedlings of the variety Riband (for Zymoseptoria tritici trials) or variety Arina (for Puccinia recondita trials) were grown in the glasshouse until 14d after seeding. One day before infection, plants were treated with different dilutions of the end-of fermentation broth (dilution in water, supplemented with0.025% Tween® 20), the spray volume used was 400 L/ha. In Table 3, the timepoint of disease evaluation is indicated in days after infection (DAI). Efficacy of the treatments is reported in percent reduction of symptoms as compared to untreated checks.

For infection with Zymoseptoria tritici (EPPO code: SEPTTR), the test plants were inoculated by spraying a spore suspension on them one day after application (1.5Mio spores per ml in water supplemented with 0.01% Tween20). After an incubation period of 4 days at 22 °C/21 °C. (day/night) and 95% rh, the inoculated test plants were kept at 22 °C/21° °C (day/night) and 70% rh in a greenhouse.

Efficacy was assessed directly when an appropriate level of disease appeared on untreated check plants (16-19 days after application).

For infection with Puccinia recondita (EPPO code: PUCCRE), the test plants were inoculated by spraying them with a spore suspension one day after application (spore suspension at 80,000 spores per ml in water supplemented with Tween20 at 0.1%). After an incubation period of 1 day at 20 °C and 95% rh, the inoculated test plants were kept at 20 °C and 60% rh in a greenhouse. The percentage leaf area covered by disease was assessed visually when an appropriate level of disease appeared on untreated check plants (9-12 days after infection).

TABLE 7
Reduction of disease symptoms on wheat caused by infection of
brown rust (Puccinia recondita) or Septoria leaf blotch
(Zymoseptoria tritici) after treatment with Streptomyces sp.
Saigon 413 and S. chrestomyceticus NRRL B-3672.

| | | | 1 d preventive application, 400 L/ha | |
|---|---|---|---|---|
| | | | disease symptom | |
| | | | reduction (%) vs. Untreated | |
| | | | Puccinia | Zymoseptoria |
| | | Assay | recondita | tritici |
| Strain | | rate | 71% disease on | 74% disease on |
| ID | test item | (% v/v) | check at 11DAI | check at 17DAI |
| CBS | Whole | 2.50 | 97 | 99 |
| 149411 | fermentation | 1.25 | 96 | 97 |
| | broth | 0.63 | 95 | 98 |
| | (liquid) + | 0.31 | 87 | 87 |
| | Tween ® 20 | 0.16 | 59 | 74 |
| | at 0.025% | 0.08 | 32 | 70 |
| | Whole | 2.50 | 97 | 96 |
| | fermentation | 1.25 | 95 | 91 |
| | broth | 0.63 | 93 | 90 |
| | (liquid) + | 0.31 | 87 | 83 |
| | Tween ® 20 | 0.16 | 59 | 79 |
| | at 0.025% | 0.08 | 25 | 46 |
| NRRL B- | Whole | 2.50 | 97 | 98 |
| 3672 | fermentation | 1.25 | 93 | 95 |
| | broth | 0.63 | 90 | 86 |
| | (liquid) + | 0.31 | 55 | 69 |
| | Tween ® 20 | 0.16 | 25 | 43 |
| | at 0.025% | 0.08 | 1 | 8 |

### Conclusions

The results in Table 7 show that both Streptomyces chrestomyceticus strains CBS 149411 and NRRL B-3672 provided control of both brown rust (Puccinia recondite) and Septoria leaf blotch (Zymoseptoria tritici) in wheat seedlings when applying end of fermentation broth. Further dilution of the broth indicates that S. chrestomyceticus CBS 149411 derived broth is more potent as compared to S. chrestomyceticus NRRL B-3672, as shown by higher control levels for example at 0.16% dilution, on both brown rust and Septoria leaf blotch.

### 3.2. Preventive and Curative Treatment of Fungal Infection by Zymoseptoria Tritici and Puccinia recondita on Wheat with Streptomyces Saigon 413 and Solatenol

Wheat seedlings of the variety Riband (for Zymoseptoria tritici trials) or variety Arina (for Puccinia recondita trials) were grown in the glasshouse until 14d after seeding. One day before infection (1d preventive assay) or 3 days after infection (3d curative assay), plants were treated with different dilution of OD40% formulation of Streptomyces Saigon 413, CBS149411. The spray volume used was 200 L/ha. In some of the treatments a tank-mix adjuvant was added (spreader and retention aid, at a typical use rate). Efficacy of the CBS 149411 drived formulation was compared to a commercial reference fungicide (Elatus™ Plus, containing SOLATENOL™, from Syngenta, Basel, Switzerland).

For infection with Zymoseptoria tritici (EPPO code: SEPTTR), the test plants were inoculated by spraying a spore suspension (1.5Mio spores per ml in water supplemented with 0.01% Tween20). After an incubation period of 4 days at 22 °C/21 °C (day/night) and 95% rh, the inoculated test plants were kept at 22 °C/21 °C (day/night) and 70% rh in a greenhouse. Efficacy was assessed directly when an appropriate level of disease appeared on untreated check plants (16-19 days after infection (DAI)).

For infection with Puccinia recondita (EPPO code: PUCCRE) The test plants were inoculated by spraying them with a spore suspension (spore suspension at 80,000 spores per ml in water supplemented with Tween20 at 0.1%). After an incubation period of 1 day at 20 °C and 95% rh, the inoculated test plants were kept at 20 °C and 60% rh in a greenhouse. The percentage leaf area covered by disease was assessed visually when an appropriate level of disease appeared on untreated check plants (9-12 days after infection (DAI)).

TABLE 8
Preventive and curative treatment of fungal infection by Zymoseptoria tritici and
Puccinia recondita on wheat with Streptomyces chrestomyceticus CBS149411 and Solatenol

| | | 1 d preventive application, 200 L/ha | | 3 d curative application, 200 L/ha | |
|---|---|---|---|---|---|
| | | disease symptom | disease symptom | disease symptom | disease symptom |
| | | reduction (%) | reduction (%) | reduction (%) | reduction (%) |
| | | vs. untreated | vs. untreated | vs. untreated | vs. untreated |
| | | Zymoseptoria | Puccinia | Zymoseptoria | Puccinia |
| | Assay | tritici | recondita | tritici | recondita |
| | rate | 100% disease on | 70% disease on | 99% disease on | 43% disease on |
| Test item | (g/ha) | chedk at 17DAI | check at 10DAI | check at 17DAI | check at 11DAI |
| SOLATENOL ™ | 27 | 97 | 99 | 82 | 92 |
| (Elatus Plus, | 9 | 70 | 96 | 61 | 84 |
| EC100) | 3 | 0 | 88 | 0 | 58 |
| | 1 | 0 | 49 | 4 | 47 |
| CBS 149411 | 600 | 96 | 82 | 31 | 0 |
| TGAI, OD40% | 300 | 77 | 67 | 0 | 0 |
| no tank-mix | 150 | 0 | 13 | 0 | 0 |
| adjuvant | 75 | 0 | 0 | 0 | 0 |
| CBS 149411 | 600 | 93 | 86 | 60 | 40 |
| TGAI, OD40% | 300 | 81 | 74 | 30 | 28 |
| with tank-mix | 150 | 36 | 43 | 10 | 20 |
| adjuvants ⁽¹⁾ | 75 | 5 | 13 | 4 | 24 |

⁽¹⁾ alkyl polyglucoside polyoxyethylene (6) C9-C11 alcohol

### Conclusions

The results in Table 8 show that Streptomyces sp. Saigon 413, CBS149411 (OD40% formulation) provided control of both brown rust and Septoria leaf blotch in wheat, similar to a conventional fungicide like SOLATENOL™. Effective disease control was provided when the S. chrestomyceticus CBS149411 OD40% was applied as a preventive spray or a curative spray. The use of tank-mix adjuvants alkyl polyglucoside and polyoxyethylene (6) C9-C11 alcohol improved efficacy of the OD40% formulation on both brown rust and Septoria leaf blotch, in a preventive and curative treatment.

### 3.3. Treatment of Wheat Infected with Yellow Rust Puccinia striiformis with Streptomyces sp. Saigon 413, CBS149411

Wheat seedlings of the variety Loft were grown in the glasshouse until 14d after seeding. One day before infection (1d preventive assay) or 4 days after infection (4d curative assay), plants were treated with one dilution of OD40% formulation. The spray volume used was 200 L/ha. A tank-mix adjuvant was added (spreader and retention aid, at a typical use rate). Efficacy of S. chrestomyceticus CBS was compared to a commercial reference fungicide (Elatus™ Plus, containing SOLATENOL™, from Syngenta, Basel, Switzerland).

Puccinia striiformis (EPPO code: PUCCST) was inoculated by spraying wheat seedling with a spore suspension (spore suspension at 80,000 spores per ml in water supplemented with Tween20 at 0.1%). After an incubation period of 48 hours at 11 °C and 95% rh, the inoculated test plants were kept at 17 °C and 60% rh in a greenhouse. The percentage leaf area covered by disease was assessed visually when an appropriate level of disease appeared on untreated check plants (16-25 days after infection (DAI)).

TABLE 9
Reduction of disease symptoms on wheat caused by infection of yellow
rust (Puccinia striiformis) after treatment of plants with
formulated samples in a preventive or curative spray timing.

| | | Application, 200 L/ha | |
|---|---|---|---|
| | | Puccinia striiformis | |
| | | 1 d preventive | 4 d curative |
| | | 29% disease on | 39% disease on |
| | | check at 22DAI | check at 21DAI |
| | Assay | disease symptom | disease symptom |
| | rate | reduction (%) | reduction (%) |
| Test item | (g/ha) | vs. Untreated | vs. Untreated |
| SOLATENOL ™ (Elatus | 75 | 100 | 100 |
| Plus, EC100) | 25 | 100 | 99 |
| | 8 | 90 | 95 |
| | 2.8 | 76 | 93 |
| | 0.9 | 0 | 84 |
| CBS 149411 | 500 | 91 | 98 |
| TGAI, formulated | | | |
| OD40% + alkyl | | | |
| polyglucoside and | | | |
| polyoxyethylene (6) | | | |
| C9-C11 alcohol | | | |

### Conclusions

The results in Table 9 show that S. chrestomyceticus CBS 149411 formulated as OD40% provided control of yellow rust in wheat, similar to a conventional fungicide like SOLATENOL™ in both a preventive and curative treatment.

### Example 3.4. Treatment of Black Sigatoka (Mycosphaerella fijensis) on Banana with Streptomyces sp. Saigon 413

Spray dried powder of S. chrestomyceticus CBS 149411 was applied to banana plants in combination with a spray oil (Orchex® 796) and an emulsifier (Emulsogen M). Banana plants (variety Dwarf Cavendish, grown 6 weeks in the glasshouse from commercial in-vitro explants) were treated twice with this triple mixture, 8 days and 1 day before infection. Efficacy of CBS 149411 powder was compared to a treatment with only spray oil plus emulsifier at the same spray timing. The fungicide MIRAVIS® Bold (with the fungicide ADEPIDYN®, from Syngenta, Basel, Switzerland), suspension concentrate (SC200), in mixture with the same spray oil and emulsifier, was used as a positive control in the experiment, with one spray at one day before infection. Orchex® 796 is an agricultural spray oil (a product from Calumet specialty products partners, Indianapolis, USA). Emulsogen M is an emulsifier for mineral oils (a product from Clariant international, Muttenz, Switzerland) and contains oleyl alcohol polyglycol ether.

Mycosphaerella fijiensis (EPPO code: MYCOFI) was inoculated by spraying banana plants with a spore suspension (spore suspension at 100,000 spores per ml in water supplemented with Tween20 at 0.1%). After an incubation period of 48 hours under a hood to retain high relative humidity (rh), the inoculated test plants were kept at 24 °C and 90% rh in a greenhouse until evaluation of symptoms. The percentage leaf area covered by disease was assessed visually when an appropriate level of disease appeared on untreated check plants (7-10 weeks after infection, (expressed as day after infection (DAI)).

### Conclusion

The results in Table 10 show that treating banana plants with S. chrestomyceticus CBS 149411 powder in combination with spray oil and emulsifier provided control of black sigatoka in banana.

TABLE 10
Reduction of disease symptoms on banana caused by infection of black
Sigatoka (Mycosphaerella fijiensis) after treatment of plants with
S. chrestomyceticus CBS149411 in a preventive spray timing.

| | | Assay rate | Disease symptom |
|---|---|---|---|
| | | fungicide | reduction (%) |
| Pathogen | Test item | (g/ha) | vs. untreated |
| | 1 d preventive 400 L/ha | | |
| Mycosphaerella fijensis | untreated + | 0 | 43 |
| evaluation at 63DAI, | 0.9% Orchex 796 + | | |
| 98% on check | 0.1% Emulsogen M | | |
| | ADEPIDYN ®, MIRAVIS ® Bold, | 75 | 97 |
| | C200 + | | |
| | 0.9% Orchex 796 + | | |
| | 0.1% Emulsogen M | | |
| | 1 d preventive + 8 d preventive application, 400 L/ha | | |
| | untreated + | 0 | 23 |
| | 0.9% Orchex 796 + | | |
| | 0.1% Emulsogen M | | |
| | CBS 149411 spray dried TGAI + | 1600 | 67 |
| | 0.9% Orchex 796 + | | |
| | 0.1% Emulsogen M | | |

### Example 3.5 Treatment of Fungal Diseases on Rice with Streptomyces Saigon 413

An OD40% or and NAD40% formulation of Streptomyces sp. Saigon 413, CBS 149411 in combination with Tween®20 at 0.05% were sprayed on rice plants one or two days before infection, with a spray volume of 400 L/ha.

For infection with Pyricularia oryzae (EPPO code: PYRIOR), 14 days old rice plants variety Koshihikari are gown in pots. One day after spray of the product, plants were inoculated with a spore suspension (ca. 85′000 spores per millilitre) in water plus 0.04% Tween20 using a paint brush. Plants were maintained at 23 °C, 14 h of light and high relative humidity (>80%) until evaluation. The percentage leaf area covered by disease was assessed visually when an appropriate level of disease appeared on untreated check plants (ca. 8 days after infection (DAI)).

For infection with Rhizoctonia solani (EPPO code: RHIZSO), 14 days old rice plants variety Koshihikari are gown in pots. One day after spray of the product plants were inoculated with a mycelium suspension (mycelium macerated with a blender and filtered through a sieve) in water plus 0.1% Tween20 using a paint brush. Plants were maintained at 23 °C, 14 h of light and high relative humidity (>80%) until evaluation. The percentage leaf area covered by disease was assessed visually when an appropriate level of disease appeared on untreated check plants (ca. 7 days after infection (DAI)).

For infection with Xanthomonas oryzae pv. oryzae (EPPO code: XANTOR). 3 weeks old rice plants variety Koshihikari were treated with the formulated test compound in a spray chamber. Two days after application rice plants were infected by cutting the upper leaf level with scissors that were previously dipped in a bacteria suspension. After an incubation period of 14 days at 23 °C and high relative humidity (>80%) a visual assessment of the disease level was performed.

The product Tilt® (with the fungicide Propiconazole, from Syngenta, Basel, Switzerland), and Amistar® (with the fungicide Azoxystrobin, from Syngenta, Basel, Switzerland), were used as positive controls in the experiment.

### Conclusion

The results in Table 11 show that Streptomyces chrestomyceticus CBS 149411 formulated as either an OD40% or an NAD40% provided control of rice blast (PYRIOR), sheath blight (RHIZSO) and bacterial blight (XANTOR) in rice.

TABLE 11
Reduction of disease symptoms on rice caused by infection of rice blast (Pyricularia
oryzae), sheath blight (Rhizoctonia solani) or bacterial blast (Xanthomonas oryzae pv.
oryzae) after treatment of plants with S. chrestomyceticys CBS149411 OD40% and
NAD40% formulations and chemical fungicides in a preventive treatment

| | | 1 d preventive application, | | 2 d preventive |
|---|---|---|---|---|
| | | 400 L/ha | | application, 400 L/ha |
| | | Pyricularia | Rhizoctonia | Xanthomonas |
| | | oryzae | solani | oryzae pv. Oryzae |
| | | Disease | Disease | Disease |
| | Assay | symptom | symptom | symptom |
| | rate | reduction (%) | reduction (%) | reduction (%) |
| Test item | (g/ha) | vs. untreated | vs. untreated | vs. untreated |
| Propiconazole (Tilt ®, | 125 | 92 | — | — |
| EC250) | 42 | 79 | — | — |
| | 14 | 38 | — | — |
| | 5 | 17 | — | — |
| Azoxystrobin (Amistar ®, | 83 | 92 | 86 | — |
| SC250) | 28 | 96 | 81 | — |
| | 9 | 85 | 86 | — |
| | 3 | 92 | 62 | — |
| CBS 149411 | 1600 | 92 | 71 | 62 |
| TGAI, formulated | 533 | 79 | 23 | 17 |
| OD40% + | 178 | 38 | 4 | 36 |
| Tween ® 20 at 0.05% | 59 | 0 | 4 | — |
| CBS 149411 | 1600 | 58 | 62 | 81 |
| TGAI, formulated | 533 | 69 | 71 | 68 |
| NAD40% + | 178 | 17 | 52 | 23 |
| Tween ® 20 at 0.05% | 59 | 0 | 23 | — |

### Example 3.6. Treatment of Powdery Mildew (Blumeria graminis f.Sp. Tritici) on Wheat with Streptomyces sp. Saigon 413 and SOLATENOL™

Streptomyces chrestomyceticus CBS 149411 TGAI powder was applied to wheat plants in combination with a tank-mix adjuvant (a methyl cellulose (3%) in water) to improve retention and spreading. The spray volume used was 200 L/ha. Efficacy of the CBS 149411 derived formulation was compared to a commercial reference fungicide (Elatus™ Plus, containing SOLATENOL™, from Syngenta, Basel, Switzerland).

Blumeria graminis f.sp. tritici (EPPO code: ERYSGT) was inoculated by dusting 14 days old wheat seedling (cultivar Arina) with spores from infected plants with good sporulation (from a disease nursery). The inoculated test plants were kept at 20 °C and 60% rh in a greenhouse. The percentage leaf area covered by disease was assessed visually when an appropriate level of disease appeared on untreated check plants (ca. 7 days after infection (DAI)).

### Conclusion

The results in Table 12 show that S. chrestomyceticus CBS 149411 TGAI in combination with a tank-mix adjuvant provided control of powdery mildew Blumeria graminis f.sp. tritici in wheat.

TABLE 12
Reduction of disease symptoms on wheat caused by infection of powdery
mildew (Blumeria graminis f.sp. tritici) after treatment of the plants
with Streptomyces chrestomyceticus CBS 149411 and SOLATENOL ™

| | | 1 d preventive application, 200 L/ha | |
|---|---|---|---|
| | | | disease symptom |
| | | Assay rate | reduction (%) |
| pathogen | Test item | (g/ha) | vs. Untreated |
| Blumeria graminis f.sp. tritici | SOLATENOL ™, Elatus | 27 | 54 |
| evaluation at 7DAI, 75% on | Plus, EC100 | | |
| check | CBS 149411 | 1600 | 34 |
| | TGAI + methyl cellulose | | |
| | 3% w/v | | |

### Example 3.7. Treatment of Fungal Infections on Tomato, Cucumber and Ground Nuts with Streptomyces sp. Saigon 413 or SOLATENOL™

An OD40% formulation of S. chrestomyceticus CBS 149411 was sprayed in combination with Tween®20 at 0.025% on tomato, cucumber and ground nut plants one day before infection, with a spray volume of 400 L/ha. Elatus™ Plus, containing SOLATENOL™, (Syngenta, Basel, Switzerland) was used as a reference fungicide

Alternaria solani (EPPO Code: ALTESO)

4-weeks old tomato plants cultivar Roter Gnom were sprayed in a spray chamber with the formulated test compound diluted in water. The test plants were inoculated by spraying them with a spore suspension two days after application. The inoculated test plants were incubated at 22/18 °C (day/night) and 95% rh in a greenhouse and the percentage leaf area covered by disease is assessed when an appropriate level of disease appears on untreated check plants (5-7 days after application).

Colletotrichum orbiculare (EPPO Code: COLLLA)

1 week old cucumber plants cultivar. Wisconsin were treated with the formulated test compound in a spray chamber. One day after application the plants were inoculated by spraying a spore suspension (1×105 conidia/ml) on the test plants. After an incubation period of 1 day at 25 °C and 100% r. h. plants were kept for 6 days at 22 °C and 70% r.h.in a greenhouse. The disease incidence was assessed 7 days after inoculation (DAI).

Mycosphaerella arachidis (EPPO Code: MYCOAR)

3 weeks old peanut plants cultivar Florunner were treated with the formulated test compound in a spray chamber. One day after application the plants were inoculated by spraying a spore suspension (3×105 conidia/ml) on the test plants. After an incubation period of 4 days at 24 °C and 95% r. h. plants were kept for 7 days at 26 °C and 70% r.h.in a greenhouse. The disease incidence was assessed 11 days after inoculation (DAI). R.h.: Relative humidity

### Conclusion

Table 13 shows that an OD40% formulation of S. chrestomyceticus CBS 149411 provided control of early blight (Alternaria solani) on tomato, anthracnose (Colletotrichum orbiculare) on cucumber and early leaf spots (Mycosphaerella arachidis) on groundnut.

TABLE 13
Reduction of disease symptoms caused by early blight (Alternaria solani)
on tomato, anthracnose (Colletotrichum orbiculare) on cucumber or early leaf
spots (Mycosphaerella arachidis) on groundnut after treatment of plants with
S. chrestomyceticus CBS 149411 OD40% formulation and SOLATENOL ™

| | | 1 d preventive application, 400 L/ha | | |
|---|---|---|---|---|
| | | | Colletotrichum | Mycosphaerella |
| | | Alternaria solani | lagenarium | arachidis |
| | | on tomato | on cucumber | on groundnut |
| | | 75% disease on | 75% disease on | 45% disease on |
| | | check at 4DAI | check at 5DAI | check at 11DAI |
| | Assay | disease symptom | disease symptom | disease symptom |
| | rate | reduction (%) | reduction (%) | reduction (%) |
| Test item | (g/ha) | vs. Untreated | vs. Untreated | vs. Untreated |
| SOLATENOL ™ | 60 | 100 | 100 | 98 |
| (Elatus ™ Plus, EC100) | 20 | 90 | 90 | 100 |
| | 6 | 80 | 60 | 89 |
| | 2 | 53 | 67 | 67 |
| | 1 | 40 | 53 | 56 |
| CBS 149411 | 4500 | 53 | 97 | 83 |
| TGAI, formulated | 1500 | 27 | 95 | 67 |
| OD40% + | 500 | 7 | 73 | 33 |
| Tween 20 at 0.025% | 167 | 0 | 40 | 0 |
| | 56 | 0 | 20 | 11 |

### Example 3.8. Treatment of Fungal Infections on Barley and Wheat with S. chrestomyceticus CBS 149411 (Streptomyces sp. Saigon 413) and a Chemical Fungicide

An OD40% and a NAD40% formulation from S. chrestomyceticus CBS 149411 was sprayed on barley and wheat plants one day before infection, with a spray volume of 200 L/ha. Elatus™ Plus, containing SOLATENOL™ (Syngenta, Basel, Switzerland) was used as a reference fungicide

Ramularia collo-cygni (EPPO Code: RAMUCC)

12 days old barley plants cultivar Venture were treated with the formulated test compound in a spray chamber. One day after application the plants were inoculated by spraying a spore suspension (3×10⁵ conidia/ml) on the test plants. After an incubation period of 2 days under a hood, plants are kept in the glasshouse for 15 days at 21°/19 °C. (day/night) and 80% relative humidity. The disease incidence was assessed 17 days after inoculation (DAI).

Parastagonospora nodorum (EPPO Code: LEPTNO)

13 days old wheat plants cv. Riband were treated with the formulated test compound in a spray chamber. One day after application the plants were inoculated by spraying a spore suspension (4×10⁵ conidia/ml) on the test plants. After an incubation period of 2 days under high relative humidity (>95%), plants are kept in the glasshouse for 5 days at 21°/19 °C. (day/night) and 70% relative humidity. The disease incidence was assessed 7 days after inoculation (DAI).

Rhynchosporium secalis (EPPO Code: RHYNSE)

14 days old barley plants cv. Pasadena were treated with the formulated test compound in a spray chamber. One day after application the plants were inoculated by spraying a spore suspension (4×10⁵ conidia/ml) on the test plants. After an incubation period of 2 days under high relative humidity (>95%), plants are kept in the glasshouse for 13 days at 21°/19 °C. (day/night) and 70% relative humidity. The disease incidence was assessed 15 days after inoculation (DAI).

### Conclusion

The results in Table 14 show that an OD40% or a NAD40% formulation of S. chrestomyceticus CBS148411 provided control of ramularia leaf spot (Ramularia collo-cygni) on barley, glume blotch (Parastagonospora nodorum) on wheat or scald (Rhynchosporium secalis) on barley

TABLE 14
Reduction of disease symptoms caused by ramularia leaf spot (Ramularia collo
-cygni) on barley, glume blotch (Parastagonospora nodorum) on wheat
or scald (Rhynchosporium secalis) on barley after treatment of plants
with an OD40% and NAD40% formulation of S. chrestomyceticus

| | | 1 d preventive application, 200 L/ha | | |
|---|---|---|---|---|
| | | Ramularia | Parastagonospora | Rhynchosporium |
| | | collo-cygni | nodorum | secalis |
| | | on barley | on wheat | on barley |
| | | 83% on check | 73% on check | 85% on check |
| | | at 20DAI | at 7DAI | at 15DAI |
| | Assay | disease symptom | disease symptom | disease symptom |
| | rate | reduction (%) | reduction (%) | reduction (%) |
| Test item | (g/ha) | vs. Untreated | vs. Untreated | vs. Untreated |
| Solatenol (Elatus | 120 | 97 | 85 | 100 |
| Plus, EC100) | 40 | 80 | 81 | 94 |
| | 12 | 32 | 44 | 60 |
| | 4 | 6 | 16 | 31 |
| CBS 149411 | 1500 | 18 | 40 | 89 |
| TGAI, formulated | 500 | 16 | 33 | 79 |
| OD40% | 166 | 12 | 32 | 35 |
| | 55 | 12 | 30 | 15 |
| CBS 149411 | 1500 | 35 | 65 | 84 |
| TGAI, formulated | 500 | 25 | 64 | 59 |
| NAD40% | 166 | 6 | 52 | 43 |
| | 55 | 0 | 43 | 18 |

### Example 3.9. Treatment of Eyespot (Oculimacula yallundae) on Wheat with S. chrestomyceticus CBS149411 (Streptomyces sp. Saigon 413) and Cyprodinil

A NAD40% formulation of S. chrestomyceticus CBS 149411 was sprayed on plants one day before infection, with a spray volume of 200 L/ha. Unix® WG750, containing Cyprodinil (Syngenta, Switzerland) was used as a reference fungicide

For infection of Oculimacula yallundae (EPPO Code: PSDCHE)

11 days old wheat plants cultivar Arina were treated with the formulated test compound in a spray chamber. One day after application the plants were inoculated by spraying a spore suspension (6×10⁵ conidia/ml) on the test plants, targeting the base of the plant in particular. After an incubation period of 2 days under a hood, plants are kept in the glasshouse at 11°/10 °C. (day/night) and 80% relative humidity. The disease incidence was assessed when symptoms had well developed on untreated checks (ca. 7 to 12 weeks after inoculation).

### Conclusion

The results in Table 15 show that S. chrestomyceticus CBS 149411 formulated as NAD40% provided an effective means to control eyespot (Oculimacula yallundae) on wheat.

TABLE 15
Reduction of disease symptoms caused by yellow
rust (Oculimacula yallundae) on wheat with
S. chrestomyceticus CBS149411 and Cyprodinil

| | Oculimacula yallundae | |
|---|---|---|
| | 1 d preventive application, 200 L/ha | |
| | | disease symptom |
| | | reduction (%) |
| Test item | Assay rate (g/ha) | vs. Untreated |
| Cyprodinil, Unix ® WG750 | 450 | 56 |
| CBS 149411 | 800 | 25 |
| TGAI, formulated NAD40% | | |

### Example 3.10. Treatment of Fungal Diseases on Wheat with Streptomyces chrestomyceticus CBS149411 Beyond the Treated Area

A NAD40% formulation of S. chrestomyceticus CBS 149411 was applied on leaf segments one day before infection, with a volume of approximately 400 L/ha. Efficacy of the CBS 149411 derived formulation was compared to a commercial reference fungicide (Elatus™ Plus, containing SOLATENOL™ Syngenta, Switzerland).

Wheat seedlings of the variety Riband (for Zymoseptoria tritici trials) or variety Arina (for Puccinia recondita trials) were grown in the glasshouse until 14d after seeding. At this time, such seedlings usually have a first leaf fully emerged (designated L1), a second leaf fully emerged (L2) and a third leaf (L3) that is partially emerged and in the process of development. Using a permanent pen, two dots were applied on the second leaf such as to generate three segments of roughly equal size: the base, middle part and tip of the leaf. The compound to be tested was diluted in water at the indicated concentration (in ppm). The diluted compound was applied to the middle segment of leaf 2 using a conventional cotton stick; the cotton stick was soaked in the diluted compound and rubbed several times on the adaxial leaf surface between the two marks. One day later, the complete plant was inoculated with a fungal spore suspension using a paint brush. The spore suspension was applied until before run-off.

For infection with Zymoseptoria tritici (EPPO code: SEPTTR), the test plants were inoculated by spraying a spore suspension on them one day after application (1.5Mio spores per ml in water supplemented with 0.01% Tween20). After an incubation period of 4 days at 22 °C/21 °C (day/night) and 95% rh, the inoculated test plants were kept at 22 °C/21 °C. (day/night) and 70% rh in a greenhouse. Efficacy was assessed directly when an appropriate level of disease appeared on untreated check plants (16-19 days after application (DAI)).

For infection with Puccinia recondita (EPPO code: PUCCRE) The test plants were inoculated by spraying them with a spore suspension one day after application (spore suspension at 80,000 spores per ml in water supplemented with Tween20 at 0.1%). After an incubation period of 1 day at 20 °C and 95% rh, the inoculated test plants were kept at 20 °C and 60% rh in a greenhouse. The percentage leaf area covered by disease was assessed visually when an appropriate level of disease appeared on untreated check plants (9-12 days after infection (DAI).

### Conclusion

The results in Table 16 show that S. chrestomyceticus CBS 149411 was able to protect the treated leaf areas of wheat plants from infection by Septoria leaf blotch (Zymoseptoria tritici) and brown rust (Puccinia recondita). The level of protection achieved by S. chrestomyceticus CBS 149411 was comparable to the effect of a conventional fungicide (example: SOLATENOL™). Similar to a SOLATENOL™, the untreated acropetal part of leaf 2 was equally well protected from both diseases. To our surprise the base of leaf 2 was also highly protected from disease (>88%) when using 2500 ppm assay rates. Similarly, the systemic leaf 3 was also protected by application of product on leaf 2. The protection of the basipetal part of leaf 2 as well as the systemic leaf 3 were found to be dependent on the application rate.

TABLE 16
Reduction of disease symptoms caused by Septoria eaf blotch (Zymoseptoria tritici) and brown rust
(Puccinia recondita) on wheat infection after treatment of plants with a NAD35% formulated S.
chrestomyceticus CBS149411 in a preventive timing on treated leaf and systemic (non-treated) leaf

| | | | | 1 d preventive application, ca 400 L/ha | | | |
|---|---|---|---|---|---|---|---|
| | | | | Zymoseptoria | Puccinia | Zymoseptoria | Puccinia |
| | | | | tritici | recondita | tritici | recondita |
| | | | | evaluated | evaluated | disease | disease |
| | | | | at 17DAI | at 11DAI | symptom | symptom |
| | Assay | | | disease | disease | reduction | reduction |
| | rate | | | cover on | cover on | (%) vs. | (%) vs. |
| Test item | (ppm) | leaf segment | treated | leaf (%) | leaf (%) | untreated | untreated |
| untreated | 0 | leaf 1 | no | 100 | 90 | 0 | 0 |
| check | | leaf 2 - base | no | 97 | 58 | 0 | 0 |
| | | leaf 2 - middle | no | 98 | 69 | 0 | 0 |
| | | leaf 2- tip | no | 100 | 63 | 0 | 0 |
| | | leaf 3 (*) | no | 49 | 37 | 0 | 0 |
| SOLATENOL ™ | 188 | leaf 1 | no | 100 | 80 | 0 | 11 |
| (Elatus ™ | | leaf 2 - base | no | 89 | 81 | 8 | 0 |
| Plus, | | leaf 2 - middle | yes | 0 | 0 | 100 | 100 |
| EC100) | | leaf 2- tip | no | 0 | 2 | 100 | 98 |
| | | leaf 3 (*) | no | 42 | 31 | 15 | 16 |
| CBS 149411 | 2500 | leaf 1 | no | 98 | 79 | 2 | 12 |
| TGAI, | | leaf 2 - base | no | 0 | 7 | 100 | 88 |
| formulated | | leaf 2 - middle | yes | 0 | 2 | 100 | 97 |
| NAD35% | | leaf 2- tip | no | 11 | 2 | 89 | 97 |
| | | leaf 3 (*) | no | 3 | 13 | 95 | 65 |
| | 250 | leaf 1 | no | 100 | 76 | 0 | 16 |
| | | leaf 2 - base | no | 62 | 63 | 36 | 0 |
| | | leaf 2 - middle | yes | 2 | 4 | 89 | 95 |
| | | leaf 2- tip | no | 12 | 2 | 88 | 97 |
| | | leaf 3 (*) | no | 48 | 40 | 3 | 0 |

(*) on leaf 3, a significant part of the leaf is new growth since the time of infection, therefore only partially infected even in untreated check.

### Example 3.11. Treatment of Fusarium Head Blight in Wheat with S. chrestomyceticus CBS149411 (Streptomyces sp. Saigon 413)

A NAD35% formulation of S. chrestomyceticus CBS149411 was applied on leaf segments one day before infection in presence of a tank-mix adjuvant, with a spray volume of approximately 220 L/ha. Miravis™ Plus, containing ADEPIDYN™ (Syngenta, Switzerland) was used as a reference fungicide.

Fusarium avenaceum (EPPO Code: GIBBAV)

Flowering wheat plants cv. Monsun were treated with the formulated test compound in a spray chamber. One day after application the plants were inoculated by spraying a spore suspension (1×10⁵ conidia/ml) on the ears. After an incubation period of 2 days with water misting, under high relative humidity (>95%) and absence of light, plants were kept in the glasshouse for 12 days at 21°/19 °C. (day/night) and 70% relative humidity. The disease incidence on the ears was assessed 14 days after inoculation. The inoculum consisted of a triple mix of three distinct isolates (ratio ca. 1:1:1), each of them confirmed to produce different mycotoxins typical for this species (EnniatinA, EnniatinA1, EnniatinB, EnniatinB1, moniliformin).

Fusarium graminearum (EPPO Code: GIBBZE)

Flowering wheat plants cultivar Monsun were treated with the formulated test compound in a spray chamber. One day after application the plants were inoculated by spraying a spore suspension (1×10⁵ conidia/ml) on the ears. After an incubation period of 2 days with water misting, under high relative humidity (>95%) and absence of light, plants were kept in the glasshouse for 5 days at 21°/19 °C (day/night) and 70% relative humidity. The disease incidence on the ears was assessed 7 days after inoculation. The inoculum consisted of a triple mix of three distinct isolates (ratio ca. 1:1:1), each of them confirmed to produce different mycotoxins typical for this species (DON=deoxynivalenol, 3- or 15-acetyldeoxynivalenol, zearalenone).

### Conclusion

The results in Table 17 show that S. chrestomyceticus CBS 149411 formulated as NAD35% provided an effective means to control Fusarium head blight (Fusarium graminearum or Fusarium avenaceum) on wheat. Fusarium species with different chemotypes (DON producer and enniatin producer) were controlled

TABLE 17
Reduction of disease symptoms caused by Fusarium head blight (Fusarium graminearum
or Fusarium avenaceum) on wheat infection after treatment of plants
with formulated samples in a preventive spray timing

| | | 1 d preventive application, 220 L/ha | |
|---|---|---|---|
| | | | disease symptom |
| | | | reduction (%) |
| pathogen | Test item | Assay rate (g/ha) | vs. Untreated |
| Fusarium avenaceum | ADEPIDYN ™, Miravis ™ | 125 | 96 |
| evaluation at 14DAI, 24% | Plus, EC62.5 | | |
| on check | CBS 149411 | 1600 | 40 |
| | TGAI, NAD35% + | | |
| | Alkyl Polyglucoside | | |
| | Polyoxyethylene (6) C9- | | |
| | C11 alcohol | | |
| Fusarium graminearum | ADEPIDYN ™, Miravis ™ | 125 | 94 |
| evaluation at 7DAI, 35% | Plus, EC62.5 | | |
| on check | | | |
| | CBS 149411 | 1600 | 75 |
| | TGAI, formulated | | |
| | NAD35% + | | |
| | Alkyl Polyglucoside | | |
| | Polyoxyethylene (6) C9- | | |
| | C11 alcohol | | |

### Example 4. Treatment of Seeds with Streptomyces chrestomyceticus CBS149411

### Methodology of Semi Field Platform as Used in Example 4.1A and 4.1B

A semi-field platform consists of a water pool system which contains pots, wherein the soil temperature is controlled. A cooling unit allows to control the water temperature independent of the outside weather conditions according to the requirements for disease establishment. Plastic pots (vol 60 L) were filled with 45 liters freshly sieved soil

### Seeds Treatments

Prior to sowing, seeds were treated according to standard procedures known to a person skilled in the art with a slurry amount, containing the test compounds, of 5 g and 10 g per kg seeds for corn and wheat respectively, using a Turbula mixer. The treated seeds were allowed to dry in open bottles at room temperature for 24 h and then stored in paper bags at room temperature until sowing.

### Example 4.1A. Effect of Streptomyces chrestomyceticus Saigon 413 on Soilborne Fusarium graminearum in Corn Under Semi-Field Conditions

In this semi-field experiment a topsoil layer was mixed with pre-inoculated substrate of the test fungus, i.e. Fusarium graminearum. A total of 100 corn seeds cultivar Arma were sown per pot. A complete randomized block design was used with 4 replicates (pots) per treatment. The pots were placed in the water basin which contained cold water (10 °C). The water temperature was kept around 10 °C for the first three weeks and then at 15° to 20 °C for the following two weeks.

The following treatments using standard procedures as disclosed above were applied to corn seeds:
spray dried broth, 200 g TGAI of Streptomyces chrestomyceticus Saigon 413 per 100 kg seeds
freeze dried supernatant, 200 g TGAI Streptomyces chrestomyceticus Saigon 413 per 100 kg seeds
Standard (reference treatment), Fludioxonil (product: Celest, FS025) at 2.5 g ai per 100 kg. seeds

Two control treatments were included in this test, i.e., an infected and a non-infected control. The activity (%) of each seed treatment was calculated based on the plant emergence (number of plants) (%) at final stand in comparison to the infected control.

### Results:

The disease pressure in the experiment was high, i.e. the final plant stand was reduced by 72% in the infected control treatment. The reference treatment provided 97% activity. The spray dried broth provided 84% activity. The freeze dried supernatant provided 69% activity.

### Example 4.1B. Effect of Seed Applied Streptomyces chrestomyceticus Saigon 413 on Soilborne Infection by Fusarium graminearum in Corn and Fusarium culmorum in Wheat Under Semi-Field Conditions

In this semi-field experiment a topsoil layer was mixed with pre-inoculated substrate of the test fungi, i.e. Fusarium graminearum (K-6102) or Fusarium culmorum. A total of 100 corn seeds cultivar Andromeda or 100 wheat seeds cv Taifun were sown per pot. A complete randomized block design was used with 4 replicates (pots) per treatment. The pots were placed in the water basin which contained cold water (12 °C).

The following treatments were applied to corn seeds and wheat seeds using standard procedures as disclosed above:
Wheat seeds were treated with a flowable concentrate (FS300) of Streptomyces chrestomyceticus Saigon 413 at 200 g TGAI per 100 kg seeds
Corn seeds were treated with an oil dispersion, OD400 of Streptomyces chrestomyceticus Saigon 413, at 200 g TGAI per 100 kg seeds
Standard (reference treatment), Fludioxonil (product: Celest, FS025)
   at 2.5 g ai per 100 kg seeds for experiment with corn
   at 5 g ai per 100 kg seeds for experiment with wheat

Two control treatments were included in this test, i.e. an infected and a non-infected control. The activity (%) of each seed treatment was calculated based on the plant emergence (%) at final plant stand in comparison to the infected control.

### Results:

The disease pressure in the experiments was moderate, i.e. the final plant stand in the infected control treatment was reduced by 19% for wheat and 34% for corn. The reference treatment provided 100% activity in the corn experiment and 93% activity in the wheat experiment.

In the wheat experiment with Fusarium culmorum, Streptomyces Saigon 413 provided 92% activity at 200 g TGAI.

In the corn experiment with Fusarium graminearum, the Streptomyces Saigon 413 provided 99% activity at 200 g TGAI.

The results show that both the oil dispersion formulation as the flowable concentrate formulation resulted in more than 90% activity.

### Example 4.1C. Effect Streptomyces Saigon 413 Applied on Corn Seeds on Soilborne Fusarium graminearum Under Controlled Conditions in the Greenhouse

Corn seeds cv Andromeda were treated according to standard procedures known to a person skilled in the art with a slurry amount of 5 g per kg seeds using a Turbula mixer. The treated seeds were allowed to dry in open bottles at room temperature for 24 h. Four replicates (soil trays) were used, each containing 25 seeds. Before sowing, the soil was incubated with fungal spores of the test pathogen, i.e. Fusarium graminearum, for 7 days at 18 °C.

The following seed applied treatments were compared:
Streptomyces chrestomyceticus Saigon 413 TGAI formulated as FS300 at 200 g TGAI per 100 kg seeds
Standard (reference treatment), Fludioxonil (product Celest, FS025) at 2.5 g ai per 100 kg seeds

Two control treatments were included in this test, i.e., an infected and a non-infected control.

The activity (%) of each seed treatment was calculated based on the plant emergence (%) at final stand in comparison to the infected control.

### Results:

The disease pressure in the experiment was moderately high, i.e. the final plant stand in the infected control treatment was reduced by 45%. The reference treatment (Fludioxonil) at 2.5 g ai provided 96% activity. Streptomyces chrestomyceticus Saigon 413 TGAI at 200 g TGAI provided 82% activity.

### Example 4.2. Effect of Seed Applied Streptomyces chrestomyceticus Saigon 413 on Soilborne Infection by Fusarium virguliforme in Soybean Under Controlled Conditions in the Greenhouse

Soybean seeds cultivar Toliman were treated using standard procedures as disclosed above. Five replicates (pots) were used each containing 5 seeds. The soil was mixed with pre-inoculated substrate of the test fungus, i.e. Fusarium virguliforme.

The following seed applied treatments were compared:
Streptomyces chrestomyceticus Saigon 413 TGAI formulated as FS300 at 200 g TGAI per 100 kg seeds.
Standard (reference treatment), ADEPIDYN® (active ingredient: Pydiflumetofen; FS500) at 40 g ai per 100 kg seeds

Two control treatments were included in this test, i.e. an infected and a non-infected control. The activity (%) calculation of the seed treatments was based on the disease index, which is derived from the severity of the symptoms on the leaves, in comparison to the infected control.

### Results:

The disease pressure in the experiment was high, i.e. the disease incidence was at 80%. The reference treatment (ADEPIDYN®) provided 89% activity at 40 g ai. Streptomyces chrestomyceticus Saigon 413 TGAI provided 58% activity at 200 g ai.

### Example 4.3. Effect of Seed Applied Streptomyces chrestomyceticus Saigon 413 on Soilborne Aphanomyces cochlioides (Oomycete) in Sugar Beet Under Controlled Conditions in the Greenhouse

Sugar beet seeds cultivar Jagger were treated according to standard procedures known in the art as disclosed above. Five replicates (pots) were used, each containing 25 seeds. At 7 days after sowing, a mycelium suspension of the plant pathogen Aphanomyces cochlioides strain K-9164 was added in each pot.

The following seed applied treatments were compared:
Streptomyces Saigon 413 TGAI formulated as FS300 at TGAI g ai per 1 million seeds
Standard (reference treatment), Hymexazol (product Tachigaren, WS070) at TGAI g ai per 1 million seeds

Two control treatments were included in this test, i.e., an infected and a non-infected control.

The activity (%) of each seed treatment was calculated based on damping off symptoms (counting of dead plants) in comparison to the infected control.

### Results:

The disease pressure in the experiment was moderately high, i.e. 56% dead plants in the infected control. The reference treatment (Hymexazol) provided 74% activity. Streptomyces Saigon 413 TGAI provided 53% activity.

### Example 4.4. Effect of Seed Applied Streptomyces chrestomyceticus Saigon 413 on Soilborne Infection by Gaeumannomyces graminis in Wheat Under Controlled Conditions in the Greenhouse

Wheat seeds cultivar Arina were treated according to standard procedures as disclosed above. 4 replicates (pots) were used, each containing 15 seeds. At sowing, the soil substrate was mixed with fungal inoculum.

The following seed applied treatment was tested:
S. chrestomyceticus (Streptomyces Saigon 413) TGAI as FS300 (FS: Flowable concentrate for seed treatment) at 100 g ai/100 kg seeds

Two control treatments were included in this test, i.e., an infected and a non-infected control.

The activity (%) of the seed treatment was calculated based on root health (severity of disease symptoms) as compared to the control treatments.

### Results:

The disease pressure in the experiment was high, i.e. 76% of unhealthy roots in the infected control. Streptomyces Saigon 413 TGAI provided 25% activity (efficacy).

The results of Experiments 4.1 to 4.4 are summarized in Table 18.

### Conclusion

The results of Experiments 4.1 to 4.4 as shown in Table 18 show that Streptomyces chrestomyceticus CBS149411 was effective in controlling diseases caused by of several fungal (Fusarium and Gaeumannomyces) infections and an oomycete infection on seeds

TABLE 18
Overview of the activity of S. chrestomyceticus CBS149411
on seeds against several Fusarium sp. on different crops,
against the oomycete Aphanomyces cochlioides on sugar beet
and against Gaeumannomyces graminis on wheat

| | Seed applied product, | Activity |
|---|---|---|
| | application rate in g | in % |
| Target, crop | ai per 100 kg seeds | (example) |
| Fusarium | Spray dried broth 200 g | 84% (4.1A) |
| graminearum, corn | Freeze dried supernatant 200 g | 69% (4.1A) |
| | OD400, 200 g | 99% (4.1B) |
| | FS300, 200 g | 82% (4.1C) |
| Fusarium culmorum, | FS300, 200 g | 92% (4.1B) |
| wheat | | |
| Fusarium virguliforme, | FS300, 200 g | 58% (4.2) |
| soybean | | |
| Aphanomyces | FS300, 28 g ai per 1 million | 53% (4.3) |
| cochlioides, sugar | seeds | |
| beet | | |
| Gaeumannomyces | FS300, 100 g | 25% (4.4) |
| graminis, wheat | | |

### Example 5. Activity of Streptomyces chrestomyceticus Saigon 413 Against Fusarium Broad Species Spectrum Using an In Vitro Bioassay

Various Fusarium spp. (see Table 19) were tested in an in vitro bioassay using petri dishes (9 cm diameter) which contained Luria Broth Agar growth medium and spray dried Streptomyces chrestomyceticus Saigon 413 (formulated as OD400) at different concentrations. Three replicates (petri dishes) were included per treatment concentration and for the control. Agar plugs were taken from Fusarium grown colonies with a cork borer (Ø 6 mm) and one plug was placed in the center of each agar plate. The fungi were incubated at 22 °C in the dark. After incubation, the mycelium growth (diameter) was measured for the efficacy and the half maximal effective concentration, i.e. EC-50, calculations.

TABLE 19
EC50 values of spray dried Streptomyces chrestomyceticus
Saigon 413 TGAI against Fusarium broad species
spectrum using an in vitro bioassay

| Fusarium spp. | EC-50 (ppm) |
|---|---|
| F. graminearum | 180 |
| F. pseudograminearum | 240 |
| F. culmorum | 210 |
| F. avenaceum | 380 |
| F. virguliforme | 400 |
| F. verticillioides | 200 |
| F. fujikuroi | 490 |
| F. subglutinans | 230 |
| F. oxysporum f. sp. cubense | 350 |
| F. oxysporum f.sp. melonis | 360 |
| F. oxysporum f.sp. | 340 |
| vasinfectum | |
| F. oxysporum f.sp. lycopersici | 350 |

### Example 6. Efficacy of Streptomyces chrestomyceticus Saigon 413 TGAI Against Botrytis cinerea and Sclerotinia sclerotiorum on Micro-Profiling Screening

Botrytis cinerea (Gray mould): Conidia of the fungus from cryogenic storage were directly mixed into nutrient broth (Vogel's minimal media). A DMSO solution of the test compounds was placed into a microtiter plate (96-well format) and the nutrient broth containing the fungal spores was added to it. The test plates were incubated at 24 °C and the inhibition of growth was determined photometrically after 72 hrs.

Sclerotinia sclerotiorum (Cottony rot, white mold, etc.): Mycelial fragments of the fungus prepared from a fresh liquid culture were directly mixed into nutrient broth (PDB potato dextrose broth). A DMSO solution of the test compounds was placed into a microtiter plate (96-well format) and the nutrient broth containing the fungal spores was added to it. The test plates were incubated at 24 °C and the inhibition of growth was determined photometrically after 72 hrs at 620 nm.

### Results:

Botrytis cinerea: 100% efficacy at 37 ppm (mg/L)
Sclerotinia sclerotiorum: 100% efficacy at 333 ppm (mg/L)

PCT
(Original in Electronic Form)
(This sheet is not part of and does not count as a sheet of the international application)

| | | |
|---|---|---|
| 0-1 | Form PCT/RO/134 | |
| | Indications Relating to Deposited | |
| | Microorganism(s) or Other Biological | |
| | Material (PCT Rule 13bis) | |
| 0-1-1 | Prepared Using | ePCT-Filing-Embedded |
| | | Version 4.12.001 MT/FOP 20231004/1.1 |
| 0-2 | International Application No. | |
| 0-3 | Applicant's or agent's file reference | 83023-WO-REG-ORG-P-1 |
| 1 | The indications made below relate to | |
| | the deposited microorganism(s) or | |
| | other biological material referred to in | |
| | the description on: | |
| 1-1 | page | 27 |
| 1-2 | line | 38-39 |
| 1-3 | Identification of deposit | |
| 1-3-1 | Name of depositary institution | CBS Westerdijk Fungal Biodiversity Institute |
| 1-3-2 | Address of depositary institution | Westerdijk Fungal Biodiversity Institute (CBS) |
| | | Uppsalalaan 8 |
| | | NL-3584 CT Utrecht |
| | | Netherlands |
| 1-3-3 | Date of deposit | 9 Sep. 2022 (09.09.2022) |
| 1-3-4 | Accession Number | CBS 149411 |
| 1-4 | Additional Indications | |
| 1-5 | Designated States for Which | All designations |
| | Indications are Made | |
| 1-6 | Separate Furnishing of Indications | |
| | These indications will be submitted to | |
| | the International Bureau later | |

FOR RECEIVING OFFICE USE ONLY

| | | |
|---|---|---|
| 0-4 | This form was received with the | yes |
| | international application: | |
| | (yes or no) | |
| 0-4-1 | Authorized officer | Vencourova, Lenka |

FOR INTERNATIONAL BUREAU USE ONLY

| | |
|---|---|
| 0-5 | This form was received by the |
| | international Bureau on: |
| 0-5-1 | Authorized officer |

1. An isolated microbial strain which comprises a genome sequence which has at least 99.8%, identity to the whole genome of Streptomyces sp. Saigon413 deposited with the Westerdijk Institute under accession number CBS149411.

2. An isolated microbial strain, optionally according to claim 1, wherein the strain comprises a nucleotide sequence which has at least 99,9% identity to SEQ ID NO: 1.

3. The microbial strain according to claim 1 wherein the microbial strain is a Streptomyces chrestomyceticus, preferably Streptomyces sp. Saigon413 deposited with the Westerdijk Institute under accession number CBS149411.

4. A composition which comprises a strain of Streptomyces, and malonomicin and at least one of the compounds selected from the group consisting of cyclothiazomycin C, streptimidone, an oligosaccharide compound according to compound I, comprising a molecular formula according to C₅₃H₉₀N₂O₄₄, further characterised by the NMR spectra listed in Table 2 and Table 3, preferably characterized by a structural Formula I, 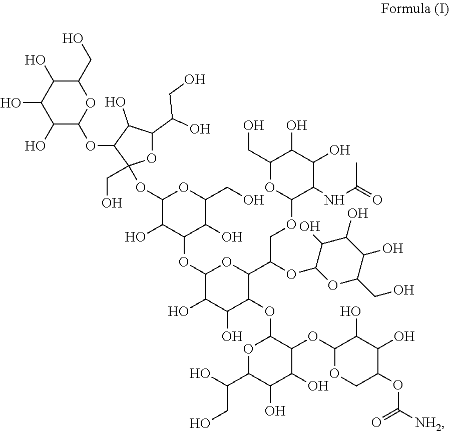 [SMILES: CC(=O)NC1C(OCC(OC2OC(CO)C(O)C(O)C2O)C2OC(OC3C(O)C(CO)OC(OC4(CO)OC(C(O)CO)C(O)C4OC4OC(CO)C(O)C(O)C4O)C3O)C(O)C(O)C2OC2OC(C(O)CO)C(O)C(O)C2OC2OCC(OC(N)=O)C(O)C2O)OC(CO)C(O)C1O]
a lipopeptide according to Formula II, or a salt thereof wherein R1=CH₃ or C₂H₅ 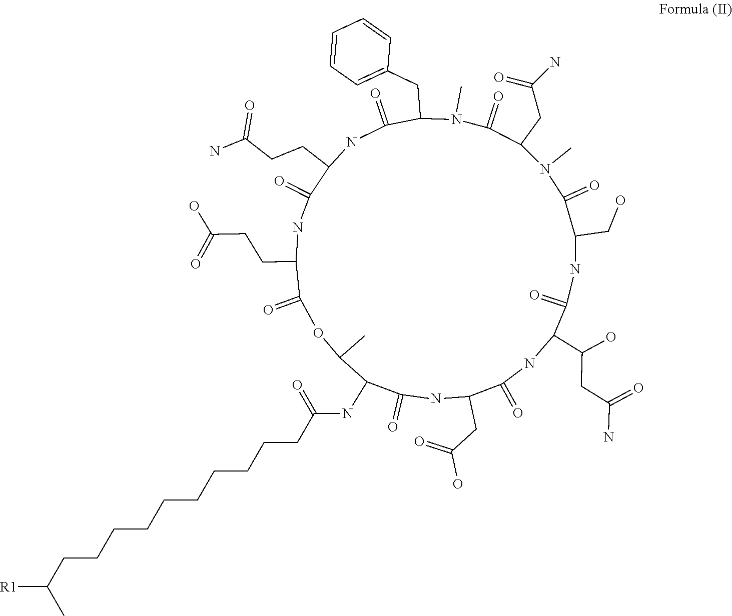
and a polyene compound characterized by a molecular formula according to C₆₇H₁₁₅NO₂₅, wherein the polyene is further characterized by the spectrum of light absorption as shown in FIG. 10.

5. The composition according to claim 4, which further comprises an auxiliary.

6. The composition according to claim 4, wherein the strain of Streptomyces comprises a genome sequence which has at least 95% identity to the whole genome of Streptomyces chrestomyceticus NRRL B-3672, or at least 95% identity to the whole genome of Streptomyces sp. Saigon413 deposited with the Westerdijk Institute under accession number CBS149411.

7. The composition according to claim 4, wherein the strain of Streptomyces comprises a nucleotide sequence which has at least 99% identity to SEQ ID NO: 1.

8. The composition according to claim 4, wherein the composition is a fermentation broth, preferably a spray dried fermentation broth or a freeze-dried fermentation broth, or a formulation.

9. The composition according to claim 4, wherein the composition comprises a cell count of the strain of Streptomyces. or the microbial strain of 10² to 10¹² cfu/g dry weight.

10. The isolated microbial strain according to claim 1, or the composition thereof, wherein the microbial strain or the strain of Streptomyces comprises at least one nucleotide sequence which encodes a protein which has at least 80% to an amino acid sequence of SEQ ID NO: 71 to SEQ ID NO: 115, preferably SEQ ID NO: 91 and/or SEQ ID NO: 92, preferably at least 80% identity to at least one of the nucleotide sequences of SEQ ID NO: 2 to 46, preferably to at least one nucleotide sequence of SEQ ID NO: 22 or 23.

11. The isolated microbial strain according to claim 1, or the composition thereof, wherein the strain or the Streptomyces chrestomyceticus comprises at least one nucleotide sequence which encodes a protein which has at least 80% to an amino acid sequence of SEQ ID NO:116 to SEQ ID NO: 134, preferably an amino acid sequence of SEQ ID NO: 136 or SEQ ID NO: 137, preferably at least one nucleotide sequence which has at least 80% identity to at least one of the nucleotide sequence(s) of SEQ ID NO: 47 to 70, preferably to at least one nucleotide sequence of SEQ ID NO: 67 or 68.

12. A process for producing the microbial strain according to claim 1, or the composition thereof comprising cultivating the microbial strain or the strain of Streptomyces in a suitable fermentation medium under suitable fermentation conditions, and optionally comprising a step of recovering the microbial strain or composition.

13. The process according to claim 12, wherein the microbial strain or the strain of Streptomyces produces malonomcin and at least one of the compounds selected from the group consisting of cyclothiazomycin C, streptimidone, an oligosaccharide compound according to compound I comprising a molecular formula according to C₅₃H₉₀N₂O₄₄, further characterised by the NMR spectra listed in Table 2 and Table 3, preferably wherein compound I is further characterized by a structural Formula I, 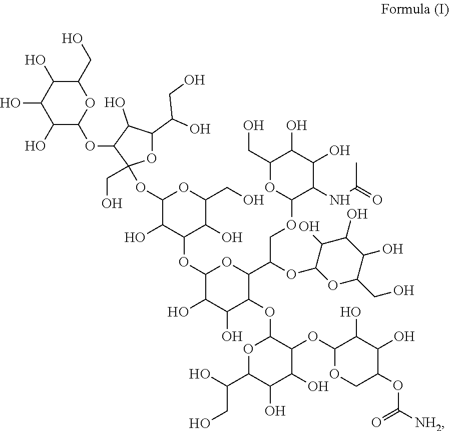 [SMILES: CC(=O)NC1C(OCC(OC2OC(CO)C(O)C(O)C2O)C2OC(OC3C(O)C(CO)OC(OC4(CO)OC(C(O)CO)C(O)C4OC4OC(CO)C(O)C(O)C4O)C3O)C(O)C(O)C2OC2OC(C(O)CO)C(O)C(O)C2OC2OCC(OC(N)=O)C(O)C2O)OC(CO)C(O)C1O]
and a lipopeptide according to Formula II, or a salt thereof wherein R₁═CH₃ or C₂H₅ 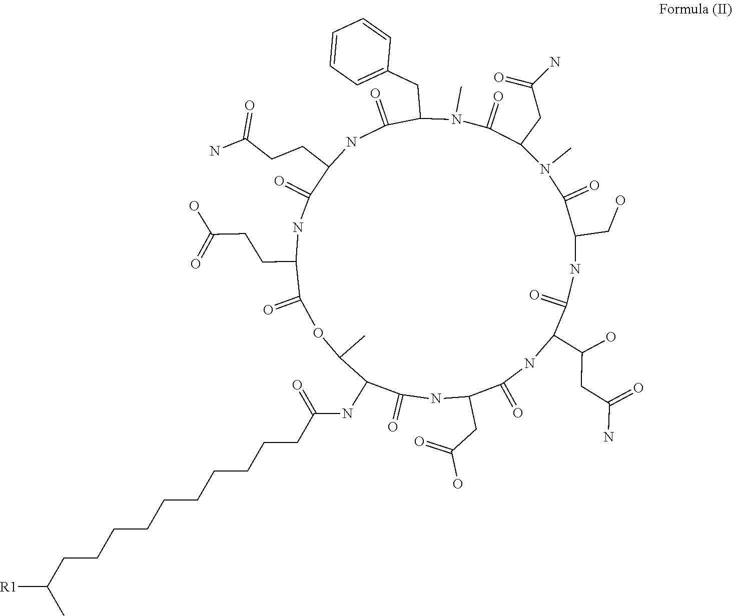
and a polyene compound characterized by a molecular formula according to C₆₇H₁₁₅NO₂₅, wherein the polyene is further characterized by the spectrum of light absorption as shown in FIG. 1.

14. A method for controlling or preventing infestation of a plant, plant propagation material and/or harvested food crops by a phytopathogenic microorganism, by treating the plant, plant propagation material and/or harvested food crops by applying an effective amount of Streptomyces chrestomyceticus, the microbial strain according to claim 1 or the composition thereof to the plant, to a part thereof or a locus thereof, the plant propagation material and/or harvested food crops.

15. The method according to claim 14, wherein the phytopathogenic microorganism is a fungus, preferably a fungus belonging to Zymoseptoria, Puccinia, Mycorsphearella, Pyricularia, Rhizoctoonia, Xanthomonas, Blumeria, Alternaria, Colletotrichum, Ramularia, Parastagonospora, Rhynchosporium, Oculimacula, Fusarium, Gaeumannomyces sp., Botrytis, or Sclerotinia, preferably a fungus belonging to Zymoseptoria tritici, Puccinia recondita, Puccinia striiformis, Mycorsphearella fijiensis, Mycorsphearella arachidis Pyricularia oryzae, Rhizoctoonia solani, Xanthomonas oryzae pv. Oryzae, Blumeria graminis f.sp. tritici, Alternaria solani, Colletotrichum lagenarium, Ramularia collo-cygni, Parastagonospora nodorum, Rhynchosporium secalis, Oculimacula yallandae, Fusarium avenaceum, F. graminearum, F. culmorum, F. virguliforme, F. subglutinans, F. pseudograminearum, F. verticillioides, F. fujikuroi, F subglutinans, F. oxysporum, for instance, F. oxysporum f. sp. Cubense, F. oxysporum f. sp. Melonis, F. oxysporum f. sp.vasinfectum, F. oxysporum f. sp. Lycopesici, Gaeumannomyces graminis Botrytis cinerea, or Sclerotinia sclerotiorum or wherein the phytopathogenic microorganism is an oomycete belonging to Aphanomyces, preferably Aphanomyces cochlioides.

16. The method according to claim 14, wherein the effective amount comprises 2×10² to 5×10¹⁷ colony forming unit (cfu)/ha, or 0.1 g to 10 kg of the Streptomyces chrestomyceticus, or of the isolated microorganism thereof or the composition thereof per hectare.

17. The method according to claim 14, wherein the plant propagation material is seed and the effective amount comprises 2×10² to 5×10¹⁵ (cfu), or 0.0001 g to 100 g Streptomyces chrestomyceticus or the isolated microbial strain thereof or of the composition thereof per kg of seed.

18. The method according to claim 14, wherein the plant comprises wheat, barley, rice, corn, soya, sugar beet banana, tomato, cucumber, and/or groundnut.

19. A plant or a plant propagation material treated with the microbial strain according to claim 1, or a composition thereof.

20. Use of a microbial strain according to claim 1, a S. chrestomyceticus or a composition thereof as a pesticide, preferably as a fungicide.

21. Use of a microbial strain according to claim 1, or a Streptomyces which has at least 95% identity to the whole genome of Streptomyces chrestomyceticus NRRL B-3672, or at least 95% identity to the whole genome of Streptomyces sp. Saigon413 deposited with the Westerdijk Institute under accession number CBS149411, for producing malonomicin and at least one of the compounds selected from the group consisting of cyclothiazomycin C, streptimidone, an oligosaccharide compound according to compound I, comprising a molecular formula according to C₅₃H₉₀N₂O₄₄, further characterised by the NMR spectra listed in Table 2 and Table 3, preferably characterized by a structural Formula I, a lipopeptide according to Formula II, or a salt thereof wherein R1=CH₃ or C₂H₅, and a polyene compound characterized by a molecular formula according to C₆₇H₁₁₅NO₂₅, wherein the polyene is further characterized by the spectrum of light absorption as shown in FIG. 10.
